SEQUENCE LISTING (959 sequences)

SEQ ID NO: 1-15 - skipped (15 entries)

SEQ ID NO: 16 - DNA, 3753 bp, Alphatorquevirus sp.
tgctacgtcactaacccacgtgtcctctacaggccaatcgcagtctatgtcgtgcacttcctgggcatggtctacataattatataaatgcttgcacttccgaatggctgagtttttgctgcccgtccgcggagaggagccacggcaggggatccgaacgtcctgagggcgggtgccggaggtgagtttacacaccgaagtcaaggggcaattcgggctcaggactggccgggctttgggcaaggctcttaaaaatgcacttttctcgaataagcagaaagaaaaggaaagtgctactgctttgcgtgccagcagctaagaaaaaaccaactgctatgagcttctggaaacctccggtacacaatgtcacggggatccaacgcatgtggtatgagtcctttcaccgtggccacgcttctttttgtggttgtgggaatcctatacttcacattactgcacttgctgaaacatatggccatccaacaggcccgagaccttctgggccaccgggagtagaccccaacccccacatccgtagagccaggcctgccccggccgctccggagccctcacaggttgattcgagaccagccctgacatggcatggggatggtggaagcgacggaggcgctggtggttccggaagcggtggacccgtggcagacttcgcagacgatggcctcgatcagctcgtcgccgccctagacgacgaagagtaaggaggcgcagacggtggaggagggggagacgaaaaacaaggacttacagacgcaggagacgctttagacgcaggggacgaaaagcaaaacttataataaaactgtggcaacctgcagtaattaaaagatgcagaataaagggatacataccactgattataagtgggaacggtacctttgccacaaactttaccagtcacataaatgacagaataatgaaaggccccttcgggggaggacacagcactatgaggttcagcctctacattttgtttgaggagcacctcagacacatgaacttctggaccagaagcaacgataacctagagctaaccagatacttgggggcttcagtaaaaatatacaggcacccagaccaagactttatagtaatatacaacagaagaacccctctaggaggcaacatctacacagcaccctctctacacccaggcaatgccattttagcaaaacacaaaatattagtaccaagtttacagacaagaccaaagggtagaaaagcaattagactaagaatagcaccccccacactctttacagacaagtggtactttcaaaaggacatagccgacctcacccttttcaacatcatggcagttgaggctgacttgcggtttccgttctgctcaccacaaactgacaacacttgcatcagcttccaggtccttagttccgtttacaacaactacctcagtattaatacctttaataatgacaactcagactcaaagttaaaagaatttttaaataaagcatttccaacaacaggcacaaaaggaacaagtttaaatgcactaaatacatttagaacagaaggatgcataagtcacccacaactaaaaaaaccaaacccacaaataaacaaaccattagagtcacaatactttgcacctttagatgccctctggggagaccccatatactataatgatctaaatgaaaacaaaagtttgaacgatatcattgagaaaatactaataaaaaacatgattacataccatgcaaaactaagagaatttccaaattcataccaaggaaacaaggccttttgccacctaacaggcatatacagcccaccatacctaaaccaaggcagaatatctccagaaatatttggactgtacacagaaataatttacaacccttacacagacaaaggaactggaaacaaagtatggatggacccactaactaaagagaacaacatatataaagaaggacagagcaaatgcctactgactgacatgcccctatggactttactttttggatatacagactggtgtaaaaaggacactaataactgggacttaccactaaactacagactagtactaatatgcccttatacctttccaaaattgtacaatgaaaaagtaaaagactatgggtacatcccgtactcctacaaattcggagcgggtcagatgccagacggcagcaactacataccctttcagtttagagcaaagtggtaccccacagtactacaccagcaacaggtaatggaggacataagcaggagcgggccctttgcacctaaggtagaaaaaccaagcactcagctggtaatgaagtactgttttaactttaactggggcggtaaccctatcattgaacagattgttaaagaccccagcttccagcccacctatgaaatacccggtaccggtaacatccctagaagaatacaagtcatcgacccgcgggtcctgggaccgcactactcgttccggtcatgggacatgcgcagacacacatttagcagagcaagtattaagagagtgtcagaacaacaagaaacttctgaccttgtattctcaggcccaaaaaagcctcgggtcgacatcccaaaacaagaaacccaagaagaaagctcacattcactccaaagagaatcgagaccgtgggagaccgaggaagaaagcgagacagaagccctctcgcaagagagccaagaggtccccttccaacagcagttgcagcagcagtaccaagagcagctcaagctcagacagggaatcaaagtcctcttcgagcagctcataaggacccaacaaggggtccatgtaaacccatgcctacggtaggtcccaggcagtggctgtttccagagagaaagccagccccagctcctagcagtggagactgggccatggagtttctcgcagcaaaaatatttgataggccagttagaagcaaccttaaagataccccttactacccatatgttaaaaaccaatacaatgtctactttgaccttaaatttgaataaacagcagcttcaaacttgcaaggccgtgggagtttcactggtcggtgtctacctctaaaggtcactaagcactccgagcgtaagcgaggagtgcgaccctcccccctggaacaacttcttcggagtccggcgctacgccttcggctgcgccggacacctcagaccccccctccacccgaaacgcttgcgcgtttcggaccttcggcgtcgggggggtcgggagctttattaaacggactccgaagtgctcttggacactgagggggtgaacagcaacgaaagtgagtggggccagacttcgccataaggcctttatcttcttgccatttgtcagtgtccggggtcgccataggcttcgggctcgtttttaggccttccggactacaaaaatcgccattttggtgacgtcacggccgccatcttaagtagttgaggcggacggtggcgtgagttcaaaggtcaccatcagccacacctactcaaaatggtggacaatttcttccgggtcaaaggttacagccgccatgttaaaacacgtgacgtatgacgtcacggccgccattttgtgacacaagatggccgacttccttcctctttttcaaaaaaaagcggaagtgccgccgcggcggcggggggcggcgcgctgcgcgcgccgcccagtagggggagccatgcgcccccccccgcgcatgcgcggggcccccccccgcggggggctccgccccccggccccccccg

SEQ ID NO: 17 - protein, 127 aa, Alphatorquevirus sp.
MSFWKPPVHNVTGIQRMWYESFHRGHASFCGCGNPILHITALAETYGHPTGPRPSGPPGVDPNPHIRRARPAPAAPEPSQVDSRPALTWHGDGGSDGGAGGSGSGGPVADFADDGLDQLVAALDDEE

SEQ ID NO: 18 - protein, 268 aa, Alphatorquevirus sp.
MSFWKPPVHNVTGIQRMWYESFHRGHASFCGCGNPILHITALAETYGHPTGPRPSGPPGVDPNPHIRRARPAPAAPEPSQVDSRPALTWHGDGGSDGGAGGSGSGGPVADFADDGLDQLVAALDDEELLKTPASSPPMKYPVPVTSLEEYKSSTRGSWDRTTRSGHGTCADTHLAEQVLRECQNNKKLLTLYSQAQKSLGSTSQNKKPKKKAHIHSKENRDRGRPRKKARQKPSRKRAKRSPSNSSCSSSTKSSSSSDRESKSSSSSS

SEQ ID NO: 19 - protein, 276 aa, Alphatorquevirus sp.
MSFWKPPVHNVTGIQRMWYESFHRGHASFCGCGNPILHITALAETYGHPTGPRPSGPPGVDPNPHIRRARPAPAAPEPSQVDSRPALTWHGDGGSDGGAGGSGSGGPVADFADDGLDQLVAALDDEEPKKASGRHPKTRNPRRKLTFTPKRIETVGDRGRKRDRSPLAREPRGPLPTAVAAAVPRAAQAQTGNQSPLRAAHKDPTRGPCKPMPTVGPRQWLFPERKPAPAPSSGDWAMEFLAAKIFDRPVRSNLKDTPYYPYVKNQYNVYFDLKFE

SEQ ID NO: 20 - protein, 167 aa, Alphatorquevirus sp.
MSFWKPPVHNVTGIQRMWPKKASGRHPKTRNPRRKLTFTPKRIETVGDRGRKRDRSPLAREPRGPLPTAVAAAVPRAAQAQTGNQSPLRAAHKDPTRGPCKPMPTVGPRQWLFPERKPAPAPSSGDWAMEFLAAKIFDRPVRSNLKDTPYYPYVKNQYNVYFDLKFE

SEQ ID NO: 21 - protein, 743 aa, Alphatorquevirus sp.
MAWGWWKRRRRWWFRKRWTRGRLRRRWPRSARRRPRRRRVRRRRRWRRGRRKTRTYRRRRRFRRRGRKAKLIIKLWQPAVIKRCRIKGYIPLIISGNGTFATNFTSHINDRIMKGPFGGGHSTMRFSLYILFEEHLRHMNFWTRSNDNLELTRYLGASVKIYRHPDQDFIVIYNRRTPLGGNIYTAPSLHPGNAILAKHKILVPSLQTRPKGRKAIRLRIAPPTLFTDKWYFQKDIADLTLFNIMAVEADLRFPFCSPQTDNTCISFQVLSSVYNNYLSINTFNNDNSDSKLKEFLNKAFPTTGTKGTSLNALNTFRTEGCISHPQLKKPNPQINKPLESQYFAPLDALWGDPIYYNDLNENKSLNDIIEKILIKNMITYHAKLREFPNSYQGNKAFCHLTGIYSPPYLNQGRISPEIFGLYTEIIYNPYTDKGTGNKVWMDPLTKENNIYKEGQSKCLLTDMPLWTLLFGYTDWCKKDTNNWDLPLNYRLVLICPYTFPKLYNEKVKDYGYIPYSYKFGAGQMPDGSNYIPFQFRAKWYPTVLHQQQVMEDISRSGPFAPKVEKPSTQLVMKYCFNFNWGGNPIIEQIVKDPSFQPTYEIPGTGNIPRRIQVIDPRVLGPHYSFRSWDMRRHTFSRASIKRVSEQQETSDLVFSGPKKPRVDIPKQETQEESSHSLQRESRPWETEEESETEALSQESQEVPFQQQLQQQYQEQLKLRQGIKVLFEQLIRTQQGVHVNPCLR

SEQ ID NO: 22 - protein, 194 aa, Alphatorquevirus sp.
MAWGWWKRRRRWWFRKRWTRGRLRRRWPRSARRRPRRRRIVKDPSFQPTYEIPGTGNIPRRIQVIDPRVLGPHYSFRSWDMRRHTFSRASIKRVSEQQETSDLVFSGPKKPRVDIPKQETQEESSHSLQRESRPWETEEESETEALSQESQEVPFQQQLQQQYQEQLKLRQGIKVLFEQLIRTQQGVHVNPCLR

SEQ ID NO: 23 - protein, 113 aa, Alphatorquevirus sp.
MAWGWWKRRRRWWFRKRWTRGRLRRRWPRSARRRPRRRRAQKSLGSTSQNKKPKKKAHIHSKENRDRGRPRKKARQKPSRKRAKRSPSNSSCSSSTKSSSSSDRESKSSSSSS

SEQ ID NO: 24-53 - skipped (30 entries)

SEQ ID NO: 54 - DNA, 2979 bp, Betatorquevirus sp.
taataaatattcaacaggaaaaccacctaatttaaattgccgaccacaaaccgtcacttagttcccctttttgcaacaacttctgcttttttccaactgccggaaaaccacataatttgcatggctaaccacaaactgatatgctaattaacttccacaaaacaacttccccttttaaaaccacacctacaaattaattattaaacacagtcacatcctgggaggtactaccacactataataccaagtgcacttccgaatggctgagtttatgccgctagacggagaacgcatcagttactgactgcggactgaacttgggcgggtgccgaaggtgagtgaaaccaccgaagtcaaggggcaattcgggctagttcagtctagcggaacgggcaagaaacttaaaattattttatttttcagatgagcgactgctttaaaccaacatgctacaacaacaaaacaaagcaaactcactggattaataacctgcatttaacccacgacctgatctgcttctgcccaacaccaactagacacttattactagctttagcagaacaacaagaaacaattgaagtgtctaaacaagaaaaagaaaaaataacaagatgccttattactacagaagaagacggtacaactacagacgtcctagatggtatggacgaggttggattagacgcccttttcgcagaagatttcgaagaaaaagaagggtaagacctacttatactactattcctctaaagcaatggcaaccgccatataaaagaacatgctatataaaaggacaagactgtttaatatactatagcaacttaagactgggaatgaatagtacaatgtatgaaaaaagtattgtacctgtacattggccgggagggggttctttttctgtaagcatgttaactttagatgccttgtatgatatacataaactttgtagaaactggtggacatccacaaaccaagacttaccactagtaagatataaaggatgcaaaataacattttatcaaagcacatttacagactacatagtaagaatacatacagaactaccagctaacagtaacaaactaacatacccaaacacacatccactaatgatgatgatgtctaagtacaaacacattatacctagtagacaaacaagaagaaaaaagaaaccatacacaaaaatatttgtaaaaccacctccgcaatttgaaaacaaatggtactttgctacagacctctacaaaattccattactacaaatacactgcacagcatgcaacttacaaaacccatttgtaaaaccagacaaattatcaaacaatgttacattatggtcactaaacaccataagcatacaaaatagaaacatgtcagtggatcaaggacaatcatggccatttaaaatactaggaacacaaagcttttatttttacttttacaccggagcaaacctaccaggtgacacaacacaaataccagtagcagacctattaccactaacaaacccaagaataaacagaccaggacaatcactaaatgaggcaaaaattacagaccatattactttcacagaatacaaaaacaaatttacaaattattggggtaacccatttaataaacacattcaagaacacctagatatgatactatactcactaaaaagtccagaagcaataaaaaacgaatggacaacagaaaacatgaaatggaaccaattaaacaatgcaggaacaatggcattaacaccatttaacgagccaatattcacacaaatacaatataacccagatagagacacaggagaagacactcaattatacctactctctaacgctacaggaacaggatgggacccaccaggaattccagaattaatactagaaggatttccactatggttaatatattggggatttgcagactttcaaaaaaacctaaaaaaagtaacaaacatagacacaaattacatgttagtagcaaaaacaaaatttacacaaaaacctggcacattctacttagtaatactaaatgacacctttgtagaaggcaatagcccatatgaaaaacaacctttacctgaagacaacattaaatggtacccacaagtacaataccaattagaagcacaaaacaaactactacaaactgggccatttacaccaaacatacaaggacaactatcagacaatatatcaatgttttataaattttactttaaatggggaggaagcccaccaaaagcaattaatgttgaaaatcctgcccaccagattcaatatcccataccccgtaacgagcatgaaacaacttcgttacagagtccaggggaagccccagaatccatcttatactccttcgactatagacacgggaactacacaacaacagctttgtcacgaattagccaagactgggcacttaaagacactgtttctaaaattacagagccagatcgacagcaactgctcaaacaagccctcgaatgcctgcaaatctcggaagaaacgcaggagaaaaaagaaaaagaagtacagcagctcatcagcaacctcagacagcagcagcagctgtacagagagcgaataatatcattattaaaggaccaataacttttaactgtgtaaaaaaggtgaaattgtttgatgataaaccaaaaaaccgtagatttacacctgaggaatttgaaactgagttacaaatagcaaaatggttaaagagacccccaagatcctttgtaaatgatcctcccttttacccatggttaccacctgaacctgttgtaaactttaagcttaattttactgaataaaggccagcattaattcacttaaggagtctgtttatttaagttaaaccttaataaacggtcaccgcctccctaatacgcaggcgcagaaagggggctccgccccctttaacccccagggggctccgccccctgaaacccccaagggggctacgcccccttacaccccc

SEQ ID NO: 55 - protein, 99 aa, Betatorquevirus sp.
MSDCFKPTCYNNKTKQTHWINNLHLTHDLICFCPTPTRHLLLALAEQQETIEVSKQEKEKITRCLITTEEDGTTTDVLDGMDEVGLDALFAEDFEEKEG

SEQ ID NO: 56 - protein, 203 aa, Betatorquevirus sp.
MSDCFKPTCYNNKTKQTHWINNLHLTHDLICFCPTPTRHLLLALAEQQETIEVSKQEKEKITRCLITTEEDGTTTDVLDGMDEVGLDALFAEDFEEKEGFNIPYPVTSMKQLRYRVQGKPQNPSYTPSTIDTGTTQQQLCHELAKTGHLKTLFLKLQSQIDSNCSNKPSNACKSRKKRRRKKKKKYSSSSATSDSSSSCTESE

SEQ ID NO: 57 - protein, 219 aa, Betatorquevirus sp.
MSDCFKPTCYNNKTKQTHWINNLHLTHDLICFCPTPTRHLLLALAEQQETIEVSKQEKEKITRCLITTEEDGTTTDVLDGMDEVGLDALFAEDFEEKEGARSTATAQTSPRMPANLGRNAGEKRKRSTAAHQQPQTAAAAVQRANNIIIKGPITFNCVKKVKLFDDKPKNRRFTPEEFETELQIAKWLKRPPRSFVNDPPFYPWLPPEPVVNFKLNFTE

SEQ ID NO: 58 - protein, 666 aa, Betatorquevirus sp.
MPYYYRRRRYNYRRPRWYGRGWIRRPFRRRFRRKRRVRPTYTTIPLKQWQPPYKRTCYIKGQDCLIYYSNLRLGMNSTMYEKSIVPVHWPGGGSFSVSMLTLDALYDIHKLCRNWWTSTNQDLPLVRYKGCKITFYQSTFTDYIVRIHTELPANSNKLTYPNTHPLMMMMSKYKHIIPSRQTRRKKKPYTKIFVKPPPQFENKWYFATDLYKIPLLQIHCTACNLQNPFVKPDKLSNNVTLWSLNTISIQNRNMSVDQGQSWPFKILGTQSFYFYFYTGANLPGDTTQIPVADLLPLTNPRINRPGQSLNEAKITDHITFTEYKNKFTNYWGNPFNKHIQEHLDMILYSLKSPEAIKNEWTTENMKWNQLNNAGTMALTPFNEPIFTQIQYNPDRDTGEDTQLYLLSNATGTGWDPPGIPELILEGFPLWLIYWGFADFQKNLKKVTNIDTNYMLVAKTKFTQKPGTFYLVILNDTFVEGNSPYEKQPLPEDNIKWYPQVQYQLEAQNKLLQTGPFTPNIQGQLSDNISMFYKFYFKWGGSPPKAINVENPAHQIQYPIPRNEHETTSLQSPGEAPESILYSFDYRHGNYTTTALSRISQDWALKDTVSKITEPDRQQLLKQALECLQISEETQEKKEKEVQQLISNLRQQQQLYRERIISLLKDQ

SEQ ID NO: 59 - protein, 148 aa, Betatorquevirus sp.
MPYYYRRRRYNYRRPRWYGRGWIRRPFRRRFRRKRRIQYPIPRNEHETTSLQSPGEAPESILYSFDYRHGNYTTTALSRISQDWALKDTVSKITEPDRQQLLKQALECLQISEETQEKKEKEVQQLISNLRQQQQLYRERIISLLKDQ

SEQ ID NO: 60 - protein, 82 aa, Betatorquevirus sp.
MPYYYRRRRYNYRRPRWYGRGWIRRPFRRRFRRKRRSQIDSNCSNKPSNACKSRKKRRRKKKKKYSSSSATSDSSSSCTESE

SEQ ID NO: 61-104 - skipped (44 entries)

SEQ ID NO: 105 - DNA, 71 bp, Artificial Sequence
  Description of Artificial Sequence: Synthetic oligonucleotide
cgggtgccgkaggtgagtttacacaccgmagtcaaggggcaattcgggctcrggactggccgggcyhtggg

SEQ ID NO: 106 - DNA, 71 bp, Artificial Sequence
  Description of Artificial Sequence: Synthetic oligonucleotide
cgggtgccggaggtgagtttacacaccgcagtcaaggggcaattcgggctcgggactggccgggctwtggg

SEQ ID NO: 107 - DNA, 71 bp, Artificial Sequence
  Description of Artificial Sequence: Synthetic oligonucleotide
cgggtgccgtaggtgagtttacacaccgcagtcaaggggcaattcgggctcgggactggccgggctatggg

SEQ ID NO: 108 - DNA, 71 bp, Artificial Sequence
  Description of Artificial Sequence: Synthetic oligonucleotide
cgggtgccggaggtgagtttacacaccgcagtcaaggggcaattcgggctcgggactggccgggccctggg

SEQ ID NO: 109 - DNA, 71 bp, Artificial Sequence
  Description of Artificial Sequence: Synthetic oligonucleotide
cgggtgccggaggtgagtttacacaccgcagtcaaggggcaattcgggctcgggactggccgggctttggg

SEQ ID NO: 110 - DNA, 71 bp, Artificial Sequence
  Description of Artificial Sequence: Synthetic oligonucleotide
cgggtgccggaggtgagtttacacaccgcagtcaaggggcaattcgggctcgggactggccgggctatggg

SEQ ID NO: 111 - DNA, 71 bp, Artificial Sequence
  Description of Artificial Sequence: Synthetic oligonucleotide
cgggtgccggaggtgagtttacacaccgaagtcaaggggcaattcgggctcaggactggccgggctttggg

SEQ ID NO: 112 - DNA, 71 bp, Artificial Sequence
  Description of Artificial Sequence: Synthetic oligonucleotide
cgggtgccggaggtgagtttacacaccgcagtcaaggggcaattcgggctcgggactggccgggcyhtggg

SEQ ID NO: 113 - DNA, 71 bp, Artificial Sequence; identical to SEQ ID NO: 107
  Description of Artificial Sequence: Synthetic oligonucleotide

SEQ ID NO: 114 - DNA, 70 bp, Artificial Sequence
  Description of Artificial Sequence: Synthetic oligonucleotide
cgggtgccggaggtgagtttacacaccgcagtcaaggggcaattcgggctcgggactggccgggcccggg

SEQ ID NO: 115 - DNA, 71 bp, Artificial Sequence; identical to SEQ ID NO: 111
  Description of Artificial Sequence: Synthetic oligonucleotide

SEQ ID NO: 116 - DNA, 69 bp, Artificial Sequence
  Description of Artificial Sequence: Synthetic oligonucleotide
cgggtgccggaggtgagtttacacaccgcagtcaaggggcaattcgggctcgggaggccgggccatggg

SEQ ID NO: 117 - DNA, 71 bp, Artificial Sequence
  Description of Artificial Sequence: Synthetic oligonucleotide
cgggtgccggaggtgagtttacacaccgcagtcaaggggcaattcgggctcgggactggccgggccccggg

SEQ ID NO: 118 - DNA, 71 bp, Artificial Sequence; identical to SEQ ID NO: 110
  Description of Artificial Sequence: Synthetic oligonucleotide

SEQ ID NO: 119 - DNA, 71 bp, Artificial Sequence
  Description of Artificial Sequence: Synthetic oligonucleotide
cgggtgccgaaggtgagtttacacaccgcagtcaaggggcaattcgggctcgggactggccgggctatggg

SEQ ID NO: 120 - DNA, 117 bp, Artificial Sequence
  Description of Artificial Sequence: Synthetic polynucleotide
  misc_feature (10)..(10): May or may not be present
  misc_feature (12)..(12): May or may not be present
  misc_feature (30)..(32): May or may not be present
  misc_feature (34)..(34): May or may not be present
  misc_feature (43)..(46): May or may not be present
  misc_feature (52)..(54): May or may not be present
  misc_feature (70)..(71): May or may not be present
  misc_feature (89)..(90): May or may not be present
  misc_feature (103)..(103): May or may not be present
cggcggsggsgcsscgcgctdcgcgcgcsgcccrsyrggggrdssmmwgcskcscccccccscgcgcatgcgcrcgggkcccccccccyvsggggggctccgcccccccggcccccc

SEQ ID NO: 121 - DNA, 169 bp, Artificial Sequence
  Description of Artificial Sequence: Synthetic polynucleotide
  modified_base (20)..(20): a, c, t, g, unknown or other
  modified_base (22)..(22): a, c, t, g, unknown or other
  modified_base (40)..(42): a, c, t, g, unknown or other
  modified_base (53)..(56): a, c, t, g, unknown or other
  modified_base (62)..(62): a, c, t, g, unknown or other
  modified_base (64)..(64): a, c, t, g, unknown or other
  modified_base (97)..(98): a, c, t, g, unknown or other
gccgccgcggcggcggsggngnsgcgcgctdcgcgcgcsnnncrccrgggggnnnncwgcsncncccccccccgcgcatgcgcgggkcccccccccnncggggggctccgccccccggcccccccccgtgctaaacccaccgcgcatgcgcgaccacgcccccgccgcc

SEQ ID NO: 122 - DNA, 79 bp, Artificial Sequence
  Description of Artificial Sequence: Synthetic oligonucleotide
  modified_base (20)..(20): a, c, t, g, unknown or other
  modified_base (22)..(22): a, c, t, g, unknown or other
  modified_base (40)..(42): a, c, t, g, unknown or other
  modified_base (53)..(56): a, c, t, g, unknown or other
  modified_base (62)..(62): a, c, t, g, unknown or other
  modified_base (64)..(64): a, c, t, g, unknown or other
gccgccgcggcggcggsggngnsgcgcgctdcgcgcgcsnnncrccrgggggnnnncwgcsncncccccccccgcgcat

SEQ ID NO: 123 - DNA, 31 bp, Artificial Sequence
  Description of Artificial Sequence: Synthetic oligonucleotide
  modified_base (18)..(19): a, c, t, g, unknown or other
gcgcgggkcccccccccnncggggggctccg

SEQ ID NO: 124 - DNA, 59 bp, Artificial Sequence
  Description of Artificial Sequence: Synthetic oligonucleotide
ccccccggcccccccccgtgctaaacccaccgcgcatgcgcgaccacgcccccgccgcc

SEQ ID NO: 125 - DNA, 156 bp, Artificial Sequence
  Description of Artificial Sequence: Synthetic polynucleotide
gcggcgggggggcggccgcgttcgcgcgccgcccaccagggggtgctgcgcgcccccccccgcgcatgcgcggggcccccccccgggggggctccgcccccccggcccccccccgtgctaaacccaccgcgcatgcgcgaccacgcccccgccgcc

SEQ ID NO: 126 - DNA, 7 bp, Artificial Sequence
  Description of Artificial Sequence: Synthetic oligonucleotide
gcggcgg

SEQ ID NO: 127 - DNA, 7 bp, Artificial Sequence
  Description of Artificial Sequence: Synthetic oligonucleotide
gggggcg

SEQ ID NO: 128 - DNA, 6 bp, Artificial Sequence
  Description of Artificial Sequence: Synthetic oligonucleotide
gccgcg

SEQ ID NO: 129 - DNA, 25 bp, Artificial Sequence
  Description of Artificial Sequence: Synthetic oligonucleotide
ttcgcgcgccgcccaccagggggtg

SEQ ID NO: 130 - DNA, 5 bp, Artificial Sequence
  Description of Artificial Sequence: Synthetic oligonucleotide
ctgcg

SEQ ID NO: 131 - DNA, 17 bp, Artificial Sequence
  Description of Artificial Sequence: Synthetic oligonucleotide
cgcccccccccgcgcat

SEQ ID NO: 132 - DNA, 17 bp, Artificial Sequence
  Description of Artificial Sequence: Synthetic oligonucleotide
gcgcggggccccccccc

SEQ ID NO: 133 - DNA, 72 bp, Artificial Sequence
  Description of Artificial Sequence: Synthetic oligonucleotide
gggggggctccgcccccccggcccccccccgtgctaaacccaccgcgcatgcgcgaccacgcccccgccgcc

SEQ ID NO: 134 - DNA, 115 bp, Artificial Sequence
  Description of Artificial Sequence: Synthetic polynucleotide
cggcggcggcggcgcgcgcgctgcgcgcgcgcgccgggggggcgccagcgcccccccccccgcgcatgcacgggtccccccccccacggggggctccgccccccggccccccccc

SEQ ID NO: 135 - DNA, 14 bp, Artificial Sequence
  Description of Artificial Sequence: Synthetic oligonucleotide
cggcggcggcggcg

SEQ ID NO: 136 - DNA, 17 bp, Artificial Sequence
  Description of Artificial Sequence: Synthetic oligonucleotide
cgcgcgctgcgcgcgcg

SEQ ID NO: 137 - DNA, 19 bp, Artificial Sequence
  Description of Artificial Sequence: Synthetic oligonucleotide
cgccgggggggcgccagcg

SEQ ID NO: 138 - DNA, 17 bp, Artificial Sequence
  Description of Artificial Sequence: Synthetic oligonucleotide
cccccccccccgcgcat

SEQ ID NO: 139 - DNA, 31 bp, Artificial Sequence
  Description of Artificial Sequence: Synthetic oligonucleotide
gcacgggtccccccccccacggggggctccg

SEQ ID NO: 140 - DNA, 17 bp, Artificial Sequence
  Description of Artificial Sequence: Synthetic oligonucleotide
ccccccggccccccccc

SEQ ID NO: 141 - DNA, 121 bp, Artificial Sequence
  Description of Artificial Sequence: Synthetic polynucleotide
ccgtcggcgggggggccgcgcgctgcgcgcgcggcccccgggggaggcacagcctcccccccccgcgcgcatgcgcgcgggtccccccccctccggggggctccgccccccggcccccccc

SEQ ID NO: 142 - DNA, 37 bp, Artificial Sequence
  Description of Artificial Sequence: Synthetic oligonucleotide
ccgtcggcgggggggccgcgcgctgcgcgcgcggccc

SEQ ID NO: 143 - DNA, 84 bp, Artificial Sequence
  Description of Artificial Sequence: Synthetic oligonucleotide
ccgggggaggcacagcctcccccccccgcgcgcatgcgcgcgggtccccccccctccggggggctccgccccccggcccccccc

SEQ ID NO: 144 - DNA, 104 bp, Artificial Sequence
  Description of Artificial Sequence: Synthetic polynucleotide
cggcggcggcgcgcgcgctacgcgcgcgcgccggggggctgccgccccccccccgcgcatgcgcggggcccccccccgcggggggctccgccccccggcccccc

SEQ ID NO: 145 - DNA, 11 bp, Artificial Sequence
  Description of Artificial Sequence: Synthetic oligonucleotide
cggcggcggcg

SEQ ID NO: 146 - DNA, 17 bp, Artificial Sequence
  Description of Artificial Sequence: Synthetic oligonucleotide
cgcgcgctacgcgcgcg

SEQ ID NO: 147 - DNA, 10 bp, Artificial Sequence
  Description of Artificial Sequence: Synthetic oligonucleotide
cgccgggggg

SEQ ID NO: 148 - DNA, 7 bp, Artificial Sequence
  Description of Artificial Sequence: Synthetic oligonucleotide
ctgccgc

SEQ ID NO: 149 - DNA, 15 bp, Artificial Sequence
  Description of Artificial Sequence: Synthetic oligonucleotide
cccccccccgcgcat

SEQ ID NO: 150 - DNA, 17 bp, Artificial Sequence
  Description of Artificial Sequence: Synthetic oligonucleotide
gcgcggggccccccccc

SEQ ID NO: 151 - DNA, 13 bp, Artificial Sequence
  Description of Artificial Sequence: Synthetic oligonucleotide
gcggggggctccg

SEQ ID NO: 152 - DNA, 14 bp, Artificial Sequence
  Description of Artificial Sequence: Synthetic oligonucleotide
ccccccggcccccc

SEQ ID NO: 153 - DNA, 122 bp, Artificial Sequence
  Description of Artificial Sequence: Synthetic polynucleotide
gccgccgcggcggcggggggcggcgcgctgcgcgcgccgcccagtagggggagccatgcgcccccccccgcgcatgcgcggggcccccccccgcggggggctccgccccccggccccccccg

SEQ ID NO: 154 - DNA, 19 bp, Artificial Sequence
  Description of Artificial Sequence: Synthetic oligonucleotide
gccgccgcggcggcggggg

SEQ ID NO: 155 - DNA, 41 bp, Artificial Sequence
  Description of Artificial Sequence: Synthetic oligonucleotide
gcggcgcgctgcgcgcgccgcccagtagggggagccatgcg

SEQ ID NO: 156 - DNA, 15 bp, Artificial Sequence
  Description of Artificial Sequence: Synthetic oligonucleotide
cccccccccgcgcat

SEQ ID NO: 157 - DNA, 17 bp, Artificial Sequence
  Description of Artificial Sequence: Synthetic oligonucleotide
gcgcggggccccccccc

SEQ ID NO: 158 - DNA, 13 bp, Artificial Sequence
  Description of Artificial Sequence: Synthetic oligonucleotide
gcggggggctccg

SEQ ID NO: 159 - DNA, 17 bp, Artificial Sequence
  Description of Artificial Sequence: Synthetic oligonucleotide
ccccccggccccccccg

SEQ ID NO: 160 - DNA, 36 bp, Artificial Sequence
  Description of Artificial Sequence: Synthetic oligonucleotide
cgcgctgcgcgcgccgcccagtagggggagccatgc

SEQ ID NO: 161 - DNA, 78 bp, Artificial Sequence
  Description of Artificial Sequence: Synthetic oligonucleotide
ccgccatcttaagtagttgaggcggacggtggcgtgagttcaaaggtcaccatcagccacacctactcaaaatggtgg

SEQ ID NO: 162 - DNA, 172 bp, Artificial Sequence
  Description of Artificial Sequence: Synthetic polynucleotide
cttaagtagttgaggcggacggtggcgtgagttcaaaggtcaccatcagccacacctactcaaaatggtggacaatttcttccgggtcaaaggttacagccgccatgttaaaacacgtgacgtatgacgtcacggccgccattttgtgacacaagatggccgacttccttcc

SEQ ID NO: 163 - DNA, 36 bp, Artificial Sequence; identical to SEQ ID NO: 160
  Description of Artificial Sequence: Synthetic oligonucleotide

SEQ ID NO: 164 - DNA, 36 bp, Artificial Sequence
  Description of Artificial Sequence: Synthetic oligonucleotide
gcgctdcgcgcgcgcgccggggggctgcgccccccc

SEQ ID NO: 165 - DNA, 36 bp, Artificial Sequence
  Description of Artificial Sequence: Synthetic oligonucleotide
gcgcttcgcgcgccgcccactagggggcgttgcgcg

SEQ ID NO: 166 - DNA, 36 bp, Artificial Sequence
  Description of Artificial Sequence: Synthetic oligonucleotide
gcgctgcgcgcgccgcccagtagggggcgcaatgcg

SEQ ID NO: 167 - DNA, 36 bp, Artificial Sequence
  Description of Artificial Sequence: Synthetic oligonucleotide
gcgctgcgcgcgcggcccccgggggaggcattgcct

SEQ ID NO: 168 - DNA, 36 bp, Artificial Sequence
  Description of Artificial Sequence: Synthetic oligonucleotide
gcgctgcgcgcgcgcgccgggggggcgccagcgccc

SEQ ID NO: 169 - DNA, 36 bp, Artificial Sequence
  Description of Artificial Sequence: Synthetic oligonucleotide
gcgcttcgcgcgcgcgccggggggctccgccccccc

SEQ ID NO: 170 - DNA, 36 bp, Artificial Sequence
  Description of Artificial Sequence: Synthetic oligonucleotide
gcgcttcgcgcgcgcgccggggggctgcgccccccc

SEQ ID NO: 171 - DNA, 36 bp, Artificial Sequence
  Description of Artificial Sequence: Synthetic oligonucleotide
gcgctacgcgcgcgcgccggggggctgcgccccccc

SEQ ID NO: 172 - DNA, 36 bp, Artificial Sequence
  Description of Artificial Sequence: Synthetic oligonucleotide
gcgctacgcgcgcgcgccggggggctctgccccccc

SEQ ID NO: 173-184 - skipped (12 entries)

SEQ ID NO: 185 - protein, 743 aa, Alphatorquevirus sp.; identical to SEQ ID NO: 21

SEQ ID NO: 186 - protein, 68 aa, Alphatorquevirus sp.
MAWGWWKRRRRWWFRKRWTRGRLRRRWPRSARRRPRRRRVRRRRRWRRGRRKTRTYRRRRRFRRRGRK

SEQ ID NO: 187 - protein, 212 aa, Alphatorquevirus sp.
AKLIIKLWQPAVIKRCRIKGYIPLIISGNGTFATNFTSHINDRIMKGPFGGGHSTMRFSLYILFEEHLRHMNFWTRSNDNLELTRYLGASVKIYRHPDQDFIVIYNRRTPLGGNIYTAPSLHPGNAILAKHKILVPSLQTRPKGRKAIRLRIAPPTLFTDKWYFQKDIADLTLFNIMAVEADLRFPFCSPQTDNTCISFQVLSSVYNNYLSI

SEQ ID NO: 188 - protein, 133 aa, Alphatorquevirus sp.
NTFNNDNSDSKLKEFLNKAFPTTGTKGTSLNALNTFRTEGCISHPQLKKPNPQINKPLESQYFAPLDALWGDPIYYNDLNENKSLNDIIEKILIKNMITYHAKLREFPNSYQGNKAFCHLTGIYSPPYLNQGR

SEQ ID NO: 189 - protein, 166 aa, Alphatorquevirus sp.
ISPEIFGLYTEIIYNPYTDKGTGNKVWMDPLTKENNIYKEGQSKCLLTDMPLWTLLFGYTDWCKKDTNNWDLPLNYRLVLICPYTFPKLYNEKVKDYGYIPYSYKFGAGQMPDGSNYIPFQFRAKWYPTVLHQQQVMEDISRSGPFAPKVEKPSTQLVMKYCFNFN

SEQ ID NO: 190 - protein, 164 aa, Alphatorquevirus sp.
WGGNPIIEQIVKDPSFQPTYEIPGTGNIPRRIQVIDPRVLGPHYSFRSWDMRRHTFSRASIKRVSEQQETSDLVFSGPKKPRVDIPKQETQEESSHSLQRESRPWETEEESETEALSQESQEVPFQQQLQQQYQEQLKLRQGIKVLFEQLIRTQQGVHVNPCLR

SEQ ID NO: 191-214 - skipped (24 entries)

SEQ ID NO: 215 - protein, 666 aa, Betatorquevirus sp.; identical to SEQ ID NO: 58

SEQ ID NO: 216 - protein, 38 aa, Betatorquevirus sp.
MPYYYRRRRYNYRRPRWYGRGWIRRPFRRRFRRKRRVR

SEQ ID NO: 217 - protein, 208 aa, Betatorquevirus sp.
PTYTTIPLKQWQPPYKRTCYIKGQDCLIYYSNLRLGMNSTMYEKSIVPVHWPGGGSFSVSMLTLDALYDIHKLCRNWWTSTNQDLPLVRYKGCKITFYQSTFTDYIVRIHTELPANSNKLTYPNTHPLMMMMSKYKHIIPSRQTRRKKKPYTKIFVKPPPQFENKWYFATDLYKIPLLQIHCTACNLQNPFVKPDKLSNNVTLWSLNT

SEQ ID NO: 218 - protein, 128 aa, Betatorquevirus sp.
ISIQNRNMSVDQGQSWPFKILGTQSFYFYFYTGANLPGDTTQIPVADLLPLTNPRINRPGQSLNEAKITDHITFTEYKNKFTNYWGNPFNKHIQEHLDMILYSLKSPEAIKNEWTTENMKWNQLNNAG

SEQ ID NO: 219 - protein, 163 aa, Betatorquevirus sp.
TMALTPFNEPIFTQIQYNPDRDTGEDTQLYLLSNATGTGWDPPGIPELILEGFPLWLIYWGFADFQKNLKKVTNIDTNYMLVAKTKFTQKPGTFYLVILNDTFVEGNSPYEKQPLPEDNIKWYPQVQYQLEAQNKLLQTGPFTPNIQGQLSDNISMFYKFYFK

SEQ ID NO: 220 - protein, 129 aa, Betatorquevirus sp.
WGGSPPKAINVENPAHQIQYPIPRNEHETTSLQSPGEAPESILYSFDYRHGNYTTTALSRISQDWALKDTVSKITEPDRQQLLKQALECLQISEETQEKKEKEVQQLISNLRQQQQLYRERIISLLKDQ

SEQ ID NO: 221-226 - skipped (6 entries)

SEQ ID NO: 227 - protein, 220 aa, Artificial Sequence
  Description of Artificial Sequence: Synthetic polypeptide
  MOD_RES (29)..(31): Any amino acid
  SITE (29)..(31): This region may encompass 0-3 residues
  MOD_RES (100)..(100): Any amino acid
  MOD_RES (125)..(129): Any amino acid
  SITE (125)..(129): This region may encompass 1-5 residues
  MOD_RES (181)..(181): Any amino acid
  MOD_RES (211)..(211): Any amino acid
LVLTQWQPNTVRRCYIRGYLPLIICGENXXXTTSRNYATHSDDTIQKGPFGGGMSTTTFSLRVLYDEYQRFMNRWTYSNEDLDLARYLGCKFTFYRHPDXDFIVQYNTNPPFKDTKLTAPSIHPXXXXXGMLMLSKRKILIPSLKTRPKGKHYVKVRIGPPKLFEDKWYTQSDLCDVPLVXLYATAADLQHPFGSPQTDNPCVTFQVLGSXYNKHLSISP

SEQ ID NO: 228 - protein, 172 aa, Artificial Sequence
  Description of Artificial Sequence: Synthetic polypeptide
  MOD_RES (38)..(38): Any amino acid
  MOD_RES (44)..(46): Any amino acid
  SITE (44)..(46): This region may encompass 0-3 residues
  MOD_RES (77)..(77): Any amino acid
  MOD_RES (79)..(79): Any amino acid
  MOD_RES (98)..(101): Any amino acid
  SITE (98)..(101): This region may encompass 0-4 residues
SNFEFPGAYTDITYNPLTDKGVGNMVWIQYLTKPDTIXDKTQSXXXKCLIEDLPLWAALYGYVDFCEKETGDSAIIXNXGRVLIRCPYTKPPLYDKTXXXXNKGFVPYSTNFGNGKMPGGSGYVPIYWRARWYPTLFHQKEVLEDIVQSGPFAYKDEKPSTQLVMKYCFNFN

SEQ ID NO: 229 - protein, 258 aa, Artificial Sequence
  Description of Artificial Sequence: Synthetic polypeptide
  MOD_RES (20)..(22): Any amino acid
  SITE (20)..(22): This region may encompass 0-3 residues
  MOD_RES (25)..(25): Any amino acid
  MOD_RES (78)..(78): Any amino acid
  MOD_RES (89)..(89): Any amino acid
  MOD_RES (91)..(91): Any amino acid
  MOD_RES (95)..(98): Any amino acid
  SITE (95)..(98): This region may encompass 1-4 residues
  MOD_RES (107)..(120): Any amino acid
  SITE (107)..(120): This region may encompass 2-14 residues
  MOD_RES (129)..(129): Any amino acid
  MOD_RES (139)..(168): Any amino acid
  SITE (139)..(168): This region may encompass 0-30 residues
  MOD_RES (201)..(204): Any amino acid
  SITE (201)..(204): This region may encompass 0-4 residues
  MOD_RES (219)..(258): Any amino acid
  SITE (219)..(258): This region may encompass 0-40 residues
WGGNPISQQVVRNPCKDSGXXXSGXGRQPRSVQVVDPKYMGPEYTFHSWDWRRGLFGEKAIKRMSEQPTDDEIFTGGXPKRPRRDPPTXQXPEEXXXXQKESSSFRXXXXXXXXXXXXXXPWESSSQEXESESQEEEEXXXXXXXXXXXXXXXXXXXXXXXXXXXXXXEQTVQQQLRQQLREQRRLRVQLQLLFQQLLKTXXXXQAGLHINPLLLSQAXXXXXXXXXXXXXXXXXXXXXXXXXXXXXXXXXXXXXXXX

SEQ ID NO: 230 - protein, 214 aa, Artificial Sequence
  Description of Artificial Sequence: Synthetic polypeptide
  MOD_RES (136)..(136): Any amino acid
  MOD_RES (138)..(141): Any amino acid
  SITE (138)..(141): This region may encompass 1-4 residues
  MOD_RES (179)..(179): Any amino acid
LKQWQPSTIRKCKIKGYLPLFQCGKGRISNNYTQYKESIVPHHEPGGGGWSIQQFTLGALYEEHLKLRNWWTKSNDGLPLVRYLGCTIKLYRSEDTDYIVTYQRCYPMTATKLTYLSTQPSRMLMNKHKIIVPSKXTXXXXNKKKKPYKKIFIKPPSQMQNKWYFQQDIANTPLLQLTXTACSLDRMYLSSDSISNNITFTSLNTNFFQNPNFQ

SEQ ID NO: 231 - protein, 187 aa, Artificial Sequence
  Description of Artificial Sequence: Synthetic polypeptide
  MOD_RES (1)..(10): Any amino acid
  SITE (1)..(10): This region may encompass 4-10 residues
  MOD_RES (38)..(45): Any amino acid
  SITE (38)..(45): This region may encompass 1-8 residues
  MOD_RES (94)..(94): Any amino acid
  MOD_RES (100)..(102): Any amino acid
  SITE (100)..(102): This region may encompass 1-3 residues
  MOD_RES (112)..(112): Any amino acid
  MOD_RES (114)..(115): Any amino acid
  SITE (114)..(115): This region may encompass 0-2 residues
  MOD_RES (124)..(139): Any amino acid
  SITE (124)..(139): This region may encompass 3-16 residues
  MOD_RES (154)..(154): Any amino acid
XXXXXXXXXXTPLYFECRYNPFKDKGTGNKVYLVSNNXXXXXXXXTGWDPPTDPDLIIEGFPLWLLLWGWLDWQKKLGKIQNIDTDYILVIQSXYYIPPXXXKLPYYVPLDXDXXFLHGRSPYXXXXXXXXXXXXXXXXPSDKQHWHPKVRFQXETINNIALTGPGTPKLPNQKSIQAHMKYKFYFK

SEQ ID NO: 232 - protein, 163 aa, Artificial Sequence
  Description of Artificial Sequence: Synthetic polypeptide
  MOD_RES (34)..(34): Any amino acid
  MOD_RES (65)..(65): Any amino acid
  MOD_RES (77)..(78): Any amino acid
  MOD_RES (86)..(87): Any amino acid
  MOD_RES (96)..(96): Any amino acid
  MOD_RES (102)..(106): Any amino acid
  SITE (102)..(106): This region may encompass 0-5 residues
  MOD_RES (125)..(125): Any amino acid
  MOD_RES (135)..(135): Any amino acid
  MOD_RES (138)..(163): Any amino acid
  SITE (138)..(163): This region may encompass 0-26 residues
WGGCPAPMETITDPCKQPKYPIPNNLLQTTSLQXPTTPIETYLYKFDERRGLLTKKAAKRIKKDXTTETTLFTDTGXXTSTTLPTXXQTETTQEEXTSEEEXXXXXETLLQQLQQLRRKQKQLRXRILQLLQLLXLLXXXXXXXXXXXXXXXXXXXXXXXXXX

SEQ ID NO: 233 - protein, 203 aa, Artificial Sequence
  Description of Artificial Sequence: Synthetic polypeptide
  MOD_RES (79)..(79): Any amino acid
  MOD_RES (104)..(104): Any amino acid
  MOD_RES (116)..(116): Any amino acid
  MOD_RES (120)..(121): Any amino acid
  MOD_RES (125)..(125): Any amino acid
  MOD_RES (170)..(170): Any amino acid
TIPLKQWQPESIRKCKIKGYGTLVLGAEGRQFYCYTNEKDEYTPPKAPGGGGFGVELFSLEYLYEQWKARNNIWTKSNXYKDLCRYTGCKITFYRHPTTDFIVXYSRQPPFEIDKXTYMXXHPQXLLLRKHKKIILSKATNPKGKLKKKIKIKPPKQMLNKWFFQKQFAXYGLVQLQAAACBLRYPRLGCCNENRLITLYYLN

SEQ ID NO: 234 - protein, 162 aa, Artificial Sequence
  Description of Artificial Sequence: Synthetic polypeptide
  MOD_RES (12)..(12): Any amino acid
  MOD_RES (20)..(20): Any amino acid
  MOD_RES (23)..(23): Any amino acid
  MOD_RES (30)..(30): Any amino acid
  MOD_RES (58)..(58): I or L
  MOD_RES (84)..(84): Any amino acid
  MOD_RES (90)..(90): Any amino acid
  MOD_RES (95)..(95): Any amino acid
  MOD_RES (105)..(105): Any amino acid
  MOD_RES (111)..(111): I or L
  MOD_RES (113)..(113): Any amino acid
  MOD_RES (154)..(154): Any amino acid
  MOD_RES (156)..(156): Any amino acid
LPIVVARYNPAXDTGKGNKXWLXSTLNGSXWAPPTTDKDLIIEGLPLWLALYGYWSYXKKVKKDKGILQSHMFVVKSPAIQPLXTATTQXTFYPXIDNSFIQGKXPYDEPXTXNQKKLWYPTLEHQQETINAIVESGPYVPKLDNQKNSTWELXYXYTFYFK

SEQ ID NO: 235 - protein, 177 aa, Artificial Sequence
  Description of Artificial Sequence: Synthetic polypeptide
  MOD_RES (16)..(16): Any amino acid
  MOD_RES (26)..(26): Any amino acid
  MOD_RES (33)..(33): Any amino acid
  MOD_RES (73)..(73): Any amino acid
  MOD_RES (81)..(82): Any amino acid
  SITE (81)..(82): This region may encompass 0-2 residues
  MOD_RES (90)..(90): Any amino acid
  MOD_RES (94)..(94): Any amino acid
  MOD_RES (119)..(124): Any amino acid
  SITE (119)..(124): This region may encompass 1-6 residues
  MOD_RES (168)..(177): Any amino acid
  SITE (168)..(177): This region may encompass 1-10 residues
WGGPQIPDQPVEDPKXQGTYPVPDTXQQTIQIXNPLKQKPETMFHDWDYRRGIITSTALKRMQENLETDSSFXSDSEETPXXKKKKRLTXELPXPQEETEEIQSCLLSLCEESTCQEEXXXXXXENLQQLIHQQQQQQQQLKHNILKLLSDLKZKQRLLQLQTGILEXXXXXXXXXX

SEQ ID NO: 236-689 - skipped (454 entries)

SEQ ID NO: 690 - DNA, 22 bp, Artificial Sequence
  Description of Artificial Sequence: Synthetic primer
attcgaatggctgagtttatgc

SEQ ID NO: 691 - DNA, 24 bp, Artificial Sequence
  Description of Artificial Sequence: Synthetic primer
cacgaattagccaagactgggcac

SEQ ID NO: 692 - DNA, 20 bp, Artificial Sequence
  Description of Artificial Sequence: Synthetic primer
gctcccactcctgatttctg

SEQ ID NO: 693 - DNA, 21 bp, Artificial Sequence
  Description of Artificial Sequence: Synthetic primer
ccttgactacggtggtttcac

SEQ ID NO: 694 - DNA, 22 bp, Artificial Sequence
  Description of Artificial Sequence: Synthetic primer
tgcaggcattcgagggcttgtt

SEQ ID NO: 695 - DNA, 20 bp, Artificial Sequence
  Description of Artificial Sequence: Synthetic primer
tttaaccccctagtcccagg

SEQ ID NO: 696-800 - skipped (105 entries)

SEQ ID NO: 801 - DNA, 156 bp, Artificial Sequence; identical to SEQ ID NO: 125
  Description of Artificial Sequence: Synthetic polynucleotide

SEQ ID NO: 802 - DNA, 150 bp, Artificial Sequence
  Description of Artificial Sequence: Synthetic polynucleotide
ccgagcgttagcgaggagtgcgaccctaccccctgggcccacttcttcggagccgcgcgctacgccttcggctgcgcgcggcacctcagacccccgctcgtgctgacacgcttgcgcgtgtcagaccacttcgggctcgcgggggtcggg

SEQ ID NO: 803 - DNA, 122 bp, Artificial Sequence; identical to SEQ ID NO: 153
  Description of Artificial Sequence: Synthetic polynucleotide

SEQ ID NO: 804 - DNA, 111 bp, Artificial Sequence
  Description of Artificial Sequence: Synthetic polynucleotide
cggcccagcggcggcgcgcgcgcttcgcgcgcgcgccggggggctccgcccccccccgcgcatgcgcggggcccccccccgcggggggctccgccccccggtccccccccg

SEQ ID NO: 805 - DNA, 115 bp, Artificial Sequence
  Description of Artificial Sequence: Synthetic polynucleotide
cggccgtgcggcggcgcgcgcgcttcgcgcgcgcgccgggggctgccgcccccccccgcgcatgcgcgcggggcccccccccgcggggggctccgccccccggcccccccccccg

SEQ ID NO: 806 - DNA, 104 bp, Artificial Sequence; identical to SEQ ID NO: 144
  Description of Artificial Sequence: Synthetic polynucleotide

SEQ ID NO: 807 - DNA, 108 bp, Artificial Sequence
  Description of Artificial Sequence: Synthetic polynucleotide
ggcggcggcgcgcgcgctacgcgcgcgcgccggggagctctgcccccccccgcgcatgcgcgcgggtcccccccccgcggggggctccgccccccggtcccccccccg

SEQ ID NO: 808-828 - skipped (21 entries)

SEQ ID NO: 829 - protein, 11 aa, Artificial Sequence
  Description of Artificial Sequence: Synthetic peptide
  MOD_RES (4)..(5): Any amino acid
  MOD_RES (7)..(7): Any amino acid
  MOD_RES (9)..(10): Any amino acid
YNPXXDXGXXN

SEQ ID NO: 830-885 - skipped (56 entries)

SEQ ID NO: 886 - DNA, 3176 bp, Gammatorquevirus sp.
taaaatggcgggagccaatcattttatactttcactttccaattaaaaatggccacgtcacaaacaaggggtggagccatttaaactatataactaagtggggtggcgaatggctgagtttaccccgctagacggtgcagggaccggatcgagcgcagcgaggaggtccccggctgcccatgggcgggagccgaggtgagtgaaaccaccgaggtctaggggcaattcgggctagggcagtctagcggaacgggcaagaaacttaaaacaatatttgttttacagatggttagtatatcctcaagtgatttttttaagaaaacgaaatttaatgaggagacgcagaaccaagtatggatgtctcaaattgctgactctcatgataatatctgcagttgctggcatccatttgctcaccttcttgcttccatatttcctcctggccacaaagatcgtgatcttactattaaccaaattcttctaagagattataaagaaaaatgccattctggtggagaagaaggagaaaattctggaccaacaacaggtttaattacaccaaaagaagaagatatagaaaaagatggcccagaaggcgccgcagaagaagaccatacagacgccctgttcgccgccgccgtagaaaacttcgaaaggtaaagagaaaaaaaaaatctttaattgttagacaatggcaaccagacagtataagaacttgtaaaattataggacagtcagctatagttgttggggctgaaggaaagcaaatgtactgttatactgtcaataagttaattaatgtgcccccaaaaacaccatatgggggaggctttggagtagaccaatacacactgaaatacttatatgaagaatacagatttgcacaaaacatttggacacaatctaatgtactgaaagacttatgcagatacataaatgttaagctaatattctacagagacaacaaaacagactttgtcctttcctatgacagaaacccaccttttcaactaacaaaatttacatacccaggagcacacccacaacaaatcatgcttcaaaaacaccacaaattcatactatcacaaatgacaaagcctaatggaagactaacaaaaaaactcaaaattaaacctcctaaacaaatgctttctaaatggttcttttcaaaacaattctgtaaataccctttactatctcttaaagcttctgcactagaccttaggcactcttacctaggctgctgtaatgaaaatccacaggtatttttttattatttaaaccatggatactacacaataacaaactggggagcacaatcctcaacagcatacagacctaactccaaggtgacagacacaacatactacagatacaaaaatgacagaaaaaatattaacattaaaagccatgaatacgaaaaaagtatatcatatgaaaacggttattttcaatctagtttcttacaaacacagtgcatatataccagtgagcgtggtgaagcctgtatagcagaaaaaccactaggaatagctatttacaatccagtaaaagacaatggagatggtaatatgatataccttgtaagcactctagcaaacacttgggaccagcctccaaaagacagtgctattttaatacaaggagtacccatatggctaggcttatttggatatttagactactgtagacaaattaaagctgacaaaacatggctagacagtcatgtactagtaattcaaagtcctgctatttttacttacccaaatccaggagcaggcaaatggtattgtccactatcacaaagttttataaatggcaatggtccgtttaatcaaccacctacactgctacaaaaagcaaagtggtttccacaaatacaataccaacaagaaattattaatagctttgtagaatcaggaccatttgttcccaaatatgcaaatcaaactgaaagcaactgggaactaaaatataaatatgtttttacatttaagtggggtggaccacaattccatgaaccagaaattgctgaccctagcaaacaagagcagtatgatgtccccgatactttctaccaaacaatacaaattgaagatccagaaggacaagaccccagatctctcatccatgattgggactacagacgaggctttattaaagaaagatctcttaaaagaatgtcaacttacttctcaactcatacagatcagcaagcaacttcagaggaagacattcccaaaaagaaaaagagaattggaccccaactcacagtcccacaacaaaaagaagaggagacactgtcatgtctcctctctctctgcaaaaaagataccttccaagaaacagagacacaagaagacctccagcagctcatcaagcagcagcaggagcagcagctcctcctcaagagaaacatcctccagctcatccacaaactaaaagagaatcaacaaatgcttcagcttcacacaggcatgttaccttaaccagatttaaacctggatttgaagagcaaacagagagagaattagcaattatatttcataggccccctagaacctacaaagaggaccttccattctatccctggctaccacctgcaccccttgtacaatttaaccttaacttcaaaggctaggccaacaatgtacacttagtaaagcatgtttattaaagcacaacccccaaaataaatgtaaaaataaaaaaaaaaaaaaaaaaataaaaaattgcaaaaattcggcgctcgcgcgcatgtgcgcctctggcgcaaatcacgcaacgctcgcgcgcccgcgtatgtctctttaccacgcacctagattggggtgcgcgcgctagcgcgcgcaccccaatgcgccccgccctcgttccgacccgcttgcgcgggtcggaccacttcgggctcgggggggcgcgcctgcggcgcttttttactaaacagactccgagccgccatttggccccctaagctccgcccccctcatgaatattcataaaggaaaccacataattagaattgccgaccacaaactgccatatgctaattagttccccttttacaaagtaaaaggggaagtgaacatagccccacacccgcaggggcaaggccccgcacccctacgtcactaaccacgcccccgccgccatcttgggtgcggcagggcgggggc

SEQ ID NO: 887 - protein, 124 aa, Gammatorquevirus sp.
MVSISSSDFFKKTKFNEETQNQVWMSQIADSHDNICSCWHPFAHLLASIFPPGHKDRDLTINQILLRDYKEKCHSGGEEGENSGPTTGLITPKEEDIEKDGPEGAAEEDHTDALFAAAVENFER

SEQ ID NO: 888 - protein, 271 aa, Gammatorquevirus sp.
MVSISSSDFFKKTKFNEETQNQVWMSQIADSHDNICSCWHPFAHLLASIFPPGHKDRDLTINQILLRDYKEKCHSGGEEGENSGPTTGLITPKEEDIEKDGPEGAAEEDHTDALFAAAVENFESGVDHNSMNQKLLTLANKSSMMSPILSTKQYKLKIQKDKTPDLSSMIGTTDEALLKKDLLKECQLTSQLIQISKQLQRKTFPKRKRELDPNSQSHNKKKRRHCHVSSLSAKKIPSKKQRHKKTSSSSSSSSRSSSSSSRETSSSSSTN

SEQ ID NO: 889 - protein, 267 aa, Gammatorquevirus sp.
MVSISSSDFFKKTKFNEETQNQVWMSQIADSHDNICSCWHPFAHLLASIFPPGHKDRDLTINQILLRDYKEKCHSGGEEGENSGPTTGLITPKEEDIEKDGPEGAAEEDHTDALFAAAVENFERSASNFRGRHSQKEKENWTPTHSPTTKRRGDTVMSPLSLQKRYLPRNRDTRRPPAAHQAAAGAAAPPQEKHPPAHPQTKRESTNASASHRHVTLTRFKPGFEEQTERELAIIFHRPPRTYKEDLPFYPWLPPAPLVQFNLNFKG

SEQ ID NO: 890 - protein, 50 aa, Gammatorquevirus sp.
MRRRRTKYGCLKLLTLMIISAVAGIHLLTFLLPYFLLATKIVILLLTKFF

SEQ ID NO: 891 - protein, 662 aa, Gammatorquevirus sp.
MPFWWRRRRKFWTNNRFNYTKRRRYRKRWPRRRRRRRPYRRPVRRRRRKLRKVKRKKKSLIVRQWQPDSIRTCKIIGQSAIVVGAEGKQMYCYTVNKLINVPPKTPYGGGFGVDQYTLKYLYEEYRFAQNIWTQSNVLKDLCRYINVKLIFYRDNKTDFVLSYDRNPPFQLTKFTYPGAHPQQIMLQKHHKFILSQMTKPNGRLTKKLKIKPPKQMLSKWFFSKQFCKYPLLSLKASALDLRHSYLGCCNENPQVFFYYLNHGYYTITNWGAQSSTAYRPNSKVTDTTYYRYKNDRKNINIKSHEYEKSISYENGYFQSSFLQTQCIYTSERGEACIAEKPLGIAIYNPVKDNGDGNMIYLVSTLANTWDQPPKDSAILIQGVPIWLGLFGYLDYCRQIKADKTWLDSHVLVIQSPAIFTYPNPGAGKWYCPLSQSFINGNGPFNQPPTLLQKAKWFPQIQYQQEIINSFVESGPFVPKYANQTESNWELKYKYVFTFKWGGPQFHEPEIADPSKQEQYDVPDTFYQTIQIEDPEGQDPRSLIHDWDYRRGFIKERSLKRMSTYFSTHTDQQATSEEDIPKKKKRIGPQLTVPQQKEEETLSCLLSLCKKDTFQETETQEDLQQLIKQQQEQQLLLKRNILQLIHKLKENQQMLQLHTGMLP

SEQ ID NO: 892 - protein, 215 aa, Gammatorquevirus sp.
MPFWWRRRRKFWTNNRFNYTKRRRYRKRWPRRRRRRRPYRRPVRRRRRKLRKWGGPQFHEPEIADPSKQEQYDVPDTFYQTIQIEDPEGQDPRSLIHDWDYRRGFIKERSLKRMSTYFSTHTDQQATSEEDIPKKKKRIGPQLTVPQQKEEETLSCLLSLCKKDTFQETETQEDLQQLIKQQQEQQLLLKRNILQLIHKLKENQQMLQLHTGMLP

SEQ ID NO: 893 - protein, 129 aa, Gammatorquevirus sp.
MPFWWRRRRKFWTNNRFNYTKRRRYRKRWPRRRRRRRPYRRPVRRRRRKLRKISKQLQRKTFPKRKRELDPNSQSHNKKKRRHCHVSSLSAKKIPSKKQRHKKTSSSSSSSSRSSSSSSRETSSSSSTN

SEQ ID NO: 894-924 - skipped (31 entries)

SEQ ID NO: 925 - protein, 662 aa, Gammatorquevirus sp.; identical to SEQ ID NO: 891

SEQ ID NO: 926 - protein, 58 aa, Gammatorquevirus sp.
MPFWWRRRRKFWTNNRFNYTKRRRYRKRWPRRRRRRRPYRRPVRRRRRKLRKVKRKKK

SEQ ID NO: 927 - protein, 202 aa, Gammatorquevirus sp.
SLIVRQWQPDSIRTCKIIGQSAIVVGAEGKQMYCYTVNKLINVPPKTPYGGGFGVDQYTLKYLYEEYRFAQNIWTQSNVLKDLCRYINVKLIFYRDNKTDFVLSYDRNPPFQLTKFTYPGAHPQQIMLQKHHKFILSQMTKPNGRLTKKLKIKPPKQMLSKWFFSKQFCKYPLLSLKASALDLRHSYLGCCNENPQVFFYYL

SEQ ID NO: 928 - protein, 79 aa, Gammatorquevirus sp.
NHGYYTITNWGAQSSTAYRPNSKVTDTTYYRYKNDRKNINIKSHEYEKSISYENGYFQSSFLQTQCIYTSERGEACIAE

SEQ ID NO: 929 - protein, 160 aa, Gammatorquevirus sp.
KPLGIAIYNPVKDNGDGNMIYLVSTLANTWDQPPKDSAILIQGVPIWLGLFGYLDYCRQIKADKTWLDSHVLVIQSPAIFTYPNPGAGKWYCPLSQSFINGNGPFNQPPTLLQKAKWFPQIQYQQEIINSFVESGPFVPKYANQTESNWELKYKYVFTFK

SEQ ID NO: 930 - protein, 163 aa, Gammatorquevirus sp.
WGGPQFHEPEIADPSKQEQYDVPDTFYQTIQIEDPEGQDPRSLIHDWDYRRGFIKERSLKRMSTYFSTHTDQQATSEEDIPKKKKRIGPQLTVPQQKEEETLSCLLSLCKKDTFQETETQEDLQQLIKQQQEQQLLLKRNILQLIHKLKENQQMLQLHTGMLP

SEQ ID NO: 931-948 - skipped (18 entries)

SEQ ID NO: 949 - protein, 21 aa, Artificial Sequence
  Description of Artificial Sequence: Synthetic peptide
  MOD_RES (1)..(1): W or F
  MOD_RES (2)..(8): Any amino acid
  MOD_RES (10)..(12): Any amino acid
  MOD_RES (14)..(14): Any amino acid
  MOD_RES (16)..(20): Any amino acid
XXXXXXXXHXXXCXCXXXXXH

SEQ ID NO: 950 - protein, 11 aa, Unknown
  Description of Unknown: Anellovirus ORF1 sequence
  MOD_RES (4)..(5): Any amino acid
  MOD_RES (7)..(7): Any amino acid
  MOD_RES (9)..(10): Any amino acid
YNPXXDXGXXN

SEQ ID NO: 951 - protein, 22 aa, Unknown
  Description of Unknown: Anellovirus ORF1 sequence
  MOD_RES (6)..(7): Any amino acid
  MOD_RES (9)..(9): Any amino acid
  MOD_RES (15)..(16): Any amino acid
YNCSPXXDXGASKRXXNTSVAK

SEQ ID NO: 952 - DNA, 51 bp, Artificial Sequence
  Description of Artificial Sequence: Synthetic oligonucleotide
  modified_base (45)..(45): a, c, t, g, unknown or other
  See specification as filed for detailed description of substitutions and preferred embodiments
aggtgagtgaaaccaccgaagtcaaggggcaattcgggctagggncagtct

SEQ ID NO: 953 - DNA, 50 bp, Artificial Sequence
  Description of Artificial Sequence: Synthetic oligonucleotide
aggtgagtttacacaccgcagtcaaggggcaattcgggctcgggactggc

SEQ ID NO: 954 - DNA, 50 bp, Artificial Sequence
  Description of Artificial Sequence: Synthetic oligonucleotide
aggtgagtgaaaccaccgaagtcaaggggcaattcgggctagatcagtct

SEQ ID NO: 955 - DNA, 50 bp, Artificial Sequence
  Description of Artificial Sequence: Synthetic oligonucleotide
aggtgagtgaaaccaccgaggtctaggggcaattcgggctagggcagtct

SEQ ID NO: 956 - protein, 6 aa, Artificial Sequence
  Description of Artificial Sequence: Synthetic 6xHis tag
HHHHHH

SEQ ID NO: 957 - DNA, 20 bp, Artificial Sequence
  Description of Artificial Sequence: Synthetic primer
agcaacaggtaatggaggac

SEQ ID NO: 958 - DNA, 19 bp, Artificial Sequence
  Description of Artificial Sequence: Synthetic primer
tgaagctggggtctttaac

SEQ ID NO: 959 - DNA, 21 bp, Artificial Sequence
  Description of Artificial Sequence: Synthetic probe
tctacctaggtgcaaagggcc

(19) United States
(12) Patent Application Publication
(10) Pub. No.: US 2023/0340527 A1
(43) Pub. Date: Oct. 26, 2023

(54) BACULOVIRUS EXPRESSION SYSTEMS

(71) Applicant: FLAGSHIP PIONEERING INNOVATIONS V, INC., Cambridge, MA (US)

(72) Inventors: Simon Delagrave, Sudbury, MA (US); Jared David Pitts, Jamaica Plain, MA (US)

(21) Appl. No.: 18/009,476

(22) PCT Filed: Jun. 11, 2021
(86) PCT No.: PCT/US21/37076

§ 371 Date: Dec. 9, 2022

Related U.S. Application Data
(60) Provisional application No. 63/038,603, filed on Jun. 12, 2020.
(60) Provisional application No. 63/147,056, filed on Feb. 8, 2021.

(51) Int. Cl. C12N 15/86

(52) U.S. Cl. CPC C12N 15/86; C12N 2710/14043; C12N 2710/14044; C12N 2750/00022; C12N 2750/00043

(57) ABSTRACT

This invention relates generally to compositions for making anellovectors and uses thereof. For instance, the disclosure provides compositions and methods for using insect cells to produce Anellovirus proteins, e.g., ORF1.

### CROSS-REFERENCE TO RELATED APPLICATIONS

This application claims the benefit of U.S. Provisional Application Nos. 63/038,603, filed Jun. 12, 2020, and 63/147,056, filed Feb. 8, 2021. The contents of the aforementioned applications are hereby incorporated by reference in their entirety.

### BACKGROUND

There is an ongoing need to develop compositions and methods for making suitable vectors to deliver therapeutic effectors to patients. For instance, Anellovirus-based vectors are a promising modality for therapeutic delivery. However, efforts in the field have been hampered by difficulties in producing a full-length, purified Anellovirus ORF1 protein. There is a need in the art for new methods of producing purified Anellovirus proteins.

### SUMMARY

The present disclosure provides compositions and methods for producing an anellovector (e.g., a synthetic anellovector) that can be used as a delivery vehicle, e.g., for delivering genetic material, for delivering an effector, e.g., a payload, or for delivering a therapeutic agent or a therapeutic effector to a eukaryotic cell (e.g., a human cell or a human tissue). Described herein are, for example, baculovirus particles and nucleic acid constructs comprising a genetic element region as described herein and elements suitable for production of baculoviruses (also referred to herein as bacmids), such as a baculovirus genome or fragment(s) thereof, as well as cells (e.g., insect cells) suitable for producing baculoviruses, e.g., cells comprising such nucleic acid constructs. Also described are donor vectors comprising a genetic element region and one or more elements suitable for transfer of the genetic element region into a bacmid backbone (e.g., cognate recombinase recognition sites, e.g., Tn7R and Tn7L sites). Also described herein are methods of producing anellovectors, e.g., using such nucleic acid constructs (e.g., bacmids and/or donor vectors), baculoviruses, and/or cells. In addition, methods of producing nucleic acid constructs (e.g., bacmids and/or donor vectors), baculoviruses, and cells suitable for producing anellovectors are provided herein.

An anellovector (e.g., produced using a composition or method as described herein) generally comprises a genetic element (e.g., a genetic element comprising or encoding an effector, e.g., an exogenous or endogenous effector, e.g., a therapeutic effector) encapsulated in a proteinaceous exterior (e.g., a proteinaceous exterior comprising an Anellovirus capsid protein, e.g., an Anellovirus ORF1 protein or a polypeptide encoded by an Anellovirus ORF1 nucleic acid, e.g., as described herein), which is capable of introducing the genetic element into a cell (e.g., a mammalian cell, e.g., a human cell). In some embodiments, the anellovector is an infectious vehicle or particle comprising a proteinaceous exterior comprising a polypeptide encoded by an Anellovirus ORF1 nucleic acid (e.g., an ORF1 nucleic acid of Alphatorquevirus, Betatorquevirus, or Gammatorquevirus, e.g., an ORF1 of Alphatorquevirus clade 1, Alphatorquevirus clade 2, Alphatorquevirus clade 3, Alphatorquevirus clade 4, Alphatorquevirus clade 5, Alphatorquevirus clade 6, or Alphatorquevirus clade 7, e.g., as described herein). The genetic element of an anellovector of the present disclosure is typically a circular and/or single-stranded DNA molecule (e.g., circular and single stranded), and generally includes a protein binding sequence that binds to the proteinaceous exterior enclosing it, or a polypeptide attached thereto, which may facilitate enclosure of the genetic element within the proteinaceous exterior and/or enrichment of the genetic element, relative to other nucleic acids, within the proteinaceous exterior. In some embodiments, the genetic element of an anellovector is produced using a baculovirus, nucleic acid construct (e.g., a bacmid and/or donor vector), and/or insect cell, e.g., as described herein.

In some instances, the genetic element comprises or encodes an effector (e.g., a nucleic acid effector, such as a non-coding RNA, or a polypeptide effector, e.g., a protein), e.g., which can be expressed in the cell. In some embodiments, the effector is a therapeutic agent or a therapeutic effector, e.g., as described herein. In some embodiments, the effector is an endogenous effector or an exogenous effector, e.g., to a wild-type Anellovirus or a target cell. In some embodiments, the effector is exogenous to a wild-type Anellovirus or a target cell. In some embodiments, the anellovector can deliver an effector into a cell by contacting the cell and introducing a genetic element encoding the effector into the cell, such that the effector is made or expressed by the cell. In certain instances, the effector is an endogenous effector (e.g., endogenous to the target cell but, e.g., provided in increased amounts by the anellovector). In other instances, the effector is an exogenous effector. The effector can, in some instances, modulate a function of the cell or modulate an activity or level of a target molecule in the cell. For example, the effector can decrease levels of a target protein in the cell (e.g., as described in Examples 3 and 4 of PCT/US19/65995). In another example, the anellovector can deliver and express an effector, e.g., an exogenous protein, in vivo (e.g., as described in Examples 19 and 28 of PCT/US19/65995). Anellovectors can be used, for example, to deliver genetic material to a target cell, tissue or subject; to deliver an effector to a target cell, tissue or subject; or for treatment of diseases and disorders, e.g., by delivering an effector that can operate as a therapeutic agent to a desired cell, tissue, or subject.

In some embodiments, the compositions and methods described herein can be used to produce the genetic element of a synthetic anellovector, e.g., in a host cell. A synthetic anellovector has at least one structural difference compared to a wild-type virus (e.g., a wild-type Anellovirus, e.g., a described herein), e.g., a deletion, insertion, substitution, modification (e.g., enzymatic modification), relative to the wild-type virus. Generally, synthetic anellovectors include an exogenous genetic element enclosed within a proteinaceous exterior, which can be used for delivering the genetic element, or an effector (e.g., an exogenous effector or an endogenous effector) encoded therein (e.g., a polypeptide or nucleic acid effector), into eukaryotic (e.g., human) cells. In embodiments, the anellovector does not cause a detectable and/or an unwanted immune or inflammatory response, e.g., does not cause more than a 1%, 5%, 10%, 15% increase in a molecular marker(s) of inflammation, e.g., TNF-alpha, IL-6, IL-12, IFN, as well as B-cell response e.g. reactive or neutralizing antibodies, e.g., the anellovector may be substantially non-immunogenic to the target cell, tissue or subject.

In some embodiments, the compositions and methods described herein can be used to produce the genetic element of an anellovector comprising: (i) a genetic element comprising a promoter element and a sequence encoding an effector (e.g., an endogenous or exogenous effector), and a protein binding sequence (e.g., an exterior protein binding sequence, e.g., a packaging signal); and (ii) a proteinaceous exterior; wherein the genetic element is enclosed within the proteinaceous exterior (e.g., a capsid); and wherein the anellovector is capable of delivering the genetic element into a eukaryotic (e.g., mammalian, e.g., human) cell. In some embodiments, the genetic element is a single-stranded and/or circular DNA. Alternatively or in combination, the genetic element has one, two, three, or all of the following properties: is circular, is single-stranded, it integrates into the genome of a cell at a frequency of less than about 0.0001%, 0.001%, 0.005%, 0.01%, 0.05%, 0.1%, 0.5%, 1%, 1.5%, or 2% of the genetic element that enters the cell, and/or it integrates into the genome of a target cell at less than 1, 2, 3, 4, 5, 6, 7, 8, 9, 10, 15, 20, 25, or 30 copies per genome. In some embodiments, integration frequency is determined by quantitative gel purification assay of genomic DNA separated from free vector, e.g., as described in Wang et al. (2004, Gene Therapy 11: 711-721, incorporated herein by reference in its entirety). In some embodiments, the genetic element is enclosed within the proteinaceous exterior. In some embodiments, the anellovector is capable of delivering the genetic element into a eukaryotic cell. In some embodiments, the genetic element comprises a nucleic acid sequence (e.g., a nucleic acid sequence of between 300-4000 nucleotides, e.g., between 300-3500 nucleotides, between 300-3000 nucleotides, between 300-2500 nucleotides, between 300-2000 nucleotides, between 300-1500 nucleotides) having at least 75% (e.g., at least 75, 76, 77, 78, 79, 80, 90, 91, 92, 93, 94, 95, 96, 97, 98, 99, or 100%) sequence identity to a sequence of a wild-type Anellovirus (e.g., a wild-type Torque Teno virus (TTV), Torque Teno mini virus (TTMV), or TTMDV sequence, e.g., a wild-type Anellovirus sequence as described herein). In some embodiments, the genetic element comprises a nucleic acid sequence (e.g., a nucleic acid sequence of at least 300 nucleotides, 500 nucleotides, 1000 nucleotides, 1500 nucleotides, 2000 nucleotides, 2500 nucleotides, 3000 nucleotides or more) having at least 75% (e.g., at least 75, 76, 77, 78, 79, 80, 90, 91, 92, 93, 94, 95, 96, 97, 98, 99, or 100%) sequence identity to a sequence of a wild-type Anellovirus (e.g., a wild-type Anellovirus sequence as described herein). In some embodiments, the nucleic acid sequence is codon-optimized, e.g., for expression in a mammalian (e.g., human) cell. In some embodiments, at least 50%, 60%, 70%, 80%, 90%, 95%, 96%, 97%, 98%, 99%, or 100% of the codons in the nucleic acid sequence are codon-optimized, e.g., for expression in a mammalian (e.g., human) cell.

In some embodiments, a nucleic acid described herein comprises one or more codon-optimized open reading frames (e.g., a sequence encoding an Anellovirus ORF1, ORF1/1, ORF1/2, ORF2, ORF2/2, ORF2/3, and/or ORF2t/3, wherein the sequence is codon optimized, e.g., for expression in an animal cell, e.g., an insect cell). In some embodiments, a nucleic acid described herein comprises a codon-optimized sequence encoding an Anellovirus ORF1 molecule. Without wishing to be bound by theory, in some embodiments, codon optimization for an insect cell increases expression of the Anellovirus ORF1 molecule in an insect cell compared to a non codon-optimized sequence.

In some embodiments, the compositions and methods described herein can be used to produce the genetic element of an infectious (e.g., to a human cell) Annellovector, vehicle, or particle comprising a capsid (e.g., a capsid comprising an Anellovirus ORF, e.g., ORF1, polypeptide) encapsulating a genetic element comprising a protein binding sequence that binds to the capsid and a heterologous (to the Anellovirus) sequence encoding a therapeutic effector. In embodiments, the Anellovector is capable of delivering the genetic element into a mammalian, e.g., human, cell. In some embodiments, the genetic element has less than about 6% (e.g., less than 10%, 9%5, 8%, 7%, 6%, 5.5%, 5%, 4.5%, 4%, 3.5%, 3%, 2.5%, 2%, 1.5%, or less) identity to a wild type Anellovirus genome sequence. In some embodiments, the genetic element has no more than 1.5%, 2%, 2.5%, 3%, 3.5%, 4%, 4.5%, 5%, 5.5% or 6% identity to a wild type Anellovirus genome sequence. In some embodiments, the genetic element has at least about 2% to at least about 5.5% (e.g., 2 to 5%, 3% to 5%, 4% to 5%) identity to a wild type Anellovirus. In some embodiments, the genetic element has greater than about 2000, 3000, 4000, 4500, or 5000 nucleotides of non-viral sequence (e.g., non Anellovirus genome sequence). In some embodiments, the genetic element has greater than about 2000 to 5000, 2500 to 4500, 3000 to 4500, 2500 to 4500, 3500, or 4000, 4500 (e.g., between about 3000 to 4500) nucleotides of non-viral sequence (e.g., non Anellovirus genome sequence). In some embodiments, the genetic element is a single-stranded, circular DNA. Alternatively or in combination, the genetic element has one, two or 3 of the following properties: is circular, is single stranded, it integrates into the genome of a cell at a frequency of less than about 0.001%, 0.005%, 0.01%, 0.05%, 0.1%, 0.5%, 1%, 1.5%, or 2% of the genetic element that enters the cell, it integrates into the genome of a target cell at less than 1, 2, 3, 4, 5, 6, 7, 8, 9, 10, 15, 20, 25, or 30 copies per genome or integrates at a frequency of less than about 0.0001%, 0.001%, 0.005%, 0.01%, 0.05%, 0.1%, 0.5%, 1%, 1.5%, or 2% of the genetic element that enters the cell (e.g., by comparing integration frequency into genomic DNA relative to genetic element sequences from cell lysates). In some embodiments, integration frequency is determined by quantitative gel purification assay of genomic DNA separated from free vector, e.g., as described in Wang et al. (2004, Gene Therapy 11: 711-721, incorporated herein by reference in its entirety).

In some embodiments, Anelloviruses or anellovectors, as described herein, can be used as effective delivery vehicles for introducing an agent, such as an effector described herein, to a target cell, e.g., a target cell in a subject to be treated therapeutically or prophylactically.

In some embodiments, the compositions and methods described herein can be used to produce the genetic element of an anellovector comprising a proteinaceous exterior comprising a polypeptide (e.g., a synthetic polypeptide, e.g., an ORF1 molecule) comprising (e.g., in series):
(i) a first region comprising an arginine-rich region, e.g., a sequence of at least about 40 amino acids comprising at least 60%, 70%, or 80% basic residues (e.g., arginine, lysine, or a combination thereof),
(ii) a second region comprising a jelly-roll domain, e.g., a sequence comprising at least 6 beta strands,
(iii) a third region comprising an N22 domain sequence described herein,
(iv) a fourth region comprising an Anellovirus ORF1 C-terminal domain (CTD) sequence described herein, and
(v) optionally wherein the polypeptide has an amino acid sequence having less than 100%, 99%, 98%, 95%, 90%, 85%, 80% sequence identity to a wild type Anellovirus ORF1 protein, e.g., as described herein.

In an aspect, the invention features an isolated nucleic acid molecule (e.g., a nucleic acid construct, e.g., a bacmid or donor vector) comprising the sequence of a genetic element comprising a promoter element operably linked to a sequence encoding an effector, e.g., a payload, and an exterior protein binding sequence. In some embodiments, the exterior protein binding sequence includes a sequence at least 75% (at least 80%, 85%, 90%, 95%, 97%, 100%) identical to a 5′UTR sequence of an Anellovirus, e.g., as disclosed herein. In embodiments, the genetic element is a single-stranded DNA, is circular, integrates at a frequency of less than about 0.001%, 0.005%, 0.01%, 0.05%, 0.1%, 0.5%, 1%, 1.5%, or 2% of the genetic element that enters the cell, and/or integrates into the genome of a target cell at less than 1, 2, 3, 4, 5, 6, 7, 8, 9, 10, 15, 20, 25, or 30 copies per genome or integrates at a frequency of less than about 0.001%, 0.005%, 0.01%, 0.05%, 0.1%, 0.5%, 1%, 1.5%, or 2% of the genetic element that enters the cell. In some embodiments, integration frequency is determined by quantitative gel purification assay of genomic DNA separated from free vector, e.g., as described in Wang et al. (2004, Gene Therapy 11: 711-721, incorporated herein by reference in its entirety). In embodiments, the effector does not originate from TTV and is not an SV40-miR-S1. In embodiments, the nucleic acid molecule does not comprise the polynucleotide sequence of TTMV-LY2. In embodiments, the promoter element is capable of directing expression of the effector in a eukaryotic (e.g., mammalian, e.g., human) cell.

In some embodiments, the nucleic acid molecule comprises a backbone region suitable for replication of the nucleic acid construct in insect cells (e.g., a baculovirus backbone region, e.g., comprising one or more baculovirus elements). In some embodiments, the nucleic acid molecule is a bacmid. In some embodiments, the nucleic acid molecule comprises a baculovirus genome or a functional fragment thereof. In some embodiments, the backbone region is suitable for replication of the nucleic acid molecule in bacterial cells (e.g., a donor vector, e.g., as described herein).

In some embodiments, the nucleic acid molecule is circular. In some embodiments, the nucleic acid molecule is linear. In some embodiments, a nucleic acid molecule described herein comprises one or more modified nucleotides (e.g., a base modification, sugar modification, or backbone modification).

In some embodiments, the nucleic acid molecule comprises a sequence encoding an ORF1 molecule (e.g., an Anellovirus ORF1 protein, e.g., as described herein). In some embodiments, the nucleic acid molecule comprises a sequence encoding an ORF2 molecule (e.g., an Anellovirus ORF2 protein, e.g., as described herein). In some embodiments, the nucleic acid molecule comprises a sequence encoding an ORF3 molecule (e.g., an Anellovirus ORF3 protein, e.g., as described herein). In an aspect, the invention features a genetic element comprising one, two, or three of: (i) a promoter element and a sequence encoding an effector, e.g., an exogenous or endogenous effector; (ii) at least 72 contiguous nucleotides (e.g., at least 72, 73, 74, 75, 76, 77, 78, 79, 80, 90, 100, or 150 nucleotides) having at least 75% (e.g., at least 75, 76, 77, 78, 79, 80, 90, 91, 92, 93, 94, 95, 96, 97, 98, 99, or 100%) sequence identity to a wild-type Anellovirus sequence; or at least 100 (e.g., at least 300, 500, 1000, 1500) contiguous nucleotides having at least 72% (e.g., at least 72, 73, 74, 75, 76, 77, 78, 79, 80, 90, 91, 92, 93, 94, 95, 96, 97, 98, 99, or 100%) sequence identity to a wild-type Anellovirus sequence; and (iii) a protein binding sequence, e.g., an exterior protein binding sequence, and wherein the nucleic acid construct is a single-stranded DNA; and wherein the nucleic acid construct is circular, integrates at a frequency of less than about 0.001%, 0.005%, 0.01%, 0.05%, 0.1%, 0.5%, 1%, 1.5%, or 2% of the genetic element that enters the cell, and/or integrates into the genome of a target cell at less than 1, 2, 3, 4, 5, 6, 7, 8, 9, 10, 15, 20, 25, or 30 copies per genome In some embodiments, a genetic element encoding an effector (e.g., an exogenous or endogenous effector, e.g., as described herein) is codon optimized. In some embodiments, the genetic element is circular. In some embodiments, the genetic element is linear. In some embodiments, a genetic element described herein comprises one or more modified nucleotides (e.g., a base modification, sugar modification, or backbone modification). In some embodiments, the genetic element comprises a sequence encoding an ORF1 molecule (e.g., an Anellovirus ORF1 protein, e.g., as described herein). In some embodiments, the genetic element comprises a sequence encoding an ORF2 molecule (e.g., an Anellovirus ORF2 protein, e.g., as described herein). In some embodiments, the genetic element comprises a sequence encoding an ORF3 molecule (e.g., an Anellovirus ORF3 protein, e.g., as described herein).

In an aspect, the invention features a host cell comprising a bacmid or donor vector, e.g., as described herein. In some embodiments, fa bacmid is a nucleic acid construct comprising a backbone region suitable for replication of the nucleic acid construct in insect cells (e.g., a baculovirus backbone region, e.g., comprising a baculovirus genome or elements thereof). In some embodiments, the backbone region of the bacmid is suitable for replication of the nucleic acid construct in bacterial cells (e.g., E. coli, e.g., DH 10Bac cells). In some embodiments, a donor vector is a nucleic acid construct (e.g., a plasmid) comprising a backbone region suitable for replication of the nucleic acid construct in bacterial cells (e.g., E. coli, e.g., DH 10Bac cells). In some embodiments, the host cell is an insect cell (e.g., an Sf9 cell), e.g., comprising the bacmid. In some embodiments, the host cell is a bacterial cell (e.g., an E. coli cell, e.g., a DH 10Bac cell), e.g., comprising the donor vector and/or the bacmid. In some embodiments, the host cell comprises: (a) a bacmid and/or donor cell comprising a sequence encoding one or more of an ORF1 molecule, an ORF2 molecule, or an ORF3 molecule (e.g., a sequence encoding an Anellovirus ORF1 polypeptide described herein); and (b) a genetic element (e.g., comprised in the same bacmid and/or donor vector, or comprised in a second bacmid and/or donor vector), wherein the genetic element comprises (i) a promoter element operably linked to a nucleic acid sequence (e.g., a DNA sequence) encoding an effector (e.g., an exogenous effector or an endogenous effector) and (ii) a protein binding sequence that binds the polypeptide of (a), wherein optionally the genetic element does not encode an ORF1 polypeptide (e.g., an ORF1 protein). For example, the host cell comprises (a) and (b) either in cis (both part of the same nucleic acid molecule) or in trans (each part of a different nucleic acid molecule). In embodiments, the genetic element of (b) is a circular, single-stranded DNA. In some embodiments, the host cell is a manufacturing cell line, e.g., as described herein. In some embodiments, the host cell is adherent or in suspension, or both. In some embodiments, the host cell or helper cell is grown in a microcarrier. In some embodiments, the host cell or helper cell is compatible with cGMP manufacturing practices. In some embodiments, the host cell or helper cell is grown in a medium suitable for promoting cell growth. In certain embodiments, once the host cell or helper cell has grown sufficiently (e.g., to an appropriate cell density), the medium may be exchanged with a medium suitable for production of anellovectors by the host cell or helper cell.

In an aspect, the invention features a pharmaceutical composition comprising an anellovector (e.g., a synthetic anellovector) as described herein. In embodiments, the pharmaceutical composition further comprises a pharmaceutically acceptable carrier or excipient. In embodiments, the pharmaceutical composition comprises a unit dose comprising about 10⁵-10¹⁴ (e.g., about 10⁶-10¹³, 10⁷-10¹², 10⁸-10¹¹, or 10⁹-10¹⁰) genome equivalents of the anellovector per kilogram of a target subject. In some embodiments, the pharmaceutical composition comprising the preparation will be stable over an acceptable period of time and temperature, and/or be compatible with the desired route of administration and/or any devices this route of administration will require, e.g., needles or syringes. In some embodiments, the pharmaceutical composition is formulated for administration as a single dose or multiple doses. In some embodiments, the pharmaceutical composition is formulated at the site of administration, e.g., by a healthcare professional. In some embodiments, the pharmaceutical composition comprises a desired concentration of anellovector genomes or genomic equivalents (e.g., as defined by number of genomes per volume).

In an aspect, the invention features a method of treating a disease or disorder in a subject, the method comprising administering to the subject an anellovector, e.g., a synthetic anellovector, e.g., as described herein.

In an aspect, the invention features a method of delivering an effector or payload (e.g., an endogenous or exogenous effector) to a cell, tissue or subject, the method comprising administering to the subject an anellovector, e.g., a synthetic anellovector, e.g., as described herein, wherein the anellovector comprises a nucleic acid sequence encoding the effector. In embodiments, the payload is a nucleic acid.

In embodiments, the payload is a polypeptide.

In an aspect, the invention features a method of delivering an anellovector to a cell, comprising contacting the anellovector, e.g., a synthetic anellovector, e.g., as described herein, with a cell, e.g., a eukaryotic cell, e.g., a mammalian cell, e.g., in vivo or ex vivo.

In an aspect, the invention features a method of making an Anellovirus ORF molecule. The method includes:
(a) providing a host cell (e.g., an insect cell, e.g., an Sf9 cell) comprising a nucleic acid molecule comprising:
   (i) a promoter operably linked to a sequence encoding an Anellovirus ORF molecule (e.g., an ORF1, ORF2, ORF2/2, ORF2/3, ORF1/1, and/or ORF1/2 molecule); and
   (ii) a backbone region suitable for replication of the nucleic acid construct in insect cells (e.g., a Baculovirus backbone region), optionally wherein the backbone region is also suitable for replication of the nucleic acid construct in bacterial cells; and
(b) incubating the host cell under conditions suitable for expression of the Anellovirus ORF molecule, thereby producing the Anellovirus ORF molecule.

In some embodiments, the host cell is an insect cell (e.g., an Sf9 cell). In some embodiments, the host cell is a bacterial cell (e.g., an E. coli cell, e.g., a DH 10Bac cell).

For example, the host cell provided in this method of manufacturing comprises (a) a nucleic acid molecule (e.g., a bacmid or donor vector) comprising a sequence encoding an Anellovirus ORF1 polypeptide described herein; and (b) a nucleic acid construct (e.g., a bacmid) capable of producing a genetic element (e.g., comprising a genetic element sequence and/or genetic element region, e.g., as described herein), e.g., wherein the genetic element comprises (i) a promoter element operably linked to a nucleic acid sequence (e.g., a DNA sequence) encoding an effector (e.g., an exogenous effector or an endogenous effector) and (i) a protein binding sequence (e.g, packaging sequence) that binds the polypeptide of (a), wherein the host cell comprises (a) and (b) either in cis or in trans. In embodiments, the genetic element of (b) is circular, single-stranded DNA. In some embodiments, the host cell is a manufacturing cell line

In some embodiments, the components of the anellovector are introduced into the host cell at the time of production (e.g., by transient transfection). In some embodiments, the host cell stably expresses the components of the anellovector (e.g., wherein one or more nucleic acids encoding the components of the anellovector are introduced into the host cell, or a progenitor thereof, e.g., by stable transfection).

In an aspect, the invention features a method of manufacturing an anellovector composition, comprising: a) providing a plurality of anellovectors described herein, or a preparation of anellovectors described herein; and b) formulating the anellovectors or preparation thereof, e.g., as a pharmaceutical composition suitable for administration to a subject.

In an aspect, the invention features a method of making a host cell, e.g., a first host cell or a producer cell (e.g., as shown in FIG. 12 of PCT/US19/65995) (e.g., an insect cell, e.g., an Sf9 cell), e.g., a population of first host cells, comprising an anellovector, the method comprising introducing a nucleic acid construct capable of producing a genetic element (e.g., a bacmid, e.g., as described herein) to a host cell and culturing the host cell under conditions suitable for production of the anellovector. In embodiments, the method further comprises introducing a helper, e.g., a helper virus, to the host cell. In embodiments, the introducing comprises transfection (e.g., chemical transfection) or electroporation of the host cell with the anellovector.

In an aspect, the invention features a method of making an anellovector, comprising providing a host cell, e.g., a first host cell or producer cell (e.g., as shown in FIG. 12 of PCT/US19/65995) (e.g., an insect cell, e.g., an Sf9 cell), comprising an anellovector, e.g., as described herein, and purifying the anellovector from the host cell. In some embodiments, the method further comprises, prior to the providing step, contacting the host cell with a nucleic acid construct (e.g., a bacmid) or an anellovector, e.g., as described herein, and incubating the host cell under conditions suitable for production of the anellovector. In embodiments, the host cell is the first host cell or producer cell described in the above method of making a host cell. In embodiments, purifying the anellovector from the host cell comprises lysing the host cell.

In some embodiments, the method further comprises a second step of contacting the anellovector produced by the first host cell or producer cell with a second host cell, e.g., a permissive cell (e.g., as shown in FIG. 12 of PCT/US19/65995), e.g., a population of second host cells. In some embodiments, the method further comprises incubating the second host cell inder conditions suitable for production of the anellovector. In some embodiments, the method further comprises purifying an anellovector from the second host cell, e.g., thereby producing an anellovector seed population. In embodiments, at least about 2-100-fold more of the anellovector is produced from the population of second host cells than from the population of first host cells. In embodiments, purifying the anellovector from the second host cell comprises lysing the second host cell. In some embodiments, the method further comprises a second step of contacting the anellovector produced by the second host cell with a third host cell, e.g., permissive cells (e.g., as shown in FIG. 12 of PCT/US19/65995), e.g., a population of third host cells. In some embodiments, the method further comprises incubating the third host cell inder conditions suitable for production of the anellovector. In some embodiments, the method further comprises purifying a anellovector from the third host cell, e.g., thereby producing an anellovector stock population. In embodiments, purifying the anellovector from the third host cell comprises lysing the third host cell. In embodiments, at least about 2-100-fold more of the anellovector is produced from the population of third host cells than from the population of second host cells.

In some embodiments, the host cell is grown in a medium suitable for promoting cell growth. In certain embodiments, once the host cell has grown sufficiently (e.g., to an appropriate cell density), the medium may be exchanged with a medium suitable for production of anellovectors by the host cell. In some embodiments, anellovectors produced by a host cell separated from the host cell (e.g., by lysing the host cell) prior to contact with a second host cell. In some embodiments, anellovectors produced by a host cell are contacted with a second host cell without an intervening purification step.

In an aspect, the invention features a method of making a pharmaceutical anellovector preparation. The method comprises (a) making an anellovector preparation as described herein, (b) evaluating the preparation (e.g., a pharmaceutical anellovector preparation, anellovector seed population or the anellovector stock population) for one or more pharmaceutical quality control parameters, e.g., identity, purity, titer, potency (e.g., in genomic equivalents per anellovector particle), and/or the nucleic acid sequence, e.g., from the genetic element comprised by the anellovector, and (c) formulating the preparation for pharmaceutical use of the evaluation meets a predetermined criterion, e.g, meets a pharmaceutical specification. In some embodiments, evaluating identity comprises evaluating (e.g., confirming) the sequence of the genetic element of the anellovector, e.g., the sequence encoding the effector. In some embodiments, evaluating purity comprises evaluating the amount of an impurity, e.g., mycoplasma, endotoxin, host cell nucleic acids (e.g., host cell DNA and/or host cell RNA), animal-derived process impurities (e.g., serum albumin or trypsin), replication-competent agents (RCA), e.g., replication-competent virus or unwanted anellovectors (e.g., an anellovector other than the desired anellovector, e.g., a synthetic anellovector as described herein), free viral capsid protein, adventitious agents, and aggregates. In some embodiments, evaluating titer comprises evaluating the ratio of functional versus non-functional (e.g., infectious vs non-infectious) anellovectors in the preparation (e.g., as evaluated by HPLC). In some embodiments, evaluating potency comprises evaluating the level of anellovector function (e.g., expression and/or function of an effector encoded therein or genomic equivalents) detectable in the preparation.

In embodiments, the formulated preparation is substantially free of pathogens, host cell contaminants or impurities; has a predetermined level of non-infectious particles or a predetermined ratio of particles:infectious units (e.g., <300:1, <200:1, <100:1, or <50:1). In some embodiments, multiple anellovectors can be produced in a single batch. In embodiments, the levels of the anellovectors produced in the batch can be evaluated (e.g., individually or together).

In an aspect, the invention features a host cell comprising:
(i) a first nucleic acid molecule comprising a nucleic acid construct (e.g., bacmid or donor vector) as described herein, and
(ii) optionally, a second nucleic acid molecule (e.g., a bacmid or donor vector) encoding one or more of an amino acid sequence chosen from ORF1, ORF2, ORF2/2, ORF2/3, ORF1/1, or ORF1/2, e.g., as described herein, or an amino acid sequence having at least about 70% (e.g., at least about 70, 80, 90, 95, 96, 97, 98, 99, or 100%) sequence identity thereto.

In an aspect, the invention features a reaction mixture comprising an anellovector described herein and a helper virus, wherein the helper virus comprises a polynucleotide, e.g., encoding an exterior protein, (e.g., an exterior protein capable of binding to the exterior protein binding sequence and, optionally, a lipid envelope), a polynucleotide encoding a replication protein (e.g., a polymerase), or any combination thereof.

In some embodiments, an anellovector (e.g., a synthetic anellovector) is isolated, e.g., isolated from a host cell and/or isolated from other constituents in a solution (e.g., a supernatant). In some embodiments, an anellovector (e.g., a synthetic anellovector) is purified, e.g., from a solution (e.g., a supernatant). In some embodiments, an anellovector is enriched in a solution relative to other constituents in the solution.

In some embodiments of any of the aforesaid anellovectors, compositions or methods, providing an anellovector comprises separating (e.g., harvesting) an anellovector from a composition comprising an anellovector-producing cell, e.g., as described herein. In other embodiments, providing an anellovector comprises obtaining an anellovector or a preparation thereof, e.g., from a third party.

In some embodiments of any of the aforesaid anellovectors, compositions or methods, the genetic element comprises an anellovector genome, e.g., as identified according to the methods described herein. In embodiments, the anellovector genome comprises a TTV-tth8 nucleic acid sequence, e.g., a TTV-tth8 nucleic acid, e.g., having deletions of at least 10%, 20%, 30%, 40%, 50%, 60%, 70%, 80%, 90%, 95%, 99%, or 100% of nucleotides 3436-3707 of the TTV-tth8 nucleic acid sequence. In embodiments, the anellovector genome comprises a TTMV-LY2 nucleic acid sequence, e.g., a TTMV-LY2 nucleic acid sequence, e.g., having deletions of at least 10%, 20%, 30%, 40%, 50%, 60%, 70%, 80%, 90%, 95%, 99%, or 100% of nucleotides 574-1371, 1432-2210, 574-2210, and/or 2610-2809 of the TTMV-LY2 nucleic acid sequence. In embodiments, the genetic element is capable of self-replication and/or self-amplification. In embodiments, the genetic element is not capable of self-replication and/or self-amplification. In embodiments, the genetic element is capable of replicating and/or being amplified in trans, e.g., in the presence of a helper, e.g., a helper virus.

Additional features of any of the aforesaid anellovectors, compositions or methods include one or more of the following enumerated embodiments.

Those skilled in the art will recognize, or be able to ascertain using no more than routine experimentation, many equivalents to the specific embodiments of the invention described herein. Such equivalents are intended to be encompassed by the following enumerated embodiments.

### Enumerated Embodiments

1. A composition comprising
(i) a nucleic acid (e.g., DNA) construct comprising:
   a) a promoter operably linked to a sequence encoding an Anellovirus ORF molecule (e.g., an ORF1, ORF2, ORF2/2, ORF2/3, ORF1/1, and/or ORF1/2 molecule); and
   b) a backbone region suitable for replication of the nucleic acid construct in insect cells (e.g., a Baculovirus backbone region), optionally wherein the backbone region is also suitable for replication of the nucleic acid construct in bacterial cells; and
(ii) an Anellovirus genetic element comprising a promoter operably linked to a sequence encoding an exogenous effector.

2. The composition of embodiment 1, wherein the Anellovirus genetic element is part of the nucleic acid construct.

3. The composition of embodiment 1, comprising a second nucleic acid construct comprising the Anellovirus genetic element.

4. A nucleic acid (e.g., DNA) construct comprising:
a) an Anellovirus genetic element region comprising a promoter operably linked to a sequence encoding an exogenous effector; and
b) a backbone region suitable for replication of the nucleic acid construct in insect cells (e.g., a Baculovirus backbone region), optionally wherein the backbone region is also suitable for replication of the nucleic acid construct in bacterial cells.

5. The nucleic acid construct of embodiment 4, further comprising one or more cassettes comprising a promoter operably linked to a sequence encoding an Anellovirus ORF molecule (e.g., an ORF1, ORF2, ORF2/2, ORF2/3, ORF1/1, and/or ORF1/2 molecule).

6. The nucleic acid construct of embodiment 5, wherein the promoter is an Anellovirus promoter or an exogenous promoter (e.g., a polyhedron promoter).

7. The nucleic acid construct of any of the preceding embodiments, wherein the nucleic acid construct is an isolated nucleic acid construct.

8. A bacterial cell comprising the nucleic acid construct or nucleic acid preparation of any of the preceding embodiments.

9. An insect cell comprising the nucleic acid construct or nucleic acid preparation of any of the preceding embodiments.

10. An insect cell comprising an Anellovirus ORF1 molecule comprising an Anellovirus ORF1 Arginine-rich region and an Anellovirus ORF1 C-terminal domain.

11. The insect cell of embodiment 10, wherein the Anellovirus ORF1 molecule comprises an Anellovirus ORF1 jelly-roll domain.

12. The insect cell of embodiment 10 or 11, wherein the Anellovirus ORF1 molecule comprises an Anellovirus ORF1 hypervariable domain.

13. The insect cell of any of embodiments 10-12, wherein the Anellovirus ORF1 molecule comprises an Anellovirus ORF1 N22 domain.

14. The insect cell of any of embodiments 9-13, comprising at least 10, 20, 30, 40, 50, 60, 70, 80, 90, 100, 500, 1000, 10,000, 50,000, or 100,000 copies of the Anellovirus ORF1 molecules.

15. An insect cell comprising the nucleic acid construct of any of embodiments 4-7, further comprising a nucleic acid construct comprising:
a) a sequence encoding an Anellovirus ORF molecule (e.g., an ORF1, ORF2, ORF2/2, ORF2/3, ORF1/1, and/or ORF1/2 molecule); and
b) a backbone region suitable for replication of the nucleic acid construct in insect cells (e.g., a Baculovirus backbone region), optionally wherein the backbone region is also suitable for replication of the nucleic acid construct in bacterial cells.

16. A composition comprising an isolated Anellovirus ORF1 molecule comprising an Anellovirus ORF1 Arginine-rich region and an Anellovirus C-terminal domain, and not comprising detectable levels of an Anellovirus genetic element.

17. The composition of embodiment 16, wherein the Anellovirus ORF1 molecule comprises an Anellovirus ORF1 jelly-roll domain.

18. The composition of any of embodiments 16-17, wherein the Anellovirus ORF1 molecule comprises an Anellovirus ORF1 hypervariable domain.

19. The composition of any of embodiments 16-18, wherein the Anellovirus ORF1 molecule comprises an Anellovirus ORF1 N22 domain.

20. The composition of any of embodiments 16-19, wherein the Anellovirus ORF1 molecule or a nucleic acid molecule encoding same comprises at least one difference (e.g., a mutation, chemical modification, or epigenetic alteration) relative to a wild-type Anellovirus ORF1 protein (e.g., as described herein), e.g., an insertion, substitution, chemical or enzymatic modification, and/or deletion, e.g., a deletion of a domain (e.g., one or more of an arginine-rich region, jelly-roll domain, HVR, N22, or CTD, e.g., as described herein).

21. A composition comprising an isolated Anellovirus ORF1 molecule having a molecular weight of at least 101 kDa, and not comprising detectable levels of an Anellovirus genetic element.

22. The composition of embodiment 21, wherein the Anellovirus ORF1 molecule or a nucleic acid molecule encoding same comprises at least one difference (e.g., a mutation, chemical modification, or epigenetic alteration) relative to a wild-type Anellovirus ORF1 protein (e.g., as described herein), e.g., an insertion, substitution, chemical or enzymatic modification, and/or deletion, e.g., a deletion of a domain (e.g., one or more of an arginine-rich region, jelly-roll domain, HVR, N22, or CTD, e.g., as described herein).

23. A baculovirus particle comprising the nucleic acid construct of any of the preceding embodiments.

24. A population of baculovirus particles comprising the nucleic acid construct of any of the preceding embodiments.

25. A reaction mixture comprising the population of baculovirus particles of embodiment 24 and a plurality of insect cells.

26. A nucleic acid construct comprising, in order:
a) a first transposase recognition sequence (e.g., a Tn7R sequence);
b) an Anellovirus genetic element region comprising a promoter operably linked to a sequence encoding an exogenous effector;
c) a second transposase recognition sequence (e.g., a Tn7L sequence); and
d) optionally, a backbone region, e.g., wherein the backbone region is suitable for replication of the DNA construct.

27. The nucleic acid construct of embodiment 26, further comprising a sequence encoding an Anellovirus ORF1, ORF2, ORF2/2, ORF2/3, ORF1/1, and/or ORF1/2 molecule.

28. A nucleic acid construct comprising, in order:
a) a first transposase recognition sequence (e.g., a Tn7R sequence);
b) a promoter operably linked to a sequence encoding an Anellovirus ORF molecule (e.g., an ORF1, ORF2, ORF2/2, ORF2/3, ORF1/1, and/or ORF1/2 molecule);
c) a second transposase recognition sequence (e.g., a Tn7L sequence); and
d) optionally, a backbone region, e.g., wherein the backbone region is suitable for replication of the DNA construct.

29. The nucleic acid construct of embodiment 28, further comprising an Anellovirus genetic element region comprising a promoter operably linked to a sequence encoding an exogenous effector.

30. A bacterial cell comprising the nucleic acid construct of any of embodiments 26-29.

31. A reaction mixture comprising the nucleic acid construct of any of embodiments 26-30 and a bacterial cell (e.g., an E. coli cell).

32. An insect cell comprising an Anellovirus genetic element.

33. An insect cell comprising an Anellovirus genetic element comprising a promoter operably linked to a sequence encoding an exogenous effector.

34. The insect cell of embodiment 32 or 33, which further comprises one or more ORF molecule (e.g., an ORF1, ORF2, ORF2/2, ORF2/3, ORF1/1, and/or ORF1/2 molecule).

35. The insect cell of embodiment 34, wherein the ORF1 molecule encloses the Anellovirus genetic element.

36. A method of making a baculovirus particle, the method comprising:
(i) providing an insect cell (e.g., an Sf9 cell) that comprises the nucleic acid construct of any of the preceding embodiments; and
(ii) incubating the insect cell under conditions suitable for production of a baculovirus particle comprising the nucleic acid construct or a copy thereof;
thereby making a baculovirus particle.

37. The method of embodiment 36, wherein providing the insect cell comprising the nucleic acid construct comprises introducing (e.g., transfecting or infecting) the nucleic acid construct into the insect cell.

38. The method of embodiment 36 or 37, further comprising isolating the baculovirus particle.

39. The method of any of embodiments 36-38, further comprising contacting the baculovirus particle with a second insect cell.

40. The method of embodiment 39, further comprising incubating the second insect cell under conditions suitable for production of a second baculovirus particle comprising the nucleic acid construct or a copy thereof.

41. A method of introducing a nucleic acid construct of any of the preceding embodiments into an insect cell, the method comprising contacting an insect cell with a baculovirus particle comprising the nucleic acid construct.

42. A method of making a baculovirus particle, the method comprising:
(i) introducing (e.g., transfecting) the nucleic construct of any of the preceding embodiments into a first insect cell;
(ii) incubating the insect cell under conditions suitable for production of a first baculovirus particle comprising the nucleic acid construct or a copy thereof;
(iii) isolating the first baculovirus particle;
(iv) contacting the first baculovirus particle with a second insect cell; and
(v) incubating the second insect cell under conditions suitable for production of a second baculovirus particle comprising the nucleic acid construct or a copy thereof.

43. A method of making an insect cell comprising a bacmid construct comprising an Anellovirus genetic element, the method comprising:
(i) providing an insect cell (e.g., an Sf9 cell) that comprises a nucleic acid construct of any of the preceding embodiments and an empty bacmid construct; and
(ii) incubating the insect cell under conditions suitable for recombination between the nucleic acid construct and the empty bacmid construct;
thereby making an insect cell comprising a bacmid construct comprising an Anellovirus genetic element.

44. A method of making an insect cell comprising a bacmid construct comprising an Anellovirus genetic element, the method comprising:
(i) providing an insect cell (e.g., an Sf9 cell) that comprises an empty bacmid construct and a nucleic acid construct comprising, in order:
   a) a first transposase recognition sequence (e.g., a Tn7R sequence);
   b) an Anellovirus genetic element region comprising a promoter operably linked to a sequence encoding an exogenous effector;
   c) a second transposase recognition sequence (e.g., a Tn7L sequence); and
   d) optionally, a backbone region, e.g., wherein the backbone region suitable for replication of the DNA construct; and
(ii) incubating the insect cell under conditions suitable for recombination between the nucleic acid construct and the empty bacmid construct;
thereby making an insect cell comprising a bacmid construct comprising an Anellovirus genetic element.

45. A method of making an Anellovector, the method comprising:
(i) providing an insect cell comprising:
   a) an Anellovirus genetic element comprising a promoter operably linked to a sequence encoding an exogenous effector, and
   b) an Anellovirus ORF1 molecule;
(ii) incubating the insect cell under conditions suitable for enclosure of the Anellovirus genetic element in a proteinaceous exterior comprising the Anellovirus ORF1 molecule.

46. The method of embodiment 45, wherein the method further comprises introducing (e.g., transfecting) the Anellovirus genetic element into the insect cell.

47. The method of embodiment 46, wherein the insect cell comprises the ORF1 molecule prior to the introducing.

48. The method of any of embodiments 45-47, wherein the insect cell comprises a nucleic acid construct (e.g., a bacmid) encoding the ORF1 molecule.

49. The method of embodiment 48, wherein the ORF1 molecule is expressed from the nucleic acid construct encoding the ORF1 molecule.

50. The method of any of embodiments 45-49, further comprising, prior to step (i), introducing into the insect cell a nucleic acid construct comprising the Anellovirus genetic element and a backbone region suitable for replication of the nucleic acid construct in insect cells (e.g., a Baculovirus backbone region), optionally wherein the backbone region is also suitable for replication of the nucleic acid construct in bacterial cells.

51. The method of any of embodiments 45-50, further comprising, prior to step (i), introducing into the insect cell a nucleic acid construct comprising a sequence encoding the Anellovirus ORF1 molecule and a backbone region suitable for replication of the nucleic acid construct in insect cells (e.g., a Baculovirus backbone region), optionally wherein the backbone region is also suitable for replication of the nucleic acid construct in bacterial cells.

52. The method of any of embodiments 45-51, further comprising, prior to step (i), introducing into the insect cell a nucleic acid construct comprising:
a sequence encoding an Anellovirus ORF2, ORF2/2, ORF2/3, ORF1/1, and/or ORF1/2 molecule, and
a backbone region suitable for replication of the nucleic acid construct in insect cells (e.g., a Baculovirus backbone region), optionally wherein the backbone region is also suitable for replication of the nucleic acid construct in bacterial cells.

53. The method of any of embodiments 45-52, wherein at least 1, 2, 3, 4, 5, 10, 20, 30, 40, 50, 60, 70, 80, 90, 100, 500, 1000, 5000, 10,000, 50,000, or 100,000 Anellovectors are produced.

54. The method of any of embodiments 45-53, further comprising isolating the Anellovectors from the insect cells.

55. The method of any of embodiments 45-54, wherein the insect cell is compatible with cGMP manufacturing practices.

56. A method of making two or more different Anellovirus ORF molecules, the method comprising:
(i) providing an insect cell comprising a nucleic acid construct encoding two or more different Anellovirus ORF molecules (e.g., two or more of an ORF1, ORF2, ORF2/2, ORF2/3, ORF1/1, and/or ORF1/2 molecule);
(ii) incubating the insect cell under conditions suitable for expression of the two or more different Anellovirus ORF molecules.

57. The method of embodiment 56, wherein the Anellovirus genetic element comprises sequences encoding all of an ORF1, ORF2, ORF2/2, ORF2/3, ORF1/1, and/or ORF1/2 molecule.

58. The method of embodiment 56, wherein the Anellovirus genetic element comprises a full length Anellovirus genome (e.g., as listed in any of Tables A1, B1, or C1), optionally wherein the full length Anellovirus genome further comprises a sequence encoding an exogenous effector.

59. The method of embodiment 56, wherein the Anellovirus genetic element comprises a full length Anellovirus coding region.

60. The method of embodiment 56, further comprising incubating the insect cell under conditions suitable for secretion of the Anellovirus ORF molecule.

61. The method of embodiment 56, further isolating the Anellovirus ORF molecule from the insect cell.

62. The method of embodiment 61, wherein the isolating step comprises lysing the insect cell.

63. The method of any of embodiments 56-62, wherein the Anellovirus ORF comprises an Anellovirus ORF1 molecule.

64. A method of making an Anellovirus ORF1 molecule, the method comprising:
(i) providing an insect cell comprising a nucleic acid construct encoding an Anellovirus ORF1 molecule, wherein:
   (a) the Anellovirus ORF1 molecule has a molecular weight of at least 101 kDa,
   (b) the Anellovirus ORF1 molecule is a full-length Anellovirus ORF1 protein,
   (c) a plurality of the Anellovirus ORF1 molecules, when in the presence of an Anellovirus genetic element, enclose the Anellovirus genetic element,
   (d) the Anellovirus ORF1 molecule is not a TTV ORF1 protein,
   (e) the Anellovirus ORF1 molecule is a Betatorquevirus or Gammatorquevirus ORF1 molecule; or
   (f) the Anellovirus ORF1 molecule comprises an Anellovirus ORF1 Arginine-rich region and an Anellovirus C-terminal domain;
(ii) incubating the insect cell under conditions suitable for expression of the Anellovirus ORF1 molecule.

65. The method of embodiment 64, further comprising incubating the insect cell under conditions suitable for secretion of the Anellovirus ORF1 molecule.

66. The method of embodiment 64, further isolating the Anellovirus ORF1 molecule from the insect cell.

67. The method of embodiment 66, wherein the isolating step comprises lysing the insect cell.

68. The method of any of the preceding embodiments, wherein the incubation step produces an amount of the Anellovirus ORF1 molecule detectable by Western blot, e.g., as described in Example 1 or 2.

69. A baculovirus particle produced according to the method of any of embodiments 36-42.

70. A composition comprising a plurality of baculovirus particles produced according to the method of any of embodiments 36-42.

71. An Anellovector produced according to the method of any of embodiments 45-68.

72. A composition comprising a plurality of Anellovectors produced according to the method of any of embodiments 45-68.

73. The composition of embodiment 72, which has a has a predetermined level of non-infectious particles or a predetermined ratio of non-infectious particles: infectious units (e.g., less than 300:1, 200:1, 100:1, or 50:1).

74. An Anellovirus ORF molecule produced according to the method of any of embodiments 45-68.

75. A composition comprising a plurality of Anellovirus ORF molecules produced according to the method of any of embodiments 45-68.

76. The composition, Anellovector, or Anellovirus ORF molecule of any of embodiments 69-75, which meets a pharmaceutical or good manufacturing practices (GMP) standard.

77. The composition, Anellovector, or Anellovirus ORF molecule of any of embodiments 69-76, which was made according to a good manufacturing practices (GMP) standard.

78. The composition, Anellovector, or Anellovirus ORF molecule of any of embodiments 69-77, which has a pathogen level below a predetermined reference value, e.g., is substantially free of pathogens.

79. The composition, Anellovector, or Anellovirus ORF molecule of any of embodiments 69-78, which has a contaminant level below a predetermined reference value, e.g., is substantially free of contaminants.

80. The composition, Anellovector, or Anellovirus ORF molecule of any of embodiments 69-79, which is substantially non-immunogenic.

81. A method of treating a subject, the method comprising administering to the subject an Anellovector of embodiment 71.

82. The nucleic acid construct, cell, composition, baculovirus particle, population, reaction mixture, Anellovector, Anellovirus ORF molecule, or method of any of the preceding embodiments, wherein the Anellovirus is not a Torque teno virus (TTV).

83. The nucleic acid construct, cell, composition, baculovirus particle, population, reaction mixture, Anellovector, Anellovirus ORF molecule, or method of any of the preceding embodiments, wherein the insect cell is a Lepidoptera cell.

84. The nucleic acid construct, cell, composition, baculovirus particle, population, reaction mixture, Anellovector, Anellovirus ORF molecule, or method of any of the preceding embodiments, wherein the insect cell is a Noctuidae cell.

85. The nucleic acid construct, cell, composition, baculovirus particle, population, reaction mixture, Anellovector, Anellovirus ORF molecule, or method of any of the preceding embodiments, wherein the insect cell is a Noctuidae cell.

86. The nucleic acid construct, cell, composition, baculovirus particle, population, reaction mixture, Anellovector, Anellovirus ORF molecule, or method of any of the preceding embodiments, wherein the insect cell is a Spodoptera cell.

87. The nucleic acid construct, cell, composition, baculovirus particle, population, reaction mixture, Anellovector, Anellovirus ORF molecule, or method of any of the preceding embodiments, wherein the insect cell is an S. frugiperda cell.

88. The nucleic acid construct, cell, composition, baculovirus particle, population, reaction mixture, Anellovector, Anellovirus ORF molecule, or method of any of the preceding embodiments, wherein the insect cell is an Sf9 cell.

89. The nucleic acid construct, cell, composition, baculovirus particle, population, reaction mixture, Anellovector, Anellovirus ORF molecule, or method of any of the preceding embodiments, wherein the Anellovirus ORF1 has a molecular weight of at least 101 kDa.

90. The nucleic acid construct, cell, composition, baculovirus particle, population, reaction mixture, Anellovector, Anellovirus ORF molecule, or method of any of the preceding embodiments, wherein the Anellovirus ORF1 molecule is a full-length Anellovirus ORF1 protein.

91. The nucleic acid construct, cell, composition, baculovirus particle, population, reaction mixture, Anellovector, Anellovirus ORF molecule, or method of any of the preceding embodiments, wherein a plurality of the Anellovirus ORF1 molecules, when in the presence of an Anellovirus genetic element, enclose the Anellovirus genetic element.

92. The nucleic acid construct, cell, composition, baculovirus particle, population, reaction mixture, Anellovector, Anellovirus ORF molecule, or method of any of the preceding embodiments, wherein the Anellovirus ORF molecule is not a TTV ORF protein.

93. The nucleic acid construct, cell, composition, baculovirus particle, population, reaction mixture, Anellovector, Anellovirus ORF molecule, or method of any of the preceding embodiments, wherein the Anellovirus ORF1 molecule is not a TTV ORF1 protein.

94. The nucleic acid construct, cell, composition, baculovirus particle, population, reaction mixture, Anellovector, Anellovirus ORF molecule, or method of any of the preceding embodiments, wherein the Anellovirus ORF molecule is a Betatorquevirus or Gammatorquevirus ORF1 molecule.

95. The nucleic acid construct, cell, composition, baculovirus particle, population, reaction mixture, Anellovector, Anellovirus ORF molecule, or method of any of the preceding embodiments, wherein the Anellovirus ORF1 molecule is a Betatorquevirus or Gammatorquevirus ORF1 molecule.

96. The nucleic acid construct, cell, composition, baculovirus particle, population, reaction mixture, Anellovector, Anellovirus ORF molecule, or method of any of the preceding embodiments, wherein the Anellovirus ORF1 molecule comprises an Anellovirus ORF1 Arginine-rich region and an Anellovirus C-terminal domain.

97. The nucleic acid construct, cell, composition, baculovirus particle, population, reaction mixture, Anellovector, Anellovirus ORF molecule, or method of any of the preceding embodiments, wherein the genetic element or genetic element region comprises:
a) a first, optionally mutant, Anellovirus genome comprising a sequence encoding an exogenous effector;
b) a second Anellovirus genome or fragment thereof, placed in tandem with the first Anellovirus genome; and
c) optionally, a spacer sequence situated between (a) and (b).

98. The nucleic acid construct, cell, composition, baculovirus particle, population, reaction mixture, Anellovector, Anellovirus ORF molecule, or method of any of the preceding embodiments, wherein the genetic element or genetic element region comprises:
a) a first Anellovirus genetic element region comprising a sequence encoding an exogenous effector;
b) a second Anellovirus genetic element region or fragment thereof; and
c) optionally, a spacer sequence situated between (a) and (b).

99. The nucleic acid construct, cell, composition, baculovirus particle, population, reaction mixture, Anellovector, Anellovirus ORF molecule, or method of any of the preceding embodiments, wherein the genetic element is circular (e.g., wherein the genetic element was generated by in vitro circularization), e.g., as described in Example 5.

100. The nucleic acid construct, cell, composition, baculovirus particle, population, reaction mixture, Anellovector, Anellovirus ORF molecule, or method of any of the preceding embodiments, wherein the arginine rich region of the Anellovirus ORF1 molecule is deleted or truncated (e.g., by at least 5, 10, 15, 20, 25, 30, 40, 50, 60, or 65 amino acid acids).

101. The nucleic acid construct, cell, composition, baculovirus particle, population, reaction mixture, Anellovector, Anellovirus ORF molecule, or method of any of the preceding embodiments, wherein the Anellovirus ORF molecules are isolated, purified, or enriched by isopycnic centrifugation, e.g., as described in Example 6.

102. The nucleic acid construct, cell, composition, baculovirus particle, population, reaction mixture, Anellovector, Anellovirus ORF molecule, or method of any of the preceding embodiments, wherein complexes each comprising an Anellovirus ORF1 molecule and a genetic element are isolated, purified, or enriched by isopycnic centrifugation, e.g., as described in Example 6.

103. The nucleic acid construct, cell, composition, baculovirus particle, population, reaction mixture, Anellovector, Anellovirus ORF molecule, or method of any of the preceding embodiments, wherein the sequence encoding an Anellovirus ORF molecule (e.g., an ORF1 molecule) is codon-optimized for expression in an animal cell (e.g., an insect cell).

104. A method of making an Anellovirus ORF molecule (e.g., an ORF1, ORF2, ORF2/2, ORF2/3, ORF1/1, and/or ORF1/2 molecule), the method comprising:
(i) providing an insect cell (e.g., an Sf9 cell) comprising a nucleic acid construct encoding the Anellovirus ORF molecule;
(ii) incubating the insect cell under conditions suitable for expression of a plurality of the Anellovirus ORF molecules; and
(iii) optionally isolating, purifying, and/or enriching the plurality of Anellovirus ORF molecules from the insect cell or other components or constituents thereof;
thereby making the Anellovirus ORF molecule.

105. The method of embodiment 104, wherein the Anellovirus ORF molecule is fused to a marker (e.g., a His tag), e.g., at its N-terminal end or at its C-terminal end (e.g., as described in Table X and/or Example 1).

106. The method of any of embodiments 104-105, wherein the insect cell further comprises a nucleic acid construct encoding one or more additional Anellovirus ORF molecules (e.g., one or more of an ORF1, ORF2, ORF2/2, ORF2/3, ORF1/1, and/or ORF1/2 molecule), and wherein the method further comprises:
incubating the insect cell under conditions suitable for expression of a plurality of the one or more additional Anellovirus ORF molecules, e.g., prior to, concurrently with, or subsequent to step (ii); and
optionally isolating, purifying, and/or enriching the plurality of the one or more additional Anellovirus ORF molecules from the insect cell or other components or constituents thereof, e.g., prior to, concurrently with, or subsequent to step (iii).

107. The method of embodiment 106, wherein the nucleic acid construct encoding the one or more additional Anellovirus ORF molecules is the same as the nucleic acid construct of (i).

108. The method of embodiment 107, wherein the nucleic acid construct of (i) comprises sequences encoding 2, 3, 4, 5, or all 6 of an Anellovirus ORF1, ORF2, ORF2/2, ORF2/3, ORF1/1, and/or ORF1/2 molecule.

109. The method of embodiment 107, wherein the nucleic acid construct of (i) encodes an Anellovirus ORF1, ORF2, ORF2/2, ORF2/3, ORF1/1, and ORF1/2 molecule.

110. The method of embodiment 107, wherein the nucleic acid construct of (i) comprises the full open reading frame region of an Anellovirus genome.

111. The method of embodiment 106, wherein the nucleic acid construct encoding the one or more additional Anellovirus ORF molecules is different from the nucleic acid construct of (i).

112. The method of any of embodiments 106-111, wherein the Anellovirus ORF molecules are from the same Anellovirus genome.

113. The method of any of embodiments 106-111, wherein the Anellovirus ORF molecules are from a plurality of Anellovirus genomes (e.g., wherein the ORF1 molecule is from one Anellovirus genome and the ORF2 molecule is from a different Anellovirus genome).

114. The method of any of embodiments 106-113, wherein one or more of the Anellovirus ORF molecules are from an Alphatorquevirus (e.g., as listed in Table Y).

115. The method of any of embodiments 106-114, wherein one or more of the Anellovirus ORF molecules are from a Betatorquevirus (e.g., as listed in Table Y).

116. The method of any of embodiments 106-115, wherein one or more of the Anellovirus ORF molecules are from a Gammatorquevirus (e.g., as listed in Table Y).

117. The method of any of embodiments 104-116, wherein the nucleic acid construct or constructs each comprises a promoter (e.g., a promoter controlling expression of one or more of the Anellovirus ORF molecules, e.g., a baculovirus polyhedron promoter).

118. The method of any of embodiments 104-117, further comprising incubating the insect cell under conditions suitable for secretion of the Anellovirus ORF molecules.

119. The method of any of embodiments 104-118, wherein the isolating step comprises lysing the insect cell.

120. The method of any of embodiments 104-119, wherein the incubation step produces an amount of the Anellovirus ORF molecule (e.g., ORF1 molecule) detectable by Western blot, e.g., as described in Example 1 or 2.

121. The method of any of embodiments 104-120, wherein the incubation step produces at least 1, 2, 3, 4, 5, or 6 mg of the Anellovirus ORF1 molecule per 1 L of cell culture (e.g., Sf9 culture).

122. The method of any of the preceding embodiments, wherein the Anellovirus ORF molecules are isolated, purified, or enriched by isopycnic centrifugation.

123. The method of any of the preceding embodiments, wherein the Anellovirus ORF molecule is an Anellovirus ORF1 molecule, and wherein the method further comprises: contacting, in vitro, the isolated, purified, or enriched Anellovirus ORF1 molecule with a genetic element under conditions suitable for enclosure of the genetic element by a proteinaceous exterior comprising the Anellovirus ORF1 molecule, e.g., as described in Example 8.

Other features, objects, and advantages of the invention will be apparent from the description and drawings, and from the claims.

Unless otherwise defined, all technical and scientific terms used herein have the same meaning as commonly understood by one of ordinary skill in the art to which this invention belongs. All publications, patent applications, patents, and other references mentioned herein are incorporated by reference in their entirety. In addition, the materials, methods, and examples are illustrative only and not intended to be limiting.

### BRIEF DESCRIPTION OF THE DRAWINGS

FIG. 1 depicts expression of Ring2 ORF1 with a C-terminal His tag in insect cells.

FIG. 2 depicts expression of Ring1 ORF1 and ORF1/1 with a C-terminal His tag in insect cells.

FIG. 3 depicts expression of Ring2 ORF1 with an N-terminal His-tag, with or without PreScission cleavage sequence, in insect cells.

FIG. 4 depicts expression of Ring1 ORFs 1/1, 1/2, 2, 2/2, and 2/3 as C-terminal His-tagged recombinant proteins in insect cells.

FIG. 5 depicts expression of individual Ring2 ORFs in insect cells. Two exposures of the same blot are shown in the middle and right panels. The left panel shows the structures of Ring2 constructs tested as indicated.

FIG. 6 depicts baculovirus-mediated co-expression of Ring2 ORF1+“FullORF”, ORF1+ORF2, ORF1+ORF2/2, and ORF1+ORF2/3 in insect cells.

FIG. 7 depicts simultaneous co-expression of multiple Ring2 proteins in insect cells using baculovirus.

FIG. 8 depicts expression of ORFs from Anellovirus genome delivered into insect cells by baculovirus and by transfection.

FIG. 9 shows that expression of Ring1 ORF2 is independent of the polyhedron promoter (arrow labeled pH) in Sf9 cells.

FIG. 10 depicts co-delivery of Ring2 ORF1-His and Ring2 genomic DNA into Sf9 cells, followed by incubation and fractionation on a CsCl linear density gradient. An anti-His tag Western blot of fractions is shown at the top of the figure, as well as a qPCR assay of each fraction. Bottom panels show transmission electron microscopy images of two individual fractions and a pool of fractions, as indicated by boxes on the Western blot. The inset in the middle panel is a zoomed-in view showing proteasome-like structures.

FIG. 11 depicts characterization of Sf9 isopycnic fractions by immunogold electron microscopy.

FIG. 12 depicts expression of ORF1 from additional Anellovirus strains.

FIG. 13 depicts a schematic of a kanamycin vector encoding the LY1 strain of TTMiniV (“Anellovector 1”).

FIG. 14 depicts a schematic of a kanamycin vector encoding the LY2 strain of TTMiniV (“Anellovector 2”).

FIG. 15 depicts transfection efficiency of synthetic anellovectors in 293T and A549 cells.

FIGS. 16A and 16B depict quantitative PCR results that illustrate successful infection of 293T cells by synthetic anellovectors.

FIGS. 17A and 17B depict quantitative PCR results that illustrate successful infection of A549 cells by synthetic anellovectors.

FIGS. 18A and 18B depict quantitative PCR results that illustrate successful infection of Raji cells by synthetic anellovectors.

FIGS. 19A and 19B depict quantitative PCR results that illustrate successful infection of Jurkat cells by synthetic anellovectors.

FIGS. 20A and 20B depict quantitative PCR results that illustrate successful infection of Chang cells by synthetic anellovectors.

FIG. 21 is a schematic showing an exemplary workflow for production of anellovectors (e.g., replication-competent or replication-deficient anellovectors as described herein).

FIG. 22 is a graph showing fold change in miR-625 expression in HEK293T cells transfected with the indicated plasmid.

FIG. 23 is a diagram showing infection of Raji B cells with anellovectors encoding a miRNA targeting n-myc interacting protein (NMI). Shown is quantification of genome equivalents of anellovectors detected after infection of Raji B cells (arrow) or control cells with NMI miRNA-encoding anellovectors.

FIG. 24 is a diagram showing infection of Raji B cells with anellovectors encoding a miRNA targeting n-myc interacting protein (NMI). The Western blot shows that anellovectors encoding the miRNA against NMI reduced NMI protein expression in Raji B cells, whereas Raji B cells infected with anellovectors lacking the miRNA showed comparable NMI protein expression to controls.

FIG. 25 is a series of graphs showing quantification of anellovector particles generated in host cells after infection with an anellovector comprising an endogenous miRNA-encoding sequence and a corresponding anellovector in which the endogenous miRNA-encoding sequence was deleted.

FIGS. 26A-26B are a series of diagrams showing constructs used to produce anellovectors expressing nano-luciferase (A) and a series of anellovector/plasmid combinations used to transfect cells (B) FIGS. 27A-27C are a series of diagrams showing nano-luciferase expression in mice injected with anellovectors. (A) Nano-luciferase expression in mice at days 0-9 after injection. (B) Nano-luciferase expression in mice injected with various anellovector/plasmid construct combinations, as indicated. (C) Quantification of nano-luciferase luminescence detected in mice after injection. Group A received a TTMV-LY2 vector±nano-luciferase. Group B received a nano-luciferase protein and TTMV-LY2 ORFs.

FIG. 28A is a gel electrophoresis image showing circularization of TTMV-LY2 plasmids pVL46-063 and pVL46-240.

FIG. 28B is a chromatogram showing copy numbers for linear and circular TTMV-LY2 constructs, as determined by size exclusion chromatography (SEC).

FIG. 28C is a schematic showing the domains of an Anellovirus ORF1 molecule and the hypervariable region to be replaced with a hypervariable domain from a different Anellovirus.

FIG. 28D is a schematic showing the domains of ORF1 and the hypervariable region that will be replaced with a protein or peptide of interest (POI) from a non-anellovirus source.

FIG. 29 is a graph showing that anellovectors based on tth8 or LY2, engineered to contain a sequence encoding human erythropoietin (hEpo), could deliver a functional transgene to mammalian cells.

FIGS. 30A and 30B are a series of graphs showing that engineered anellovectors administered to mice were detectable seven days after intravenous injection.

FIG. 31 is a graph showing that hGH mRNA was detected in the cellular fraction of whole blood seven days after intravenous administration of an engineered anellovector encoding hGH.

FIG. 32 is a graph showing the ability of an in vitro circularized (IVC) TTV-tth8 genome (IVC TTV-tth8) compared to a TTV-tth8 genome in a plasmid to yield TTV-tth8 genome copies at the expected density in HEK293T cells.

FIG. 33 is a series of graphs showing the ability of an in vitro circularized (IVC) LY2 genome (WT LY2 IVC) and a wild-type LY2 genome in plasmid (WT LY2 Plasmid) to yield LY2 genome copies at the expected density in Jurkat cells.

The following detailed description of the embodiments of the invention will be better understood when read in conjunction with the appended drawings. For the purpose of illustrating the invention, there are shown in the drawings embodiments that are presently exemplified. It should be understood, however, that the invention is not limited to the precise arrangement and instrumentalities of the embodiments shown in the drawings.

### DETAILED DESCRIPTION OF THE INVENTION

### Definitions

The present invention will be described with respect to particular embodiments and with reference to certain figures but the invention is not limited thereto but only by the claims. Terms as set forth hereinafter are generally to be understood in their common sense unless indicated otherwise.

Where the term “comprising” is used in the present description and claims, it does not exclude other elements. For the purposes of the present invention, the term “consisting of” is considered to be a preferred embodiment of the term “comprising of”. If hereinafter a group is defined to comprise at least a certain number of embodiments, this is to be understood to preferably also disclose a group which consists only of these embodiments.

Where an indefinite or definite article is used when referring to a singular noun, e.g. “a”, “an” or “the”, this includes a plural of that noun unless something else is specifically stated.

The wording “compound, composition, product, etc. for treating, modulating, etc.” is to be understood to refer a compound, composition, product, etc. per se which is suitable for the indicated purposes of treating, modulating, etc. The wording “compound, composition, product, etc. for treating, modulating, etc.” additionally discloses that, as an embodiment, such compound, composition, product, etc. is for use in treating, modulating, etc.

The wording “compound, composition, product, etc. for use in . . . ”, “use of a compound, composition, product, etc in the manufacture of a medicament, pharmaceutical composition, veterinary composition, diagnostic composition, etc. for . . . ”, or “compound, composition, product, etc. for use as a medicament . . . ” indicates that such compounds, compositions, products, etc. are to be used in therapeutic methods which may be practiced on the human or animal body. They are considered as an equivalent disclosure of embodiments and claims pertaining to methods of treatment, etc. If an embodiment or a claim thus refers to “a compound for use in treating a human or animal being suspected to suffer from a disease”, this is considered to be also a disclosure of a “use of a compound in the manufacture of a medicament for treating a human or animal being suspected to suffer from a disease” or a “method of treatment by administering a compound to a human or animal being suspected to suffer from a disease”. The wording “compound, composition, product, etc. for treating, modulating, etc.” is to be understood to refer a compound, composition, product, etc. per se which is suitable for the indicated purposes of treating, modulating, etc.

If hereinafter examples of a term, value, number, etc. are provided in parentheses, this is to be understood as an indication that the examples mentioned in the parentheses can constitute an embodiment. For example, if it is stated that “in embodiments, the nucleic acid molecule comprises a nucleic acid sequence having at least about 70%, 75%, 80%, 85%, 90%, 95%, 96%, 97%, 98%, 99%, or 100% sequence identity to the Anellovirus ORF1-encoding nucleotide sequence of Table 1 (e.g., nucleotides 571-2613 of the nucleic acid sequence of Table 1)”, then some embodiments relate to nucleic acid molecules comprising a nucleic acid sequence having at least about 70%, 75%, 80%, 85%, 90%, 95%, 96%, 97%, 98%, 99%, or 100% sequence identity to nucleotides 571-2613 of the nucleic acid sequence of Table 1.

The term “amplification,” as used herein, refers to replication of a nucleic acid molecule or a portion thereof, to produce one or more additional copies of the nucleic acid molecule or a portion thereof (e.g., a genetic element or a genetic element region). In some embodiments, amplification results in partial replication of a nucleic acid sequence. In some embodiments, amplification occurs via rolling circle replication.

As used herein, the term “anellovector” refers to a vehicle comprising a genetic element, e.g., a circular DNA, enclosed in a proteinaceous exterior, e.g, the genetic element is substantially protected from digestion with DNAse I by a proteinaceous exterior. A “synthetic anellovector,” as used herein, generally refers to an anellovector that is not naturally occurring, e.g., has a sequence that is different relative to a wild-type virus (e.g., a wild-type Anellovirus as described herein). In some embodiments, the synthetic anellovector is engineered or recombinant, e.g., comprises a genetic element that comprises a difference or modification relative to a wild-type viral genome (e.g., a wild-type Anellovirus genome as described herein). In some embodiments, enclosed within a proteinaceous exterior encompasses 100% coverage by a proteinaceous exterior, as well as less than 100% coverage, e.g., 95%, 90%, 85%, 80%, 70%, 60%, 50% or less. For example, gaps or discontinuities (e.g., that render the proteinaceous exterior permeable to water, ions, peptides, or small molecules) may be present in the proteinaceous exterior, so long as the genetic element is retained in the proteinaceous exterior or protected from digestion with DNAse I, e.g., prior to entry into a host cell. In some embodiments, the anellovector is purified, e.g., it is separated from its original source and/or substantially free (>50%, >60%, >70%, >80%, >90%) of other components. In some embodiments, the anellovector is capable of introducing the genetic element into a target cell (e.g., via infection). In some embodiments, the anellovector is an infective synthetic Anellovirus viral particle.

As used herein, the term “antibody molecule” refers to a protein, e.g., an immunoglobulin chain or fragment thereof, comprising at least one immunoglobulin variable domain sequence. The term “antibody molecule” encompasses full-length antibodies and antibody fragments (e.g., scFvs). In some embodiments, an antibody molecule is a multispecific antibody molecule, e.g., the antibody molecule comprises a plurality of immunoglobulin variable domain sequences, wherein a first immunoglobulin variable domain sequence of the plurality has binding specificity for a first epitope and a second immunoglobulin variable domain sequence of the plurality has binding specificity for a second epitope. In embodiments, the multispecific antibody molecule is a bispecific antibody molecule. A bispecific antibody molecule is generally characterized by a first immunoglobulin variable domain sequence which has binding specificity for a first epitope and a second immunoglobulin variable domain sequence that has binding specificity for a second epitope.

The term “backbone” or “backbone region,” as used herein, refers to a region within a nucleic acid molecule (e.g., within a bacmid or donor vector, e.g., as described herein) that comprises one or more elements involved in (e.g., necessary and/or sufficient for) replication and/or maintenance of the nucleic acid molecule in a host cell. In some embodiments, a backbone region, such as a “baculovirus backbone region,” comprises one or more baculoviral elements (e.g., a baculovirus genome or a functional fragment thereof), e.g., suitable for replication of the nucleic acid construct in insect cells (e.g., Sf9 cells). In some embodiments, the backbone further comprises a selectable marker. In some embodiments, a nucleic acid molecule comprises a genetic element region and a backbone region (e.g., a baculovirus backbone region and/or a backbone region suitable for replication in bacterial cells).

The term “bacmid”, as used herein, refers to a nucleic acid molecule comprising sufficient baculovirus backbone elements such that it is suitable for replication in insect cells, and furthermore is suitable for replication in bacterial cells. In some embodiments, the nucleic acid molecule is suitable for replication in bacterial cells (e.g., E. coli cells, e.g., DH 10Bac cells).

As used herein, a nucleic acid “encoding” refers to a nucleic acid sequence encoding an amino acid sequence or a polynucleotide, e.g., an mRNA or functional polynucleotide (e.g., a non-coding RNA, e.g., an siRNA or miRNA).

An “exogenous” agent (e.g., an effector, a nucleic acid (e.g., RNA), a gene, payload, protein) as used herein refers to an agent that is either not comprised by, or not encoded by, a corresponding wild-type virus, e.g., an Anellovirus as described herein. In some embodiments, the exogenous agent does not naturally exist, such as a protein or nucleic acid that has a sequence that is altered (e.g., by insertion, deletion, or substitution) relative to a naturally occurring protein or nucleic acid. In some embodiments, the exogenous agent does not naturally exist in the host cell. In some embodiments, the exogenous agent exists naturally in the host cell but is exogenous to the virus. In some embodiments, the exogenous agent exists naturally in the host cell, but is not present at a desired level or at a desired time.

A “heterologous” agent or element (e.g., an effector, a nucleic acid sequence, an amino acid sequence), as used herein with respect to another agent or element (e.g., an effector, a nucleic acid sequence, an amino acid sequence), refers to agents or elements that are not naturally found together, e.g., in a wild-type virus, e.g., an Anellovirus. In some embodiments, a heterologous nucleic acid sequence may be present in the same nucleic acid as a naturally occurring nucleic acid sequence (e.g., a sequence that is naturally occurring in the Anellovirus). In some embodiments, a heterologous agent or element is exogenous relative to an Anellovirus from which other (e.g., the remainder of) elements of the anellovector are based.

As used herein, the term “genetic element” refers to a nucleic acid molecule that is or can be enclosed within (e.g, protected from DNAse I digestion by) a proteinaceous exterior, e.g., to form an anellovector as described herein. It is understood that the genetic element can be produced as naked DNA and optionally further assembled into a proteinaceous exterior. It is also understood that an anellovector can insert its genetic element into a cell, resulting in the genetic element being present in the cell and the proteinaceous exterior not necessarily entering the cell.

As used herein, “genetic element construct” refers to a nucleic acid construct (e.g., a plasmid, bacmid, donor vector, cosmid, or minicircle) comprising a genetic element sequence, or fragment thereof. In some embodiments, a bacmid or donor vector as described herein is a genetic element construct comprising a genetic element sequence, or fragment thereof.

The term “genetic element region,” as used herein, refers to a region of a construct that comprises the sequence of a genetic element. In some embodiments, the genetic element region comprises a sequence having sufficient identity to a wild-type Anellovirus sequence, or a fragment thereof, to be enclosed by a proteinaceous exterior, thereby forming an anellovector (e.g., a sequence having at least 70%, 75%, 80%, 85%, 90%, 95%, 96%, 97%, 98%, 99%, or 100% sequence identity to the wild-type Anellovirus sequence or fragment thereof). In embodiments, the genetic element region comprises a protein binding sequence, e.g., as described herein (e.g., a 5′ UTR, 3′ UTR, and/or a GC-rich region as described herein, or a sequence having at least 70%, 75%, 80%, 85%, 90%, 95%, 96%, 97%, 98%, 99%, or 100% sequence identity thereto). In some embodiments, the genetic element region can undergo rolling circle replication. In some embodiments, the genetic element comprises a Rep protein binding site. In some embodiments, the genetic element comprises a Rep protein displacement site. In some embodiments, the construct comprising a genetic element region is not enclosed in a proteinaceous exterior, but a genetic element produced from the construct can be enclosed in a proteinaceous exterior. In some embodiments, the construct comprising the genetic element region further comprises a vector backbone (e.g., a bacmid backbone or a donor vector backbone). In some embodiments, the construct (e.g., bacmid) comprises one or more baculovirus elements (e.g., a baculovirus genome, e.g., comprising the genetic element region).

As used herein, the term “mutant” when used with respect to a genome (e.g., an Anellovirus genome), or a fragment thereof, refers to a sequence having at least one change relative to a corresponding wild-type Anellovirus sequence. In some embodiments, the mutant genome or fragment thereof comprises at least one single nucleotide polymorphism, addition, deletion, or frameshift relative to the corresponding wild-type Anellovirus sequence. In some embodiments, the mutant genome or fragment thereof comprises a deletion of at least one Anellovirus ORF (e.g., one or more of ORF1, ORF2, ORF2/2, ORF2/3, ORF1/1, and/or ORF1/2) relative to the corresponding wild-type Anellovirus sequence. In some embodiments, the mutant genome or fragment thereof comprises a deletion of all Anellovirus ORFs (e.g., all of ORF1, ORF2, ORF2/2, ORF2/3, ORF1/1, and ORF1/2) relative to the corresponding wild-type Anellovirus sequence. In some embodiments, the mutant genome or fragment thereof comprises a deletion of at least one Anellovirus noncoding region (e.g., one or more of a 5′ UTR, 3′ UTR, and/or GC-rich region) relative to the corresponding wild-type Anellovirus sequence. In some embodiments, the mutant genome or fragment thereof comprises or encodes an exogenous effector.

As used herein the term “ORF molecule” refers to a polypeptide having an activity and/or a structural feature of an Anellovirus ORF protein (e.g., an Anellovirus ORF1, ORF2, ORF2/2, ORF2/3, ORF1/1, and/or ORF1/2 protein), or a functional fragment thereof. When used generically (i.e., “ORF molecule”), the polypeptide may comprise an activity and/or structural feature of any of the Anellovirus ORFs described herein (e.g., an Anellovirus ORF1, ORF2, ORF2/2, ORF2/3, ORF1/1, and/or ORF1/2), or a functional fragment thereof. When used with a modifier to indicate a particular open reading frame (e.g., “ORF1 molecule,” “ORF2 molecule,” “ORF2/2 molecule,” “ORF2/3 molecule,” “ORF1/1 molecule,” or “ORF1/2 molecule”), it is generally meant that the polypeptide comprises an activity and/or structural feature of the corresponding Anellovirus ORF protein, or a functional fragment thereof (for example, as defined below for “ORF1 molecule”). For example, an “ORF2 molecule” comprises an activity and/or structural feature of an Anellovirus ORF2 protein, or a functional fragment thereof.

As used herein, the term “ORF1 molecule” refers to a polypeptide having an activity and/or a structural feature of an Anellovirus ORF1 protein (e.g., an Anellovirus ORF1 protein as described herein), or a functional fragment thereof. An ORF1 molecule may, in some instances, comprise one or more of (e.g., 1, 2, 3 or 4 of): a first region comprising at least 60% basic residues (e.g., at least 60% arginine residues), a second region composing at least about six beta strands (e.g., at least 4, 5, 6, 7, 8, 9, 10, 11, or 12 beta strands), a third region comprising a structure or an activity of an Anellovirus N22 domain (e.g., as described herein, e.g., an N22 domain from an Anellovirus ORF1 protein as described herein), and/or a fourth region comprising a structure or an activity of an Anellovirus C-terminal domain (CTD) (e.g., as described herein, e.g., a CTD from an Anellovirus ORF1 protein as described herein). In some instances, the ORF1 molecule comprises, in N-terminal to C-terminal order, the first, second, third, and fourth regions. In some instances, an anellovector comprises an ORF1 molecule comprising, in N-terminal to C-terminal order, the first, second, third, and fourth regions. An ORF1 molecule may, in some instances, comprise a polypeptide encoded by an Anellovirus ORF1 nucleic acid. An ORF1 molecule may, in some instances, further comprise a heterologous sequence, e.g., a hypervariable region (HVR), e.g., an HVR from an Anellovirus ORF1 protein, e.g., as described herein. An “Anellovirus ORF1 protein,” as used herein, refers to an ORF1 protein encoded by an Anellovirus genome (e.g., a wild-type Anellovirus genome, e.g., as described herein).

As used herein, the term “ORF2 molecule” refers to a polypeptide having an activity and/or a structural feature of an Anellovirus ORF2 protein (e.g., an Anellovirus ORF2 protein as described herein), or a functional fragment thereof. An “Anellovirus ORF2 protein,” as used herein, refers to an ORF2 protein encoded by an Anellovirus genome (e.g., a wild-type Anellovirus genome, e.g., as described herein).

As used herein, the term “proteinaceous exterior” refers to an exterior component that is predominantly (e.g., >50%, >60%, >70%, >80%, >90%) protein.

As used herein, the term “regulatory nucleic acid” refers to a nucleic acid sequence that modifies expression, e.g., transcription and/or translation, of a DNA sequence that encodes an expression product. In embodiments, the expression product comprises RNA or protein.

As used herein, the term “regulatory sequence” refers to a nucleic acid sequence that modifies transcription of a target gene product. In some embodiments, the regulatory sequence is a promoter or an enhancer.

As used herein, the term “Rep” or “replication protein” refers to a protein, e.g., a viral protein, that promotes viral genome replication. In some embodiments, the replication protein is an Anellovirus Rep protein.

As used herein, the term “Rep binding site” refers to a nucleic acid sequence within a nucleic acid molecule that is recognized and bound by a Rep protein (e.g., an Anellovirus Rep protein). In some embodiments, a Rep binding site comprises a 5′ UTR (e.g., comprising a hairpin loop). In some embodiments, a Rep binding site comprises an origin of replication (ORI).

As used herein, the term “Rep displacement site” refers to a nucleic acid sequence within a nucleic acid molecule that is capable of causing a Rep protein (e.g., an Anellovirus Rep protein) associated with (e.g., bound to) the nucleic acid molecule to release the nucleic acid molecule upon reaching the Rep displacement site. In some embodiments, a Rep displacement site comprises a 5′ UTR (e.g., comprising a hairpin loop). In some embodiments, a Rep displacement site comprises an origin of replication (ORI).

As used herein, a “substantially non-pathogenic” organism, particle, or component, refers to an organism, particle (e.g., a virus or an anellovector, e.g., as described herein), or component thereof that does not cause or induce an unacceptable disease or pathogenic condition, e.g., in a host organism, e.g., a mammal, e.g., a human. In some embodiments, administration of an anellovector to a subject can result in minor reactions or side effects that are acceptable as part of standard of care.

As used herein, the term “non-pathogenic” refers to an organism or component thereof that does not cause or induce an unacceptable disease or pathogenic condition, e.g., in a host organism, e.g., a mammal, e.g., a human.

As used herein, a “substantially non-integrating” genetic element refers to a genetic element, e.g., a genetic element in a virus or anellovector, e.g., as described herein, wherein less than about 0.01%, 0.05%, 0.1%, 0.5%, or 1% of the genetic element that enter into a host cell (e.g., a eukaryotic cell) or organism (e.g., a mammal, e.g., a human) integrate into the genome. In some embodiments the genetic element does not detectably integrate into the genome of, e.g., a host cell. In some embodiments, integration of the genetic element into the genome can be detected using techniques as described herein, e.g., nucleic acid sequencing, PCR detection and/or nucleic acid hybridization. In some embodiments, integration frequency is determined by quantitative gel purification assay of genomic DNA separated from free vector, e.g., as described in Wang et al. (2004, Gene Therapy 11: 711-721, incorporated herein by reference in its entirety).

As used herein, a “substantially non-immunogenic” organism, particle, or component, refers to an organism, particle (e.g., a virus or anellovector, e.g., as described herein), or component thereof, that does not cause or induce an undesired or untargeted immune response, e.g., in a host tissue or organism (e.g., a mammal, e.g., a human). In embodiments, the substantially non-immunogenic organism, particle, or component does not produce a clinically significant immune response. In embodiments, the substantially non-immunogenic anellovector does not produce a clinically significant immune response against a protein comprising an amino acid sequence or encoded by a nucleic acid sequence of an Anellovirus or anellovector genetic element. In embodiments, an immune response (e.g., an undesired or untargeted immune response) is detected by assaying antibody (e.g., neutralizing antibody) presence or level (e.g., presence or level of an anti-anellovector antibody, e.g., presence or level of an antibody against an anellovector as described herein) in a subject, e.g., according to the anti-TTV antibody detection method described in Tsuda et al. (1999; J. Virol. Methods 77: 199-206; incorporated herein by reference) and/or the method for determining anti-TTV IgG levels described in Kakkola et al. (2008; Virology 382: 182-189; incorporated herein by reference). Antibodies (e.g., neutralizing antibodies) against an Anellovirus or an anellovector based thereon can also be detected by methods in the art for detecting anti-viral antibodies, e.g., methods of detecting anti-AAV antibodies, e.g., as described in Calcedo et al. (2013; Front. Immunol. 4(341): 1-7; incorporated herein by reference).

A “subsequence” as used herein refers to a nucleic acid sequence or an amino acid sequence that is comprised in a larger nucleic acid sequence or amino acid sequence, respectively. In some instances, a subsequence may comprise a domain or functional fragment of the larger sequence. In some instances, the subsequence may comprise a fragment of the larger sequence capable of forming secondary and/or tertiary structures when isolated from the larger sequence similar to the secondary and/or tertiary structures formed by the subsequence when present with the remainder of the larger sequence. In some instances, a subsequence can be replaced by another sequence (e.g., a subseqence comprising an exogenous sequence or a sequence heterologous to the remainder of the larger sequence, e.g., a corresponding subsequence from a different Anellovirus).

A “transposase recognition sequence,” as used herein, refers to a nucleic acid sequence recognized (e.g., capable of being specifically bound) by a transposase enzyme. In some embodiments, the transposase is a Tn7 transposase. In some embodiments, a nucleic acid molecule (e.g., a bacmid or donor vector, e.g., as described herein) comprises a first transposase recognition sequence (e.g., a Tn7R or a Tn7L sequence). In some embodiments, a nucleic acid molecule (e.g., a bacmid or donor vector, e.g., as described herein) comprises a second transposase recognition sequence (e.g., a cognate sequence to the first transposase recognition sequence, e.g., the other of a Tn7R or a Tn7L sequence).

This invention relates generally to anellovectors, e.g., synthetic anellovectors, and uses thereof. The present disclosure provides anellovectors, compositions comprising anellovectors, and methods of making or using anellovectors. Anellovectors are generally useful as delivery vehicles, e.g., for delivering a therapeutic agent to a eukaryotic cell. Generally, an anellovector will include a genetic element comprising a nucleic acid sequence (e.g., encoding an effector, e.g., an exogenous effector or an endogenous effector) enclosed within a proteinaceous exterior. An anellovector may include one or more deletions of sequences (e.g., regions or domains as described herein) relative to an Anellovirus sequence (e.g., as described herein). Anellovectors can be used as a substantially non-immunogenic vehicle for delivering the genetic element, or an effector encoded therein (e.g., a polypeptide or nucleic acid effector, e.g., as described herein), into eukaryotic cells, e.g., to treat a disease or disorder in a subject comprising the cells.

### TABLE OF CONTENTS

### I. Compositions and Methods for Making Anellovectors

A. Baculovirus Expression Systems

B. Insect Cells

C. Components and Assembly of Anellovectors
i. ORF1 molecules for assembly of anellovectors
ii. ORF2 molecules for assembly of anellovectors

D. Genetic Element Constructs
i. Plasmids
ii. Circular nucleic acid constructs
iii. In vitro circularization
iv. Cis/trans constructs
v. Expression cassettes
vi. Design and production of a genetic element construct

C. Effectors

D. Host Cells
i. Introduction of genetic elements into host cells
ii. Methods for providing Anellovirus protein(s) in cis or trans
iii. Helpers
iv. Exemplary cell types

E. Culture Conditions

F. Harvest

G. Enrichment and Purification

### II. Anellovectors

A. Anelloviruses

B. ORF1 molecules

C. ORF2 molecules

D. Genetic elements

E. Protein binding sequences

F. 5′ UTR Regions

G. GC-rich regions

H. Effectors

I. Regulatory Sequences

J. Replication Proteins

K. Other Sequences

L. Proteinaceous exterior

### III. Nucleic Acid Constructs

### IV. Compositions

V. Host cells

### VI. Methods of use

### VII. Administration/Delivery

### I. Compositions and Methods for Making Anellovectors

The present disclosure provides, in some aspects, compositions (e.g., bacmids, donor vectors, baculovirus particles, and cells comprising same) and methods that can be used for producing anellovectors, e.g., as described herein. In some embodiments, the compositions and methods described herein can be used to produce a genetic element or a genetic element construct. In some embodiments, the compositions and methods described herein can be used to produce one or more Anellovirus ORF molecules (e.g., an ORF1, ORF2, ORF2/2, ORF2/3, ORF1/1, or ORF1/2 molecule, or a functional fragment or splice variant thereof). In some embodiments, the compositions and methods described herein can be used to produce a proteinaceous exterior or a component thereof (e.g., an ORF1 molecule), e.g., in a host cell (e.g., an insect cell, e.g., an Sf9 cell).

In some embodiments, a bacmid is a nucleic acid construct comprising a genetic element sequence (e.g., a genetic element region) and one or baculoviral elements (e.g., a baculovirus genome). In some embodiments, the bacmid can be utilized in a baculoviral expression system, e.g., in an insect cell, e.g., as described herein, for example, to produce a genetic element construct and/or an Anellovirus ORF molecule (e.g., an ORF1, ORF2, ORF2/2, ORF2/3, ORF1/1, or ORF1/2 molecule, or a functional fragment or splice variant thereof).

Without wishing to be bound by theory, rolling circle amplification may occur via Rep protein binding to a Rep binding site (e.g., comprising a 5′ UTR, e.g., comprising a hairpin loop and/or an origin of replication, e.g., as described herein) positioned 5′ relative to (or within the 5′ region of) the genetic element region (e.g., in a bacmid, e.g., as described herein). The Rep protein may then proceed through the genetic element region, resulting in the synthesis of the genetic element. The genetic element may then be circularized and then enclosed within a proteinaceous exterior to form an anellovector.

### Baculovirus Expression Systems

A viral expression system, e.g., a baculovirus expression system, may be used to express proteins (e.g., for production of anellovectors), e.g., as described herein. Baculoviruses are rod-shaped viruses with a circular, supercoiled double-stranded DNA genome. Genera of baculoviruses include: Alphabaculovirus (nucleopolyhedroviruses (NPVs) isolated from Lepidoptera), Betabaculoviruses (granuloviruses (GV) isolated from Lepidoptera), Gammabaculoviruses (NPVs isolated from Hymenoptera) and Deltabaculoviruses (NPVs isolated from Diptera). While GVs typically contain only one nucleocapsid per envelope, NPVs typically contain either single (SNPV) or multiple (MNPV) nucleocapsids per envelope. The enveloped virions are further occluded in granulin matrix in GVs and polyhedrin in NPVs. Baculoviruses typically have both lytic and occluded life cycles. In some embodiments, the lytic and occluded life cycles manifest independently throughout the three phases of virus replication: early, late, and very late phase. In some embodiments, during the early phase, viral DNA replication takes place following viral entry into the host cell, early viral gene expression and shut-off of the host gene expression machinery. In some embodiments, in the late phase late genes that code for viral DNA replication are expressed, viral particles are assembled, and extracellular virus (EV) is produced by the host cell. In some embodiments, in the very late phase the polyhedrin and p10 genes are expressed, occluded viruses (OV) are produced by the host cell, and the host cell is lysed. Since baculoviruses infect insect species, they can be used as biological agents to produce exogenous proteins in baculoviruses-permissive insect cells or larvae. Different isolates of baculovirus, such as Autographa californica multiple nuclear polyhedrosis virus (AcMNPV) and Bombyx mori (silkworm) nuclear polyhedrosis virus (BmNPV) may be used in exogenous protein expression. Various baculoviral expression systems are commercially available, e.g., from ThermoFisher.

In some embodiments, the proteins described herein (e.g., an Anellovirus ORF molecule, e.g., ORF1, ORF2, ORF2/2, ORF2/3, ORF1/1, or ORF1/2, or a functional fragment or splice variant thereof) may be expressed using a baculovirus expression vector (e.g., a bacmid) that comprises one or more components described herein. For example, a baculovirus expression vector may include one or more of (e.g., all of) a selectable marker (e.g., kanR), an origin of replication (e.g., one or both of a bacterial origin of replication and an insect cell origin of replication), a recombinase recognition site (e.g., an att site), and a promoter. In some embodiments, a baculovirus expression vector (e.g., a bacmid as described herein) can be produced by replacing the naturally occurring wild-type polyhedrin gene, which encodes for baculovirus occlusion bodies, with genes encoding the proteins described herein. In some embodiments, the genes encoding the proteins described herein are cloned into a baculovirus expression vector (e.g., a bacmid as described herein) containing a baculovirus promoter. In some embodiments, the baculoviral vector comprises one or more non-baculoviral promoters, e.g., a mammalian promoter or an Anellovirus promoter. In some embodiments, the genes encoding the proteins described herein are cloned into a donor vector (e.g., as described herein), which is then contacted with an empty baculovirus expression vector (e.g., an empty bacmid) such that the genes encoding the proteins described herein are transferred (e.g., by homologous recombination or transposase activity) from the donor vector into the baculovirus expression vector (e.g., bacmid). In some embodiments, the baculovirus promoter is flanked by baculovirus DNA from the nonessential polyhedrin gene locus. In some embodiments, a protein described herein is under the transcriptional control of the AcNPV polyhedrin promoter in the very late phase of viral replication. In some embodiments, a strong promoters suitable for use in baculoviral expression in insect cells include, but are not limited to, baculovirus p10 promoters, polyhedrin (polh) promoters, p6.9 promoters and capsid protein promoters. Weak promoters suitable for use in baculoviral expression in insect cells include ie1, ie2, ie0, et1, 39K (aka pp31) and gp64 promoters of baculoviruses.

In some embodiments, a recombinant baculovirus is produced by homologous recombination between a baculoviral genome (e.g., a wild-type or mutant baculoviral genome), and a transfer vector. In some embodiments, one or more genes encoding a protein described herein are cloned into the transfer vector. In some embodiments, the transfer vector further contains a baculovirus promoter flanked by DNA from a nonessential gene locus, e.g., polyhedrin gene. In some embodiments, one or more genes encoding a protein described herein are inserted into the baculoviral genome by homologous recombination between the baculoviral genome and the transfer vector. In some embodiments, the baculoviral genome is linearized at one or more unique sites. In some embodiments, the linearized sites are located near the target site for insertion of genes encoding the proteins described herein into the baculoviral genome. In some embodiments, a linearized baculoviral genome missing a fragment of the baculoviral genome downstream from a gene, e.g., polyhedrin gene, can be used for homologous recombination. In some embodiments, the baculoviral genome and transfer vector are co-transfected into insect cells. In some embodiments, the method of producing the recombinant baculovirus comprises the steps of preparing the baculoviral genome for performing homologous recombination with a transfer vector containing the genes encoding one or more protein described herein and co-transfecting the transfer vector and the baculoviral genome DNA into insect cells. In some embodiments, the baculoviral genome comprises a region homologous to a region of the transfer vector. These homologous regions may enhance the probability of recombination between the baculoviral genome and the transfer vector. In some embodiments, the homology region in the transfer vector is located upstream or downstream of the promoter. In some embodiments, to induce homologous recombination, the baculoviral genome, and transfer vector are mixed at a weight ratio of about 1:1 to 10:1.

In some embodiments, a recombinant baculovirus is generated by a method comprising site-specific transposition with Tn7, e.g., whereby the genes encoding the proteins described herein are inserted into bacmid DNA, e.g., propagated in bacteria, e.g., E. coli (e.g., DH 10Bac cells). In some embodiments, the genes encoding the proteins described herein are cloned into a pFASTBAC® vector and transformed into competent cells, e.g., DH10BAC® competent cells, containing the bacmid DNA with a mini-attTn7 target site. In some embodiments, the baculovirus expression vector, e.g., pFASTBAC® vector, may have a promoter, e.g., a dual promoter (e.g., polyhedrin promoter, p10 promoter). Commercially available pFASTBAC® donor plasmids include: pFASTBAC 1, pFASTBAC HT, and pFASTBAC DUAL. In some embodiments, recombinant bacmid DNA containing-colonies are identified and bacmid DNA is isolated to transfect insect cells.

In some embodiments, a baculoviral vector is introduced into an insect cell together with a helper nucleic acid. The introduction may be concurrent or sequential. In some embodiments, the helper nucleic acid provides one or more baculoviral proteins, e.g., to promote packaging of the baculoviral vector.

In some embodiments, recombinant baculovirus produced in insect cells (e.g., by homologous recombination) is expanded and used to infect insect cells (e.g., in the mid-logarithmic growth phase) for recombinant protein expression. In some embodiments, recombinant bacmid DNA produced by site-specific transposition in bacteria, e.g., E. coli, is used to transfect insect cells with a transfection agent, e.g., Cellfectin® II. Additional information on baculovirus expression systems is discussed in U.S. patent application Ser. Nos. 14/447,341, 14/277,892, and 12/278,916, which are hereby incorporated by reference.

### Insect Cells

The proteins described herein may be expressed in insect cells infected or transfected with recombinant baculovirus or bacmid DNA, e.g., as described above. In some embodiments, insect cells include: the Sf9 and Sf21 cells derived from Spodoptera frugiperda and the Tn-368 and High Five™ BTI-TN-5B1-4 cells (also referred to as Hi5 cells) derived from Trichoplusia ni. In some embodiments, insect cell lines Sf21 and Sf9, derived from the ovaries of the pupal fall army worm Spodoptera frugiperda, can be used for the expression of recombinant proteins using the baculovirus expression system. In some embodiments, Sf21 and Sf9 insect cells may be cultured in commercially available serum-supplemented or serum-free media. Suitable media for culturing insect cells include: Grace's Supplemented (TNM-FH), IPL-41, TC-100, Schneider's Drosophila, SF-900 II SFM, and EXPRESS-FIVE™ SFM. In some embodiments, some serum-free media formulations utilize a phosphate buffer system to maintain a culture pH in the range of 6.0-6.4 (Licari et al. Insect cell hosts for baculovirus expression vectors contain endogenous exoglycosidase activity. Biotechnology Progress 9: 146-152 (1993) and Drugmand et al. Insect cells as factories for biomanufacturing. Biotechnology Advances 30:1140-1157 (2012)) for both cultivation and recombinant protein production. In some embodiments, a pH of 6.0-6.8 for cultivating various insect cell lines may be used. In some embodiments, insect cells are cultivated in suspension or as a monolayer at a temperature between 25° to 30° C. with aeration. Additional information on insect cells is discussed, for example, in U.S. patent application Ser. Nos. 14/564,512 and 14/775,154, each of which is hereby incorporated by reference.

### Components and Assembly of Anellovectors

The compositions and methods herein can be used to produce anellovectors. As described herein, an anellovector generally comprises a genetic element (e.g., a single-stranded, circular DNA molecule, e.g., comprising a 5′ UTR region as described herein) enclosed within a proteinaceous exterior (e.g., comprising a polypeptide encoded by an Anellovirus ORF1 nucleic acid, e.g., as described herein). In some embodiments, the genetic element comprises one or more sequences encoding Anellovirus ORFs (e.g., one or more of an Anellovirus ORF1, ORF2, ORF2/2, ORF2/3, ORF1/1, or ORF1/2). As used herein, an Anellovirus ORF or ORF molecule (e.g., an Anellovirus ORF1, ORF2, ORF2/2, ORF2/3, ORF1/1, or ORF1/2) includes a polypeptide comprising an amino acid sequence having at least 70%, 75%, 80%, 85%, 90%, 95%, 96%, 97%, 98%, 99%, or 100% sequence identity to a corresponding Anellovirus ORF sequence, e.g., as described in PCT/US2018/037379 or PCT/US19/65995 (each of which is incorporated by reference herein in their entirety). In embodiments, the genetic element comprises a sequence encoding an Anellovirus ORF1, or a splice variant or functional fragment thereof (e.g., a jelly-roll region, e.g., as described herein). In some embodiments, the proteinaceous exterior comprises a polypeptide encoded by an Anellovirus ORF1 nucleic acid (e.g., an Anellovirus ORF1 molecule or a splice variant or functional fragment thereof).

In some embodiments, an anellovector is assembled by enclosing a genetic element (e.g., as described herein) within a proteinaceous exterior (e.g., as described herein). In some embodiments, the genetic element is enclosed within the proteinaceous exterior in a host cell (e.g., an insect cell, e.g., an Sf9 cell). In some embodiments, the host cell expresses one or more polypeptides comprised in the proteinaceous exterior (e.g., a polypeptide encoded by an Anellovirus ORF1 nucleic acid, e.g., an ORF1 molecule). For example, in some embodiments, the host cell comprises a nucleic acid sequence encoding an Anellovirus ORF1 molecule, e.g., a splice variant or a functional fragment of an Anellovirus ORF1 polypeptide (e.g., a wild-type Anellovirus ORF1 protein or a polypeptide encoded by a wild-type Anellovirus ORF1 nucleic acid, e.g., as described herein). In embodiments, the nucleic acid sequence encoding the Anellovirus ORF1 molecule is comprised in a nucleic acid construct (e.g., a plasmid, viral vector, virus, minicircle, bacmid, or artificial chromosome) comprised in the host cell. In embodiments, the nucleic acid sequence encoding the Anellovirus ORF1 molecule is integrated into the genome of the host cell.

In some embodiments, the host cell comprises the genetic element and/or a nucleic acid construct comprising the sequence of the genetic element. In some embodiments, the nucleic acid construct is selected from a plasmid, viral nucleic acid, minicircle, bacmid, or artificial chromosome. In some embodiments, the genetic element is excised from the nucleic acid construct (e.g., bacmid) and, optionally, converted from a double-stranded form to a single-stranded form (e.g., by denaturation). In some embodiments, the genetic element is generated by a polymerase based on a template sequence in the nucleic acid construct (e.g., bacmid). In some embodiments, the polymerase produces a single-stranded copy of the genetic element sequence, which can optionally be circularized to form a genetic element as described herein.

In some embodiments, the host cell comprises a genetic element construct (e.g., a bacmid, plasmid, or minicircle) and a bacmid comprising one or more sequences encoding Anellovirus ORF molecules (e.g., ORF1, ORF2, ORF2/2, ORF2/3, ORF1/1, and/or ORF1/2 ORF molecules), or functional fragments thereof. In some embodiments, proteinaceous exterior proteins are expressed from the bacmid. In embodiments, the proteinaceous exterior proteins expressed from the bacmid enclose a genetic element, thereby forming an anellovector. In some embodiments, the bacmid comprises a backbone suitable for replication of the nucleic acid construct in insect cells (e.g., Sf9 cells), e.g., a baculovirus backbone region. In some embodiments, the bacmid comprises a backbone region suitable for replication of the genetic element construct in bacterial cells (e.g., E. coli cells, e.g., DH 10Bac cells). In some embodiments, the genetic element construct comprises a backbone suitable for replication of the nucleic acid construct in insect cells (e.g., Sf9 cells), e.g., a baculovirus backbone region. In some embodiments, the genetic element construct comprises a backbone region suitable for replication of the genetic element construct in bacterial cells (e.g., E. coli cells, e.g., DH 10Bac cells). In some embodiments, the bacmid is introduced into the host cell via a baculovirus particle. In embodiments, the bacmid is produced by a producer cell, e.g., an insect cell (e.g., an Sf9 cell) or a bacterial cell (e.g., an E. coli cell, e.g., a DH 10Bac cell). In embodiments, the producer cell comprises a bacmid and/or a donor vector, e.g., as described herein. In embodiments, the producer cell further comprises sufficient cellular machinery for replication of the bacmid and/or donor vector.

### ORF1 Molecules, e.g., for Assembly of Anellovectors

An anellovector can be made, for example, by enclosing a genetic element within a proteinaceous exterior. In some embodiments, the enclosure occurs in an insect cell (e.g., an Sf9 cell). The proteinaceous exterior of an Anellovector generally comprises a polypeptide encoded by an Anellovirus ORF1 nucleic acid (e.g., an Anellovirus ORF1 molecule or a splice variant or functional fragment thereof, e.g., as described herein). In some embodiments, the Anellovirus ORF1 nucleic acid is comprised in a bacmid or donor vector, e.g., as described herein. In some embodiments, the bacmid further comprises a sequence encoding an exogenous effector (e.g., a genetic element region, e.g., as described herein). In other embodiments, the bacmid does not comprise a sequence encoding an exogenous effector (e.g., the bacmid does not comprise a genetic element region, e.g., as described herein). An ORF1 molecule may, in some embodiments, comprise one or more of: a first region comprising an arginine rich region, e.g., a region having at least 60% basic residues (e.g., at least 60%, 65%, 70%, 75%, 80%, 85%, 90%, 95%, or 100% basic residues; e.g., between 60%-90%, 60%-80%, 70%-90%, or 70-80% basic residues), and a second region comprising jelly-roll domain, e.g., at least six beta strands (e.g., 4, 5, 6, 7, 8, 9, 10, 11, or 12 beta strands). In embodiments, the proteinaceous exterior comprises one or more (e.g., 1, 2, 3, 4, or all 5) of an Anellovirus ORF1 arginine-rich region, jelly-roll region, N22 domain, hypervariable region, and/or C-terminal domain. In some embodiments, the proteinaceous exterior comprises an Anellovirus ORF1 jelly-roll region (e.g., as described herein). In some embodiments, the proteinaceous exterior comprises an Anellovirus ORF1 arginine-rich region (e.g., as described herein). In some embodiments, the proteinaceous exterior comprises an Anellovirus ORF1 N22 domain (e.g., as described herein). In some embodiments, the proteinaceous exterior comprises an Anellovirus hypervariable region (e.g., as described herein). In some embodiments, the proteinaceous exterior comprises an Anellovirus ORF1 C-terminal domain (e.g., as described herein).

In some embodiments, the anellovector comprises an ORF1 molecule and/or a nucleic acid encoding an ORF1 molecule. Generally, an ORF1 molecule comprises a polypeptide having the structural features and/or activity of an Anellovirus ORF1 protein (e.g., an Anellovirus ORF1 protein as described herein), or a functional fragment thereof. In some embodiments, the ORF1 molecule comprises a truncation relative to an Anellovirus ORF1 protein (e.g., an Anellovirus ORF1 protein as described herein). In some embodiments, the ORF1 molecule is truncated by at least 10, 20, 30, 40, 50, 60, 70, 80, 90, 100, 150, 200, 250, 300, 350, 400, 450, 500, 550, 600, 650, or 700 amino acids of the Anellovirus ORF1 protein. In some embodiments, an ORF1 molecule comprises an amino acid sequence having at least 75%, 80%, 85%, 90%, 95%, 96%, 97%, 98%, or 99% sequence identity to an Alphatorquevirus, Betatorquevirus, or Gammatorquevirus ORF1 protein, e.g., as described herein. An ORF1 molecule can generally bind to a nucleic acid molecule, such as DNA (e.g., a genetic element, e.g., as described herein). In some embodiments, an ORF1 molecule localizes to the nucleus of a cell. In certain embodiments, an ORF1 molecule localizes to the nucleolus of a cell.

Without wishing to be bound by theory, an ORF1 molecule may be capable of binding to other ORF1 molecules, e.g., to form a proteinaceous exterior (e.g., as described herein). Such an ORF1 molecule may be described as having the capacity to form a capsid. In some embodiments, the proteinaceous exterior may enclose a nucleic acid molecule (e.g., a genetic element as described herein, e.g., produced using a bacmid, donor vector, baculovirus, or cell comprising same, e.g., as described herein). In some embodiments, a plurality of ORF1 molecules may form a multimer, e.g., to produce a proteinaceous exterior. In some embodiments, the multimer may be a homomultimer. In other embodiments, the multimer may be a heteromultimer.

ORF2 Molecules, e.g., for Assembly of Anellovectors

Producing an anellovector using the compositions or methods described herein may involve expression of an Anellovirus ORF2 molecule (e.g., as described herein), or a splice variant or functional fragment thereof. In some embodiments, the anellovector comprises an ORF2 molecule, or a splice variant or functional fragment thereof, and/or a nucleic acid encoding an ORF2 molecule, or a splice variant or functional fragment thereof. In some embodiments, the anellovector does not comprise an ORF2 molecule, or a splice variant or functional fragment thereof, and/or a nucleic acid encoding an ORF2 molecule, or a splice variant or functional fragment thereof. In some embodiments, producing the anellovector comprises expression of an ORF2 molecule, or a splice variant or functional fragment thereof, but the ORF2 molecule is not incorporated into the anellovector.

Constructs Comprising ORF Molecule-Encoding Sequences

In some embodiments, one or more ORF molecules (e.g., ORF1, ORF1, ORF2, ORF2/2, ORF2/3, ORF1/1, and ORF1/2 molecules), or splice variants or functional fragments thereof, are provided by a nucleic acid construct (e.g., a bacmid) that may be separate from the genetic element or the nucleic acid construct comprising the genetic element region. The nucleic acid construct encoding the one or more ORF molecules, or splice variants or fragments thereof, may comprise a backbone region. In some embodiments, the backbone region is suitable for replication of the nucleic acid construct in insect cells (e.g., Sf9 cells), e.g., a baculovirus backbone region. In embodiments, the nucleic acid construct is a bacmid. In some embodiments, the backbone region is suitable for replication of the nucleic acid construct in bacterial cells. In some embodiments, the nucleic acid construct encodes an ORF1 molecule, or a splice variant or functional fragment thereof. In some embodiments, the nucleic acid construct further comprises a genetic element region (e.g., comprising the sequence of a genetic element to be enclosed by a proteinaceous exterior, e.g., comprising an ORF1 molecule or a splice variant or functional fragment thereof, e.g., encoded by the nucleic acid construct).

In some embodiments, the methods described herein involve a host cell (e.g., an insect cell, e.g., an Sf9 cell) comprising a nucleic acid construct encoding one or more ORF molecules, e.g., as described above. The host cell may express the one or more ORF molecules. In some embodiments, the host cell expresses polypeptides capable of forming an anellovector proteinaceous exterior (e.g., ORF1 molecules, or splice variants or functional fragments thereof), e.g., from the nucleic acid construct. In some embodiments, a genetic element construct (e.g., as described herein) is introduced into the host cell (e.g., prior to, concurrently with, or after expression of the polypeptides capable of forming the anellovector proteinaceous exterior). In other embodiments, the nucleic acid construct encoding the one or more ORF molecules is also a genetic element constructs (e.g., comprises a genetic element region, e.g., as described herein). In some embodiments, a genetic element (e.g., produced from the genetic element construct, e.g., as described herein) is enclosed within the proteinaceous exterior in the host cell, thereby producing an anellovector. In some embodiments, the anellovector is harvested from the host cell or the surrounding supernatant, e.g., as described herein. In some embodiments, the host cell produces baculovirus particles comprising the nucleic acid construct.

In some embodiments, the methods described herein involve a host cell (e.g., a bacterial cell, e.g., an E. coli cell, e.g., a DH 10Bac cell) comprising a nucleic acid construct (e.g., a donor vector) comprising a sequence encoding an Anellovirus ORF molecule (e.g., an ORF1, ORF2, ORF2/2, ORF2/3, ORF1/1, and/or ORF1/2 molecule), or a functional fragment or splice variant thereof, and/or a genetic element region. In some embodiments, the host cell further comprises a bacmid (e.g., a bacmid lacking a sequence encoding an Anellovirus ORF molecule and/or a genetic element region). In some embodiments, the sequence encoding the Anellovirus ORF molecule, or functional fragment or splice variant thereof, is transferred into the bacmid within the host cell. In some embodiments, the host cell produces baculovirus particles comprising the bacmid comprising the transferred sequence encoding the Anellovirus ORF molecule, or functional fragment or splice variant thereof. In some embodiments, the genetic element region is transferred into the bacmid within the host cell. In some embodiments, the host cell produces baculovirus particles comprising the bacmid comprising the transferred genetic element region.

### Genetic Element Constructs, e.g., for Assembly of Anellovectors

The genetic element of an anellovector as described herein may be produced from a genetic element construct that comprises a genetic element region and optionally other sequence such as a bacmid (e.g., comprising a baculovirus genome or a fragment thereof, e.g., one or more baculovirus elements) or donor vector backbone. Generally, the genetic element construct comprises an Anellovirus 5′ UTR (e.g., as described herein). A genetic element construct may be any nucleic acid construct suitable for delivery of the sequence of the genetic element into a host cell in which the genetic element can be enclosed within a proteinaceous exterior. In some embodiments, the genetic element construct comprises a promoter. In some embodiments, the genetic element construct is a linear nucleic acid molecule. In some embodiments, the genetic element construct is a circular nucleic acid molecule (e.g., a plasmid, bacmid, donor vector, or a minicircle, e.g., as described herein). In some embodiments, the genetic element construct comprises baculovirus sequences (e.g., such that an insect cell comprising the genetic element construct can produce a baculovirus comprising the genetic element sequence of the genetic element construct, or a fragment thereof). The genetic element construct may, in some embodiments, be double-stranded. In other embodiments, the genetic element is single-stranded. In some embodiments, the genetic element construct comprises DNA. In some embodiments, the genetic element construct comprises RNA. In some embodiments, the genetic element construct comprises one or more modified nucleotides.

In some aspects, the present disclosure provides a method for replication and propagation of the anellovector as described herein (e.g., in a cell culture system), which may comprise one or more of the following steps: (a) introducing (e.g., transfecting) a genetic element (e.g., linearized) into a cell line sensitive to anellovector infection; (b) harvesting the cells and optionally isolating cells showing the presence of the genetic element; (c) culturing the cells obtained in step (b) (e.g., for at least three days, such as at least one week or longer), depending on experimental conditions and gene expression; and (d) harvesting the cells of step (c), e.g., as described herein.

Plasmids

In some embodiments, the genetic element construct is a plasmid. The plasmid will generally comprise the sequence of a genetic element as described herein as well as an origin of replication suitable for replication in a host cell (e.g., a bacterial origin of replication for replication in bacterial cells) and a selectable marker (e.g., an antibiotic resistance gene). In some embodiments, the sequence of the genetic element can be excised from the plasmid. In some embodiments, the plasmid is capable of replication in a bacterial cell. In some embodiments, the plasmid is capable of replication in a mammalian cell (e.g., a human cell). In some embodiments, a plasmid is at least 300, 400, 500, 600, 700, 800, 900, 1000, 2000, 3000, 4000, or 5000 bp in length. In some embodiments, the plasmid is less than 600, 700, 800, 900, 1000, 2000, 3000, 4000, 5000, 6000, 7000, 8000, 9000, or 10,000 bp in length. In some embodiments, the plasmid has a length between 300-400, 400-500, 500-600, 600-700, 700-800, 800-900, 900-1000, 1000-1500, 1500-2000, 2000-2500, 2500-3000, 3000-4000, or 4000-5000 bp. In some embodiments, the genetic element can be excised from a plasmid (e.g., by in vitro circularization), for example, to form a minicircle, e.g., as described herein. In embodiments, excision of the genetic element separates the genetic element sequence from the plasmid backbone (e.g., separates the genetic element from a bacterial backbone). In some embodiments, the plasmid comprises one or more baculoviral elements (e.g., a baculovirus genome).

Small Circular Nucleic Acid Constructs

In some embodiments, the genetic element construct is a circular nucleic acid construct, e.g., lacking a backbone (e.g., lacking a bacterial origin of replication and/or selectable marker). In embodiments, the genetic element is a double-stranded circular nucleic acid construct. In embodiments, the double-stranded circular nucleic acid construct is produced by in vitro circularization (IVC), e.g., as described herein. In embodiments, the double-stranded circular nucleic acid construct can be introduced into a host cell, in which it can be converted into or used as a template for generating single-stranded circular genetic elements, e.g., as described herein. In some embodiments, the circular nucleic acid construct does not comprise a plasmid backbone or a functional fragment thereof. In some embodiments, the circular nucleic acid construct is at least 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, or 4500 bp in length. In some embodiments, the circular nucleic acid construct is less than 2900, 3000, 3100, 3200, 3300, 3400, 3500, 3600, 3700, 3800, 3900, 4000, 4100, 4200, 4300, 4400, 4500, 4600, 4700, 4800, 4900, 5000, 5500, or 6000 bp in length. In some embodiments, the circular nucleic acid construct is between 2000-2100, 2100-2200, 2200-2300, 2300-2400, 2400-2500, 2500-2600, 2600-2700, 2700-2800, 2800-2900, 2900-3000, 3000-3100, 3100-3200, 3200-3300, 3300-3400, 3400-3500, 3500-3600, 3600-3700, 3700-3800, 3800-3900, 3900-4000, 4000-4100, 4100-4200, 4200-4300, 4300-4400, or 4400-4500 bp in length. In some embodiments, the circular nucleic acid construct is a minicircle.

In Vitro Circularization

In some instances, the genetic element to be packaged into a proteinaceous exterior is a single stranded circular DNA. The genetic element may, in some instances, be introduced into a host cell via a genetic element construct having a form other than a single stranded circular DNA. For example, the genetic element construct may be a double-stranded circular DNA. The double-stranded circular DNA may then be converted into a single-stranded circular DNA in the host cell (e.g., a host cell comprising a suitable enzyme for rolling circle replication, e.g., an Anellovirus Rep protein, e.g., Rep68/78, Rep60, RepA, RepB, Pre, MobM, TraX, TrwC, Mob02281, Mob02282, NikB, ORF50240, NikK, TecH, OrfJ, or Tral, e.g., as described in Wawrzyniak et al. 2017, Front. Microbiol. 8: 2353; incorporated herein by reference with respect to the listed enzymes). In some embodiments, the double-stranded circular DNA is produced by in vitro circularization (IVC), e.g., as described in Example 35 of PCT/US19/65995.

Generally, in vitro circularized DNA constructs can be produced by digesting a plasmid comprising the sequence of a genetic element to be packaged, such that the genetic element sequence is excised as a linear DNA molecule. The resultant linear DNA can then be ligated, e.g., using a DNA ligase, to form a double-stranded circular DNA. In some instances, a double-stranded circular DNA produced by in vitro circularization can undergo rolling circle replication, e.g., as described herein. Without wishing to be bound by theory, it is contemplated that in vitro circularization results in a double-stranded DNA construct that can undergo rolling circle replication without further modification, thereby being capable of producing single-stranded circular DNA of a suitable size to be packaged into an anellovector, e.g., as described herein. In some embodiments, the double-stranded DNA construct is smaller than a plasmid (e.g., a bacterial plasmid). In some embodiments, the double-stranded DNA construct is excised from a plasmid (e.g., a bacterial plasmid) and then circularized, e.g., by in vitro circularization.

Cis/Trans Constructs

In some embodiments, a genetic element construct (e.g., a bacmid or donor vector) as described herein comprises one or more sequences encoding one or more Anellovirus ORFs, e.g., proteinaceous exterior components (e.g., polypeptides encoded by an Anellovirus ORF1 nucleic acid, e.g., as described herein). For example, the genetic element construct may comprise a nucleic acid sequence encoding an Anellovirus ORF1 molecule. Such genetic element constructs can be suitable for introducing the genetic element and the Anellovirus ORF(s) into a host cell in cis. In other embodiments, a genetic element construct as described herein does not comprise sequences encoding one or more Anellovirus ORFs, e.g., proteinaceous exterior components (e.g., polypeptides encoded by an Anellovirus ORF1 nucleic acid, e.g., as described herein). For example, the genetic element construct may not comprise a nucleic acid sequence encoding an Anellovirus ORF1 molecule. Such genetic element constructs can be suitable for introducing the genetic element into a host cell, with the one or more Anellovirus ORFs to be provided in trans (e.g., via introduction of a second nucleic acid construct encoding one or more of the Anellovirus ORFs, or via an Anellovirus ORF cassette integrated into the genome of the host cell). In some embodiments, the genetic element construct comprises a backbone suitable for replication of the nucleic acid construct in insect cells (e.g., Sf9 cells), e.g., a baculovirus backbone region. In some embodiments, the genetic element construct comprises a backbone region suitable for replication of the genetic element construct in bacterial cells (e.g., E. coli cells, e.g., DH 10Bac cells).

In some embodiments, the genetic element construct (e.g., bacmid or donor vector) comprises a sequence encoding an Anellovirus ORF1 molecule, or a splice variant or functional fragment thereof (e.g., a jelly-roll region, e.g., as described herein). In embodiments, the portion of the genetic element that does not comprise the sequence of the genetic element comprises the sequence encoding the Anellovirus ORF1 molecule, or splice variant or functional fragment thereof (e.g., in a cassette comprising a promoter and the sequence encoding the Anellovirus ORF1 molecule, or splice variant or functional fragment thereof). In further embodiments, the portion of the construct comprising the sequence of the genetic element comprises a sequence encoding an Anellovirus ORF1 molecule, or a splice variant or functional fragment thereof (e.g., a jelly-roll region, e.g., as described herein). In embodiments, enclosure of such a genetic element in a proteinaceous exterior (e.g., as described herein) produces a replication-component anellovector (e.g., an anellovector that upon infecting a cell, enables the cell to produce additional copies of the anellovector without introducing further nucleic acid constructs, e.g., encoding one or more Anellovirus ORFs as described herein, into the cell).

In other embodiments, the genetic element does not comprise a sequence encoding an Anellovirus ORF1 molecule, or a splice variant or functional fragment thereof (e.g., a jelly-roll region, e.g., as described herein). In embodiments, enclosure of such a genetic element in a proteinaceous exterior (e.g., as described herein) produces a replication-incompetent anellovector (e.g., an anellovector that, upon infecting a cell, does not enable the infected cell to produce additional anellovectors, e.g., in the absence of one or more additional constructs, e.g., encoding one or more Anellovirus ORFs as described herein).

Expression Cassettes

In some embodiments, a genetic element construct (e.g., bacmid or donor vector) comprises one or more cassettes for expression of a polypeptide or noncoding RNA (e.g., a miRNA or an siRNA). In some embodiments, the genetic element construct comprises a cassette for expression of an effector (e.g., an exogenous or endogenous effector), e.g., a polypeptide or noncoding RNA, as described herein. In some embodiments, the genetic element construct comprises a cassette for expression of an Anellovirus protein (e.g., an Anellovirus ORF1, ORF2, ORF2/2, ORF2/3, ORF1/1, or ORF1/2, or a functional fragment thereof). The expression cassettes may, in some embodiments, be located within the genetic element sequence. In embodiments, an expression cassette for an effector is located within the genetic element sequence. In embodiments, an expression cassette for an Anellovirus protein is located within the genetic element sequence. In other embodiments, the expression cassettes are located at a position within the genetic element construct outside of the sequence of the genetic element (e.g., in the backbone). In embodiments, an expression cassette for an Anellovirus protein is located at a position within the genetic element construct outside of the sequence of the genetic element (e.g., in the backbone).

A polypeptide expression cassette generally comprises a promoter and a coding sequence encoding a polypeptide, e.g., an effector (e.g., an exogenous or endogenous effector as described herein) or an Anellovirus protein (e.g., a sequence encoding an Anellovirus ORF1, ORF2, ORF2/2, ORF2/3, ORF1/1, or ORF1/2, or a functional fragment thereof). Exemplary promoters that can be included in an polypeptide expression cassette (e.g., to drive expression of the polypeptide) include, without limitation, constitutive promoters (e.g., CMV, RSV, PGK, EF1a, or SV40), cell or tissue-specific promoters (e.g., skeletal α-actin promoter, myosin light chain 2A promoter, dystrophin promoter, muscle creatine kinase promoter, liver albumin promoter, hepatitis B virus core promoter, osteocalcin promoter, bone sialoprotein promoter, CD2 promoter, immunoglobulin heavy chain promoter, T cell receptor a chain promoter, neuron-specific enolase (NSE) promoter, or neurofilament light-chain promoter), and inducible promoters (e.g., zinc-inducible sheep metallothionine (MT) promoter; the dexamethasone (Dex)-inducible mouse mammary tumor virus (MMTV) promoter; the T7 polymerase promoter system, tetracycline-repressible system, tetracycline-inducible system, RU486-inducible system, rapamycin-inducible system), e.g., as described herein. In some embodiments, the expression cassette further comprises an enhancer, e.g., as described herein.

Design and Production of a Genetic Element Construct

Various methods are available for synthesizing a genetic element construct (e.g., a bacmid or donor vector). For instance, the genetic element construct sequence may be divided into smaller overlapping pieces (e.g., in the range of about 100 bp to about 10 kb segments or individual ORFs) that are easier to synthesize. These DNA segments are synthesized from a set of overlapping single-stranded oligonucleotides. The resulting overlapping synthons are then assembled into larger pieces of DNA, e.g., the genetic element construct. The segments or ORFs may be assembled into the genetic element construct, e.g., by in vitro recombination or unique restriction sites at 5′ and 3′ ends to enable ligation.

The genetic element construct can be synthesized with a design algorithm that parses the construct sequence into oligo-length fragments, creating suitable design conditions for synthesis that take into account the complexity of the sequence space. Oligos are then chemically synthesized on semiconductor-based, high-density chips, where over 200,000 individual oligos are synthesized per chip. The oligos are assembled with an assembly techniques, such as BioFab®, to build longer DNA segments from the smaller oligos. This is done in a parallel fashion, so hundreds to thousands of synthetic DNA segments are built at one time.

Each genetic element construct or segment of the genetic element construct may be sequence verified. In some embodiments, high-throughput sequencing of RNA or DNA can take place using AnyDot.chips (Genovoxx, Germany), which allows for the monitoring of biological processes (e.g., miRNA expression or allele variability (SNP detection). Other high-throughput sequencing systems include those disclosed in Venter, J., et al. Science 16 Feb. 2001; Adams, M. et al, Science 24 Mar. 2000; and M. J, Levene, et al. Science 299:682-686, January 2003; as well as US Publication Application No. 20030044781 and 2006/0078937. Overall such systems involve sequencing a target nucleic acid molecule having a plurality of bases by the temporal addition of bases via a polymerization reaction that is measured on a molecule of nucleic acid, i.e., the activity of a nucleic acid polymerizing enzyme on the template nucleic acid molecule to be sequenced is followed in real time. In some embodiments, shotgun sequencing is performed.

A genetic element construct can be designed such that factors for replicating or packaging may be supplied in cis or in trans, relative to the genetic element. For example, when supplied in cis, the genetic element may comprise one or more genes encoding an Anellovirus ORF1, ORF1/1, ORF1/2, ORF2, ORF2/2, ORF2/3, or ORF2t/3, e.g., as described herein. In some embodiments, replication and/or packaging signals can be incorporated into a genetic element, for example, to induce amplification and/or encapsulation. In some embodiments, an effector is inserted into a specific site in the genome. In some embodiments, one or more viral ORFs are replaced with an effector.

In another example, when replication or packaging factors are supplied in trans, the genetic element may lack genes encoding one or more of an Anellovirus ORF1, ORF1/1, ORF1/2, ORF2, ORF2/2, ORF2/3, or ORF2t/3, e.g., as described herein; this protein or proteins may be supplied, e.g., by another nucleic acid, e.g., a helper nucleic acid. In some embodiments, minimal cis signals (e.g., 5′ UTR and/or GC-rich region) are present in the genetic element. In some embodiments, the genetic element does not encode replication or packaging factors (e.g., replicase and/or capsid proteins). Such factors may, in some embodiments, be supplied by one or more helper nucleic acids (e.g., a helper viral nucleic acid, a helper plasmid, or a helper nucleic acid integrated into the host cell genome). In some embodiments, the helper nucleic acids express proteins and/or RNAs sufficient to induce amplification and/or packaging, but may lack their own packaging signals. In some embodiments, the genetic element and the helper nucleic acid are introduced into the host cell (e.g., concurrently or separately), resulting in amplification and/or packaging of the genetic element but not of the helper nucleic acid.

In some embodiments, the genetic element construct may be designed using computer-aided design tools.

General methods of making constructs are described in, for example, Khudyakov & Fields, Artificial DNA: Methods and Applications, CRC Press (2002); in Zhao, Synthetic Biology: Tools and Applications, (First Edition), Academic Press (2013); and Egli & Herdewijn, Chemistry and Biology of Artificial Nucleic Acids, (First Edition), Wiley-VCH (2012).

Effectors

The compositions (e.g., bacmids, donor vectors, cells, and baculoviruses) and methods described herein can be used to produce a genetic element of an anellovector comprising a sequence encoding an effector (e.g., an exogenous effector or an endogenous effector), e.g., as described herein. The effector may be, in some instances, an endogenous effector or an exogenous effector. In some embodiments, the effector is a therapeutic effector. In some embodiments, the effector comprises a polypeptide (e.g., a therapeutic polypeptide or peptide, e.g., as described herein). In some embodiments, the effector comprises a non-coding RNA (e.g., an miRNA, siRNA, shRNA, mRNA, lncRNA, RNA, DNA, antisense RNA, or gRNA). In some embodiments, the effector comprises a regulatory nucleic acid, e.g., as described herein.

In some embodiments, the effector-encoding sequence may be inserted into the genetic element, e.g., at a non-coding region, e.g., a noncoding region disposed 3′ of the open reading frames and 5′ of the GC-rich region of the genetic element, in the 5′ noncoding region upstream of the TATA box, in the 5′ UTR, in the 3′ noncoding region downstream of the poly-A signal, or upstream of the GC-rich region. In some embodiments, the effector-encoding sequence may be inserted into the genetic element, e.g., in a coding sequence (e.g., in a sequence encoding an Anellovirus ORF1, ORF1/1, ORF1/2, ORF2, ORF2/2, ORF2/3, and/or ORF2t/3, e.g., as described herein). In some embodiments, the effector-encoding sequence replaces all or a part of the open reading frame. In some embodiments, the genetic element comprises a regulatory sequence (e.g., a promoter or enhancer, e.g., as described herein) operably linked to the effector-encoding sequence.

In some embodiments, the genetic element comprising the effector is produced from a genetic element construct (e.g., a bacmid) as described herein, e.g., by rolling circle replication of a genetic element sequence disposed thereon. In some embodiments, the bacmid comprises one copy of the effector-encoding sequence. In some embodiments, the bacmid comprises one or more (e.g., 1, 2, 3, 4, 5, or all) sequences encoding Anellovirus ORF proteins (e.g., ORF1, ORF2, ORF2/2, ORF2/3, ORF1/1, or ORF1/2), or functional fragments thereof. In some embodiments, the bacmid does not comprise one or more sequences encoding Anellovirus ORF proteins (e.g., ORF1, ORF2, ORF2/2, ORF2/3, ORF1/1, or ORF1/2), or functional fragments thereof. In embodiments, the bacmid does not comprise any sequences encoding Anellovirus ORF proteins or functional fragments thereof.

### Host Cells

The anellovectors described herein can be produced, for example, in a host cell (e.g., an insect cell, e.g., an Sf9 cell). Generally, a host cell is provided that comprises an anellovector genetic element and the components of an anellovector proteinaceous exterior (e.g., a polypeptide encoded by an Anellovirus ORF1 nucleic acid or an Anellovirus ORF1 molecule). In some embodiments, the host cell comprises a bacmid encoding one or more of the components of the anellovector proteinaceous exterior. In embodiments, the components of the anellovector proteinaceous exterior are expressed in the host cell from the bacmid. In some embodiments, the host cell comprises a bacmid (e.g., the same bacmid or a different bacmid) comprising the sequence of the anellovector genetic element. In embodiments, the genetic element is produced from the bacmid comprising its sequence. The host cell is then incubated under conditions suitable for enclosure of the genetic element within the proteinaceous exterior (e.g., culture conditions as described herein). In some embodiments, the host cell is further incubated under conditions suitable for release of the anellovector from the host cell, e.g., into the surrounding supernatant. In some embodiments, the host cell is lysed for harvest of anellovectors from the cell lysate. In some embodiments, an anellovector may be introduced to a host cell line grown to a high cell density.

Introduction of Genetic Elements into Host Cells

The genetic element, or a nucleic acid construct comprising the sequence of a genetic element, may be introduced into a host cell (e.g., an insect cell, e.g., an Sf9 cell), e.g., a host cell comprising one or more components of an anellovector proteinaceous exterior (e.g., an ORF1 molecule or a functional fragment thereof). In some embodiments, the genetic element itself is introduced into the host cell. In some embodiments, a genetic element construct comprising the sequence of the genetic element (e.g., as described herein) is introduced into the host cell. A genetic element or genetic element construct can be introduced into a host cell, for example, using methods known in the art. For example, a genetic element or genetic element construct can be introduced into a host cell by transfection (e.g., stable transfection or transient transfection). In embodiments, the genetic element or genetic element construct is introduced into the host cell by lipofectamine transfection. In embodiments, the genetic element or genetic element construct is introduced into the host cell by calcium phosphate transfection. In some embodiments, the genetic element or genetic element construct is introduced into the host cell by electroporation. In some embodiments, the genetic element or genetic element construct is introduced into the host cell using a gene gun. In some embodiments, the genetic element or genetic element construct is introduced into the host cell by nucleofection. In some embodiments, the genetic element or genetic element construct is introduced into the host cell by PEI transfection. In some embodiments, the genetic element is introduced into the host cell by contacting the host cell with an anellovector comprising the genetic element

In embodiments, the genetic element construct is capable of replication once introduced into the host cell. In embodiments, the genetic element can be produced from the genetic element construct once introduced into the host cell. In some embodiments, the genetic element is produced in the host cell by a polymerase, e.g., using the genetic element construct as a template.

In some embodiments, the genetic elements or vectors comprising the genetic elements are introduced (e.g., transfected) into cell lines that express a viral polymerase protein in order to achieve expression of the anellovector. To this end, cell lines that express an anellovector polymerase protein may be utilized as appropriate host cells. Host cells may be similarly engineered to provide other viral functions or additional functions.

To prepare the anellovector disclosed herein, a genetic element construct may be used to transfect host cells that provide anellovector proteins and functions required for replication and production. Alternatively, host cells may be transfected with a second construct (e.g., a bacmid) providing anellovector proteins and functions before, during, or after transfection by the genetic element or vector comprising the genetic element disclosed herein. In some embodiments, the second construct may be useful to complement production of an incomplete viral particle. The second construct (e.g., bacmid) may have a conditional growth defect, such as host range restriction or temperature sensitivity, e.g., which allows the subsequent selection of transfectant viruses. In some embodiments, the second construct may provide one or more replication proteins utilized by the host cells to achieve expression of the anellovector. In some embodiments, the host cells may be transfected with vectors encoding viral proteins such as the one or more replication proteins. In some embodiments, the second construct comprises an antiviral sensitivity.

The genetic element or vector comprising the genetic element disclosed herein can, in some instances, be replicated and produced into anellovectors using techniques known in the art. For example, various viral culture methods are described, e.g., in U.S. Pat. Nos. 4,650,764; 5,166,057; 5,854,037; European Patent Publication EP 0702085A1; U.S. patent application Ser. No. 09/152,845; International Patent Publications PCT WO97/12032; WO96/34625; European Patent Publication EP-A780475; WO 99/02657; WO 98/53078; WO 98/02530; WO 99/15672; WO 98/13501; WO 97/06270; and EPO 780 47SA1, each of which is incorporated by reference herein in its entirety.

Methods for Providing Anellovirus Protein(s) in Cis or Trans

In some embodiments (e.g., cis embodiments described herein), the genetic element construct (e.g., bacmid) further comprises one or more expression cassettes comprising a coding sequence for an Anellovirus ORF (e.g., an Anellovirus ORF1, ORF2, ORF2/2, ORF2/3, ORF1/1, or ORF1/2, or a functional fragment thereof). In embodiments, the genetic element construct comprises an expression cassette comprising a coding sequence for an Anellovirus ORF1, or a splice variant or functional fragment thereof. Such genetic element constructs, which comprise expression cassettes for the effector as well as the one or more Anellovirus ORFs, may be introduced into host cells. Host cells (e.g., insect cells, e.g., Sf9 cells) comprising such genetic element constructs may, in some instances, be capable of producing the genetic elements and components for proteinaceous exteriors, and for enclosure of the genetic elements within proteinaceous exteriors, without requiring additional nucleic acid constructs or integration of expression cassettes into the host cell genome. In other words, such genetic element constructs may be used for cis anellovector production methods in host cells, e.g., as described herein.

In some embodiments (e.g., trans embodiments described herein), the genetic element construct does not comprise an expression cassette comprising a coding sequence for one or more Anellovirus ORFs (e.g., an Anellovirus ORF1, ORF2, ORF2/2, ORF2/3, ORF1/1, or ORF1/2, or a functional fragment thereof). In embodiments, the genetic element construct is a bacmid. In embodiments, the genetic element construct is not a bacmid. In embodiments, the genetic element construct does not comprise an expression cassette comprising a coding sequence for an Anellovirus ORF1, or a splice variant or functional fragment thereof. Such genetic element constructs, which comprise expression cassettes for the effector but lack expression cassettes for one or more Anellovirus ORFs (e.g., Anellovirus ORF1 or a splice variant or functional fragment thereof), may be introduced into host cells. Host cells comprising such genetic element constructs may, in some instances, require additional nucleic acid constructs or integration of expression cassettes into the host cell genome for production of one or more components of the anellovector (e.g., the proteinaceous exterior proteins). In some embodiments, host cells (e.g., insect cells, e.g., Sf9 cells) comprising such genetic element constructs are incapable of enclosure of the genetic elements within proteinaceous exteriors in the absence of an additional nucleic construct encoding an Anellovirus ORF1 molecule. In other words, such genetic element constructs may be used for trans anellovector production methods in host cells, e.g., as described herein.

Helpers

In some embodiments, a helper construct is introduced into a host cell (e.g., a host cell comprising a genetic element construct or a genetic element as described herein), e.g., an insect cell (e.g., an Sf9 cell). In some embodiments, the helper construct is introduced into the host cell prior to introduction of the genetic element construct. In some embodiments, the helper construct is introduced into the host cell concurrently with the introduction of the genetic element construct. In some embodiments, the helper construct is introduced into the host cell after introduction of the genetic element construct.

Exemplary Cell Types

Exemplary host cells suitable for production of anellovectors include, without limitation, insect cells (e.g., Sf9 cells, Sf21 cells, or Hi5 cells), e.g., as described herein. In some embodiments, the insect cell is derived from Bombyx mori, Mamestra brassicae, Spodoptera frugiperda, Trichoplusia ni, or Drosophila melanogaster.

In some embodiments, the anellovector is cultivated in continuous insect cell line (e.g., immortalized cell lines that can be serially propagated).

### Culture Conditions

Host cells (e.g., insect cells, e.g., Sf9 cells) comprising a genetic element and components of a proteinaceous exterior can be incubated under conditions suitable for enclosure of the genetic element within the proteinaceous exterior, thereby producing an anellovector. Suitable culture conditions include those described, e.g., in Examples 1, 9-11, and 13-15. In some embodiments, the host cells are incubated in liquid media (e.g., Grace's Supplemented (TNM-FH), IPL-41, TC-100, Schneider's Drosophila, SF-900 II SFM, or and EXPRESS-FIVE™ SFM). In some embodiments, the host cells are incubated in adherent culture. In some embodiments, the host cells are incubated in suspension culture. In some embodiments, the host cells are incubated in a tube, bottle, microcarrier, or flask. In some embodiments, the host cells are incubated in a dish or well (e.g., a well on a plate). In some embodiments, the host cells are incubated under conditions suitable for proliferation of the host cells. In some embodiments, the host cells are incubated under conditions suitable for the host cells to release anellovectors produced therein into the surrounding supernatant.

The production of anellovector-containing cell cultures according to the present invention can be carried out in different scales (e.g., in flasks, roller bottles or bioreactors). The media used for the cultivation of the cells to be infected generally comprise the standard nutrients required for cell viability, but may also comprise additional nutrients dependent on the cell type. Optionally, the medium can be protein-free and/or serum-free. Depending on the cell type the cells can be cultured in suspension or on a substrate. In some embodiments, different media is used for growth of the host cells and for production of anellovectors.

### Harvest

Anellovectors produced by host cells (e.g., insect cells, e.g., Sf9 cells) can be harvested, e.g., according to methods known in the art. For example, anellovectors released into the surrounding supernatant by host cells in culture can be harvested from the supernatant (e.g., as described in Example 10). In some embodiments, the supernatant is separated from the host cells to obtain the anellovectors. In some embodiments, the host cells are lysed before or during harvest. In some embodiments, the anellovectors are harvested from the host cell lysates (e.g., as described in Example 16). In some embodiments, the anellovectors are harvested from both the host cell lysates and the supernatant. In some embodiments, the purification and isolation of anellovectors is performed according to known methods in virus production, for example, as described in Rinaldi, et al., DNA Vaccines: Methods and Protocols (Methods in Molecular Biology), 3rd ed. 2014, Humana Press (incorporated herein by reference in its entirety). In some embodiments, the anellovector may be harvested and/or purified by separation of solutes based on biophysical properties, e.g., ion exchange chromatography or tangential flow filtration, prior to formulation with a pharmaceutical excipient.

### Enrichment and Purification

Harvested anellovectors can be purified and/or enriched, e.g., to produce an anellovector preparation. In some embodiments, the harvested anellovectors are isolated from other constituents or contaminants present in the harvest solution, e.g., using methods known in the art for purifying viral particles (e.g., purification by sedimentation, chromatography, and/or ultrafiltration). In some embodiments, the purification steps comprise removing one or more of serum, host cell DNA, host cell proteins, particles lacking the genetic element, and/or phenol red from the preparation. In some embodiments, the harvested anellovectors are enriched relative to other constituents or contaminants present in the harvest solution, e.g., using methods known in the art for enriching viral particles.

In some embodiments, the resultant preparation or a pharmaceutical composition comprising the preparation will be stable over an acceptable period of time and temperature, and/or be compatible with the desired route of administration and/or any devices this route of administration will require, e.g., needles or syringes.

### II. Anellovectors

In some aspects, the invention described herein comprises compositions and methods of using and making an anellovector, anellovector preparations, and therapeutic compositions. In some embodiments, the anellovectors are made using a bacmid as described herein. In certain embodiments, the genetic element of an anellovector. In some embodiments, the anellovector comprises one or more nucleic acids or polypeptides comprising a sequence, structure, and/or function that is based on an Anellovirus (e.g., an Anellovirus as described herein), or fragments or portions thereof, or other substantially non-pathogenic virus, e.g., a symbiotic virus, commensal virus, native virus. In some embodiments, an Anellovirus-based anellovector comprises at least one element exogenous to that Anellovirus, e.g., an exogenous effector or a nucleic acid sequence encoding an exogenous effector disposed within a genetic element of the anellovector. In some embodiments, an Anellovirus-based anellovector comprises at least one element heterologous to another element from that Anellovirus, e.g., an effector-encoding nucleic acid sequence that is heterologous to another linked nucleic acid sequence, such as a promoter element. In some embodiments, an anellovector comprises a genetic element (e.g., circular DNA, e.g., single stranded DNA), which comprise at least one element that is heterologous relative to the remainder of the genetic element and/or the proteinaceous exterior (e.g., an exogenous element encoding an effector, e.g., as described herein). An anellovector may be a delivery vehicle (e.g., a substantially non-pathogenic delivery vehicle) for a payload into a host, e.g., a human. In some embodiments, the anellovector is capable of replicating in a eukaryotic cell, e.g., a mammalian cell, e.g., a human cell. In some embodiments, the anellovector is substantially non-pathogenic and/or substantially non-integrating in the mammalian (e.g., human) cell. In some embodiments, the anellovector is substantially non-immunogenic in a mammal, e.g., a human. In some embodiments, the anellovector is replication-deficient. In some embodiments, the anellovector is replication-competent.

In some embodiments the anellovector comprises a curon, or a component thereof (e.g., a genetic element, e.g., comprising a sequence encoding an effector, and/or a proteinaceous exterior), e.g., as described in PCT Application No. PCT/US2018/037379, which is incorporated herein by reference in its entirety. In some embodiments the anellovector comprises an anellovector, or a component thereof (e.g., a genetic element, e.g., comprising a sequence encoding an effector, and/or a proteinaceous exterior), e.g., as described in PCT Application No. PCT/US19/65995, which is incorporated herein by reference in its entirety.

In an aspect, the invention includes an anellovector comprising (i) a genetic element comprising a promoter element, a sequence encoding an effector, (e.g., an endogenous effector or an exogenous effector, e.g., a payload), and a protein binding sequence (e.g., an exterior protein binding sequence, e.g., a packaging signal), wherein the genetic element is a single-stranded DNA, and has one or both of the following properties: is circular and/or integrates into the genome of a eukaryotic cell at a frequency of less than about 0.001%, 0.005%, 0.01%, 0.05%, 0.1%, 0.5%, 1%, 1.5%, or 2% of the genetic element that enters the cell; and (ii) a proteinaceous exterior; wherein the genetic element is enclosed within the proteinaceous exterior; and wherein the anellovector is capable of delivering the genetic element into a eukaryotic cell.

In some embodiments of the anellovector described herein, the genetic element integrates at a frequency of less than about 0.001%, 0.005%, 0.01%, 0.05%, 0.1%, 0.5%, 1%, 1.5%, or 2% of the genetic element that enters a cell. In some embodiments, less than about 0.01%, 0.05%, 0.1%, 0.5%, 1%, 2%, 3%, 4%, or 5% of the genetic elements from a plurality of the anellovectors administered to a subject will integrate into the genome of one or more host cells in the subject. In some embodiments, the genetic elements of a population of anellovectors, e.g., as described herein, integrate into the genome of a host cell at a frequency less than that of a comparable population of AAV viruses, e.g., at about a 50%, 60%, 70%, 75%, 80%, 85%, 90%, 95%, 100%, or more lower frequency than the comparable population of AAV viruses.

In an aspect, the invention includes an anellovector comprising: (i) a genetic element comprising a promoter element and a sequence encoding an effector (e.g., an endogenous effector or an exogenous effector, e.g., a payload), and a protein binding sequence (e.g., an exterior protein binding sequence), wherein the genetic element has at least 75% (e.g., at least 75, 76, 77, 78, 79, 80, 90, 91, 92, 93, 94, 95, 96, 97, 98, 99, or 100%) sequence identity to a wild-type Anellovirus sequence (e.g., a wild-type Torque Teno virus (TTV), Torque Teno mini virus (TTMV), or TTMDV sequence, e.g., a wild-type Anellovirus sequence as described herein); and (ii) a proteinaceous exterior; wherein the genetic element is enclosed within the proteinaceous exterior; and wherein the anellovector is capable of delivering the genetic element into a eukaryotic cell.

In one aspect, the invention includes an anellovector comprising:
a) a genetic element comprising (i) a sequence encoding an exterior protein (e.g., a non-pathogenic exterior protein), (ii) an exterior protein binding sequence that binds the genetic element to the non-pathogenic exterior protein, and (iii) a sequence encoding an effector (e.g., an endogenous or exogenous effector); and
b) a proteinaceous exterior that is associated with, e.g., envelops or encloses, the genetic element.

In some embodiments, the anellovector includes sequences or expression products from (or having >70%, 75%, 80%, 85%, 90%, 95%, 97%, 98%, 99%, 100% homology to) a non-enveloped, circular, single-stranded DNA virus. Animal circular single-stranded DNA viruses generally refer to a subgroup of single strand DNA (ssDNA) viruses, which infect eukaryotic non-plant hosts, and have a circular genome. Thus, animal circular ssDNA viruses are distinguishable from ssDNA viruses that infect prokaryotes (i.e. Microviridae and Inoviridae) and from ssDNA viruses that infect plants (i.e. Geminiviridae and Nanoviridae). They are also distinguishable from linear ssDNA viruses that infect non-plant eukaryotes (i.e. Parvoviridiae).

In some embodiments, the anellovector modulates a host cellular function, e.g., transiently or long term. In certain embodiments, the cellular function is stably altered, such as a modulation that persists for at least about 1 hr to about 30 days, or at least about 2 hrs, 6 hrs, 12 hrs, 18 hrs, 24 hrs, 2 days, 3, days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 60 days, or longer or any time therebetween. In certain embodiments, the cellular function is transiently altered, e.g., such as a modulation that persists for no more than about 30 mins to about 7 days, or no more than about 1 hr, 2 hrs, 3 hrs, 4 hrs, 5 hrs, 6 hrs, 7 hrs, 8 hrs, 9 hrs, 10 hrs, 11 hrs, 12 hrs, 13 hrs, 14 hrs, 15 hrs, 16 hrs, 17 hrs, 18 hrs, 19 hrs, 20 hrs, 21 hrs, 22 hrs, 24 hrs, 36 hrs, 48 hrs, 60 hrs, 72 hrs, 4 days, 5 days, 6 days, 7 days, or any time therebetween.

In some embodiments, the genetic element comprises a promoter element. In embodiments, the promoter element is selected from an RNA polymerase II-dependent promoter, an RNA polymerase III-dependent promoter, a PGK promoter, a CMV promoter, an EF-1α promoter, an SV40 promoter, a CAGG promoter, or a UBC promoter, TTV viral promoters, Tissue specific, U6 (pollIII), minimal CMV promoter with upstream DNA binding sites for activator proteins (TetR-VP16, Gal4-VP16, dCas9-VP16, etc). In embodiments, the promoter element comprises a TATA box. In embodiments, the promoter element is endogenous to a wild-type Anellovirus, e.g., as described herein.

In some embodiments, the genetic element comprises one or more of the following characteristics: single-stranded, circular, negative strand, and/or DNA. In embodiments, the genetic element comprises an episome. In some embodiments, the portions of the genetic element excluding the effector have a combined size of about 2.5-5 kb (e.g., about 2.8-4 kb, about 2.8-3.2 kb, about 3.6-3.9 kb, or about 2.8-2.9 kb), less than about 5 kb (e.g., less than about 2.9 kb, 3.2 kb, 3.6 kb, 3.9 kb, or 4 kb), or at least 100 nucleotides (e.g., at least 1 kb).

The anellovectors, compositions comprising anellovectors, methods using such anellovectors, etc., as described herein are, in some instances, based in part on the examples which illustrate how different effectors, for example miRNAs (e.g. against IFN or miR-625), shRNA, etc and protein binding sequences, for example DNA sequences that bind to capsid protein such as Q99153, are combined with proteinaceious exteriors, for example a capsid disclosed in Arch Virol (2007) 152: 1961-1975, to produce anellovectors which can then be used to deliver an effector to cells (e.g., animal cells, e.g., human cells or non-human animal cells such as pig or mouse cells). In embodiments, the effector can silence expression of a factor such as an interferon. The examples further describe how anellovectors can be made by inserting effectors into sequences derived, e.g., from an Anellovirus. It is on the basis of these examples that the description hereinafter contemplates various variations of the specific findings and combinations considered in the examples. For example, the skilled person will understand from the examples that the specific miRNAs are used just as an example of an effector and that other effectors may be, e.g., other regulatory nucleic acids or therapeutic peptides. Similarly, the specific capsids used in the examples may be replaced by substantially non-pathogenic proteins described hereinafter. The specifc Anellovirus sequences described in the examples may also be replaced by the Anellovirus sequences described hereinafter. These considerations similarly apply to protein binding sequences, regulatory sequences such as promoters, and the like. Independent thereof, the person skilled in the art will in particular consider such embodiments which are closely related to the examples.

In some embodiments, an anellovector, or the genetic element comprised in the anellovector, is introduced into a cell (e.g., a human cell). In some embodiments, the effector (e.g., an RNA, e.g., an miRNA), e.g., encoded by the genetic element of an anellovector, is expressed in a cell (e.g., a human cell), e.g., once the anellovector or the genetic element has been introduced into the cell. In embodiments, introduction of the anellovector, or genetic element comprised therein, into a cell modulates (e.g., increases or decreases) the level of a target molecule (e.g., a target nucleic acid, e.g., RNA, or a target polypeptide) in the cell, e.g., by altering the expression level of the target molecule by the cell. In embodiments, introduction of the anellovector, or genetic element comprised therein, decreases level of interferon produced by the cell. In embodiments, introduction of the anellovector, or genetic element comprised therein, into a cell modulates (e.g., increases or decreases) a function of the cell. In embodiments, introduction of the anellovector, or genetic element comprised therein, into a cell modulates (e.g., increases or decreases) the viability of the cell. In embodiments, introduction of the anellovector, or genetic element comprised therein, into a cell decreases viability of a cell (e.g., a cancer cell).

In some embodiments, an anellovector (e.g., a synthetic anellovector) described herein induces an antibody prevalence of less than 70% (e.g., less than about 60%, 50%, 40%, 30%, 20%, or 10% antibody prevalence). In embodiments, antibody prevalence is determined according to methods known in the art. In embodiments, antibody prevalence is determined by detecting antibodies against an Anellovirus (e.g., as described herein), or an anellovector based thereon, in a biological sample, e.g., according to the anti-TTV antibody detection method described in Tsuda et al. (1999; J. Virol. Methods 77: 199-206; incorporated herein by reference) and/or the method for determining anti-TTV IgG seroprevalence described in Kakkola et al. (2008; Virology 382: 182-189; incorporated herein by reference). Antibodies against an Anellovirus or an anellovector based thereon can also be detected by methods in the art for detecting anti-viral antibodies, e.g., methods of detecting anti-AAV antibodies, e.g., as described in Calcedo et al. (2013; Front. Immunol. 4(341): 1-7; incorporated herein by reference).

In some embodiments, a replication deficient, replication defective, or replication incompetent genetic element does not encode all of the necessary machinery or components required for replication of the genetic element. In some embodiments, a replication defective genetic element does not encode a replication factor. In some embodiments, a replication defective genetic element does not encode one or more ORFs (e.g., ORF1, ORF1/1, ORF1/2, ORF2, ORF2/2, ORF2/3, and/or ORF2t/3, e.g., as described herein). In some embodiments, the machinery or components not encoded by the genetic element may be provided in trans (e.g., using a helper, e.g., a helper virus or helper plasmid, or encoded in a nucleic acid comprised by the host cell, e.g., integrated into the genome of the host cell), e.g., such that the genetic element can undergo replication in the presence of the machinery or components provided in trans.

In some embodiments, a packaging deficient, packaging defective, or packaging incompetent genetic element cannot be packaged into a proteinaceous exterior (e.g., wherein the proteinaceous exterior comprises a capsid or a portion thereof, e.g., comprising a polypeptide encoded by an ORF1 nucleic acid, e.g., as described herein). In some embodiments, a packaging deficient genetic element is packaged into a proteinaceous exterior at an efficiency less than 10% (e.g., less than 10%, 9%, 8%, 7%, 6%, 5%, 4%, 3%, 2%, 1%, 0.5%, 0.1%, 0.01%, or 0.001%) compared to a wild-type Anellovirus (e.g., as described herein). In some embodiments, the packaging defective genetic element cannot be packaged into a proteinaceous exterior even in the presence of factors (e.g., ORF1, ORF1/1, ORF1/2, ORF2, ORF2/2, ORF2/3, or ORF2t/3) that would permit packaging of the genetic element of a wild-type Anellovirus (e.g., as described herein). In some embodiments, a packaging deficient genetic element is packaged into a proteinaceous exterior at an efficiency less than 10% (e.g., less than 10%, 9%, 8%, 7%, 6%, 5%, 4%, 3%, 2%, 1%, 0.5%, 0.1%, 0.01%, or 0.001%) compared to a wild-type Anellovirus (e.g., as described herein), even in the presence of factors (e.g., ORF1, ORF1/1, ORF1/2, ORF2, ORF2/2, ORF2/3, or ORF2t/3) that would permit packaging of the genetic element of a wild-type Anellovirus (e.g., as described herein).

In some embodiments, a packaging competent genetic element can be packaged into a proteinaceous exterior (e.g., wherein the proteinaceous exterior comprises a capsid or a portion thereof, e.g., comprising a polypeptide encoded by an ORF1 nucleic acid, e.g., as described herein). In some embodiments, a packaging competent genetic element is packaged into a proteinaceous exterior at an efficiency of at least 20% (e.g., at least 20%, 30%, 40%, 50%, 60%, 70%, 80%, 85%, 90%, 95%, 96%, 97%, 98%, 99%, 100%, or higher) compared to a wild-type Anellovirus (e.g., as described herein). In some embodiments, the packaging competent genetic element can be packaged into a proteinaceous exterior in the presence of factors (e.g., ORF1, ORF1/1, ORF1/2, ORF2, ORF2/2, ORF2/3, or ORF2t/3) that would permit packaging of the genetic element of a wild-type Anellovirus (e.g., as described herein). In some embodiments, a packaging competent genetic element is packaged into a proteinaceous exterior at an efficiency of at least 20% (e.g., at least 20%, 30%, 40%, 50%, 60%, 70%, 80%, 85%, 90%, 95%, 96%, 97%, 98%, 99%, 100%, or higher) compared to a wild-type Anellovirus (e.g., as described herein) in the presence of factors (e.g., ORF1, ORF1/1, ORF1/2, ORF2, ORF2/2, ORF2/3, or ORF2t/3) that would permit packaging of the genetic element of a wild-type Anellovirus (e.g., as described herein).

### Anelloviruses

In some embodiments, an anellovector, e.g., as described herein, comprises sequences or expression products derived from an Anellovirus. In some embodiments, an anellovector includes one or more sequences or expression products that are exogenous relative to the Anellovirus. In some embodiments, an anellovector includes one or more sequences or expression products that are endogenous relative to the Anellovirus. In some embodiments, an anellovector includes one or more sequences or expression products that are heterologous relative to one or more other sequences or expression products in the anellovector. Anelloviruses generally have single-stranded circular DNA genomes with negative polarity. Anelloviruses have not generally been linked to any human disease. However, attempts to link Anellovirus infection with human disease are confounded by the high incidence of asymptomatic Anellovirus viremia in control cohort population(s), the remarkable genomic diversity within the anellovirus viral family, the historical inability to propagate the agent in vitro, and the lack of animal model(s) of Anellovirus disease (Yzebe et al., Panminerva Med. (2002) 44:167-177; Biagini, P., Vet. Microbiol. (2004) 98:95-101).

Anelloviruses are generally transmitted by oronasal or fecal-oral infection, mother-to-infant and/or in utero transmission (Gerner et al., Ped. Infect. Dis. J. (2000) 19:1074-1077). Infected persons can, in some instances, be characterized by a prolonged (months to years) Anellovirus viremia. Humans may be co-infected with more than one genogroup or strain (Saback, et al., Scad. J. Infect. Dis. (2001) 33:121-125). There is a suggestion that these genogroups can recombine within infected humans (Rey et al., Infect. (2003) 31:226-233). The double stranded isoform (replicative) intermediates have been found in several tissues, such as liver, peripheral blood mononuclear cells and bone marrow (Kikuchi et al., J. Med. Virol. (2000) 61:165-170; Okamoto et al., Biochem. Biophys. Res. Commun. (2002) 270:657-662; Rodriguez-Inigo et al., Am. J. Pathol. (2000) 156:1227-1234).

In some embodiments, the genetic element comprises a nucleotide sequence encoding an amino acid sequence or a functional fragment thereof or a sequence having at least about 60%, 70% 80%, 85%, 90% 95%, 96%, 97%, 98%, 99%, or 100% sequence identity to any one of the amino acid sequences described herein, e.g., an Anellovirus amino acid sequence.

In some embodiments, an anellovector as described herein comprises one or more nucleic acid molecules (e.g., a genetic element as described herein) comprising a sequence having at least about 70%, 75%, 80%, 85%, 90%, 95%, 96%, 97%, 98%, 99%, or 100% sequence identity to an Anellovirus sequence, e.g., as described herein, or a fragment thereof.

In some embodiments, an anellovector as described herein comprises one or more nucleic acid molecules (e.g., a genetic element as described herein) comprising a sequence having at least about 70%, 75%, 80%, 85%, 90%, 95%, 96%, 97%, 98%, 99%, or 100% sequence identity to one or more of a TATA box, cap site, initiator element, transcriptional start site, 5′ UTR conserved domain, ORF1, ORF1/1, ORF1/2, ORF2, ORF2/2, ORF2/3, ORF2t/3, three open-reading frame region, poly(A) signal, GC-rich region, or any combination thereof, of an Anellovirus, e.g., as described herein. In some embodiments, the nucleic acid molecule comprises a sequence encoding a capsid protein, e.g., an ORF1, ORF1/1, ORF1/2, ORF2, ORF2/2, ORF2/3, ORF2t/3 sequence of any of the Anelloviruses described herein. In embodiments, the nucleic acid molecule comprises a sequence encoding a capsid protein comprising an amino acid sequence having at least about 70%, 75%, 80%, 85%, 90%, 95%, 96%, 97%, 98%, 99%, or 100% sequence identity to an Anellovirus ORF1 protein (or a splice variant or functional fragment thereof) or a polypeptide encoded by an Anellovirus ORF1 nucleic acid.

In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus ORF1 nucleic acid sequence of Table A1. In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus ORF1/1 nucleotide sequence of Table A1. In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus ORF1/2 nucleotide sequence of Table A1. In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus ORF2 nucleotide sequence of Table A1. In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus ORF2/2 nucleotide sequence of Table A1. In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus ORF2/3 nucleotide sequence of Table A1. In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus ORF2t/3 nucleotide sequence of Table A1. In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus TATA box nucleotide sequence of Table A1. In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus initiator element nucleotide sequence of Table A1. In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus transcriptional start site nucleotide sequence of Table A1. In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus 5′ UTR conserved domain nucleotide sequence of Table A1. In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus three open-reading frame region nucleotide sequence of Table A1. In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus poly(A) signal nucleotide sequence of Table A1. In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus GC-rich nucleotide sequence of Table A1.

In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus ORF1 nucleic acid sequence of Table B1. In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus ORF1/1 nucleotide sequence of Table B1. In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus ORF1/2 nucleotide sequence of Table B1. In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus ORF2 nucleotide sequence of Table B1. In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus ORF2/2 nucleotide sequence of Table B1. In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus ORF2/3 nucleotide sequence of Table B1. In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus TATA box nucleotide sequence of Table B1. In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus initiator element nucleotide sequence of Table B1. In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus transcriptional start site nucleotide sequence of Table B1. In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus 5′ UTR conserved domain nucleotide sequence of Table B1. In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus three open-reading frame region nucleotide sequence of Table B1. In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus poly(A) signal nucleotide sequence of Table B1. In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus GC-rich nucleotide sequence of Table B1.

In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus ORF1 nucleic acid sequence of Table C1. In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus ORF1/1 nucleotide sequence of Table C1. In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus ORF1/2 nucleotide sequence of Table C1. In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus ORF2 nucleotide sequence of Table C1. In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus ORF2/2 nucleotide sequence of Table C1. In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus ORF2/3 nucleotide sequence of Table C1. In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus TAIP nucleotide sequence of Table C1. In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus TATA box nucleotide sequence of Table C1. In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus initiator element nucleotide sequence of Table C1. In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus transcriptional start site nucleotide sequence of Table C1. In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus 5′ UTR conserved domain nucleotide sequence of Table C1. In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus three open-reading frame region nucleotide sequence of Table C1. In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus poly(A) signal nucleotide sequence of Table C1. In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus GC-rich nucleotide sequence of Table C1.

In some embodiments, the genetic element comprises a nucleotide sequence encoding an amino acid sequence or a functional fragment thereof or a sequence having at least about 60%, 70% 80%, 85%, 90% 95%, 96%, 97%, 98%, 99%, or 100% sequence identity to any one of the amino acid sequences described herein, e.g., an Anellovirus amino acid sequence.

In some embodiments, an anellovector as described herein comprises one or more nucleic acid molecules (e.g., a genetic element as described herein) comprising a sequence having at least about 70%, 75%, 80%, 85%, 90%, 95%, 96%, 97%, 98%, 99%, or 100% sequence identity to an Anellovirus sequence, e.g., as described herein, or a fragment thereof. In embodiments, the anellovector comprises a nucleic acid sequence selected from a sequence as shown in any of Tables A1-M2, or a sequence having at least 70%, 75%, 80%, 85%, 90%, 95%, 96%, 97%, 98%, 99%, or 100% sequence identity thereto. In embodiments, the anellovector comprises a polypeptide comprising a sequence as shown in any of Tables Tables A2-M2, or a sequence having at least 70%, 75%, 80%, 85%, 90%, 95%, 96%, 97%, 98%, 99%, or 100% sequence identity thereto.

In some embodiments, an anellovector as described herein comprises one or more nucleic acid molecules (e.g., a genetic element as described herein) comprising a sequence having at least about 70%, 75%, 80%, 85%, 90%, 95%, 96%, 97%, 98%, 99%, or 100% sequence identity to one or more of a TATA box, cap site, initiator element, transcriptional start site, 5′ UTR conserved domain, ORF1, ORF1/1, ORF1/2, ORF2, ORF2/2, ORF2/3, ORF2t/3, three open-reading frame region, poly(A) signal, GC-rich region, or any combination thereof, of any of the Anelloviruses described herein (e.g., an Anellovirus sequence as annotated, or as encoded by a sequence listed, in any of Tables A-M). In some embodiments, the nucleic acid molecule comprises a sequence encoding a capsid protein, e.g., an ORF1, ORF1/1, ORF1/2, ORF2, ORF2/2, ORF2/3, ORF2t/3 sequence of any of the Anelloviruses described herein (e.g., an Anellovirus sequence as annotated, or as encoded by a sequence listed, in any of Tables A-M). In embodiments, the nucleic acid molecule comprises a sequence encoding a capsid protein comprising an amino acid sequence having at least about 70%, 75%, 80%, 85%, 90%, 95%, 96%, 97%, 98%, 99%, or 100% sequence identity to an Anellovirus ORF1 or ORF2 protein (e.g., an ORF1 or ORF2 amino acid sequence as shown in any of Tables A2-M2, or an ORF1 or ORF2 amino acid sequence encoded by a nucleic acid sequence as shown in any of Tables A1-M1). In embodiments, the nucleic acid molecule comprises a sequence encoding a capsid protein comprising an amino acid sequence having at least about 70%, 75%, 80%, 85%, 90%, 95%, 96%, 97%, 98%, 99%, or 100% sequence identity to an Anellovirus ORF1 protein (e.g., an ORF1 amino acid sequence as shown in any of Tables A2-M2, or an ORF1 amino acid sequence encoded by a nucleic acid sequence as shown in any of Tables A1-M1).

In some embodiments, an anellovector as described herein is a chimeric anellovector. In some embodiments, a chimeric anellovector further comprises one or more elements, polypeptides, or nucleic acids from a virus other than an Anellovirus.

In embodiments, the chimeric anellovector comprises a plurality of polypeptides (e.g., Anellovirus ORF1, ORF1/1, ORF1/2, ORF2, ORF2/2, ORF2/3, and/or ORF2t/3) comprising sequences from a plurality of different Anelloviruses (e.g., as described herein). For example, a chimeric anellovector may comprise an ORF1 molecule from one Anellovirus (e.g., a Ring1 ORF1 molecule, or an ORF1 molecule having at least 75%, 80%, 85%, 90%, 95%, 96%, 97%, 98%, or 99% amino acid sequence identity thereto) and an ORF2 molecule from a different Anellovirus (e.g., a Ring2 ORF2 molecule, or an ORF2 molecule having at least 75%, 80%, 85%, 90%, 95%, 96%, 97%, 98%, or 99% amino acid sequence identity thereto). In another example, a chimeric anellovector may comprise a first ORF1 molecule from one Anellovirus (e.g., a Ring1 ORF1 molecule, or an ORF1 molecule having at least 75%, 80%, 85%, 90%, 95%, 96%, 97%, 98%, or 99% amino acid sequence identity thereto) and a second ORF1 molecule from a different Anellovirus (e.g., a Ring2 ORF1 molecule, or an ORF1 molecule having at least 75%, 80%, 85%, 90%, 95%, 96%, 97%, 98%, or 99% amino acid sequence identity thereto).

In some embodiments, the anellovector comprises a chimeric polypeptide (e.g., Anellovirus ORF1, ORF1/1, ORF1/2, ORF2, ORF2/2, ORF2/3, and/or ORF2t/3), e.g., comprising at least one portion from an Anellovirus (e.g., as described herein) and at least one portion from a different virus (e.g., as described herein).

In some embodiments, the anellovector comprises a chimeric polypeptide (e.g., Anellovirus ORF1, ORF1/1, ORF1/2, ORF2, ORF2/2, ORF2/3, and/or ORF2t/3), e.g., comprising at least one portion from one Anellovirus (e.g., as described herein) and at least one portion from a different Anellovirus (e.g., as described herein). In embodiments, the anellovector comprises a chimeric ORF1 molecule comprising at least one portion of an ORF1 molecule from one Anellovirus (e.g., as described herein), or an ORF1 molecule having at least 75%, 80%, 85%, 90%, 95%, 96%, 97%, 98%, or 99% amino acid sequence identity thereto, and at least one portion of an ORF1 molecule from a different Anellovirus (e.g., as described herein), or an ORF1 molecule having at least 75%, 80%, 85%, 90%, 95%, 96%, 97%, 98%, or 99% amino acid sequence identity thereto. In embodiments, the chimeric ORF1 molecule comprises an ORF1 jelly-roll domain from one Anellovirus, or a sequence having at least 75%, 80%, 85%, 90%, 95%, 96%, 97%, 98%, or 99% sequence identity thereto, and an ORF1 amino acid subsequence (e.g., as described herein) from a different Anellovirus, or a sequence having at least 75%, 80%, 85%, 90%, 95%, 96%, 97%, 98%, or 99% sequence identity thereto. In embodiments, the chimeric ORF1 molecule comprises an ORF1 arginine-rich region from one Anellovirus, or a sequence having at least 75%, 80%, 85%, 90%, 95%, 96%, 97%, 98%, or 99% sequence identity thereto, and an ORF1 amino acid subsequence (e.g., as described herein) from a different Anellovirus, or a sequence having at least 75%, 80%, 85%, 90%, 95%, 96%, 97%, 98%, or 99% sequence identity thereto. In embodiments, the chimeric ORF1 molecule comprises an ORF1 hypervariable domain from one Anellovirus, or a sequence having at least 75%, 80%, 85%, 90%, 95%, 96%, 97%, 98%, or 99% sequence identity thereto, and an ORF1 amino acid subsequence (e.g., as described herein) from a different Anellovirus, or a sequence having at least 75%, 80%, 85%, 90%, 95%, 96%, 97%, 98%, or 99% sequence identity thereto. In embodiments, the chimeric ORF1 molecule comprises an ORF1 N22 domain from one Anellovirus, or a sequence having at least 75%, 80%, 85%, 90%, 95%, 96%, 97%, 98%, or 99% sequence identity thereto, and an ORF1 amino acid subsequence (e.g., as described herein) from a different Anellovirus, or a sequence having at least 75%, 80%, 85%, 90%, 95%, 96%, 97%, 98%, or 99% sequence identity thereto. In embodiments, the chimeric ORF1 molecule comprises an ORF1 C-terminal domain from one Anellovirus, or a sequence having at least 75%, 80%, 85%, 90%, 95%, 96%, 97%, 98%, or 99% sequence identity thereto, and an ORF1 amino acid subsequence (e.g., as described herein) from a different Anellovirus, or a sequence having at least 75%, 80%, 85%, 90%, 95%, 96%, 97%, 98%, or 99% sequence identity thereto. In embodiments, the anellovector comprises a chimeric ORF1/1 molecule comprising at least one portion of an ORF1/1 molecule from one Anellovirus (e.g., as described herein), or an ORF1/1 molecule having at least 75%, 80%, 85%, 90%, 95%, 96%, 97%, 98%, or 99% amino acid sequence identity thereto, and at least one portion of an ORF1/1 molecule from a different Anellovirus (e.g., as described herein), or an ORF1/1 molecule having at least 75%, 80%, 85%, 90%, 95%, 96%, 97%, 98%, or 99% amino acid sequence identity thereto. In embodiments, the anellovector comprises a chimeric ORF1/2 molecule comprising at least one portion of an ORF1/2 molecule from one Anellovirus (e.g., as described herein), or an ORF1/2 molecule having at least 75%, 80%, 85%, 90%, 95%, 96%, 97%, 98%, or 99% amino acid sequence identity thereto, and at least one portion of an ORF1/2 molecule from a different Anellovirus (e.g., as described herein), or an ORF1/2 molecule having at least 75%, 80%, 85%, 90%, 95%, 96%, 97%, 98%, or 99% amino acid sequence identity thereto. In embodiments, the anellovector comprises a chimeric ORF2 molecule comprising at least one portion of an ORF2 molecule from one Anellovirus (e.g., as described herein), or an ORF2 molecule having at least 75%, 80%, 85%, 90%, 95%, 96%, 97%, 98%, or 99% amino acid sequence identity thereto, and at least one portion of an ORF2 molecule from a different Anellovirus (e.g., as described herein), or an ORF2 molecule having at least 75%, 80%, 85%, 90%, 95%, 96%, 97%, 98%, or 99% amino acid sequence identity thereto. In embodiments, the anellovector comprises a chimeric ORF2/2 molecule comprising at least one portion of an ORF2/2 molecule from one Anellovirus (e.g., as described herein), or an ORF2/2 molecule having at least 75%, 80%, 85%, 90%, 95%, 96%, 97%, 98%, or 99% amino acid sequence identity thereto, and at least one portion of an ORF2/2 molecule from a different Anellovirus (e.g., as described herein), or an ORF2/2 molecule having at least 75%, 80%, 85%, 90%, 95%, 96%, 97%, 98%, or 99% amino acid sequence identity thereto. In embodiments, the anellovector comprises a chimeric ORF2/3 molecule comprising at least one portion of an ORF2/3 molecule from one Anellovirus (e.g., as described herein), or an ORF2/3 molecule having at least 75%, 80%, 85%, 90%, 95%, 96%, 97%, 98%, or 99% amino acid sequence identity thereto, and at least one portion of an ORF2/3 molecule from a different Anellovirus (e.g., as described herein), or an ORF2/3 molecule having at least 75%, 80%, 85%, 90%, 95%, 96%, 97%, 98%, or 99% amino acid sequence identity thereto. In embodiments, the anellovector comprises a chimeric ORF2T/3 molecule comprising at least one portion of an ORF2T/3 molecule from one Anellovirus (e.g., as described herein), or an ORF2T/3 molecule having at least 75%, 80%, 85%, 90%, 95%, 96%, 97%, 98%, or 99% amino acid sequence identity thereto, and at least one portion of an ORF2T/3 molecule from a different Anellovirus (e.g., as described herein), or an ORF2T/3 molecule having at least 75%, 80%, 85%, 90%, 95%, 96%, 97%, 98%, or 99% amino acid sequence identity thereto.

Additional exemplary Anellovirus genomes, for which sequences or subsequences comprised therein can be utilized in the compositions and methods described herein (e.g., to form a genetic element of an anellovector or as the genetic element sequence in a bacmid, e.g., as described herein) are described, for example, in PCT Application Nos. PCT/US2018/037379 and PCT/US19/65995 (incorporated herein by reference in their entirety). In some embodiments, the exemplary Anellovirus sequences comprise a nucleic acid sequence as listed in any of Tables A1, A3, A5, A7, A9, A11, B1-B5, 1, 3, 5, 7, 9, 11, 13, 15, or 17 of PCT/US19/65995, incorporated herein by reference. In some embodiments, the exemplary Anellovirus sequences comprise an amino acid sequence as listed in any of Tables A2, A4, A6, A8, A10, A12, C1-C5, 2, 4, 6, 8, 10, 12, 14, 16, or 18 of PCT/US19/65995, incorporated herein by reference. In some embodiments, the exemplary Anellovirus sequences comprise an ORF1 molecule sequence, or a nucleic acid sequence encoding same, e.g., as listed in any of Tables 21, 23, 25, 27, 29, 31, 33, 35, D2, D4, D6, D8, D10, or 37A-37C of PCT/US19/65995, incorporated herein by reference.

TABLE A1
Exemplary Anellovirus nucleic acid sequence (Alphatorquevirus, Clade 3)

| Name | Ring1 |
|---|---|
| Genus/Clade | Alphatorquevirus, Clade 3 |
| Accession Number | AJ620231.1 |

Full Sequence: 3753 bp
1 10 20 30 40 50
| | | | | |
TGCTACGTCACTAACCCACGTGTCCTCTACAGGCCAATCGCAGTCTATGT
CGTGCACTTCCTGGGCATGGTCTACATAATTATATAAATGCTTGCACTTC
CGAATGGCTGAGTTTTTGCTGCCCGTCCGCGGAGAGGAGCCACGGCAGGG
GATCCGAACGTCCTGAGGGCGGGTGCCGGAGGTGAGTTTACACACCGAAG
TCAAGGGGCAATTCGGGCTCAGGACTGGCCGGGCTTTGGGCAAGGCTCTT
AAAAATGCACTTTTCTCGAATAAGCAGAAAGAAAAGGAAAGTGCTACTGC
TTTGCGTGCCAGCAGCTAAGAAAAAACCAACTGCTATGAGCTTCTGGAAA
CCTCCGGTACACAATGTCACGGGGATCCAACGCATGTGGTATGAGTCCTT
TCACCGTGGCCACGCTTCTTTTTGTGGTTGTGGGAATCCTATACTTCACA
TTACTGCACTTGCTGAAACATATGGCCATCCAACAGGCCCGAGACCTTCT
GGGCCACCGGGAGTAGACCCCAACCCCCACATCCGTAGAGCCAGGCCTGC
CCCGGCCGCTCCGGAGCCCTCACAGGTTGATTCGAGACCAGCCCTGACAT
GGCATGGGGATGGTGGAAGCGACGGAGGCGCTGGTGGTTCCGGAAGCGGT
GGACCCGTGGCAGACTTCGCAGACGATGGCCTCGATCAGCTCGTCGCCGC
CCTAGACGACGAAGAGTAAGGAGGCGCAGACGGTGGAGGAGGGGGAGACG
AAAAACAAGGACTTACAGACGCAGGAGACGCTTTAGACGCAGGGGACGAA
AAGCAAAACTTATAATAAAACTGTGGCAACCTGCAGTAATTAAAAGATGC
AGAATAAAGGGATACATACCACTGATTATAAGTGGGAACGGTACCTTTGC
CACAAACTTTACCAGTCACATAAATGACAGAATAATGAAAGGCCCCTTCG
GGGGAGGACACAGCACTATGAGGTTCAGCCTCTACATTTTGTTTGAGGAG
CACCTCAGACACATGAACTTCTGGACCAGAAGCAACGATAACCTAGAGCT
AACCAGATACTTGGGGGCTTCAGTAAAAATATACAGGCACCCAGACCAAG
ACTTTATAGTAATATACAACAGAAGAACCCCTCTAGGAGGCAACATCTAC
ACAGCACCCTCTCTACACCCAGGCAATGCCATTTTAGCAAAACACAAAAT
ATTAGTACCAAGTTTACAGACAAGACCAAAGGGTAGAAAAGCAATTAGAC
TAAGAATAGCACCCCCCACACTCTTTACAGACAAGTGGTACTTTCAAAAG
GACATAGCCGACCTCACCCTTTTCAACATCATGGCAGTTGAGGCTGACTT
GCGGTTTCCGTTCTGCTCACCACAAACTGACAACACTTGCATCAGCTTCC
AGGTCCTTAGTTCCGTTTACAACAACTACCTCAGTATTAATACCTTTAAT
AATGACAACTCAGACTCAAAGTTAAAAGAATTTTTAAATAAAGCATTTCC
AACAACAGGCACAAAAGGAACAAGTTTAAATGCACTAAATACATTTAGAA
CAGAAGGATGCATAAGTCACCCACAACTAAAAAAACCAAACCCACAAATA
AACAAACCATTAGAGTCACAATACTTTGCACCTTTAGATGCCCTCTGGGG
AGACCCCATATACTATAATGATCTAAATGAAAACAAAAGTTTGAACGATA
TCATTGAGAAAATACTAATAAAAAACATGATTACATACCATGCAAAACTA
AGAGAATTTCCAAATTCATACCAAGGAAACAAGGCCTTTTGCCACCTAAC
AGGCATATACAGCCCACCATACCTAAACCAAGGCAGAATATCTCCAGAAA
TATTTGGACTGTACACAGAAATAATTTACAACCCTTACACAGACAAAGGA
ACTGGAAACAAAGTATGGATGGACCCACTAACTAAAGAGAACAACATATA
TAAAGAAGGACAGAGCAAATGCCTACTGACTGACATGCCCCTATGGACTT
TACTTTTTGGATATACAGACTGGTGTAAAAAGGACACTAATAACTGGGAC
TTACCACTAAACTACAGACTAGTACTAATATGCCCTTATACCTTTCCAAA
ATTGTACAATGAAAAAGTAAAAGACTATGGGTACATCCCGTACTCCTACA
AATTCGGAGCGGGTCAGATGCCAGACGGCAGCAACTACATACCCTTTCAG
TTTAGAGCAAAGTGGTACCCCACAGTACTACACCAGCAACAGGTAATGGA
GGACATAAGCAGGAGCGGGCCCTTTGCACCTAAGGTAGAAAAACCAAGCA
CTCAGCTGGTAATGAAGTACTGTTTTAACTTTAACTGGGGCGGTAACCCT
ATCATTGAACAGATTGTTAAAGACCCCAGCTTCCAGCCCACCTATGAAAT
ACCCGGTACCGGTAACATCCCTAGAAGAATACAAGTCATCGACCCGCGGG
TCCTGGGACCGCACTACTCGTTCCGGTCATGGGACATGCGCAGACACACA
TTTAGCAGAGCAAGTATTAAGAGAGTGTCAGAACAACAAGAAACTTCTGA
CCTTGTATTCTCAGGCCCAAAAAAGCCTCGGGTCGACATCCCAAAACAAG
AAACCCAAGAAGAAAGCTCACATTCACTCCAAAGAGAATCGAGACCGTGG
GAGACCGAGGAAGAAAGCGAGACAGAAGCCCTCTCGCAAGAGAGCCAAGA
GGTCCCCTTCCAACAGCAGTTGCAGCAGCAGTACCAAGAGCAGCTCAAGC
TCAGACAGGGAATCAAAGTCCTCTTCGAGCAGCTCATAAGGACCCAACAA
GGGGTCCATGTAAACCCATGCCTACGGTAGGTCCCAGGCAGTGGCTGTTT
CCAGAGAGAAAGCCAGCCCCAGCTCCTAGCAGTGGAGACTGGGCCATGGA
GTTTCTCGCAGCAAAAATATTTGATAGGCCAGTTAGAAGCAACCTTAAAG
ATACCCCTTACTACCCATATGTTAAAAACCAATACAATGTCTACTTTGAC
CTTAAATTTGAATAAACAGCAGCTTCAAACTTGCAAGGCCGTGGGAGTTT
CACTGGTCGGTGTCTACCTCTAAAGGTCACTAAGCACTCCGAGCGTAAGC
GAGGAGTGCGACCCTCCCCCCTGGAACAACTTCTTCGGAGTCCGGCGCTA
CGCCTTCGGCTGCGCCGGACACCTCAGACCCCCCCTCCACCCGAAACGCT
TGCGCGTTTCGGACCTTCGGCGTCGGGGGGGTCGGGAGCTTTATTAAACG
GACTCCGAAGTGCTCTTGGACACTGAGGGGGTGAACAGCAACGAAAGTGA
GTGGGGCCAGACTTCGCCATAAGGCCTTTATCTTCTTGCCATTTGTCAGT
GTCCGGGGTCGCCATAGGCTTCGGGCTCGTTTTTAGGCCTTCCGGACTAC
AAAAATCGCCATTTTGGTGACGTCACGGCCGCCATCTTAAGTAGTTGAGG
CGGACGGTGGCGTGAGTTCAAAGGTCACCATCAGCCACACCTACTCAAAA
TGGTGGACAATTTCTTCCGGGTCAAAGGTTACAGCCGCCATGTTAAAACA
CGTGACGTATGACGTCACGGCCGCCATTTTGTGACACAAGATGGCCGACT
TCCTTCCTCTTTTTCAAAAAAAAGCGGAAGTGCCGCCGCGGCGGCGGGGG
GCGGCGCGCTGCGCGCGCCGCCCAGTAGGGGGAGCCATGCGCCCCCCCCC
GCGCATGCGCGGGGCCCCCCCCCGCGGGGGGCTCCGCCCCCCGGCCCCCC
CCG (SEQ ID NO: 16)

Annotations:

| Putative Domain | Base range |
|---|---|
| TATA Box | 83-88 |
| Cap Site | 104-111 |
| Transcriptional Start Site | 111 |
| 5′ UTR Conserved Domain | 170-240 |
| ORF2 | 336-719 |
| ORF2/2 | 336-715; 2363-2789 |
| ORF2/3 | 336-715; 2565-3015 |
| ORF2t/3 | 336-388; 2565-3015 |
| ORF1 | 599-2830 |
| ORF1/1 | 599-715; 2363-2830 |
| ORF1/2 | 599-715; 2565-2789 |
| Three open-reading frame region | 2551-2786 |
| Poly(A) Signal | 3011-3016 |
| GC-rich region | 3632-3753 |

TABLE A2
Exemplary Anellovirus amino acid sequences
(Alphatorquevirus, Clade 3)
Ring1 (Alphatorquevirus Clade 3)

| | |
|---|---|
| ORF2 | MSFWKPPVHNVTGIQRMWYESFHRGHASFCGCGNPILHITAL |
| | AETYGHPTGPRPSGPPGVDPNPHIRRARPAPAAPEPSQVDSR |
| | PALTWHGDGGSDGGAGGSGSGGPVADFADDGLDQLVAALDDE |
| | E |
| | (SEQ ID NO: 17) |
| ORF2/2 | MSFWKPPVHNVTGIQRMWYESFHRGHASFCGCGNPILHITAL |
| | AETYGHPTGPRPSGPPGVDPNPHIRRARPAPAAPEPSQVDSR |
| | PALTWHGDGGSDGGAGGSGSGGPVADFADDGLDQLVAALDDE |
| | ELLKTPASSPPMKYPVPVTSLEEYKSSTRGSWDRTTRSGHGT |
| | CADTHLAEQVLRECQNNKKLLTLYSQAQKSLGSTSQNKKPKK |
| | KAHIHSKENRDRGRPRKKARQKPSRKRAKRSPSNSSCSSSTK |
| | SSSSSDRESKSSSSSS |
| | (SEQ ID NO: 18) |
| ORF2/3 | MSFWKPPVHNVTGIQRMWYESFHRGHASFCGCGNPILHITAL |
| | AETYGHPTGPRPSGPPGVDPNPHIRRARPAPAAPEPSQVDSR |
| | PALTWHGDGGSDGGAGGSGSGGPVADFADDGLDQLVAALDDE |
| | EPKKASGRHPKTRNPRRKLTFTPKRIETVGDRGRKRDRSPLA |
| | REPRGPLPTAVAAAVPRAAQAQTGNQSPLRAAHKDPTRGPCK |
| | PMPTVGPRQWLFPERKPAPAPSSGDWAMEFLAAKIFDRPVRS |
| | NLKDTPYYPYVKNQYNVYFDLKFE |
| | (SEQ ID NO: 19) |
| ORF2t/3 | MSFWKPPVHNVTGIQRMWPKKASGRHPKTRNPRRKLTFTPKR |
| | IETVGDRGRKRDRSPLAREPRGPLPTAVAAAVPRAAQAQTGN |
| | QSPLRAAHKDPTRGPCKPMPTVGPRQWLFPERKPAPAPSSGD |
| | WAMEFLAAKIFDRPVRSNLKDTPYYPYVKNQYNVYFDLKFE |
| | (SEQ ID NO: 20) |
| ORF1 | MAWGWWKRRRRWWFRKRWTRGRLRRRWPRSARRRPRRRRVRR |
| | RRRWRRGRRKTRTYRRRRRFRRRGRKAKLIIKLWQPAVIKRC |
| | RIKGYIPLIISGNGTFATNFTSHINDRIMKGPFGGGHSTMRF |
| | SLYILFEEHLRHMNFWTRSNDNLELTRYLGASVKIYRHPDQD |
| | FIVIYNRRTPLGGNIYTAPSLHPGNAILAKHKILVPSLQTRP |
| | KGRKAIRLRIAPPTLFTDKWYFQKDIADLTLFNIMAVEADLR |
| | FPFCSPQTDNTCISFQVLSSVYNNYLSINTFNNDNSDSKLKE |
| | FLNKAFPTTGTKGTSLNALNTFRTEGCISHPQLKKPNPQINK |
| | PLESQYFAPLDALWGDPIYYNDLNENKSLNDIIEKILIKNMI |
| | TYHAKLREFPNSYQGNKAFCHLTGIYSPPYLNQGRISPEIFG |
| | LYTEIIYNPYTDKGTGNKVWMDPLTKENNIYKEGQSKCLLTD |
| | MPLWTLLFGYTDWCKKDTNNWDLPLNYRLVLICPYTFPKLYN |
| | EKVKDYGYIPYSYKFGAGQMPDGSNYIPFQFRAKWYPTVLHQ |
| | QQVMEDISRSGPFAPKVEKPSTQLVMKYCFNFNWGGNPIIEQ |
| | IVKDPSFQPTYEIPGTGNIPRRIQVIDPRVLGPHYSFRSWDM |
| | RRHTFSRASIKRVSEQQETSDLVFSGPKKPRVDIPKQETQEE |
| | SSHSLQRESRPWETEEESETEALSQESQEVPFQQQLQQQYQE |
| | QLKLRQGIKVLFEQLIRTQQGVHVNPCLR |
| | (SEQ ID NO: 21) |
| ORF1/1 | MAWGWWKRRRRWWFRKRWTRGRLRRRWPRSARRRPRRRRIVK |
| | DPSFQPTYEIPGTGNIPRRIQVIDPRVLGPHYSFRSWDMRRH |
| | TFSRASIKRVSEQQETSDLVFSGPKKPRVDIPKQETQEESSH |
| | SLQRESRPWETEEESETEALSQESQEVPFQQQLQQQYQEQLK |
| | LRQGIKVLFEQLIRTQQGVHVNPCLR |
| | (SEQ ID NO: 22) |
| ORF1/2 | MAWGWWKRRRRWWFRKRWTRGRLRRRWPRSARRRPRRRRAQK |
| | SLGSTSQNKKPKKKAHIHSKENRDRGRPRKKARQKPSRKRAK |
| | RSPSNSSCSSSTKSSSSSDRESKSSSSSS |
| | (SEQ ID NO: 23) |

TABLE B1
Exemplary Anellovirus nucleic acid sequence (Betatorquevirus)

| Name | Ring2 |
|---|---|
| Genus/Clade | Betatorquevirus |
| Accession Number | JX134045.1 |

Full Sequence: 2797 bp
1 10 20 30 40 50
| | | | | |
TAATAAATATTCAACAGGAAAACCACCTAATTTAAATTGCCGACCACAAA
CCGTCACTTAGTTCCCCTTTTTGCAACAACTTCTGCTTTTTTCCAACTGC
CGGAAAACCACATAATTTGCATGGCTAACCACAAACTGATATGCTAATTA
ACTTCCACAAAACAACTTCCCCTTTTAAAACCACACCTACAAATTAATTA
TTAAACACAGTCACATCCTGGGAGGTACTACCACACTATAATACCAAGTG
CACTTCCGAATGGCTGAGTTTATGCCGCTAGACGGAGAACGCATCAGTTA
CTGACTGCGGACTGAACTTGGGCGGGTGCCGAAGGTGAGTGAAACCACCG
AAGTCAAGGGGCAATTCGGGCTAGTTCAGTCTAGCGGAACGGGCAAGAAA
CTTAAAATTATTTTATTTTTCAGATGAGCGACTGCTTTAAACCAACATGC
TACAACAACAAAACAAAGCAAACTCACTGGATTAATAACCTGCATTTAAC
CCACGACCTGATCTGCTTCTGCCCAACACCAACTAGACACTTATTACTAG
CTTTAGCAGAACAACAAGAAACAATTGAAGTGTCTAAACAAGAAAAAGAA
AAAATAACAAGATGCCTTATTACTACAGAAGAAGACGGTACAACTACAGA
CGTCCTAGATGGTATGGACGAGGTTGGATTAGACGCCCTTTTCGCAGAAG
ATTTCGAAGAAAAAGAAGGGTAAGACCTACTTATACTACTATTCCTCTAA
AGCAATGGCAACCGCCATATAAAAGAACATGCTATATAAAAGGACAAGAC
TGTTTAATATACTATAGCAACTTAAGACTGGGAATGAATAGTACAATGTA
TGAAAAAAGTATTGTACCTGTACATTGGCCGGGAGGGGGTTCTTTTTCTG
TAAGCATGTTAACTTTAGATGCCTTGTATGATATACATAAACTTTGTAGA
AACTGGTGGACATCCACAAACCAAGACTTACCACTAGTAAGATATAAAGG
ATGCAAAATAACATTTTATCAAAGCACATTTACAGACTACATAGTAAGAA
TACATACAGAACTACCAGCTAACAGTAACAAACTAACATACCCAAACACA
CATCCACTAATGATGATGATGTCTAAGTACAAACACATTATACCTAGTAG
ACAAACAAGAAGAAAAAAGAAACCATACACAAAAATATTTGTAAAACCAC
CTCCGCAATTTGAAAACAAATGGTACTTTGCTACAGACCTCTACAAAATT
CCATTACTACAAATACACTGCACAGCATGCAACTTACAAAACCCATTTGT
AAAACCAGACAAATTATCAAACAATGTTACATTATGGTCACTAAACACCA
TAAGCATACAAAATAGAAACATGTCAGTGGATCAAGGACAATCATGGCCA
TTTAAAATACTAGGAACACAAAGCTTTTATTTTTACTTTTACACCGGAGC
AAACCTACCAGGTGACACAACACAAATACCAGTAGCAGACCTATTACCAC
TAACAAACCCAAGAATAAACAGACCAGGACAATCACTAAATGAGGCAAAA
ATTACAGACCATATTACTTTCACAGAATACAAAAACAAATTTACAAATTA
TTGGGGTAACCCATTTAATAAACACATTCAAGAACACCTAGATATGATAC
TATACTCACTAAAAAGTCCAGAAGCAATAAAAAACGAATGGACAACAGAA
AACATGAAATGGAACCAATTAAACAATGCAGGAACAATGGCATTAACACC
ATTTAACGAGCCAATATTCACACAAATACAATATAACCCAGATAGAGACA
CAGGAGAAGACACTCAATTATACCTACTCTCTAACGCTACAGGAACAGGA
TGGGACCCACCAGGAATTCCAGAATTAATACTAGAAGGATTTCCACTATG
GTTAATATATTGGGGATTTGCAGACTTTCAAAAAAACCTAAAAAAAGTAA
CAAACATAGACACAAATTACATGTTAGTAGCAAAAACAAAATTTACACAA
AAACCTGGCACATTCTACTTAGTAATACTAAATGACACCTTTGTAGAAGG
CAATAGCCCATATGAAAAACAACCTTTACCTGAAGACAACATTAAATGGT
ACCCACAAGTACAATACCAATTAGAAGCACAAAACAAACTACTACAAACT
GGGCCATTTACACCAAACATACAAGGACAACTATCAGACAATATATCAAT
GTTTTATAAATTTTACTTTAAATGGGGAGGAAGCCCACCAAAAGCAATTA
ATGTTGAAAATCCTGCCCACCAGATTCAATATCCCATACCCCGTAACGAG
CATGAAACAACTTCGTTACAGAGTCCAGGGGAAGCCCCAGAATCCATCTT
ATACTCCTTCGACTATAGACACGGGAACTACACAACAACAGCTTTGTCAC
GAATTAGCCAAGACTGGGCACTTAAAGACACTGTTTCTAAAATTACAGAG
CCAGATCGACAGCAACTGCTCAAACAAGCCCTCGAATGCCTGCAAATCTC
GGAAGAAACGCAGGAGAAAAAAGAAAAAGAAGTACAGCAGCTCATCAGCA
ACCTCAGACAGCAGCAGCAGCTGTACAGAGAGCGAATAATATCATTATTA
AAGGACCAATAACTTTTAACTGTGTAAAAAAGGTGAAATTGTTTGATGAT
AAACCAAAAAACCGTAGATTTACACCTGAGGAATTTGAAACTGAGTTACA
AATAGCAAAATGGTTAAAGAGACCCCCAAGATCCTTTGTAAATGATCCTC
CCTTTTACCCATGGTTACCACCTGAACCTGTTGTAAACTTTAAGCTTAAT
TTTACTGAATAAAGGCCAGCATTAATTCACTTAAGGAGTCTGTTTATTTA
AGTTAAACCTTAATAAACGGTCACCGCCTCCCTAATACGCAGGCGCAGAA
AGGGGGCTCCGCCCCCTTTAACCCCCAGGGGGCTCCGCCCCCTGAAACCC
CCAAGGGGGCTACGCCCCCTTACACCCCC (SEQ ID NO: 54)

Annotations:

| Putative Domain | Base range |
|---|---|
| TATA Box | 237-243 |
| Cap Site | 260-267 |
| Transcriptional Start Site | 267 |
| 5′ UTR Conserved Domain | 323-393 |
| ORF2 | 424-723 |
| ORF2/2 | 424-719; 2274-2589 |
| ORF2/3 | 424-719; 2449-2812 |
| ORF1 | 612-2612 |
| ORF1/1 | 612-719; 2274-2612 |
| ORF1/2 | 612-719; 2449-2589 |
| Three open-reading frame region | 2441-2586 |
| Poly(A) Signal | 2808-2813 |
| GC-rich region | 2868-2929 |

TABLE B2
Exemplary Anellovirus amino acid sequences
(Betatorquevirus)
Ring2 (Betatorquevirus)

| | |
|---|---|
| ORF2 | MSDCFKPTCYNNKTKQTHWINNLHLTHDLICFCPTPTRHLLLA |
| | LAEQQETIEVSKQEKEKITRCLITTEEDGTTTDVLDGMDEVGL |
| | DALFAEDFEEKEG |
| | (SEQ ID NO: 55) |
| ORF2/2 | MSDCFKPTCYNNKTKQTHWINNLHLTHDLICFCPTPTRHLLLA |
| | LAEQQETIEVSKQEKEKITRCLITTEEDGTTTDVLDGMDEVGL |
| | DALFAEDFEEKEGFNIPYPVTSMKQLRYRVQGKPQNPSYTPST |
| | IDTGTTQQQLCHELAKTGHLKTLFLKLQSQIDSNCSNKPSNAC |
| | KSRKKRRRKKKKKYSSSSATSDSSSSCTESE |
| | (SEQ ID NO: 56) |
| ORF2/3 | MSDCFKPTCYNNKTKQTHWINNLHLTHDLICFCPTPTRHLLLA |
| | LAEQQETIEVSKQEKEKITRCLITTEEDGTTTDVLDGMDEVGL |
| | DALFAEDFEEKEGARSTATAQTSPRMPANLGRNAGEKRKRSTA |
| | AHQQPQTAAAAVQRANNIIIKGPITFNCVKKVKLFDDKPKNRR |
| | FTPEEFETELQIAKWLKRPPRSFVNDPPFYPWLPPEPVVNFKL |
| | NFTE |
| | (SEQ ID NO: 57) |
| ORF1 | MPYYYRRRRYNYRRPRWYGRGWIRRPFRRRFRRKRRVRPTYTT |
| | IPLKQWQPPYKRTCYIKGQDCLIYYSNLRLGMNSTMYEKSIVP |
| | VHWPGGGSFSVSMLTLDALYDIHKLCRNWWTSTNQDLPLVRYK |
| | GCKITFYQSTFTDYIVRIHTELPANSNKLTYPNTHPLMMMMSK |
| | YKHIIPSRQTRRKKKPYTKIFVKPPPQFENKWYFATDLYKIPL |
| | LQIHCTACNLQNPFVKPDKLSNNVTLWSLNTISIQNRNMSVDQ |
| | GQSWPFKILGTQSFYFYFYTGANLPGDTTQIPVADLLPLTNPR |
| | INRPGQSLNEAKITDHITFTEYKNKFTNYWGNPFNKHIQEHLD |
| | MILYSLKSPEAIKNEWTTENMKWNQLNNAGTMALTPFNEPIFT |
| | QIQYNPDRDTGEDTQLYLLSNATGTGWDPPGIPELILEGFPLW |
| | LIYWGFADFQKNLKKVTNIDTNYMLVAKTKFTQKPGTFYLVIL |
| | NDTFVEGNSPYEKQPLPEDNIKWYPQVQYQLEAQNKLLQTGPF |
| | TPNIQGQLSDNISMFYKFYFKWGGSPPKAINVENPAHQIQYPI |
| | PRNEHETTSLQSPGEAPESILYSFDYRHGNYTTTALSRISQDW |
| | ALKDTVSKITEPDRQQLLKQALECLQISEETQEKKEKEVQQLI |
| | SNLRQQQQLYRERIISLLKDQ |
| | (SEQ ID NO: 58) |
| ORF1/1 | MPYYYRRRRYNYRRPRWYGRGWIRRPFRRRFRRKRRIQYPIPR |
| | NEHETTSLQSPGEAPESILYSFDYRHGNYTTTALSRISQDWAL |
| | KDTVSKITEPDRQQLLKQALECLQISEETQEKKEKEVQQLISN |
| | LRQQQQLYRERIISLLKDQ |
| | (SEQ ID NO: 59) |
| ORF1/2 | MPYYYRRRRYNYRRPRWYGRGWIRRPFRRRFRRKRRSQIDSNC |
| | SNKPSNACKSRKKRRRKKKKKYSSSSATSDSSSSCTESE |
| | (SEQ ID NO: 60) |

TABLE C1
Exemplary Anellovirus nucleic acid sequence (Gammatorquevirus)

| Name | Ring4 |
|---|---|
| Genus/Clade | Gammatorquevirus |
| Accession Number | |
| Full Sequence: 3176 bp | |

1 10 20 30 40 50
| | | | | |
TAAAATGGCGGGAGCCAATCATTTTATACTTTCACTTTCCAATTAAAAAT
GGCCACGTCACAAACAAGGGGTGGAGCCATTTAAACTATATAACTAAGTG
GGGTGGCGAATGGCTGAGTTTACCCCGCTAGACGGTGCAGGGACCGGATC
GAGCGCAGCGAGGAGGTCCCCGGCTGCCCATGGGCGGGAGCCGAGGTGAG
TGAAACCACCGAGGTCTAGGGGCAATTCGGGCTAGGGCAGTCTAGCGGAA
CGGGCAAGAAACTTAAAACAATATTTGTTTTACAGATGGTTAGTATATCC
TCAAGTGATTTTTTTAAGAAAACGAAATTTAATGAGGAGACGCAGAACCA
AGTATGGATGTCTCAAATTGCTGACTCTCATGATAATATCTGCAGTTGCT
GGCATCCATTTGCTCACCTTCTTGCTTCCATATTTCCTCCTGGCCACAAA
GATCGTGATCTTACTATTAACCAAATTCTTCTAAGAGATTATAAAGAAAA
ATGCCATTCTGGTGGAGAAGAAGGAGAAAATTCTGGACCAACAACAGGTT
TAATTACACCAAAAGAAGAAGATATAGAAAAAGATGGCCCAGAAGGCGCC
GCAGAAGAAGACCATACAGACGCCCTGTTCGCCGCCGCCGTAGAAAACTT
CGAAAGGTAAAGAGAAAAAAAAAATCTTTAATTGTTAGACAATGGCAACC
AGACAGTATAAGAACTTGTAAAATTATAGGACAGTCAGCTATAGTTGTTG
GGGCTGAAGGAAAGCAAATGTACTGTTATACTGTCAATAAGTTAATTAAT
GTGCCCCCAAAAACACCATATGGGGGAGGCTTTGGAGTAGACCAATACAC
ACTGAAATACTTATATGAAGAATACAGATTTGCACAAAACATTTGGACAC
AATCTAATGTACTGAAAGACTTATGCAGATACATAAATGTTAAGCTAATA
TTCTACAGAGACAACAAAACAGACTTTGTCCTTTCCTATGACAGAAACCC
ACCTTTTCAACTAACAAAATTTACATACCCAGGAGCACACCCACAACAAA
TCATGCTTCAAAAACACCACAAATTCATACTATCACAAATGACAAAGCCT
AATGGAAGACTAACAAAAAAACTCAAAATTAAACCTCCTAAACAAATGCT
TTCTAAATGGTTCTTTTCAAAACAATTCTGTAAATACCCTTTACTATCTC
TTAAAGCTTCTGCACTAGACCTTAGGCACTCTTACCTAGGCTGCTGTAAT
GAAAATCCACAGGTATTTTTTTATTATTTAAACCATGGATACTACACAAT
AACAAACTGGGGAGCACAATCCTCAACAGCATACAGACCTAACTCCAAGG
TGACAGACACAACATACTACAGATACAAAAATGACAGAAAAAATATTAAC
ATTAAAAGCCATGAATACGAAAAAAGTATATCATATGAAAACGGTTATTT
TCAATCTAGTTTCTTACAAACACAGTGCATATATACCAGTGAGCGTGGTG
AAGCCTGTATAGCAGAAAAACCACTAGGAATAGCTATTTACAATCCAGTA
AAAGACAATGGAGATGGTAATATGATATACCTTGTAAGCACTCTAGCAAA
CACTTGGGACCAGCCTCCAAAAGACAGTGCTATTTTAATACAAGGAGTAC
CCATATGGCTAGGCTTATTTGGATATTTAGACTACTGTAGACAAATTAAA
GCTGACAAAACATGGCTAGACAGTCATGTACTAGTAATTCAAAGTCCTGC
TATTTTTACTTACCCAAATCCAGGAGCAGGCAAATGGTATTGTCCACTAT
CACAAAGTTTTATAAATGGCAATGGTCCGTTTAATCAACCACCTACACTG
CTACAAAAAGCAAAGTGGTTTCCACAAATACAATACCAACAAGAAATTAT
TAATAGCTTTGTAGAATCAGGACCATTTGTTCCCAAATATGCAAATCAAA
CTGAAAGCAACTGGGAACTAAAATATAAATATGTTTTTACATTTAAGTGG
GGTGGACCACAATTCCATGAACCAGAAATTGCTGACCCTAGCAAACAAGA
GCAGTATGATGTCCCCGATACTTTCTACCAAACAATACAAATTGAAGATC
CAGAAGGACAAGACCCCAGATCTCTCATCCATGATTGGGACTACAGACGA
GGCTTTATTAAAGAAAGATCTCTTAAAAGAATGTCAACTTACTTCTCAAC
TCATACAGATCAGCAAGCAACTTCAGAGGAAGACATTCCCAAAAAGAAAA
AGAGAATTGGACCCCAACTCACAGTCCCACAACAAAAAGAAGAGGAGACA
CTGTCATGTCTCCTCTCTCTCTGCAAAAAAGATACCTTCCAAGAAACAGA
GACACAAGAAGACCTCCAGCAGCTCATCAAGCAGCAGCAGGAGCAGCAGC
TCCTCCTCAAGAGAAACATCCTCCAGCTCATCCACAAACTAAAAGAGAAT
CAACAAATGCTTCAGCTTCACACAGGCATGTTACCTTAACCAGATTTAAA
CCTGGATTTGAAGAGCAAACAGAGAGAGAATTAGCAATTATATTTCATAG
GCCCCCTAGAACCTACAAAGAGGACCTTCCATTCTATCCCTGGCTACCAC
CTGCACCCCTTGTACAATTTAACCTTAACTTCAAAGGCTAGGCCAACAAT
GTACACTTAGTAAAGCATGTTTATTAAAGCACAACCCCCAAAATAAATGT
AAAAATAAAAAAAAAAAAAAAAAAATAAAAAATTGCAAAAATTCGGCGCT
CGCGCGCATGTGCGCCTCTGGCGCAAATCACGCAACGCTCGCGCGCCCGC
GTATGTCTCTTTACCACGCACCTAGATTGGGGTGCGCGCGCTAGCGCGCG
CACCCCAATGCGCCCCGCCCTCGTTCCGACCCGCTTGCGCGGGTCGGACC
ACTTCGGGCTCGGGGGGGCGCGCCTGCGGCGCTTTTTTACTAAACAGACT
CCGAGCCGCCATTTGGCCCCCTAAGCTCCGCCCCCCTCATGAATATTCAT
AAAGGAAACCACATAATTAGAATTGCCGACCACAAACTGCCATATGCTAA
TTAGTTCCCCTTTTACAAAGTAAAAGGGGAAGTGAACATAGCCCCACACC
CGCAGGGGCAAGGCCCCGCACCCCTACGTCACTAACCACGCCCCCGCCGC
CATCTTGGGTGCGGCAGGGCGGGGGC (SEQ ID NO: 886)

Annotations:

| Putative Domain | Base range |
|---|---|
| TATA Box | 87-93 |
| Cap Site | 110-117 |
| Transcriptional Start Site | 117 |
| 5′ UTR Conserved Domain | 185-254 |
| ORF2 | 286-660 |
| ORF2/2 | 286-656; 1998-2442 |
| ORF2/3 | 286-656; 2209-2641 |
| TAIP | 385-484 |
| ORF1 | 501-2489 |
| ORF1/1 | 501-656; 1998-2489 |
| ORF1/2 | 501-656; 2209-2442 |
| Three open-reading frame region | 2209-2439 |
| Poly(A) Signal | 2672-2678 |
| GC-rich region | 3076-3176 |

TABLE C2
Exemplary Anellovirus amino acid sequences (Gammatorquevirus)
Ring4 (Gammatorquevirus)

| | |
|---|---|
| ORF2 | MVSISSSDFFKKTKFNEETQNQVWMSQIADSHDNICSCWHPFAHLLASIFPP |
| | GHKDRDLTINQILLR |
| | DYKEKCHSGGEEGENSGPTTGLITPKEEDIEKDGPEGAAEEDHTDALFAAAV |
| | ENFER (SEQ ID NO: 887) |
| ORF2/2 | MVSISSSDFFKKTKFNEETQNQVWMSQIADSHDNICSCWHPFAHLLASIFPP |
| | GHKDRDLTINQILLRDYKEKCHSGGEEGENSGPTTGLITPKEEDIEKDGPEGA |
| | AEEDHTDALFAAAVENFESGVDHNSMNQKLLTLANKSSMMSPILSTKQYKL |
| | KIQKDKTPDLSSMIGTTDEALLKKDLLKECQLTSQLIQISKQLQRKTFPKRKR |
| | ELDPNSQSHNKKKRRHCHVSSLSAKKIPSKKQRHKKTSSSSSSSSRSSSSSSR |
| | ETSSSSSTN (SEQ ID NO: 888) |
| ORF2/3 | MVSISSSDFFKKTKFNEETQNQVWMSQIADSHDNICSCWHPFAHLLASIFPP |
| | GHKDRDLTINQILLRDYKEKCHSGGEEGENSGPTTGLITPKEEDIEKDGPEGA |
| | AEEDHTDALFAAAVENFERSASNFRGRHSQKEKENWTPTHSPTTKRRGDTV |
| | MSPLSLQKRYLPRNRDTRRPPAAHQAAAGAAAPPQEKHPPAHPQTKRESTN |
| | ASASHRHVTLTRFKPGFEEQTERELAIIFHRPPRTYKEDLPFYPWLPPAPLVQ |
| | FNLNFKG (SEQ ID NO: 889) |
| TAIP | MRRRRTKYGCLKLLTLMIISAVAGIHLLTFLLPYFLLATKIVILLLTKFF |
| | (SEQ ID NO: 890) |
| ORF1 | MPFWWRRRRKFWTNNRFNYTKRRRYRKRWPRRRRRRRPYRRPVRRRRRK |
| | LRKVKRKKKSLIVRQWQPDSIRTCKIIGQSAIVVGAEGKQMYCYTVNKLINV |
| | PPKTPYGGGFGVDQYTLKYLYEEYRFAQNIWTQSNVLKDLCRYINVKLIFY |
| | RDNKTDFVLSYDRNPPFQLTKFTYPGAHPQQIMLQKHHKFILSQMTKPNGR |
| | LTKKLKIKPPKQMLSKWFFSKQFCKYPLLSLKASALDLRHSYLGCCNENPQ |
| | VFFYYLNHGYYTITNWGAQSSTAYRPNSKVTDTTYYRYKNDRKNINIKSHE |
| | YEKSISYENGYFQSSFLQTQCIYTSERGEACIAEKPLGIAIYNPVKDNGDGNM |
| | IYLVSTLANTWDQPPKDSAILIQGVPIWLGLFGYLDYCRQIKADKTWLDSHV |
| | LVIQSPAIFTYPNPGAGKWYCPLSQSFINGNGPFNQPPTLLQKAKWFPQIQYQ |
| | QEIINSFVESGPFVPKYANQTESNWELKYKYVFTFKWGGPQFHEPEIADPSK |
| | QEQYDVPDTFYQTIQIEDPEGQDPRSLIHDWDYRRGFIKERSLKRMSTYFST |
| | HTDQQATSEEDIPKKKKRIGPQLTVPQQKEEETLSCLLSLCKKDTFQETETQE |
| | DLQQLIKQQQEQQLLLKRNILQLIHKLKENQQMLQLHTGMLP (SEQ ID NO: |
| | 891) |
| ORF1/1 | MPFWWRRRRKFWTNNRFNYTKRRRYRKRWPRRRRRRRPYRRPVRRRRRK |
| | LRKWGGPQFHEPEIADPSKQEQYDVPDTFYQTIQIEDPEGQDPRSLIHDWDY |
| | RRGFIKERSLKRMSTYFSTHTDQQATSEEDIPKKKKRIGPQLTVPQQKEEETL |
| | SCLLSLCKKDTFQETETQEDLQQLIKQQQEQQLLLKRNILQLIHKLKENQQM |
| | LQLHTGMLP (SEQ ID NO: 892) |
| ORF1/2 | MPFWWRRRRKFWTNNRFNYTKRRRYRKRWPRRRRRRRPYRRPVRRRRRK |
| | LRKISKQLQRKTFPKRKR |
| | ELDPNSQSHNKKKRRHCHVSSLSAKKIPSKKQRHKKTSSSSSSSSRSSSSSSR |
| | ETSSSSSTN (SEQ ID NO: 893) |

In some embodiments, an anellovector comprises a nucleic acid comprising a sequence listed in PCT Application No. PCT/US2018/037379, incorporated herein by reference in its entirety. In some embodiments, an anellovector comprises a polypeptide comprising a sequence listed in PCT Application No. PCT/US2018/037379, incorporated herein by reference in its entirety. In some embodiments, an anellovector comprises a nucleic acid comprising a sequence listed in PCT Application No. PCT/US19/65995, incorporated herein by reference in its entirety. In some embodiments, an anellovector comprises a polypeptide comprising a sequence listed in PCT Application No. PCT/US19/65995, incorporated herein by reference in its entirety.

### ORF1 Molecules

In some embodiments, the anellovector comprises an ORF1 molecule and/or a nucleic acid encoding an ORF1 molecule. Generally, an ORF1 molecule comprises a polypeptide having the structural features and/or activity of an Anellovirus ORF1 protein (e.g., an Anellovirus ORF1 protein as described herein). In some embodiments, the ORF1 molecule comprises a truncation relative to an Anellovirus ORF1 protein (e.g., an Anellovirus ORF1 protein as described herein). An ORF1 molecule may be capable of binding to other ORF1 molecules, e.g., to form a proteinaceous exterior (e.g., as described herein), e.g., a capsid. In some embodiments, the proteinaceous exterior may enclose a nucleic acid molecule (e.g., a genetic element as described herein). In some embodiments, a plurality of ORF1 molecules may form a multimer, e.g., to form a proteinaceous exterior. In some embodiments, the multimer may be a homomultimer. In other embodiments, the multimer may be a heteromultimer.

An ORF1 molecule may, in some embodiments, comprise one or more of: a first region comprising an arginine rich region, e.g., a region having at least 60% basic residues (e.g., at least 60%, 65%, 70%, 75%, 80%, 85%, 90%, 95%, or 100% basic residues; e.g., between 60%-90%, 60%-80%, 70%-90%, or 70-80% basic residues), and a second region comprising jelly-roll domain, e.g., at least six beta strands (e.g., 4, 5, 6, 7, 8, 9, 10, 11, or 12 beta strands).

Arginine-Rich Region

An arginine rich region has at least 70% (e.g., at least about 70, 80, 90, 95, 96, 97, 98, 99, or 100%) sequence identity to an arginine-rich region sequence described herein or a sequence of at least about 40 amino acids comprising at least 60%, 70%, or 80% basic residues (e.g., arginine, lysine, or a combination thereof).

Jelly Roll Domain

A jelly-roll domain or region comprises (e.g., consists of) a polypeptide (e.g., a domain or region comprised in a larger polypeptide) comprising one or more (e.g., 1, 2, or 3) of the following characteristics:
(i) at least 30% (e.g., at least 30%, 35%, 40%, 45%, 50%, 55%, 60%, 65%, 70%, 75%, 80%, 90%, or more) of the amino acids of the jelly-roll domain are part of one or more β-sheets;
(ii) the secondary structure of the jelly-roll domain comprises at least four (e.g., at least 4, 5, 6, 7, 8, 9, 10, 11, or 12) β-strands; and/or
(iii) the tertiary structure of the jelly-roll domain comprises at least two (e.g., at least 2, 3, or 4) β-sheets; and/or
(iv) the jelly-roll domain comprises a ratio of β-sheets to α-helices of at least 2:1, 3:1, 4:1, 5:1, 6:1, 7:1, 8:1, 9:1, or 10:1.

In certain embodiments, a jelly-roll domain comprises two β-sheets.

In certain embodiments, one or more (e.g., 1, 2, 3, 4, 5, 6, 7, 8, 9, or 10) of the β-sheets comprises about eight (e.g., 4, 5, 6, 7, 8, 9, 10, 11, or 12) β-strands. In certain embodiments, one or more (e.g., 1, 2, 3, 4, 5, 6, 7, 8, 9, or 10) of the β-sheets comprises eight β-strands. In certain embodiments, one or more (e.g., 1, 2, 3, 4, 5, 6, 7, 8, 9, or 10) of the β-sheets comprises seven β-strands. In certain embodiments, one or more (e.g., 1, 2, 3, 4, 5, 6, 7, 8, 9, or 10) of the β-sheets comprises six β-strands. In certain embodiments, one or more (e.g., 1, 2, 3, 4, 5, 6, 7, 8, 9, or 10) of the β-sheets comprises five β-strands. In certain embodiments, one or more (e.g., 1, 2, 3, 4, 5, 6, 7, 8, 9, or 10) of the β-sheets comprises four β-strands.

In some embodiments, the jelly-roll domain comprises a first β-sheet in antiparallel orientation to a second β-sheet. In certain embodiments, the first β-sheet comprises about four (e.g., 3, 4, 5, or 6) β-strands. In certain embodiments, the second β-sheet comprises about four (e.g., 3, 4, 5, or 6) β-strands. In embodiments, the first and second β-sheet comprise, in total, about eight (e.g., 6, 7, 8, 9, 10, 11, or 12) β-strands.

In certain embodiments, a jelly-roll domain is a component of a capsid protein (e.g., an ORF1 molecule as described herein). In certain embodiments, a jelly-roll domain has self-assembly activity. In some embodiments, a polypeptide comprising a jelly-roll domain binds to another copy of the polypeptide comprising the jelly-roll domain. In some embodiments, a jelly-roll domain of a first polypeptide binds to a jelly-roll domain of a second copy of the polypeptide.

N22 Domain

An ORF1 molecule may also include a third region comprising the structure or activity of an Anellovirus N22 domain (e.g., as described herein, e.g., an N22 domain from an Anellovirus ORF1 protein as described herein), and/or a fourth region comprising the structure or activity of an Anellovirus C-terminal domain (CTD) (e.g., as described herein, e.g., a CTD from an Anellovirus ORF1 protein as described herein). In some embodiments, the ORF1 molecule comprises, in N-terminal to C-terminal order, the first, second, third, and fourth regions.

Hypervariable Region (HVR)

The ORF1 molecule may, in some embodiments, further comprise a hypervariable region (HVR), e.g., an HVR from an Anellovirus ORF1 protein, e.g., as described herein. In some embodiments, the HVR is positioned between the second region and the third region. In some embodiments, the HVR comprises at least about 55 (e.g., at least about 45, 50, 51, 52, 53, 54, 55, 56, 57, 58, 59, 60, or 65) amino acids (e.g., about 45-160, 50-160, 55-160, 60-160, 45-150, 50-150, 55-150, 60-150, 45-140, 50-140, 55-140, or 60-140 amino acids).

Exemplary ORF1 Sequences

Exemplary Anellovirus ORF1 amino acid sequences, and the sequences of exemplary ORF1 domains, are provided in the tables below. In some embodiments, a polypeptide (e.g., an ORF1 molecule) described herein comprises an amino acid sequence having at least about 70%, 75%, 80%, 85%, 90%, 95%, 96%, 97%, 98%, 99%, or 100% sequence identity to one or more Anellovirus ORF1 subsequences, e.g., as described in any of Tables N-Z). In some embodiments, an anellovector described herein comprises an ORF1 molecule comprising an amino acid sequence having at least about 70%, 75%, 80%, 85%, 90%, 95%, 96%, 97%, 98%, 99%, or 100% sequence identity to one or more Anellovirus ORF1 subsequences, e.g., as described in any of Tables N-Z. In some embodiments, an anellovector described herein comprises a nucleic acid molecule (e.g., a genetic element) encoding an ORF1 molecule comprising an amino acid sequence having at least about 70%, 75%, 80%, 85%, 90%, 95%, 96%, 97%, 98%, 99%, or 100% sequence identity to one or more Anellovirus ORF1 subsequences, e.g., as described in any of Tables N-Z.

In some embodiments, the one or more Anellovirus ORF1 subsequences comprises one or more of an arginine (Arg)-rich domain, a jelly-roll domain, a hypervariable region (HVR), an N22 domain, or a C-terminal domain (CTD) (e.g., as listed in any of Tables N-Z), or sequences having at least about 70%, 75%, 80%, 85%, 90%, 95%, 96%, 97%, 98%, 99%, or 100% sequence identity thereto. In some embodiments, the ORF1 molecule comprises a plurality of subsequences from different Anelloviruses (e.g., any combination of ORF1 subsequences selected from the Alphatorquevirus Clade 1-7 subsequences listed in Tables N-Z). In embodiments, the ORF1 molecule comprises one or more of an Arg-rich domain, a jelly-roll domain, an N22 domain, and a CTD from one Anellovirus, and an HVR from another. In embodiments, the ORF1 molecule comprises one or more of a jelly-roll domain, an HVR, an N22 domain, and a CTD from one Anellovirus, and an Arg-rich domain from another. In embodiments, the ORF1 molecule comprises one or more of an Arg-rich domain, an HVR, an N22 domain, and a CTD from one Anellovirus, and a jelly-roll domain from another. In embodiments, the ORF1 molecule comprises one or more of an Arg-rich domain, a jelly-roll domain, an HVR, and a CTD from one Anellovirus, and an N22 domain from another. In embodiments, the ORF1 molecule comprises one or more of an Arg-rich domain, a jelly-roll domain, an HVR, and an N22 domain from one Anellovirus, and a CTD from another.

Additional exemplary Anelloviruses for which the ORF1 molecules, or splice variants or functional fragments thereof, can be utilized in the compositions and methods described herein (e.g., to form the proteinaceous exterior of an anellovector, e.g., by enclosing a genetic element) are described, for example, in PCT Application Nos. PCT/US2018/037379 and PCT/US19/65995 (incorporated herein by reference in their entirety).

TABLE N
Exemplary Anellovirus ORF1 amino acid subsequence
(Alphatorquevirus, Clade 3)

| | |
|---|---|
| Name | Ring1 |
| Genus/Clade | Alphatorquevirus, Clade 3 |
| Accession Number | AJ620231.1 |
| Protein Accession Number | CAF05750.1 |
| Full Sequence: 743 AA | |

1 10 20 30 40 50
| | | | | |
MAWGWWKRRRRWWFRKRWTRGRLRRRWPRSARRRPRRRRVRRRRRWRRGR
RKTRTYRRRRRFRRRGRKAKLIIKLWQPAVIKRCRIKGYIPLIISGNGTF
ATNFTSHINDRIMKGPFGGGHSTMRFSLYILFEEHLRHMNFWTRSNDNLE
LTRYLGASVKIYRHPDQDFIVIYNRRTPLGGNIYTAPSLHPGNAILAKHK
ILVPSLQTRPKGRKAIRLRIAPPTLFTDKWYFQKDIADLTLFNIMAVEAD
LRFPFCSPQTDNTCISFQVLSSVYNNYLSINTFNNDNSDSKLKEFLNKAF
PTTGTKGTSLNALNTFRTEGCISHPQLKKPNPQINKPLESQYFAPLDALW
GDPIYYNDLNENKSLNDIIEKILIKNMITYHAKLREFPNSYQGNKAFCHL
TGIYSPPYLNQGRISPEIFGLYTEIIYNPYTDKGTGNKVWMDPLTKENNI
YKEGQSKCLLTDMPLWTLLFGYTDWCKKDTNNWDLPLNYRLVLICPYTFP
KLYNEKVKDYGYIPYSYKFGAGQMPDGSNYIPFQFRAKWYPTVLHQQQVM
EDISRSGPFAPKVEKPSTQLVMKYCFNFNWGGNPIIEQIVKDPSFQPTYE
IPGTGNIPRRIQVIDPRVLGPHYSFRSWDMRRHTFSRASIKRVSEQQETS
DLVFSGPKKPRVDIPKQETQEESSHSLQRESRPWETEEESETEALSQESQ
EVPFQQQLQQQYQEQLKLRQGIKVLFEQLIRTQQGVHVNPCLR
(SEQ ID NO: 185)

Annotations:

| Putative Domain | AA range |
|---|---|
| Arg-Rich Region | 1-68 |
| Jelly-roll domain | 69-280 |
| Hypervariable Region | 281-413 |
| N22 | 414-579 |
| C-terminal Domain | 580-743 |

TABLE O
Exemplary Anellovirus ORF1 amino acid subsequence
(Alphatorquevirus, Clade 3)
Ring1 ORF1 (Alphatorquevirus Clade 3)

| | |
|---|---|
| Arg-Rich | MAWGWWKRRRRWWFRKRWTRGRLRRRWPRSARRRPRRRRVRRRR |
| Region | RWRRGRRKTRTYRRRRRFRRRGRK (SEQ ID NO: 186) |
| Jelly-roll | AKLIIKLWQPAVIKRCRIKGYIPLIISGNGTFATNFTSHINDRIMKGPFGG |
| Domain | GHSTMRFSLYILFEEHLRHMNFWTRSNDNLELTRYLGASVKIYRHPDQ |
| | DFIVIYNRRTPLGGNIYTAPSLHPGNAILAKHKILVPSLQTRPKGRKAIRL |
| | RIAPPTLFTDKWYFQKDIADLTLFNIMAVEADLRFPFCSPQTDNTCISFQ |
| | VLSSVYNNYLSI (SEQ ID NO: 187) |
| Hypervariable | NTFNNDNSDSKLKEFLNKAFPTTGTKGTSLNALNTFRTEGCISHPQLKK |
| domain | PNPQINKPLESQYFAPLDALWGDPIYYNDLNENKSLNDIIEKILIKNMIT |
| | YHAKLREFPNSYQGNKAFCHLTGIYSPPYLNQGR (SEQ ID NO: 188) |
| N22 | ISPEIFGLYTEIIYNPYTDKGTGNKVWMDPLTKENNIYKEGQSKCLLTD |
| | MPLWTLLFGYTDWCKKDTNNWDLPLNYRLVLICPYTFPKLYNEKVKD |
| | YGYIPYSYKFGAGQMPDGSNYIPFQFRAKWYPTVLHQQQVMEDISRSG |
| | PFAPKVEKPSTQLVMKYCFNFN (SEQ ID NO: 189) |
| C-terminal | WGGNPIIEQIVKDPSFQPTYEIPGTGNIPRRIQVIDPRVLGPHYSFRSWD |
| domain | MRRHTFSRASIKRVSEQQETSDLVFSGPKKPRVDIPKQETQEESSHSLQR |
| | ESRPWETEEESETEALSQESQEVPFQQQLQQQYQEQLKLRQGIKVLFEQ |
| | LIRTQQGVHVNPCLR (SEQ ID NO: 190) |

TABLE P
Exemplary Anellovirus ORF1 amino acid subsequence
(Betatorquevirus)

| | |
|---|---|
| Name | Ring2 |
| Genus/Clade | Betatorquevirus |
| Accession Number | JX134045.1 |
| Protein Accession Number | AGG91484.1 |

Full Sequence: 666 AA
1 10 20 30 40 50
| | | | | |
MPYYYRRRRYNYRRPRWYGRGWIRRPFRRRFRRKRRVRPTYTTIPLKQWQ
PPYKRTCYIKGODCLIYYSNLRLGMNSTMYEKSIVPVHWPGGGSFSVSML
TLDALYDIHKLCRNWWTSTNQDLPLVRYKGCKITFYQSTFTDYIVRIHTE
LPANSNKLTYPNTHPLMMMMSKYKHIIPSRQTRRKKKPYTKIFVKPPPQF
ENKWYFATDLYKIPLLQIHCTACNLQNPFVKPDKLSNNVTLWSLNTISIQ
NRNMSVDQGQSWPFKILGTQSFYFYFYTGANLPGDTTQIPVADLLPLTNP
RINRPGQSLNEAKITDHITFTEYKNKFTNYWGNPFNKHIQEHLDMILYSL
KSPEAIKNEWTTENMKWNQLNNAGTMALTPFNEPIFTQIQYNPDRDTGED
TQLYLLSNATGTGWDPPGIPELILEGFPLWLIYWGFADFQKNLKKVTNID
TNYMLVAKTKFTQKPGTFYLVILNDTFVEGNSPYEKQPLPEDNIKWYPQV
QYQLEAQNKLLQTGPFTPNIQGQLSDNISMFYKFYFKWGGSPPKAINVEN
PAHQIQYPIPRNEHETTSLOSPGEAPESILYSFDYRHGNYTTTALSRISQ
DWALKDTVSKITEPDRQQLLKQALECLQISEETQEKKEKEVQQLISNLRQ
QQQLYRERIISLLKDQ (SEQ ID NO: 215)

Annotations:

| Putative Domain | AA range |
|---|---|
| Arg-Rich Region | 1-38 |
| Jelly-roll domain | 39-246 |
| Hypervariable Region | 247-374 |
| N22 | 375-537 |
| C-terminal Domain | 538-666 |

TABLE Q
Exemplary Anellovirus ORF1 amino acid subsequence
(Betatorquevirus)
Ring2 ORF1 (Betatorquevirus)

| | |
|---|---|
| Arg-Rich | MPYYYRRRRYNYRRPRWYGRGWIRRPFRRRFRRKRRVR (SEQ ID NO: |
| Region | 216) |
| Jelly-roll | PTYTTIPLKQWQPPYKRTCYIKGQDCLIYYSNLRLGMNSTMYEKSIVPV |
| Domain | FYQSTFTDYIVRIHTELPANSNKLTYPNTHPLMMMMSKYKHIIPSRQTR |
| | HWPGGGSFSVSMLTLDALYDIHKLCRNWWTSTNQDLPLVRYKGCKIT |
| | RKKKPYTKIFVKPPPQFENKWYFATDLYKIPLLQIHCTACNLQNPFVKP |
| | DKLSNNVTLWSLNT (SEQ ID NO: 217) |
| Hypervariable | ISIQNRNMSVDQGQSWPFKILGTQSFYFYFYTGANLPGDTTQIPVADLL |
| domain | PLTNPRINRPGQSLNEAKITDHITFTEYKNKFTNYWGNPFNKHIQEHLD |
| | MILYSLKSPEAIKNEWTTENMKWNQLNNAG (SEQ ID NO: 218) |
| N22 | TMALTPFNEPIFTQIQYNPDRDTGEDTQLYLLSNATGTGWDPPGIPELIL |
| | EGFPLWLIYWGFADFQKNLKKVTNIDTNYMLVAKTKFTQKPGTFYLVI |
| | LNDTFVEGNSPYEKQPLPEDNIKWYPQVQYQLEAQNKLLQTGPFTPNI |
| | QGQLSDNISMFYKFYFK (SEQ ID NO: 219) |
| C-terminal | WGGSPPKAINVENPAHQIQYPIPRNEHETTSLQSPGEAPESILYSFDYRH |
| domain | GNYTTTALSRISQDWALKDTVSKITEPDRQQLLKQALECLQISEETQEK |
| | KEKEVQQLISNLRQQQQLYRERIISLLKDQ (SEQ ID NO: 220) |

TABLE R
Exemplary Anellovirus ORF1 amino acid subsequence
(Gammatorquevirus)

| | |
|---|---|
| Name | Ring4 |
| Genus/Clade | Gammatorquevirus |
| Accession Number | |
| Protein Accession Number | |
| Full Sequence: 662 AA | |

1 10 20 30 40 50
| | | | | |
MPFWWRRRRKFWTNNRFNYTKRRRYRKRWPRRRRRRRPYRRPVRRRRRKL
RKVKRKKKSLIVROWQPDSIRTCKIIGQSAIVVGAEGKQMYCYTVNKLIN
VPPKTPYGGGFGVDQYTLKYLYEEYRFAQNIWTQSNVLKDLCRYINVKLI
FYRDNKTDFVLSYDRNPPFQLTKFTYPGAHPQQIMLQKHHKFILSQMTKP
NGRLTKKLKIKPPKQMLSKWFFSKQFCKYPLLSLKASALDLRHSYLGCCN
ENPQVFFYYLNHGYYTITNWGAQSSTAYRPNSKVTDTTYYRYKNDRKNIN
IKSHEYEKSISYENGYFQSSFLQTQCIYTSERGEACIAEKPLGIAIYNPV
KDNGDGNMIYLVSTLANTWDQPPKDSAILIQGVPIWLGLFGYLDYCRQIK
ADKTWLDSHVLVIQSPAIFTYPNPGAGKWYCPLSQSFINGNGPFNQPPTL
LQKAKWFPQIQYQQEIINSFVESGPFVPKYANQTESNWELKYKYVFTFKW
GGPQFHEPEIADPSKQEQYDVPDTFYQTIQIEDPEGQDPRSLIHDWDYRR
GFIKERSLKRMSTYFSTHTDQQATSEEDIPKKKKRIGPQLTVPQQKEEET
LSCLLSLCKKDTFQETETQEDLQQLIKQQQEQQLLLKRNILQLIHKLKEN
QQMLQLHTGMLP (SEQ ID NO: 925)

Annotations:

| Putative Domain | AA range |
|---|---|
| Arg-Rich Region | 1-58 |
| Jelly-roll domain | 59-260 |
| Hypervariable Region | 261-339 |
| N22 | 340-499 |
| C-terminal Domain | 500-662 |

TABLE S
Exemplary Anellovirus ORF1 amino acid subsequence
(Gammatorquevirus)
Ring4 (Gammatorquevirus)

| | |
|---|---|
| Arg-Rich | MPFWWRRRRKFWTNNRFNYTKRRRYRKRWPRRRRRRRPYRRPVRRR |
| Region | RRKLRKVKRKKK (SEQ ID NO: 926) |
| Jelly-roll | SLIVRQWQPDSIRTCKIIGQSAIVVGAEGKQMYCYTVNKLINVPPKTPY |
| Domain | GGGFGVDQYTLKYLYEEYRFAQNIWTQSNVLKDLCRYINVKLIFYRDN |
| | KTDFVLSYDRNPPFQLTKFTYPGAHPQQIMLQKHHKFILSQMTKPNGR |
| | LTKKLKIKPPKQMLSKWFFSKQFCKYPLLSLKASALDLRHSYLGCCNE |
| | NPQVFFYYL (SEQ ID NO: 927) |
| Hypervariable | NHGYYTITNWGAQSSTAYRPNSKVTDTTYYRYKNDRKNINIKSHEYEK |
| domain | SISYENGYFQSSFLQTQCIYTSERGEACIAE (SEQ ID NO: 928) |
| N22 | KPLGIAIYNPVKDNGDGNMIYLVSTLANTWDQPPKDSAILIQGVPIWLG |
| | LFGYLDYCRQIKADKTWLDSHVLVIQSPAIFTYPNPGAGKWYCPLSQS |
| | FINGNGPFNQPPTLLQKAKWFPQIQYQQEIINSFVESGPFVPKYANQTES |
| | NWELKYKYVFTFK (SEQ ID NO: 929) |
| C-terminal | WGGPQFHEPEIADPSKQEQYDVPDTFYQTIQIEDPEGQDPRSLIHDWDY |
| domain | RRGFIKERSLKRMSTYFSTHTDQQATSEEDIPKKKKRIGPQLTVPQQKE |
| | EETLSCLLSLCKKDTFQETETQEDLQQLIKQQQEQQLLLKRNILQLIHKL |
| | KENQQMLQLHTGMLP (SEQ ID NO: 930) |

In some embodiments, the first region can bind to a nucleic acid molecule (e.g., DNA). In some embodiments, the basic residues are selected from arginine, histidine, or lysine, or a combination thereof. In some embodiments, the first region comprises at least 60%, 65%, 70%, 75%, 80%, 85%, 90%, 95%, or 100% arginine residues (e.g., between 60%-90%, 60%-80%, 70%-90%, or 70-80% arginine residues). In some embodiments, the first region comprises about 30-120 amino acids (e.g., about 40-120, 40-100, 40-90, 40-80, 40-70, 50-100, 50-90, 50-80, 50-70, 60-100, 60-90, or 60-80 amino acids). In some embodiments, the first region comprises the structure or activity of a viral ORF1 arginine-rich region (e.g., an arginine-rich region from an Anellovirus ORF1 protein, e.g., as described herein). In some embodiments, the first region comprises a nuclear localization signal.

In some embodiments, the second region comprises a jelly-roll domain, e.g., the structure or activity of a viral ORF1 jelly-roll domain (e.g., a jelly-roll domain from an Anellovirus ORF1 protein, e.g., as described herein). In some embodiments, the second region is capable of binding to the second region of another ORF1 molecule, e.g., to form a proteinaceous exterior (e.g., capsid) or a portion thereof.

In some embodiments, the fourth region is exposed on the surface of a proteinaceous exterior (e.g., a proteinaceous exterior comprising a multimer of ORF1 molecules, e.g., as described herein).

In some embodiments, the first region, second region, third region, fourth region, and/or HVR each comprise fewer than four (e.g., 0, 1, 2, or 3) beta sheets.

In some embodiments, one or more of the first region, second region, third region, fourth region, and/or HVR may be replaced by a heterologous amino acid sequence (e.g., the corresponding region from a heterologous ORF1 molecule). In some embodiments, the heterologous amino acid sequence has a desired functionality, e.g., as described herein.

In some embodiments, the ORF1 molecule comprises a plurality of conserved motifs (e.g., motifs comprising about 5, 6, 7, 8, 9, 10, 11, 12, 13, 14, 15, 16, 17, 18, 19, 20, 25, 30, 35, 40, 45, 50, 60, 70, 80, 90, 100, or more amino acids) (e.g., as shown in FIG. 34 of PCT/US19/65995). In some embodiments, the conserved motifs may show 60, 70, 80, 85, 90, 95, or 100% sequence identity to an ORF1 protein of one or more wild-type Anellovirus clades (e.g., Alphatorquevirus, clade 1; Alphatorquevirus, clade 2; Alphatorquevirus, clade 3; Alphatorquevirus, clade 4; Alphatorquevirus, clade 5; Alphatorquevirus, clade 6; Alphatorquevirus, clade 7; Betatorquevirus; and/or Gammatorquevirus). In embodiments, the conserved motifs each have a length between 1-1000 (e.g., between 5-10, 5-15, 5-20, 10-15, 10-20, 15-20, 5-50, 5-100, 10-50, 10-100, 10-1000, 50-100, 50-1000, or 100-1000) amino acids. In certain embodiments, the conserved motifs consist of about 2-4% (e.g., about 1-8%, 1-6%, 1-5%, 1-4%, 2-8%, 2-6%, 2-5%, or 2-4%) of the sequence of the ORF1 molecule, and each show 100% sequence identity to the corresponding motifs in an ORF1 protein of the wild-type Anellovirus clade. In certain embodiments, the conserved motifs consist of about 5-10% (e.g., about 1-20%, 1-10%, 5-20%, or 5-10%) of the sequence of the ORF1 molecule, and each show 80% sequence identity to the corresponding motifs in an ORF1 protein of the wild-type Anellovirus clade. In certain embodiments, the conserved motifs consist of about 10-50% (e.g., about 10-20%, 10-30%, 10-40%, 10-50%, 20-40%, 20-50%, or 30-50%) of the sequence of the ORF1 molecule, and each show 60% sequence identity to the corresponding motifs in an ORF1 protein of the wild-type Anellovirus clade. In some embodiments, the conserved motifs comprise one or more amino acid sequences as listed in Table 19.

In some embodiments, an ORF1 molecule or a nucleic acid molecule encoding same comprises at least one difference (e.g., a mutation, chemical modification, or epigenetic alteration) relative to a wild-type ORF1 protein, e.g., as described herein.

Conserved ORF1 Motif in N22 Domain

In some embodiments, a polypeptide (e.g., an ORF1 molecule) described herein comprises the amino acid sequence YNPX²DXGX²N (SEQ ID NO: 829), wherein Xⁿ is a contiguous sequence of any n amino acids. For example, X² indicates a contiguous sequence of any two amino acids. In some embodiments, the YNPX²DXGX²N (SEQ ID NO: 829) is comprised within the N22 domain of an ORF1 molecule, e.g., as described herein. In some embodiments, a genetic element described herein comprises a nucleic acid sequence (e.g., a nucleic acid sequence encoding an ORF1 molecule, e.g., as described herein) encoding the amino acid sequence YNPX²DXGX²N (SEQ ID NO: 829), wherein Xⁿ is a contiguous sequence of any n amino acids.

In some embodiments, a polypeptide (e.g., an ORF1 molecule) comprises a conserved secondary structure, e.g., flanking and/or comprising a portion of the YNPX²DXGX²N (SEQ ID NO: 829) motif, e.g., in an N22 domain. In some embodiments, the conserved secondary structure comprises a first beta strand and/or a second beta strand. In some embodiments, the first beta strand is about 5-6 (e.g., 3, 4, 5, 6, 7, or 8) amino acids in length. In some embodiments, the first beta strand comprises the tyrosine (Y) residue at the N-terminal end of the YNPX²DXGX²N (SEQ ID NO: 829) motif. In some embodiments, the YNPX²DXGX²N (SEQ ID NO: 829) motif comprises a random coil (e.g., about 8-9 amino acids of random coil). In some embodiments, the second beta strand is about 7-8 (e.g., 5, 6, 7, 8, 9, or 10) amino acids in length. In some embodiments, the second beta strand comprises the asparagine (N) residue at the C-terminal end of the YNPX²DXGX²N (SEQ ID NO: 829) motif.

Exemplary YNPX²DXGX²N (SEQ ID NO: 829) motif-flanking secondary structures are described in Example 47 and FIG. 48 of PCT/US19/65995; incorporated herein by reference in its entirety. In some embodiments, an ORF1 molecule comprises a region comprising one or more (e.g., 1, 2, 3, 4, 5, 6, 7, 8, 9, 10, or all) of the secondary structural elements (e.g., beta strands) shown in FIG. 48 of PCT/US19/65995. In some embodiments, an ORF1 molecule comprises a region comprising one or more (e.g., 1, 2, 3, 4, 5, 6, 7, 8, 9, 10, or all) of the secondary structural elements (e.g., beta strands) shown in FIG. 48 of PCT/US19/65995, flanking a YNPX²DXGX²N (SEQ ID NO: 829) motif (e.g., as described herein).

Conserved Secondary Structural Motif in ORF1 Jelly-Roll Domain

In some embodiments, a polypeptide (e.g., an ORF1 molecule) described herein comprises one or more secondary structural elements comprised by an Anellovirus ORF1 protein (e.g., as described herein). In some embodiments, an ORF1 molecule comprises one or more secondary structural elements comprised by the jelly-roll domain of an Anellovirus ORF1 protein (e.g., as described herein). Generally, an ORF1 jelly-roll domain comprises a secondary structure comprising, in order in the N-terminal to C-terminal direction, a first beta strand, a second beta strand, a first alpha helix, a third beta strand, a fourth beta strand, a fifth beta strand, a second alpha helix, a sixth beta strand, a seventh beta strand, an eighth beta strand, and a ninth beta strand. In some embodiments, an ORF1 molecule comprises a secondary structure comprising, in order in the N-terminal to C-terminal direction, a first beta strand, a second beta strand, a first alpha helix, a third beta strand, a fourth beta strand, a fifth beta strand, a second alpha helix, a sixth beta strand, a seventh beta strand, an eighth beta strand, and/or a ninth beta strand.

In some embodiments, a pair of the conserved secondary structural elements (i.e., the beta strands and/or alpha helices) are separated by an interstitial amino acid sequence, e.g., comprising a random coil sequence, a beta strand, or an alpha helix, or a combination thereof. Interstitial amino acid sequences between the conserved secondary structural elements may comprise, for example, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more amino acids. In some embodiments, an ORF1 molecule may further comprise one or more additional beta strands and/or alpha helices (e.g., in the jelly-roll domain). In some embodiments, consecutive beta strands or consecutive alpha helices may be combined. In some embodiments, the first beta strand and the second beta strand are comprised in a larger beta strand. In some embodiments, the third beta strand and the fourth beta strand are comprised in a larger beta strand. In some embodiments, the fourth beta strand and the fifth beta strand are comprised in a larger beta strand. In some embodiments, the sixth beta strand and the seventh beta strand are comprised in a larger beta strand. In some embodiments, the seventh beta strand and the eighth beta strand are comprised in a larger beta strand. In some embodiments, the eighth beta strand and the ninth beta strand are comprised in a larger beta strand.

In some embodiments, the first beta strand is about 5-7 (e.g., 3, 4, 5, 6, 7, 8, 9, or 10) amino acids in length. In some embodiments, the second beta strand is about 15-16 (e.g., 13, 14, 15, 16, 17, 18, or 19) amino acids in length. In some embodiments, the first alpha helix is about 15-17 (e.g., 13, 14, 15, 16, 17, 18, 19, or 20) amino acids in length. In some embodiments, the third beta strand is about 3-4 (e.g., 1, 2, 3, 4, 5, or 6) amino acids in length. In some embodiments, the fourth beta strand is about 10-11 (e.g., 8, 9, 10, 11, 12, or 13) amino acids in length. In some embodiments, the fifth beta strand is about 6-7 (e.g., 4, 5, 6, 7, 8, 9, or 10) amino acids in length. In some embodiments, the second alpha helix is about 8-14 (e.g., 5, 6, 7, 8, 9, 10, 11, 12, 13, 14, 15, 16, or 17) amino acids in length. In some embodiments, the second alpha helix may be broken up into two smaller alpha helices (e.g., separated by a random coil sequence). In some embodiments, each of the two smaller alpha helices are about 4-6 (e.g., 2, 3, 4, 5, 6, 7, or 8) amino acids in length. In some embodiments, the sixth beta strand is about 4-5 (e.g., 2, 3, 4, 5, 6, or 7) amino acids in length. In some embodiments, the seventh beta strand is about 5-6 (e.g., 3, 4, 5, 6, 7, 8, or 9) amino acids in length. In some embodiments, the eighth beta strand is about 7-9 (e.g., 5, 6, 7, 8, 9, 10, 11, 12, or 13) amino acids in length. In some embodiments, the ninth beta strand is about 5-7 (e.g., 3, 4, 5, 6, 7, 8, 9, or 10) amino acids in length.

Exemplary jelly-roll domain secondary structures are described in Example 47 and FIG. 47 of PCT/US19/65995. In some embodiments, an ORF1 molecule comprises a region comprising one or more (e.g., 1, 2, 3, 4, 5, 6, 7, 8, 9, 10, or all) of the secondary structural elements (e.g., beta strands and/or alpha helices) of any of the jelly-roll domain secondary structures shown in FIG. 47 of PCT/US19/65995.

Consensus ORF1 Domain Sequences

In some embodiments, an ORF1 molecule, e.g., as described herein, comprises one or more of a jelly-roll domain, N22 domain, and/or C-terminal domain (CTD). In some embodiments, the jelly-roll domain comprises an amino acid sequence having a jelly-roll domain consensus sequence as described herein (e.g., as listed in any of Tables 37A-37C). In some embodiments, the N22 domain comprises an amino acid sequence having a N22 domain consensus sequence as described herein (e.g., as listed in any of Tables 37A-37C). In some embodiments, the CTD domain comprises an amino acid sequence having a CTD domain consensus sequence as described herein (e.g., as listed in any of Tables 37A-37C). In some embodiments, the amino acids listed in any of Tables 37A-37C in the format “(X_{a-b})” comprise a contiguous series of amino acids, in which the series comprises at least a, and at most b, amino acids. In certain embodiments, all of the amino acids in the series are identical. In other embodiments, the series comprises at least two (e.g., at least 2, 3, 4, 5, 6, 7, 8, 9, 10, 11, 12, 13, 14, 15, 16, 17, 18, 19, 20, or 21) different amino acids.

TABLE 37A
Alphatorquevius ORF1 domain consensus sequences

| Domain | Sequence | SEQ ID NO: |
|---|---|---|
| Jelly-Roll | LVLTQWQPNTVRRCYIRGYLPLIICGEN(X₀₋₃)TTSRNYATHS | 227 |
| | DDTIQKGPFGGGMSTTTFSLRVLYDEYQRFMNRWTYSNED | |
| | LDLARYLGCKFTFYRHPDXDFIVQYNTNPPFKDTKLTAPSIH | |
| | P(X₁₋₅)GMLMLSKRKILIPSLKTRPKGKHYVKVRIGPPKLFED | |
| | KWYTQSDLCDVPLVXLYATAADLQHPFGSPQTDNPCVTFQ | |
| | VLGSXYNKHLSISP; | |
| | wherein X = any amino acid. | |
| N22 | SNFEFPGAYTDITYNPLTDKGVGNMVWIQYLTKPDTIXDKT | 228 |
| | QS(X₀₋₃)KCLIEDLPLWAALYGYVDFCEKETGDSAIIXNXGRV | |
| | LIRCPYTKPPLYDKT(X₀₋₄)NKGFVPYSTNFGNGKMPGGSGY | |
| | VPIYWRARWYPTLFHQKEVLEDIVQSGPFAYKDEKPSTQLV | |
| | MKYCFNFN; | |
| | wherein X = any amino acid. | |
| CTD | WGGNPISQQVVRNPCKDSG(X₀₋₃)SGXGRQPRSVQVVDPKY | 229 |
| | MGPEYTFHSWDWRRGLFGEKAIKRMSEQPTDDEIFTGGXPK | |
| | RPRRDPPTXQXPEE(X₁₋₄)QKESSSFR(X₂₋₁₄)PWESSSQEXES | |
| | ESQEEEE(X₀₋₃₀)EQTVQQQLRQQLREQRRLRVQLQLLFQQLLK | |
| | T(X₀₋₄)QAGLHINPLLLSQA(X₀₋₄₀)*; | |
| | wherein X = any amino acid. | |

TABLE 37B
Betatorquevius ORF1 domain consensus sequences

| Domain | Sequence | SEQ ID NO: |
|---|---|---|
| Jelly-Roll | LKQWQPSTIRKCKIKGYLPLFQCGKGRISNNYTQYKESIVPH | 230 |
| | HEPGGGGWSIQQFTLGALYEEHLKLRNWWTKSNDGLPLVR | |
| | YLGCTIKLYRSEDTDYIVTYQRCYPMTATKLTYLSTQPSRM | |
| | LMNKHKIIVPSKXT(X₁₋₄)NKKKKPYKKIFIKPPSQMQNKWYF | |
| | QQDIANTPLLQLTXTACSLDRMYLSSDSISNNITFTSLNTNFF | |
| | QNPNFQ; | |
| | wherein X = any amino acid. | |
| N22 | (X₄₋₁₀)TPLYFECRYNPFKDKGTGNKVYLVSNN(X₁₋₈)TGWDPP | 231 |
| | TDPDLIIEGFPLWLLLWGWLDWQKKLGKIQNIDTDYILVIQS | |
| | XYYIPP(X₁₋₃)KLPYYVPLDXD(X₀₋₂)FLHGRSPY(X₃₋₁₆)PSDK | |
| | QHWHPKVRFQXETINNIALTGPGTPKLPNQKSIQAHMKYKFYF | |
| | K; | |
| | wherein X = any amino acid. | |
| CTD | WGGCPAPMETITDPCKQPKYPIPNNLLQTTSLQXPTTPIETYL | 232 |
| | YKFDERRGLLTKKAAKRIKKDXTTETTLFTDTGXXTSTTLPT | |
| | XXQTETTQEEXTSEEE(X₀₋₅)ETLLQQLQQLRRKQKQLRXRIL | |
| | QLLQLLXLL(X₀₋₂₆)*; | |
| | wherein X = any amino acid. | |

TABLE 37C
Gammatorquevius ORF1 domain consensus sequences

| Domain | Sequence | SEQ ID NO: |
|---|---|---|
| Jelly-Roll | TIPLKQWQPESIRKCKIKGYGTLVLGAEGRQFYCYTNEKDE | 233 |
| | YTPPKAPGGGGFGVELFSLEYLYEQWKARNNIWTKSNXYK | |
| | DLCRYTGCKITFYRHPTTDFIVXYSRQPPFEIDKXTYMXXHP | |
| | QXLLLRKHKKIILSKATNPKGKLKKKIKIKPPKQMLNKWFF | |
| | QKQFAXYGLVQLQAAACBLRYPRLGCCNENRLITLYYLN; | |
| | wherein X = any amino acid. | |
| N22 | LPIVVARYNPAXDTGKGNKXWLXSTLNGSXWAPPTTDKDL | 234 |
| | IIEGLPLWLALYGYWSYJKKVKKDKGILQSHMFVVKSPAIQP | |
| | LXTATTQXTFYPXIDNSFIQGKXPYDEPJTXNQKKLWYPTLE | |
| | HQQETINAIVESGPYVPKLDNQKNSTWELXYXYTFYFK; | |
| | wherein X = any amino acid. | |
| CTD | WGGPQIPDQPVEDPKXQGTYPVPDTXQQTIQIXNPLKQKPE | 235 |
| | TMFHDWDYRRGIITSTALKRMQENLETDSSFXSDSEETP(X₀₋₂) | |
| | KKKKRLTXELPXPQEETEEIQSCLLSLCEESTCQEE(X₁₋₆)ENL | |
| | QQLIHQQQQQQQQLKHNILKLLSDLKZKQRLLQLQTGILE | |
| | (X₁₋₁₀)*; | |
| | wherein X = any amino acid. | |

In some embodiments, the jelly-roll domain comprises a jelly-roll domain amino acid sequence as listed in any of Tables 21, 23, 25, 27, 29, 31, 33, 35, D2, D4, D6, D8, D10, or 37A-37C, or an amino acid sequence having at least 70%, 75%, 80%, 8%, 90%, 95%, 96%, 97%, 98%, 99%, or 100% sequence identity thereto. In some embodiments, the N22 domain comprises a N22 domain amino acid sequence as listed in any of Tables 21, 23, 25, 27, 29, 31, 33, 35, D2, D4, D6, D8, D10, or 37A-37C, or an amino acid sequence having at least 70%, 75%, 80%, 8%, 90%, 95%, 96%, 97%, 98%, 99%, or 100% sequence identity thereto. In some embodiments, the CTD domain comprises a CTD domain amino acid sequence as listed in any of Tables 21, 23, 25, 27, 29, 31, 33, 35, D2, D4, D6, D8, D10, or 37A-37C, or an amino acid sequence having at least 70%, 75%, 80%, 8%, 90%, 95%, 96%, 97%, 98%, 99%, or 100% sequence identity thereto.

Identification of ORF1 Protein Sequences

In some embodiments, an Anellovirus ORF1 protein sequence, or a nucleic acid sequence encoding an ORF1 protein, can be identified from the genome of an Anellovirus (e.g., a putative Anellovirus genome identified, for example, by nucleic acid sequencing techniques, e.g., deep sequencing techniques). In some embodiments, an ORF1 protein sequence is identified by one or more (e.g., 1, 2, or all 3) of the following selection criteria:
(i) Length Selection: Protein sequences (e.g., putative Anellovirus ORF1 sequences passing the criteria described in (ii) or (iii) below) may be size-selected for those greater than about 600 amino acid residues to identify putative Anellovirus ORF1 proteins. In some embodiments, an Anellovirus ORF1 protein sequence is at least about 600, 650, 700, 750, 800, 850, 900, 950, or 1000 amino acid residues in length. In some embodiments, an Alphatorquevirus ORF1 protein sequence is at least about 700, 710, 720, 730, 740, 750, 760, 770, 780, 790, 800, 900, or 1000 amino acid residues in length. In some embodiments, a Betatorquevirus ORF1 protein sequence is at least about 650, 660, 670, 680, 690, 700, 750, 800, 900, or 1000 amino acid residues in length. In some embodiments, a Gammatorquevirus ORF1 protein sequence is at least about 650, 660, 670, 680, 690, 700, 750, 800, 900, or 1000 amino acid residues in length. In some embodiments, a nucleic acid sequence encoding an Anellovirus ORF1 protein is at least about 1800, 1900, 2000, 2100, 2200, 2300, 2400, or 2500 nucleotides in length. In some embodiments, a nucleic acid sequence encoding an Alphatorquevirus ORF1 protein sequence is at least about 2100, 2150, 2200, 2250, 2300, 2400, or 2500 nucleotides in length. In some embodiments, a nucleic acid sequence encoding a Betatorquevirus ORF1 protein sequence is at least about 1900, 1950, 2000, 2500, 2100, 2150, 2200, 2250, 2300, 2400, or 2500 or 1000 nucleotides in length. In some embodiments, a nucleic acid sequence encoding a Gammatorquevirus ORF1 protein sequence is at least about 1900, 1950, 2000, 2500, 2100, 2150, 2200, 2250, 2300, 2400, or 2500 or 1000 nucleotides in length.
(ii) Presence of ORF1 motif: Protein sequences (e.g., putative Anellovirus ORF1 sequences passing the criteria described in (i) above or (iii) below) may be filtered to identify those that contain the conserved ORF1 motif in the N22 domain described above. In some embodiments, a putative Anellovirus ORF1 sequence comprises the sequence YNPXXDXGXXN. In some embodiments, a putative Anellovirus ORF1 sequence comprises the sequence Y[NCS]PXXDX[GASKR]XX[NTSVAK].
(iii) Presence of arginine-rich region: Protein sequences (e.g., putative Anellovirus ORF1 sequences passing the criteria described in (i) and/or (ii) above) may be filtered for those that include an arginine-rich region (e.g., as described herein). In some embodiments, a putative Anellovirus ORF1 sequence comprises a contiguous sequence of at least about 30, 35, 40, 45, 50, 55, 60, 65, or 70 amino acids that comprises at least 30% (e.g., at least about 20%, 25%, 30%, 35%, 40%, 45%, or 50%) arginine residues. In some embodiments, a putative Anellovirus ORF1 sequence comprises a contiguous sequence of about 35-40, 40-45, 45-50, 50-55, 55-60, 60-65, or 65-70 amino acids that comprises at least 30% (e.g., at least about 20%, 25%, 30%, 35%, 40%, 45%, or 50%) arginine residues. In some embodiments, the arginine-rich region is positioned at least about 30, 40, 50, 60, 70, or 80 amino acids downstream of the start codon of the putative Anellovirus ORF1 protein. In some embodiments, the arginine-rich region is positioned at least about 50 amino acids downstream of the start codon of the putative Anellovirus ORF1 protein.

### ORF2 Molecules

In some embodiments, the anellovector comprises an ORF2 molecule and/or a nucleic acid encoding an ORF2 molecule. Generally, an ORF2 molecule comprises a polypeptide having the structural features and/or activity of an Anellovirus ORF2 protein (e.g., an Anellovirus ORF2 protein as described herein, e.g., as listed in any of Tables A2, A4, A6, A8, A10, A12, C1-C5, 2, 4, 6, 8, 10, 12, 14, 16, or 18), or a functional fragment thereof. In some embodiments, an ORF2 molecule comprises an amino acid sequence having at least 70%, 75%, 80%, 85%, 90%, 95%, 96%, 97%, 98%, 99%, or 100% sequence identity to an Anellovirus ORF2 protein sequence as shown in any of Tables A2, A4, A6, A8, A10, A12, C1-C5, 2, 4, 6, 8, 10, 12, 14, 16, or 18.

In some embodiments, an ORF2 molecule comprises an amino acid sequence having at least 75%, 80%, 85%, 90%, 95%, 96%, 97%, 98%, or 99% sequence identity to an Alphatorquevirus, Betatorquevirus, or Gammatorquevirus ORF2 protein. In some embodiments, an ORF2 molecule (e.g., an ORF2 molecule having at least 75%, 80%, 85%, 90%, 95%, 96%, 97%, 98%, or 99% sequence identity to an Alphatorquevirus ORF2 protein) has a length of 250 or fewer amino acids (e.g., about 150-200 amino acids). In some embodiments, an ORF2 molecule (e.g., an ORF2 molecule having at least 75%, 80%, 85%, 90%, 95%, 96%, 97%, 98%, or 99% sequence identity to a Betatorquevirus ORF2 protein) has a length of about 50-150 amino acids. In some embodiments, an ORF2 molecule (e.g., an ORF2 molecule having at least 75%, 80%, 85%, 90%, 95%, 96%, 97%, 98%, or 99% sequence identity to a Gammatorquevirus ORF2 protein) has a length of about 100-200 amino acids (e.g., about 100-150 amino acids). In some embodiments, the ORF2 molecule comprises a helix-turn-helix motif (e.g., a helix-turn-helix motif comprising two alpha helices flanking a turn region). In some embodiments, the ORF2 molecule does not comprise the amino acid sequence of the ORF2 protein of TTV isolate TA278 or TTV isolate SANBAN. In some embodiments, an ORF2 molecule has protein phosphatase activity. In some embodiments, an ORF2 molecule or a nucleic acid molecule encoding same comprises at least one difference (e.g., a mutation, chemical modification, or epigenetic alteration) relative to a wild-type ORF2 protein, e.g., as described herein (e.g., as shown in any of Tables A2, A4, A6, A8, A10, A12, C1-C5, 2, 4, 6, 8, 10, 12, 14, 16, or 18).

Conserved ORF2 Motif

In some embodiments, a polypeptide (e.g., an ORF2 molecule) described herein comprises the amino acid sequence [W/F]X⁷HX³CX¹CX⁵H (SEQ ID NO: 949), wherein Xⁿ is a contiguous sequence of any n amino acids. In embodiments, X⁷ indicates a contiguous sequence of any seven amino acids. In embodiments, X³ indicates a contiguous sequence of any three amino acids. In embodiments, X¹ indicates any single amino acid. In embodiments, X⁵ indicates a contiguous sequence of any five amino acids. In some embodiments, the [W/F] can be either tryptophan or phenylalanine. In some embodiments, the [W/F]X⁷HX³CX¹CX⁵H (SEQ ID NO: 949) is comprised within the N22 domain of an ORF2 molecule, e.g., as described herein. In some embodiments, a genetic element described herein comprises a nucleic acid sequence (e.g., a nucleic acid sequence encoding an ORF2 molecule, e.g., as described herein) encoding the amino acid sequence [W/F]X⁷HX³CX¹CX⁵H (SEQ ID NO: 949), wherein Xⁿ is a contiguous sequence of any n amino acids.

### Genetic Elements

In some embodiments, the anellovector comprises a genetic element. In some embodiments, the genetic element has one or more of the following characteristics: is substantially non-integrating with a host cell's genome, is an episomal nucleic acid, is a single stranded DNA, is circular, is about 1 to 10 kb, exists within the nucleus of the cell, can be bound by endogenous proteins, produces an effector, such as a polypeptide or nucleic acid (e.g., an RNA, iRNA, microRNA) that targets a gene, activity, or function of a host or target cell. In one embodiment, the genetic element is a substantially non-integrating DNA. In some embodiments, the genetic element comprises a packaging signal, e.g., a sequence that binds a capsid protein. In some embodiments, outside of the packaging or capsid-binding sequence, the genetic element has less than 70%, 60%, 50%, 40%, 30%, 20%, 10%, 5% sequence identity to a wild type Anellovirus nucleic acid sequence, e.g., has less than 70%, 60%, 50%, 40%, 30%, 20%, 10%, 5% sequence identity to an Anellovirus nucleic acid sequence, e.g., as described herein. In some embodiments, outside of the packaging or capsid-binding sequence, the genetic element has less than 500 450, 400, 350, 300, 250, 200, 150, or 100 contiguous nucleotides that are at least 70%, 75%, 80%, 8%, 90%, 95%, 96%, 97%, 98%, 99%, or 100% identical to an Anellovirus nucleic acid sequence. In certain embodiments, the genetic element is a circular, single stranded DNA that comprises a promoter sequence, a sequence encoding a therapeutic effector, and a capsid binding protein.

In some embodiments, the genetic element has a length less than 20 kb (e.g., less than about 19 kb, 18 kb, 17 kb, 16 kb, 15 kb, 14 kb, 13 kb, 12 kb, 11 kb, 10 kb, 9 kb, 8 kb, 7 kb, 6 kb, 5 kb, 4 kb, 3 kb, 2 kb, 1 kb, or less). In some embodiments, the genetic element has, independently or in addition to, a length greater than 1000b (e.g., at least about 1.1 kb, 1.2 kb, 1.3 kb, 1.4 kb, 1.5 kb, 1.6 kb, 1.7 kb, 1.8 kb, 1.9 kb, 2 kb, 2.1 kb, 2.2 kb, 2.3 kb, 2.4 kb, 2.5 kb, 2.6 kb, 2.7 kb, 2.8 kb, 2.9 kb, 3 kb, 3.1 kb, 3.2 kb, 3.3 kb, 3.4 kb, 3.5 kb, 3.6 kb, 3.7 kb, 3.8 kb, 3.9 kb, 4 kb, 4.1 kb, 4.2 kb, 4.3 kb, 4.4 kb, 4.5 kb, 4.6 kb, 4.7 kb, 4.8 kb, 4.9 kb, 5 kb, or greater). In some embodiments, the genetic element has a length of about 2.5-4.6, 2.8-4.0, 3.0-3.8, or 3.2-3.7 kb. In some embodiments, the genetic element has a length of about 1.5-2.0, 1.5-2.5, 1.5-3.0, 1.5-3.5, 1.5-3.8, 1.5-3.9, 1.5-4.0, 1.5-4.5, or 1.5-5.0 kb. In some embodiments, the genetic element has a length of about 2.0-2.5, 2.0-3.0, 2.0-3.5, 2.0-3.8, 2.0-3.9, 2.0-4.0, 2.0-4.5, or 2.0-5.0 kb. In some embodiments, the genetic element has a length of about 2.5-3.0, 2.5-3.5, 2.5-3.8, 2.5-3.9, 2.5-4.0, 2.5-4.5, or 2.5-5.0 kb. In some embodiments, the genetic element has a length of about 3.0-5.0, 3.5-5.0, 4.0-5.0, or 4.5-5.0 kb. In some embodiments, the genetic element has a length of about 1.5-2.0, 2.0-2.5, 2.5-3.0, 3.0-3.5, 3.1-3.6, 3.2-3.7, 3.3-3.8, 3.4-3.9, 3.5-4.0, 4.0-4.5, or 4.5-5.0 kb. In some embodiments, the genetic element has a length between about 3.6-3.9 kb. In some embodiments, the genetic element has a length between about 2.8-2.9 kb. In some embodiments, the genetic element has a length between about 2.0-3.2 kb.

In some embodiments, the genetic element comprises one or more of the features described herein, e.g., a sequence encoding a substantially non-pathogenic protein, a protein binding sequence, one or more sequences encoding a regulatory nucleic acid, one or more regulatory sequences, one or more sequences encoding a replication protein, and other sequences.

In embodiments, the genetic element was produced from a double-stranded circular DNA (e.g., produced by in vitro circularization). In some embodiments, the genetic element was produced by rolling circle replication from the double-stranded circular DNA. In embodiments, the rolling circle replication occurs in a cell (e.g., a host cell, e.g., a mammalian cell, e.g., a human cell, e.g., a HEK293T cell, an A549 cell, or a Jurkat cell). In embodiments, the genetic element can be amplified exponentially by rolling circle replication in the cell. In embodiments, the genetic element can be amplified linearly by rolling circle replication in the cell. In embodiments, the double-stranded circular DNA or genetic element is capable of yielding at least 2, 4, 8, 16, 32, 64, 128, 256, 518, 1024 or more times the original quantity by rolling circle replication in the cell. In embodiments, the double-stranded circular DNA was introduced into the cell, e.g., as described herein.

In some embodiments, the double-stranded circular DNA and/or the genetic element does not comprise one or more bacterial plasmid elements (e.g., a bacterial origin of replication or a selectable marker, e.g., a bacterial resistance gene). In some embodiments, the double-stranded circular DNA and/or the genetic element does not comprise a bacterial plasmid backbone.

In one embodiment, the invention includes a genetic element comprising a nucleic acid sequence (e.g., a DNA sequence) encoding (i) a substantially non-pathogenic exterior protein, (ii) an exterior protein binding sequence that binds the genetic element to the substantially non-pathogenic exterior protein, and (iii) a regulatory nucleic acid. In such an embodiment, the genetic element may comprise one or more sequences with at least about 60%, 70% 80%, 85%, 90% 95%, 96%, 97%, 98% and 99% nucleotide sequence identity to any one of the nucleotide sequences to a native viral sequence (e.g., a native Anellovirus sequence, e.g., as described herein).

### Protein Binding Sequence

A strategy employed by many viruses is that the viral capsid protein recognizes a specific protein binding sequence in its genome. For example, in viruses with unsegmented genomes, such as the L-A virus of yeast, there is a secondary structure (stem-loop) and a specific sequence at the 5′ end of the genome that are both used to bind the viral capsid protein. However, viruses with segmented genomes, such as Reoviridae, Orthomyxoviridae (influenza), Bunyaviruses and Arenaviruses, need to package each of the genomic segments. Some viruses utilize a complementarity region of the segments to aid the virus in including one of each of the genomic molecules. Other viruses have specific binding sites for each of the different segments. See for example, Curr Opin Struct Biol. 2010 February; 20(1): 114-120; and Journal of Virology (2003), 77(24), 13036-13041.

In some embodiments, the genetic element encodes a protein binding sequence that binds to the substantially non-pathogenic protein. In some embodiments, the protein binding sequence facilitates packaging the genetic element into the proteinaceous exterior. In some embodiments, the protein binding sequence specifically binds an arginine-rich region of the substantially non-pathogenic protein. In some embodiments, the genetic element comprises a protein binding sequence as described in Example 8 of PCT/US19/65995.

In some embodiments, the genetic element comprises a protein binding sequence having at least 70%, 80%, 85%, 90%, 95%, 96%, 97%, 98%, 99%, or 100% sequence identity to a 5′ UTR conserved domain or GC-rich domain of an Anellovirus sequence, e.g., as described herein.

In embodiments, the protein binding sequence has at least about 70%, 75%, 80%, 85%, 90%, 95%, 96%, 97%, 98%, 99%, or 100% sequence identity to an Anellovirus 5′ UTR conserved domain nucleotide sequence, e.g., as described herein.

### 5′ UTR Regions

In some embodiments, a nucleic acid molecule as described herein (e.g., a genetic element, genetic element construct, or genetic element region) comprises a 5′ UTR sequence, e.g., a 5′ UTR conserved domain sequence as described herein (e.g., in any of Tables A1, B1, or C1), or a sequence having at least 75%, 80%, 85%, 90%, 95%, 96%, 97%, 98%, or 99% sequence identity thereto.

In some embodiments, the 5′ UTR sequence comprises the nucleic acid sequence AGGTGAGTGAAACCACCGAAGTCAAGGGGCAATTCGGGCTAGGGX₁CAGTCT, or a nucleic acid sequence having at least 85%, 90%, 95%, 96%, 97%, 98%, or 99% sequence identity thereto. In some embodiments, the 5′ UTR sequence comprises the nucleic acid sequence AGGTGAGTGAAACCACCGAAGTCAAGGGGCAATTCGGGCTAGGGX₁CAGTCT, or a nucleic acid sequence having no more than 1, 2, 3, 4, 5, 6, 7, 8, 9, or 10 nucleotide differences (e.g., substitutions, deletions, or additions) relative thereto. In embodiments, X₁ is A. In embodiments, X₁ is absent.

In some embodiments, the 5′ UTR sequence comprises the nucleic acid sequence of the 5′ UTR of an Alphatorquevirus (e.g., Ring1), or a sequence having at least 75%, 80%, 85%, 90%, 95%, 96%, 97%, 98%, or 99% sequence identity thereto. In embodiments, the 5′ UTR sequence comprises the nucleic acid sequence of the 5′ UTR conserved domain listed in Table A1, or a sequence having at least 75%, 80%, 85%, 90%, 95%, 96%, 97%, 98%, or 99% sequence identity thereto. In some embodiments, the nucleic acid molecule comprises a nucleic acid sequence having at least 95% sequence identity to the 5′ UTR conserved domain listed in Table A1. In some embodiments, the nucleic acid molecule comprises a nucleic acid sequence having at least 95.775% sequence identity to the 5′ UTR conserved domain listed in Table A1. In some embodiments, the nucleic acid molecule comprises a nucleic acid sequence having at least 97% sequence identity to the 5′ UTR conserved domain listed in Table A1. In some embodiments, the nucleic acid molecule comprises a nucleic acid sequence having at least 97.183% sequence identity to the 5′ UTR conserved domain listed in Table A1. In some embodiments, the 5′ UTR sequence comprises the nucleic acid sequence AGGTGAGTTTACACACCGCAGTCAAGGGGCAATTCGGGCTCGGGACTGGC, or a nucleic acid sequence having at least 85%, 90%, 95%, 96%, 97%, 98%, or 99% sequence identity thereto. In some embodiments, the 5′ UTR sequence comprises the nucleic acid sequence AGGTGAGTTTACACACCGCAGTCAAGGGGCAATTCGGGCTCGGGACTGGC, or a nucleic acid sequence having no more than 1, 2, 3, 4, 5, 6, 7, 8, 9, or 10 nucleotide differences (e.g., substitutions, deletions, or additions) relative thereto.

In some embodiments, the 5′ UTR sequence comprises the nucleic acid sequence of the 5′ UTR of an Betatorquevirus (e.g., Ring2), or a sequence having at least 75%, 80%, 85%, 86%, 87%, 88%, 89%, 90%, 91%, 92%, 93%, 94%, 95%, 96%, 97%, 98%, or 99% sequence identity thereto. In embodiments, the 5′ UTR sequence comprises the nucleic acid sequence of the 5′ UTR conserved domain listed in Table B1, or a sequence having at least 75%, 80%, 85%, 86%, 87%, 88%, 89%, 90%, 91%, 92%, 93%, 94%, 95%, 96%, 97%, 98%, or 99% sequence identity thereto. In some embodiments, the nucleic acid molecule comprises a nucleic acid sequence having at least 85% sequence identity to the 5′ UTR conserved domain listed in Table B1. In some embodiments, the nucleic acid molecule comprises a nucleic acid sequence having at least 87% sequence identity to the 5′ UTR conserved domain listed in Table B1. In some embodiments, the nucleic acid molecule comprises a nucleic acid sequence having at least 87.324% sequence identity to the 5′ UTR conserved domain listed in Table B1. In some embodiments, the nucleic acid molecule comprises a nucleic acid sequence having at least 88% sequence identity to the 5′ UTR conserved domain listed in Table B1. In some embodiments, the nucleic acid molecule comprises a nucleic acid sequence having at least 88.732% sequence identity to the 5′ UTR conserved domain listed in Table B1. In some embodiments, the nucleic acid molecule comprises a nucleic acid sequence having at least 91% sequence identity to the 5′ UTR conserved domain listed in Table B1. In some embodiments, the nucleic acid molecule comprises a nucleic acid sequence having at least 91.549% sequence identity to the 5′ UTR conserved domain listed in Table B1. In some embodiments, the nucleic acid molecule comprises a nucleic acid sequence having at least 92% sequence identity to the 5′ UTR conserved domain listed in Table B1. In some embodiments, the nucleic acid molecule comprises a nucleic acid sequence having at least 92.958% sequence identity to the 5′ UTR conserved domain listed in Table B1. In some embodiments, the nucleic acid molecule comprises a nucleic acid sequence having at least 94% sequence identity to the 5′ UTR conserved domain listed in Table B1. In some embodiments, the nucleic acid molecule comprises a nucleic acid sequence having at least 94.366% sequence identity to the 5′ UTR conserved domain listed in Table B1. In some embodiments, the nucleic acid molecule comprises a nucleic acid sequence having at least 95% sequence identity to the 5′ UTR conserved domain listed in Table B1. In some embodiments, the nucleic acid molecule comprises a nucleic acid sequence having at least 95.775% sequence identity to the 5′ UTR conserved domain listed in Table B1. In some embodiments, the nucleic acid molecule comprises a nucleic acid sequence having at least 97% sequence identity to the 5′ UTR conserved domain listed in Table B1. In some embodiments, the nucleic acid molecule comprises a nucleic acid sequence having at least 97.183% sequence identity to the 5′ UTR conserved domain listed in Table B1. In some embodiments, the 5′ UTR sequence comprises the nucleic acid sequence AGGTGAGTGAAACCACCGAAGTCAAGGGGCAATTCGGGCTAGATCAGTCT, or a nucleic acid sequence having at least 85%, 90%, 95%, 96%, 97%, 98%, or 99% sequence identity thereto. In some embodiments, the 5′ UTR sequence comprises the nucleic acid sequence AGGTGAGTGAAACCACCGAAGTCAAGGGGCAATTCGGGCTAGATCAGTCT, or a nucleic acid sequence having no more than 1, 2, 3, 4, 5, 6, 7, 8, 9, or 10 nucleotide differences (e.g., substitutions, deletions, or additions) relative thereto.

In some embodiments, the 5′ UTR sequence comprises the nucleic acid sequence of the 5′ UTR of an Gammatorquevirus (e.g., Ring4), or a sequence having at least 75%, 80%, 85%, 90%, 95%, 96%, 97%, 98%, or 99% sequence identity thereto. In embodiments, the 5′ UTR sequence comprises the nucleic acid sequence of the 5′ UTR conserved domain listed in Table C1, or a sequence having at least 75%, 80%, 85%, 90%, 95%, 96%, 97%, 98%, or 99% sequence identity thereto. In some embodiments, the nucleic acid molecule comprises a nucleic acid sequence having at least 97% sequence identity to the 5′ UTR conserved domain listed in Table C1. In some embodiments, the nucleic acid molecule comprises a nucleic acid sequence having at least 97.183% sequence identity to the 5′ UTR conserved domain listed in Table C1. In some embodiments, the 5′ UTR sequence comprises the nucleic acid sequence AGGTGAGTGAAACCACCGAGGTCTAGGGGCAATTCGGGCTAGGGCAGTCT, or a nucleic acid sequence having at least 85%, 90%, 95%, 96%, 97%, 98%, or 99% sequence identity thereto. In some embodiments, the 5′ UTR sequence comprises the nucleic acid sequence AGGTGAGTGAAACCACCGAGGTCTAGGGGCAATTCGGGCTAGGGCAGTCT, or a nucleic acid sequence having no more than 1, 2, 3, 4, 5, 6, 7, 8, 9, or 10 nucleotide differences (e.g., substitutions, deletions, or additions) relative thereto.

In some embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to an Anellovirus 5′ UTR sequence, e.g., a nucleic acid sequence shown in Table 38. In some embodiments, the genetic element (e.g., protein-binding sequence of the genetic element) comprises a nucleic acid sequence of the Consensus 5′ UTR sequence shown in Table 38, wherein X₁, X₂, X₃, X₄, and X₅ are each independently any nucleotide, e.g., wherein X₁=G or T, X₂=C or A, X₃=G or A, X₄=T or C, and X₅=A, C, or T). In embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to the Consensus 5′ UTR sequence shown in Table 38. In embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to the exemplary TTV 5′ UTR sequence shown in Table 38. In embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to the TTV-CT30F 5′ UTR sequence shown in Table 38. In embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to the TTV-HD23a 5′ UTR sequence shown in Table 38. In embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to the TTV-JA20 5′ UTR sequence shown in Table 38. In embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to the TTV-TJN02 5′ UTR sequence shown in Table 38. In embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to the TTV-tth8 5′ UTR sequence shown in Table 38.

In embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to the Alphatorquevirus Consensus 5′ UTR sequence shown in Table 38. In embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to the Alphatorquevirus Clade 1 5′ UTR sequence shown in Table 38. In embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to the Alphatorquevirus Clade 2 5′ UTR sequence shown in Table 38. In embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to the Alphatorquevirus Clade 3 5′ UTR sequence shown in Table 38. In embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to the Alphatorquevirus Clade 4 5′ UTR sequence shown in Table 38. In embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to the Alphatorquevirus Clade 5 5′ UTR sequence shown in Table 38. In embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to the Alphatorquevirus Clade 6 5′ UTR sequence shown in Table 38. In embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to the Alphatorquevirus Clade 7 5′ UTR sequence shown in Table 38.

TABLE 38
Exemplary 5' UTR sequences from Anelloviruses

| Source | Sequence | SEQ ID NO: |
|---|---|---|
| Consensus | CGGGTGCCGX₁AGGTGAGTTTACACACCGX₂AGT | 105 |
| | CAAGGGGCAATTCGGGCTCX₃GGACTGGCCGGG | |
| | CX₄X₅TGGG | |
| | X₁ = G or T | |
| | X₂ = C or A | |
| | X₃ = G or A | |
| | X₄ = T or C | |
| | X₅ = A, C, or T | |
| Exemplary TTV Sequence | CGGGTGCCGGAGGTGAGTTTACACACCGCAGTC | 106 |
| | AAGGGGCAATTCGGGCTCGGGACTGGCCGGGCT | |
| | WTGGG | |
| TTV-CT30F | CGGGTGCCGTAGGTGAGTTTACACACCGCAGTC | 107 |
| | AAGGGGCAATTCGGGCTCGGGACTGGCCGGGCT | |
| | ATGGG | |
| TTV-HD23a | CGGGTGCCGGAGGTGAGTTTACACACCGCAGTC | 108 |
| | AAGGGGCAATTCGGGCTCGGGACTGGCCGGGCC | |
| | CTGGG | |
| TTV-JA20 | CGGGTGCCGGAGGTGAGTTTACACACCGCAGTC | 109 |
| | AAGGGGCAATTCGGGCTCGGGACTGGCCGGGCT | |
| | TTGGG | |
| TTV-TJN02 | CGGGTGCCGGAGGTGAGTTTACACACCGCAGTC | 110 |
| | AAGGGGCAATTCGGGCTCGGGACTGGCCGGGCT | |
| | ATGGG | |
| TTV-tth8 | CGGGTGCCGGAGGTGAGTTTACACACCGAAGTC | 111 |
| | AAGGGGCAATTCGGGCTCAGGACTGGCCGGGCT | |
| | TTGGG | |
| Alphatorquevirus | CGGGTGCCGGAGGTGAGTTTACACACCGCAGTC | 112 |
| Consensus 5' UTR | AAGGGGCAATTCGGGCTCGGGACTGGCCGGGC | |
| | X₁X₂TGGG; wherein X₁ comprises T | |
| | or C, and wherein X₂ comprises | |
| | A, C, or T. | |
| Alphatorquevirus | CGGGTGCCGTAGGTGAGTTTACACACCGCAGTC | 113 |
| Clade 1 5′ UTR (e.g., | AAGGGGCAATTCGGGCTCGGGACTGGCCGGGCT | |
| TTV-CT30F) | ATGGG | |
| Alphatorquevirus | CGGGTGCCGGAGGTGAGTTTACACACCGCAGTC | 114 |
| Clade 2 5′ UTR (e.g., | AAGGGGCAATTCGGGCTCGGGACTGGCCGGGCC | |
| TTV-P13-1) | CGGG | |
| Alphatorquevirus | CGGGTGCCGGAGGTGAGTTTACACACCGAAGTC | 115 |
| Clade 3 5′ UTR (e.g., | AAGGGGCAATTCGGGCTCAGGACTGGCCGGGCT | |
| TTV-tth8) | TTGGG | |
| Alphatorquevirus | CGGGTGCCGGAGGTGAGTTTACACACCGCAGTC | 116 |
| Clade 4 5′ UTR (e.g., | AAGGGGCAATTCGGGCTCGGGAGGCCGGGCCAT | |
| TTV-HD20a) | GGG | |
| Alphatorquevirus | CGGGTGCCGGAGGTGAGTTTACACACCGCAGTC | 117 |
| Clade 5 5′ UTR (e.g., | AAGGGGCAATTCGGGCTCGGGACTGGCCGGGCC | |
| TTV-16) | CCGGG | |
| Alphatorquevirus | CGGGTGCCGGAGGTGAGTTTACACACCGCAGTC | 118 |
| Clade 6 5′ UTR (e.g., | AAGGGGCAATTCGGGCTCGGGACTGGCCGGGCT | |
| TTV-TJN02) | ATGGG | |
| Alphatorquevirus | CGGGTGCCGAAGGTGAGTTTACACACCGCAGTC | 119 |
| Clade 7 5′ UTR (e.g., | AAGGGGCAATTCGGGCTCGGGACTGGCCGGGCT | |
| TTV-HD16d) | ATGGG | |

In some embodiments, an Anellovirus 5′ UTR sequence can be identified within the genome of an Anellovirus (e.g., a putative Anellovirus genome identified, for example, by nucleic acid sequencing techniques, e.g., deep sequencing techniques). In some embodiments, an Anellovirus 5′ UTR sequence is identified by one or both of the following steps:
(i) Identification of circularization junction point: In some embodiments, a 5′ UTR will be positioned near a circularization junction point of a full-length, circularized Anellovirus genome. A circularization junction point can be identified, for example, by identifying overlapping regions of the sequence. In some embodiments, a overlapping region of the sequence can be trimmed from the sequence to produce a full-length Anellovirus genome sequence that has been circularized. In some embodiments, a genome sequence is circularized in this manner using software. Without wishing to be bound by theory, computationally circularizing a genome may result in the start position for the sequence being oriented in a non-biological. Landmarks within the sequence can be used to re-orient sequences in the proper direction. For example, landmark sequence may include sequences having substantial homology to one or more elements within an Anellovirus genome as described herein (e.g., one or more of a TATA box, cap site, initiator element, transcriptional start site, 5′ UTR conserved domain, ORF1, ORF1/1, ORF1/2, ORF2, ORF2/2, ORF2/3, ORF2t/3, three open-reading frame region, poly(A) signal, or GC-rich region of an Anellovirus, e.g., as described herein).
(ii) Identification of 5′ UTR sequence: Once a putative Anellovirus genome sequence has been obtained, the sequence (or portions thereof, e.g., having a length between about 40-50, 50-60, 60-70, 70-80, 80-90, or 90-100 nucleotides) can be compared to one or more Anellovirus 5′ UTR sequences (e.g., as described herein) to identify sequences having substantial homology thereto. In some embodiments, a putative Anellovirus 5′ UTR region has at least 50%, 60%, 70%, 75%, 80%, 85%, 90%, 95%, 96%, 97%, 98%, 99%, or 100% sequence identity to an Anellovirus 5′ UTR sequence as described herein.

### GC-Rich Regions

In some embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to a nucleic acid sequence shown in Table 39. In embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to a GC-rich sequence shown in Table 39.

In embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to a 36-nucleotide GC-rich sequence as shown in Table 39 (e.g., 36-nucleotide consensus GC-rich region sequence 1, 36-nucleotide consensus GC-rich region sequence 2, TTV Clade 1 36-nucleotide region, TTV Clade 3 36-nucleotide region, TTV Clade 3 isolate GH1 36-nucleotide region, TTV Clade 3 sle1932 36-nucleotide region, TTV Clade 4 ctdc002 36-nucleotide region, TTV Clade 5 36-nucleotide region, TTV Clade 6 36-nucleotide region, or TTV Clade 7 36-nucleotide region). In embodiments, the genetic element (e.g., protein-binding sequence of the genetic element) comprises a nucleic acid sequence comprising at least 10, 15, 20, 25, 30, 31, 32, 33, 34, 35, or 36 consecutive nucleotides of a 36-nucleotide GC-rich sequence as shown in Table 39 (e.g., 36-nucleotide consensus GC-rich region sequence 1, 36-nucleotide consensus GC-rich region sequence 2, TTV Clade 1 36-nucleotide region, TTV Clade 3 36-nucleotide region, TTV Clade 3 isolate GH1 36-nucleotide region, TTV Clade 3 sle1932 36-nucleotide region, TTV Clade 4 ctdc002 36-nucleotide region, TTV Clade 5 36-nucleotide region, TTV Clade 6 36-nucleotide region, or TTV Clade 7 36-nucleotide region).

In embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to an Alphatorquevirus GC-rich region sequence, e.g., selected from TTV-CT30F, TTV-P13-1, TTV-tth8, TTV-HD20a, TTV-16, TTV-TJN02, or TTV-HD16d, e.g., as listed in Table 39. In embodiments, the genetic element (e.g., protein-binding sequence of the genetic element) comprises a nucleic acid sequence comprising at least 10, 15, 20, 25, 30, 35, 40, 45, 50, 60, 70, 80, 90, 100, 104, 105, 108, 110, 111, 115, 120, 122, 130, 140, 145, 150, 155, or 156 consecutive nucleotides of an Alphatorquevirus GC-rich region sequence, e.g., selected from TTV-CT30F, TTV-P13-1, TTV-tth8, TTV-HD20a, TTV-16, TTV-TJN02, or TTV-HD16d, e.g., as listed in Table 39.

In embodiments, the 36-nucleotide GC-rich sequence is selected from:

(i)
(SEQ ID NO: 160)
CGCGCTGCGCGCGCCGCCCAGTAGGGGGAGCCATGC,
(ii)
(SEQ ID NO: 164)
GCGCTX1CGCGCGCGCGCCGGGGGGCTGCGCCCCCCC,

wherein X₁ is selected from T, G, or A;

(iii)
(SEQ ID NO: 165)
GCGCTTCGCGCGCCGCCCACTAGGGGGCGTTGCGCG;
(iv)
(SEQ ID NO: 166)
GCGCTGCGCGCGCCGCCCAGTAGGGGGCGCAATGCG;
(v)
(SEQ ID NO: 167)
GCGCTGCGCGCGCGGCCCCCGGGGGAGGCATTGCCT;
(vi)
(SEQ ID NO: 168)
GCGCTGCGCGCGCGCGCCGGGGGGGCGCCAGCGCCC;
(vii)
(SEQ ID NO: 169)
GCGCTTCGCGCGCGCGCCGGGGGGCTCCGCCCCCCC;
(viii)
(SEQ ID NO: 170)
GCGCTTCGCGCGCGCGCCGGGGGGCTGCGCCCCCCC;
(ix)
(SEQ ID NO: 171)
GCGCTACGCGCGCGCGCCGGGGGGCTGCGCCCCCCC;
or
(x)
(SEQ ID NO: 172)
GCGCTACGCGCGCGCGCCGGGGGGCTCTGCCCCCCC.

In embodiments, the genetic element (e.g., protein-binding sequence of the genetic element) comprises the nucleic acid sequence CGCGCTGCGCGCGCCGCCCAGTAGGGGGAGCCATGC (SEQ ID NO: 160).

In embodiments, the genetic element (e.g., protein-binding sequence of the genetic element) comprises a nucleic acid sequence of the Consensus GC-rich sequence shown in Table 39, wherein X₁, X₄, X₅, X₆, X₇, X₁₂, X₁₃, X₁₄, X₁₅, X₂₀, X₂₁, X₂₂, X₂₆, X₂₉, X₃₀, and X₃₃ are each independently any nucleotide and wherein X₂, X₃, X₈, X₉, X₁₀, X₁₁, X₁₆, X₁₇, X₁₈, X₁₉, X₂₃, X₂₄, X₂₅, X₂₇, X₂₈, X₃₁, X₃₂, and X₃₄ are each independently absent or any nucleotide. In some embodiments, one or more of (e.g., all of) X₁ through X₃₄ are each independently the nucleotide (or absent) specified in Table 39. In embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to an exemplary TTV GC-rich sequence shown in Table 39 (e.g., the full sequence, Fragment 1, Fragment 2, Fragment 3, or any combination thereof, e.g., Fragments 1-3 in order). In embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to a TTV-CT30F GC-rich sequence shown in Table 39 (e.g., the full sequence, Fragment 1, Fragment 2, Fragment 3, Fragment 4, Fragment 5, Fragment 6, Fragment 7, Fragment 8, or any combination thereof, e.g., Fragments 1-7 in order). In embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to a TTV-HD23a GC-rich sequence shown in Table 39 (e.g., the full sequence, Fragment 1, Fragment 2, Fragment 3, Fragment 4, Fragment 5, Fragment 6, or any combination thereof, e.g., Fragments 1-6 in order). In embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to a TTV-JA20 GC-rich sequence shown in Table 39 (e.g., the full sequence, Fragment 1, Fragment 2, or any combination thereof, e.g., Fragments 1 and 2 in order). In embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to a TTV-TJN02 GC-rich sequence shown in Table 39 (e.g., the full sequence, Fragment 1, Fragment 2, Fragment 3, Fragment 4, Fragment 5, Fragment 6, Fragment 7, Fragment 8, or any combination thereof, e.g., Fragments 1-8 in order). In embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to a TTV-tth8 GC-rich sequence shown in Table 39 (e.g., the full sequence, Fragment 1, Fragment 2, Fragment 3, Fragment 4, Fragment 5, Fragment 6, Fragment 7, Fragment 8, Fragment 9, or any combination thereof, e.g., Fragments 1-6 in order). In embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to Fragment 7 shown in Table 39. In embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to Fragment 8 shown in Table 39. In embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to Fragment 9 shown in Table 39.

TABLE 39
Exemplary GC-rich sequences from Anelloviruses

| | | | SEQ ID |
|---|---|---|---|
| Source | | Sequence | NO: |
| Consensus | | CGGCGGX₁GGX₂GX₃X₄X₅CGCGCTX₆CGCGC | 120 |
| | | GCX₇X₈X₉X₁₀CX₁₁X₁₂X₁₃X₁₄GGGGX₁₅X₁₆X₁₇X₁₈ | |
| | | X₁₉X₂₀X₂₁GCX₂₂X₂₃X₂₄X₂₅CCCCCCCX₂₆CGCGC | |
| | | ATX₂₇X₂₈GCX₂₉CGGGX₃₀CCCCCCCCCX₃₁X₃₂X₃₃ | |
| | | GGGGGGCTCCGX34CCCCCCGGCCCCCC | |
| | | X₁ = G or C | |
| | | X₂ = G, C, or absent | |
| | | X₃ = C or absent | |
| | | X₄ = G or C | |
| | | X₅ = G or C | |
| | | X₆ = T, G, or A | |
| | | X₇ = G or C | |
| | | X₈ = G or absent | |
| | | X₉ = C or absent | |
| | | X₁₀ = C or absent | |
| | | X₁₁ = G, A, or absent | |
| | | X₁₂ = G or C | |
| | | X₁₃ = C or T | |
| | | X₁₄ = G or A | |
| | | X₁₅ = G or A | |
| | | X₁₆ = A, G, T, or absent | |
| | | X₁₇ = G, C, or absent | |
| | | X₁₈ = G, C, or absent | |
| | | X₁₉ = C, A, or absent | |
| | | X₂₀ = C or A | |
| | | X₂₁ = T or A | |
| | | X₂₂ = G or C | |
| | | X₂₃ = G, T, or absent | |
| | | X₂₄ = C or absent | |
| | | X₂₅ = G, C, or absent | |
| | | X₂₆ = G or C | |
| | | X₂₇ = G or absent | |
| | | X₂₈ = C or absent | |
| | | X₂₉ = G or A | |
| | | X₃₀ = G or T | |
| | | X₃₁ = C, T, or absent | |
| | | X₃₂ = G, C, A, or absent | |
| | | X₃₃ = G or C | |
| | | X₃₄ = C or absent | |
| Exemplary TTV | Full sequence | GCCGCCGCGGCGGCGGSGGNGNSGCGCGCT | 121 |
| Sequence | | DCGCGCGCSNNNCRCCRGGGGGNNNNCWG | |
| | | CSNCNCCCCCCCCCGCGCATGCGCGGGKCC | |
| | | CCCCCCCNNCGGGGGGCTCCGCCCCCCGGC | |
| | | CCCCCCCCGTGCTAAACCCACCGCGCATGC | |
| | | GCGACCACGCCCCCGCCGCC | |
| | Fragment 1 | GCCGCCGCGGCGGCGGSGGNGNSGCGCGCT | 122 |
| | | DCGCGCGCSNNNCRCCRGGGGGNNNNCWG | |
| | | CSNCNCCCCCCCCCGCGCAT | |
| | Fragment 2 | GCGCGGGKCCCCCCCCCNNCGGGGGGCTC | 123 |
| | | CG | |
| | Fragment 3 | CCCCCCGGCCCCCCCCCGTGCTAAACCCAC | 124 |
| | | CGCGCATGCGCGACCACGCCCCCGCCGCC | |
| TTV-CT30F | Full sequence | GCGGCGG-GGGGGCG-GCCGCG- | 125 |
| | | TTCGCGCGCCGCCCACCAGGGGGTG-- | |
| | | CTGCG-CGCCCCCCCCCGCGCAT | |
| | | GCGCGGGGCCCCCCCCC -- | |
| | | GGGGGGGCTCCGCCCCCCCGGCCCCCCCCC | |
| | | GTGCTAAACCCACCGCGCATGCGCGACCAC | |
| | | GCCCCCGCCGCC | |
| | Fragment 1 | GCGGCGG | 126 |
| | Fragment 2 | GGGGGCG | 127 |
| | Fragment 3 | GCCGCG | 128 |
| | Fragment 4 | TTCGCGCGCCGCCCACCAGGGGGTG | 129 |
| | Fragment 5 | CTGCG | 130 |
| | Fragment 6 | CGCCCCCCCCCGCGCAT | 131 |
| | Fragment 7 | GCGCGGGGCCCCCCCCC | 132 |
| | Fragment 8 | GGGGGGGCTCCGCCCCCCCGGCCCCCCCCC | 133 |
| | | GTGCTAAACCCACCGCGCATGCGCGACCAC | |
| | | GCCCCCGCCGCC | |
| TTV-HD23a | Full sequence | CGGCGGCGGCGGCG- | 134 |
| | | CGCGCGCTGCGCGCGCG--- | |
| | | CGCCGGGGGGGCGCCAGCG- | |
| | | CCCCCCCCCCCGCGCAT | |
| | | GCACGGGTCCCCCCCCCCACGGGGGGCTCC | |
| | | G CCCCCCGGCCCCCCCCC | |
| | Fragment 1 | CGGCGGCGGCGGCG | 135 |
| | Fragment 2 | CGCGCGCTGCGCGCGCG | 136 |
| | Fragment 3 | CGCCGGGGGGGCGCCAGCG | 137 |
| | Fragment 4 | CCCCCCCCCCCGCGCAT | 138 |
| | Fragment 5 | GCACGGGTCCCCCCCCCCACGGGGGGCTCC | 139 |
| | | G | |
| | Fragment 6 | CCCCCCGGCCCCCCCCC | 140 |
| TTV-JA20 | Full sequence | CCGTCGGCGGGGGGGCCGCGCGCTGCGCG | 141 |
| | | CGCGGCCC- | |
| | | CCGGGGGAGGCACAGCCTCCCCCCCCCGCG | |
| | | CGCATGCGCGCGGGTCCCCCCCCCTCCGGG | |
| | | GGGCTCCGCCCCCCGGCCCCCCCC | |
| | Fragment 1 | CCGTCGGCGGGGGGGCCGCGCGCTGCGCG | 142 |
| | | CGCGGCCC | |
| | Fragment 2 | CCGGGGGAGGCACAGCCTCCCCCCCCCGCG | 143 |
| | | CGCATGCGCGCGGGTCCCCCCCCCTCCGGG | |
| | | GGGCTCCGCCCCCCGGCCCCCCCC | |
| TTV-TJN02 | Full sequence | CGGCGGCGGCG-CGCGCGCTACGCGCGCG-- | 144 |
| | | -CGCCGGGGGG----CTGCCGC- | |
| | | CCCCCCCCCGCGCAT | |
| | | GCGCGGGGCCCCCCCCC- | |
| | | GCGGGGGGCTCCG CCCCCCGGCCCCCC | |
| | Fragment 1 | CGGCGGCGGCG | 145 |
| | Fragment 2 | CGCGCGCTACGCGCGCG | 146 |
| | Fragment 3 | CGCCGGGGGG | 147 |
| | Fragment 4 | CTGCCGC | 148 |
| | Fragment 5 | CCCCCCCCCGCGCAT | 149 |
| | Fragment 6 | GCGCGGGGCCCCCCCCC | 150 |
| | Fragment 7 | GCGGGGGGCTCCG | 151 |
| | Fragment 8 | CCCCCCGGCCCCCC | 152 |
| TTV-tth8 | Full sequence | GCCGCCGCGGCGGCGGGGG- | 153 |
| | | GCGGCGCGCTGCGCGCGCCGCCCAGTAGG | |
| | | GGGAGCCATGCG---CCCCCCCCCGCGCAT | |
| | | GCGCGGGGCCCCCCCCC- | |
| | | GCGGGGGGCTCCG | |
| | | CCCCCCGGCCCCCCCCG | |
| | Fragment 1 | GCCGCCGCGGCGGCGGGGG | 154 |
| | Fragment 2 | GCGGCGCGCTGCGCGCGCCGCCCAGTAGG | 155 |
| | | GGGAGCCATGCG | |
| | Fragment 3 | CCCCCCCCCGCGCAT | 156 |
| | Fragment 4 | GCGCGGGGCCCCCCCCC | 157 |
| | Fragment 5 | GCGGGGGGCTCCG | 158 |
| | Fragment 6 | CCCCCCGGCCCCCCCCG | 159 |
| | Fragment 7 | CGCGCTGCGCGCGCCGCCCAGTAGGGGGA | 160 |
| | | GCCATGC | |
| | Fragment 8 | CCGCCATCTTAAGTAGTTGAGGCGGACGGT | 161 |
| | | GGCGTGAGTTCAAAGGTCACCATCAGCCAC | |
| | | ACCTACTCAAAATGGTGG | |
| | Fragment 9 | CTTAAGTAGTTGAGGCGGACGGTGGCGTGA | 162 |
| | | GTTCAAAGGTCACCATCAGCCACACCTACT | |
| | | CAAAATGGTGGACAATTTCTTCCGGGTCAA | |
| | | AGGTTACAGCCGCCATGTTAAAACACGTGA | |
| | | CGTATGACGTCACGGCCGCCATTTTGTGAC | |
| | | ACAAGATGGCCGACTTCCTTCC | |
| Additional | 36-nucleotide | CGCGCTGCGCGCGCCGCCCAGTAGGGGGA | 163 |
| GC-rich region | consensus GC- | GCCATGC | |
| | rich region | | |
| | sequence 1 | | |
| | 36-nucleotide | GCGCTX₁CGCGCGCGCGCCGGGGGGCTGCG | 164 |
| | region | CCCCCCC, wherein X₁ is selected | |
| | consensus | from T, G, or A | |
| | sequence 2 | | |
| | TTV Clade 1 | GCGCTTCGCGCGCCGCCCACTAGGGGGCGT | 165 |
| | 36-nucleotide | TGCGCG | |
| | region | | |
| | TTV Clade 3 | GCGCTGCGCGCGCCGCCCAGTAGGGGGCG | 166 |
| | 36-nucleotide | CAATGCG | |
| | region | | |
| | TTV Clade 3 | GCGCTGCGCGCGCGGCCCCCGGGGGAGGC | 167 |
| | isolate GH1 36- | ATTGCCT | |
| | nucleotide | | |
| | region | | |
| | TTV Clade 3 | GCGCTGCGCGCGCGCGCCGGGGGGGCGCC | 168 |
| | sle1932 36- | AGCGCCC | |
| | nucleotide | | |
| | region | | |
| | TTV Clade 4 | GCGCTTCGCGCGCGCGCCGGGGGGCTCCGC | 169 |
| | ctdc002 36- | CCCCCC | |
| | nucleotide | | |
| | region | | |
| | TTV Clade 5 | GCGCTTCGCGCGCGCGCCGGGGGGCTGCGC | 170 |
| | 36-nucleotide | CCCCCC | |
| | region | | |
| | TTV Clade 6 | GCGCTACGCGCGCGCGCCGGGGGGCTGCG | 171 |
| | 36-nucleotide | CCCCCCC | |
| | region | | |
| | TTV Clade 7 | GCGCTACGCGCGCGCGCCGGGGGGCTCTGC | 172 |
| | 36-nucleotide | CCCCCC | |
| | region | | |
| Additional | TTV-CT30F | GCGGCGGGGGGGCGGCCGCGTTCGCGCGC | 801 |
| Alphatorquevirus | | CGCCCACCAGGGGGTGCTGCGCGCCCCCCC | |
| GC-rich region | | CCGCGCATGCGCGGGGCCCCCCCCCGGGG | |
| sequences | | GGGCTCCGCCCCCCCGGCCCCCCCCCGTGC | |
| | | TAAACCCACCGCGCATGCGCGACCACGCCC | |
| | | CCGCCGCC | |
| | TTV-P13-1 | CCGAGCGTTAGCGAGGAGTGCGACCCTACC | 802 |
| | | CCCTGGGCCCACTTCTTCGGAGCCGCGCGC | |
| | | TACGCCTTCGGCTGCGCGCGGCACCTCAGA | |
| | | CCCCCGCTCGTGCTGACACGCTTGCGCGTG | |
| | | TCAGACCACTTCGGGCTCGCGGGGGTCGGG | |
| | TTV-tth8 | GCCGCCGCGGCGGCGGGGGGCGGCGCGCT | 803 |
| | | GCGCGCGCCGCCCAGTAGGGGGAGCCATG | |
| | | CGCCCCCCCCCGCGCATGCGCGGGGCCCCC | |
| | | CCCCGCGGGGGGCTCCGCCCCCCGGCCCCC | |
| | | CCCG | |
| | TTV-HD20a | CGGCCCAGCGGCGGCGCGCGCGCTTCGCGC | 804 |
| | | GCGCGCCGGGGGGCTCCGCCCCCCCCCGCG | |
| | | CATGCGCGGGGCCCCCCCCCGCGGGGGGCT | |
| | | CCGCCCCCCGGTCCCCCCCCG | |
| | TTV-16 | CGGCCGTGCGGCGGCGCGCGCGCTTCGCGC | 805 |
| | | GCGCGCCGGGGGCTGCCGCCCCCCCCCGCG | |
| | | CATGCGCGCGGGGCCCCCCCCCGCGGGGG | |
| | | GCTCCGCCCCCCGGCCCCCCCCCCCG | |
| | TTV-TJN02 | CGGCGGCGGCGCGCGCGCTACGCGCGCGC | 806 |
| | | GCCGGGGGGCTGCCGCCCCCCCCCCGCGCA | |
| | | TGCGCGGGGCCCCCCCCCGCGGGGGGCTCC | |
| | | GCCCCCCGGCCCCCC | |
| | TTV-HD16d | GGCGGCGGCGCGCGCGCTACGCGCGCGCG | 807 |
| | | CCGGGGAGCTCTGCCCCCCCCCGCGCATGC | |
| | | GCGCGGGTCCCCCCCCCGCGGGGGGCTCCG | |
| | | CCCCCCGGTCCCCCCCCCG | |

### Effectors

In some embodiments, the genetic element may include one or more sequences that encode an effector, e.g., a functional effector, e.g., an endogenous effector or an exogenous effector, e.g., a therapeutic polypeptide or nucleic acid, e.g., cytotoxic or cytolytic RNA or protein. In some embodiments, the functional nucleic acid is a non-coding RNA. In some embodiments, the functional nucleic acid is a coding RNA. The effector may modulate a biological activity, for example increasing or decreasing enzymatic activity, gene expression, cell signaling, and cellular or organ function. Effector activities may also include binding regulatory proteins to modulate activity of the regulator, such as transcription or translation. Effector activities also may include activator or inhibitor functions. For example, the effector may induce enzymatic activity by triggering increased substrate affinity in an enzyme, e.g., fructose 2,6-bisphosphate activates phosphofructokinase 1 and increases the rate of glycolysis in response to the insulin. In another example, the effector may inhibit substrate binding to a receptor and inhibit its activation, e.g., naltrexone and naloxone bind opioid receptors without activating them and block the receptors' ability to bind opioids. Effector activities may also include modulating protein stability/degradation and/or transcript stability/degradation. For example, proteins may be targeted for degradation by the polypeptide co-factor, ubiquitin, onto proteins to mark them for degradation. In another example, the effector inhibits enzymatic activity by blocking the enzyme's active site, e.g., methotrexate is a structural analog of tetrahydrofolate, a coenzyme for the enzyme dihydrofolate reductase that binds to dihydrofolate reductase 1000-fold more tightly than the natural substrate and inhibits nucleotide base synthesis.

In some embodiments, the sequence encoding an effector is part of the genetic element, e.g., it can be inserted at an insert site as described herein. In embodiments, the sequence encoding an effector is inserted into the genetic element at a noncoding region, e.g., a noncoding region disposed 3′ of the open reading frames and 5′ of the GC-rich region of the genetic element, in the 5′ noncoding region upstream of the TATA box, in the 5′ UTR, in the 3′ noncoding region downstream of the poly-A signal, or upstream of the GC-rich region. In embodiments, the sequence encoding an effector is inserted into the genetic element at about nucleotide 3588 of a TTV-tth8 plasmid, e.g., as described herein or at about nucleotide 2843 of a TTMV-LY2 plasmid, e.g., as described herein. In embodiments, the sequence encoding an effector is inserted into the genetic element at or within nucleotides 336-3015 of a TTV-tth8 plasmid, e.g., as described herein, or at or within nucleotides 242-2812 of a TTV-LY2 plasmid, e.g., as described herein. In some embodiments, the sequence encoding an effector replaces part or all of an open reading frame (e.g., an ORF as described herein, e.g., an ORF1, ORF1/1, ORF1/2, ORF2, ORF2/2, ORF2/3, and/or ORF2t/3).

In some embodiments, the sequence encoding an effector comprises 100-2000, 100-1000, 100-500, 100-200, 200-2000, 200-1000, 200-500, 500-1000, 500-2000, or 1000-2000 nucleotides. In some embodiments, the effector is a nucleic acid or protein payload, e.g., as described herein.

Regulatory Nucleic Acids

In some embodiments, the effector is a regulatory nucleic acid. Regulatory nucleic acids modify expression of an endogenous gene and/or an exogenous gene. In one embodiment, the regulatory nucleic acid targets a host gene. The regulatory nucleic acids may include, but are not limited to, a nucleic acid that hybridizes to an endogenous gene (e.g., miRNA, siRNA, mRNA, lncRNA, RNA, DNA, an antisense RNA, gRNA as described herein elsewhere), nucleic acid that hybridizes to an exogenous nucleic acid such as a viral DNA or RNA, nucleic acid that hybridizes to an RNA, nucleic acid that interferes with gene transcription, nucleic acid that interferes with RNA translation, nucleic acid that stabilizes RNA or destabilizes RNA such as through targeting for degradation, and nucleic acid that modulates a DNA or RNA binding factor. In embodiments, the regulatory nucleic acid encodes an miRNA. In some embodiments, the regulatory nucleic acid is endogenous to a wild-type Anellovirus. In some embodiments, the regulatory nucleic acid is exogenous to a wild-type Anellovirus.

In some embodiments, the regulatory nucleic acid comprises RNA or RNA-like structures typically containing 5-500 base pairs (depending on the specific RNA structure, e.g., miRNA 5-30 bps, lncRNA 200-500 bps) and may have a nucleobase sequence identical (or complementary) or nearly identical (or substantially complementary) to a coding sequence in an expressed target gene within the cell, or a sequence encoding an expressed target gene within the cell.

In some embodiments, the regulatory nucleic acid comprises a nucleic acid sequence, e.g., a guide RNA (gRNA). In some embodiments, the DNA targeting moiety comprises a guide RNA or nucleic acid encoding the guide RNA. A gRNA short synthetic RNA can be composed of a “scaffold” sequence necessary for binding to the incomplete effector moiety and a user-defined ˜20 nucleotide targeting sequence for a genomic target. In practice, guide RNA sequences are generally designed to have a length of between 17-24 nucleotides (e.g., 19, 20, or 21 nucleotides) and complementary to the targeted nucleic acid sequence. Custom gRNA generators and algorithms are available commercially for use in the design of effective guide RNAs. Gene editing has also been achieved using a chimeric “single guide RNA” (“sgRNA”), an engineered (synthetic) single RNA molecule that mimics a naturally occurring crRNA-tracrRNA complex and contains both a tracrRNA (for binding the nuclease) and at least one crRNA (to guide the nuclease to the sequence targeted for editing). Chemically modified sgRNAs have also been demonstrated to be effective in genome editing; see, for example, Hendel et al. (2015) Nature Biotechnol., 985-991.

The regulatory nucleic acid comprises a gRNA that recognizes specific DNA sequences (e.g., sequences adjacent to or within a promoter, enhancer, silencer, or repressor of a gene).

Certain regulatory nucleic acids can inhibit gene expression through the biological process of RNA interference (RNAi). RNAi molecules comprise RNA or RNA-like structures typically containing 15-50 base pairs (such as about 18-25 base pairs) and having a nucleobase sequence identical (complementary) or nearly identical (substantially complementary) to a coding sequence in an expressed target gene within the cell. RNAi molecules include, but are not limited to: short interfering RNAs (siRNAs), double-strand RNAs (dsRNA), micro RNAs (miRNAs), short hairpin RNAs (shRNA), meroduplexes, and dicer substrates (U.S. Pat. Nos. 8,084,599 8,349,809 and 8,513,207).

Long non-coding RNAs (lncRNA) are defined as non-protein coding transcripts longer than 100 nucleotides. This somewhat arbitrary limit distinguishes lncRNAs from small regulatory RNAs such as microRNAs (miRNAs), short interfering RNAs (siRNAs), and other short RNAs. In general, the majority (˜78%) of lncRNAs are characterized as tissue-specific. Divergent lncRNAs that are transcribed in the opposite direction to nearby protein-coding genes (comprise a significant proportion ˜20% of total lncRNAs in mammalian genomes) may possibly regulate the transcription of the nearby gene.

The genetic element may encode regulatory nucleic acids with a sequence substantially complementary, or fully complementary, to all or a fragment of an endogenous gene or gene product (e.g., mRNA). The regulatory nucleic acids may complement sequences at the boundary between introns and exons to prevent the maturation of newly-generated nuclear RNA transcripts of specific genes into mRNA for transcription. The regulatory nucleic acids that are complementary to specific genes can hybridize with the mRNA for that gene and prevent its translation. The antisense regulatory nucleic acid can be DNA, RNA, or a derivative or hybrid thereof.

The length of the regulatory nucleic acid that hybridizes to the transcript of interest may be between 5 to 30 nucleotides, between about 10 to 30 nucleotides, or about 11, 12, 13, 14, 15, 16, 17, 18, 19, 20, 21, 22, 23, 24, 25, 26, 27, 28, 29, 30 or more nucleotides. The degree of identity of the regulatory nucleic acid to the targeted transcript should be at least 75%, at least 80%, at least 85%, at least 90%, or at least 95%.

The genetic element may encode a regulatory nucleic acid, e.g., a micro RNA (miRNA) molecule identical to about 5 to about 25 contiguous nucleotides of a target gene. In some embodiments, the miRNA sequence targets a mRNA and commences with the dinucleotide AA, comprises a GC-content of about 30-70% (about 30-60%, about 40-60%, or about 45%-55%), and does not have a high percentage identity to any nucleotide sequence other than the target in the genome of the mammal in which it is to be introduced, for example as determined by standard BLAST search.

siRNAs and shRNAs resemble intermediates in the processing pathway of the endogenous microRNA (miRNA) genes (Bartel, Cell 116:281-297, 2004). In some embodiments, siRNAs can function as miRNAs and vice versa (Zeng et al., Mol Cell 9:1327-1333, 2002; Doench et al., Genes Dev 17:438-442, 2003). MicroRNAs, like siRNAs, use RISC to downregulate target genes, but unlike siRNAs, most animal miRNAs do not cleave the mRNA. Instead, miRNAs reduce protein output through translational suppression or polyA removal and mRNA degradation (Wu et al., Proc Natl Acad Sci USA 103:4034-4039, 2006). Known miRNA binding sites are within mRNA 3′ UTRs; miRNAs seem to target sites with near-perfect complementarity to nucleotides 2-8 from the miRNA's 5′ end (Rajewsky, Nat Genet 38 Suppl:S8-13, 2006; Lim et al., Nature 433:769-773, 2005). This region is known as the seed region. Because siRNAs and miRNAs are interchangeable, exogenous siRNAs downregulate mRNAs with seed complementarity to the siRNA (Birmingham et al., Nat Methods 3:199-204, 2006. Multiple target sites within a 3′ UTR give stronger downregulation (Doench et al., Genes Dev 17:438-442, 2003).

Lists of known miRNA sequences can be found in databases maintained by research organizations, such as Wellcome Trust Sanger Institute, Penn Center for Bioinformatics, Memorial Sloan Kettering Cancer Center, and European Molecule Biology Laboratory, among others. Known effective siRNA sequences and cognate binding sites are also well represented in the relevant literature. RNAi molecules are readily designed and produced by technologies known in the art. In addition, there are computational tools that increase the chance of finding effective and specific sequence motifs (Lagana et al., Methods Mol. Bio., 2015, 1269:393-412).

The regulatory nucleic acid may modulate expression of RNA encoded by a gene. Because multiple genes can share some degree of sequence homology with each other, in some embodiments, the regulatory nucleic acid can be designed to target a class of genes with sufficient sequence homology. In some embodiments, the regulatory nucleic acid can contain a sequence that has complementarity to sequences that are shared amongst different gene targets or are unique for a specific gene target. In some embodiments, the regulatory nucleic acid can be designed to target conserved regions of an RNA sequence having homology between several genes thereby targeting several genes in a gene family (e.g., different gene isoforms, splice variants, mutant genes, etc.). In some embodiments, the regulatory nucleic acid can be designed to target a sequence that is unique to a specific RNA sequence of a single gene.

In some embodiments, the genetic element may include one or more sequences that encode regulatory nucleic acids that modulate expression of one or more genes.

In one embodiment, the gRNA described elsewhere herein are used as part of a CRISPR system for gene editing. For the purposes of gene editing, the anellovector may be designed to include one or multiple guide RNA sequences corresponding to a desired target DNA sequence; see, for example, Cong et al. (2013) Science, 339:819-823; Ran et al. (2013) Nature Protocols, 8:2281-2308. At least about 16 or 17 nucleotides of gRNA sequence generally allow for Cas9-mediated DNA cleavage to occur; for Cpf1 at least about 16 nucleotides of gRNA sequence is needed to achieve detectable DNA cleavage.

### Therapeutic Effectors (e.g., Peptides or Polypeptides)

In some embodiments, the genetic element comprises a therapeutic expression sequence, e.g., a sequence that encodes a therapeutic peptide or polypeptide, e.g., an intracellular peptide or intracellular polypeptide, a secreted polypeptide, or a protein replacement therapeutic. In some embodiments, the genetic element includes a sequence encoding a protein e.g., a therapeutic protein. Some examples of therapeutic proteins may include, but are not limited to, a hormone, a cytokine, an enzyme, an antibody (e.g., one or a plurality of polypeptides encoding at least a heavy chain or a light chain), a transcription factor, a receptor (e.g., a membrane receptor), a ligand, a membrane transporter, a secreted protein, a peptide, a carrier protein, a structural protein, a nuclease, or a component thereof.

In some embodiments, the genetic element includes a sequence encoding a peptide e.g., a therapeutic peptide. The peptides may be linear or branched. The peptide has a length from about 5 to about 500 amino acids, about 15 to about 400 amino acids, about 20 to about 325 amino acids, about 25 to about 250 amino acids, about 50 to about 200 amino acids, or any range there between.

In some embodiments, the polypeptide encoded by the therapeutic expression sequence may be a functional variant or fragment thereof of any of the above, e.g., a protein having at least 80%, 85%, 90%, 95%, 967%, 98%, 99% identity to a protein sequence which disclosed in a table herein by reference to its UniProt ID.

In some embodiments, the therapeutic expression sequence may encode an antibody or antibody fragment that binds any of the above, e.g., an antibody against a protein having at least 80%, 85%, 90%, 95%, 967%, 98%, 99% identity to a protein sequence which disclosed in a table herein by reference to its UniProt ID. The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity. An “antibody fragment” refers to a molecule that includes at least one heavy chain or light chain and binds an antigen. Examples of antibody fragments include but are not limited to Fv, Fab, Fab′, Fab′-SH, F(ab′)₂; diabodies; linear antibodies; single-chain antibody molecules (e.g. scFv); and multispecific antibodies formed from antibody fragments.

Exemplary Intracellular Polypeptide Effectors

In some embodiments, the effector comprises a cytosolic polypeptide or cytosolic peptide. In some embodiments, the effector comprises cytosolic peptide is a DPP-4 inhibitor, an activator of GLP-1 signaling, or an inhibitor of neutrophil elastase. In some embodiments, the effector increases the level or activity of a growth factor or receptor thereof (e.g., an FGF receptor, e.g., FGFR3). In some embodiments, the effector comprises an inhibitor of n-myc interacting protein activity (e.g., an n-myc interacting protein inhibitor); an inhibitor of EGFR activity (e.g., an EGFR inhibitor); an inhibitor of IDH1 and/or IDH2 activity (e.g., an IDH1 inhibitor and/or an IDH2 inhibitor); an inhibitor of LRP5 and/or DKK2 activity (e.g., an LRP5 and/or DKK2 inhibitor); an inhibitor of KRAS activity; an activator of HTT activity; or inhibitor of DPP-4 activity (e.g., a DPP-4 inhibitor).

In some embodiments, the effector comprises a regulatory intracellular polypeptide. In some embodiments, the regulatory intracellular polypeptide binds one or more molecule (e.g., protein or nucleic acid) endogenous to the target cell. In some embodiments, the regulatory intracellular polypeptide increases the level or activity of one or more molecule (e.g., protein or nucleic acid) endogenous to the target cell. In some embodiments, the regulatory intracellular polypeptide decreases the level or activity of one or more molecule (e.g., protein or nucleic acid) endogenous to the target cell.

Exemplary Secreted Polypeptide Effectors

Exemplary secreted therapeutics are described herein, e.g., in the tables below.

TABLE 50
Exemplary cytokines and cytokine receptors

| Cytokine | Cytokine receptor(s) | Entrez Gene ID | UniProt ID |
|---|---|---|---|
| IL-1α, IL-1β, or a | IL-1 type 1 receptor, IL-1 type | 3552, 3553 | P01583, P01584 |
| heterodimer thereof | 2 receptor | | |
| IL-1Ra | IL-1 type 1 receptor, IL-1 type | 3454, 3455 | P17181, P48551 |
| | 2 receptor | | |
| IL-2 | IL-2R | 3558 | P60568 |
| IL-3 | IL-3 receptor α + β c (CD131) | 3562 | P08700 |
| IL-4 | IL-4R type I, IL-4R type II | 3565 | P05112 |
| IL-5 | IL-5R | 3567 | P05113 |
| IL-6 | IL-6R (sIL-6R) gp130 | 3569 | P05231 |
| IL-7 | IL-7R and SIL-7R | 3574 | P13232 |
| IL-8 | CXCR1 and CXCR2 | 3576 | P10145 |
| IL-9 | IL-9R | 3578 | P15248 |
| IL-10 | IL-10R1/IL-10R2 complex | 3586 | P22301 |
| IL-11 | IL-11Rα 1 gp130 | 3589 | P20809 |
| IL-12 (e.g., p35, p40, or a | IL-12Rβ1 and IL-12Rβ2 | 3593, 3592 | P29459, P29460 |
| heterodimer thereof) | | | |
| IL-13 | IL-13R1α1 and IL-13R1α2 | 3596 | P35225 |
| IL-14 | IL-14R | 30685 | P40222 |
| IL-15 | IL-15R | 3600 | P40933 |
| IL-16 | CD4 | 3603 | Q14005 |
| IL-17A | IL-17RA | 3605 | Q16552 |
| IL-17B | IL-17RB | 27190 | Q9UHF5 |
| IL-17C | IL-17RA to IL-17RE | 27189 | Q9P0M4 |
| e | SEF | 53342 | Q8TAD2 |
| IL-17F | IL-17RA, IL-17RC | 112744 | Q96PD4 |
| IL-18 | IL-18 receptor | 3606 | Q14116 |
| IL-19 | IL-20R1/IL-20R2 | 29949 | Q9UHD0 |
| IL-20 | L-20R1/IL-20R2 and IL-22R1/ | 50604 | Q9NYY1 |
| | IL-20R2 | | |
| IL-21 | IL-21R | 59067 | Q9HBE4 |
| IL-22 | IL-22R | 50616 | Q9GZX6 |
| IL-23 (e.g., p19, p40, or a | IL-23R | 51561 | Q9NPF7 |
| heterodimer thereof) | | | |
| IL-24 | IL-20R1/IL-20R2 and IL- | 11009 | Q13007 |
| | 22R1/IL-20R2 | | |
| IL-25 | IL-17RA and IL-17RB | 64806 | Q9H293 |
| IL-26 | IL-10R2 chain and IL-20R1 | 55801 | Q9NPH9 |
| | chain | | |
| IL-27 (e.g., p28, EBI3, or | WSX-1 and gp130 | 246778 | Q8NEV9 |
| a heterodimer thereof) | | | |
| IL-28A, IL-28B, and IL29 | IL-28R1/IL-10R2 | 282617, 282618 | Q8IZI9, Q8IU54 |
| IL-30 | IL6R/gp130 | 246778 | Q8NEV9 |
| IL-31 | IL-31RA/OSMRβ | 386653 | Q6EBC2 |
| IL-32 | | 9235 | P24001 |
| IL-33 | ST2 | 90865 | O95760 |
| IL-34 | Colony-stimulating factor 1 | 146433 | Q6ZMJ4 |
| | receptor | | |
| IL-35 (e.g., p35, EBI3, or | IL-12Rβ2/gp130; IL- | 10148 | Q14213 |
| a heterodimer thereof) | 12Rβ2/IL-12Rβ2; | | |
| | gp130/gp130 | | |
| IL-36 | IL-36Ra | 27179 | Q9UHA7 |
| IL-37 | IL-18Rα and IL-18BP | 27178 | Q9NZH6 |
| IL-38 | IL-1R1, IL-36R | 84639 | Q8WWZ1 |
| IFN-α | IFNAR | 3454 | P17181 |
| IFN-β | IFNAR | 3454 | P17181 |
| IFN-γ | IFNGR1/IFNGR2 | 3459 | P15260 |
| TGF-β | TβR-I and TβR-II | 7046, 7048 | P36897, P37173 |
| TNF-α | TNFR1, TNFR2 | 7132, 7133 | P19438, P20333 |

In some embodiments, an effector described herein comprises a cytokine of Table 50, or a functional variant thereof, e.g., a homolog (e.g., ortholog or paralog) or fragment thereof. In some embodiments, an effector described herein comprises a protein having at least 80%, 85%, 90%, 95%, 967%, 98%, 99% sequence identity to an amino acid sequence listed in Table 50 by reference to its UniProt ID. In some embodiments, the functional variant binds to the corresponding cytokine receptor with a Kd of no more than 10%, 20%, 30%, 40%, or 50% higher or lower than the Kd of the corresponding wild-type cytokine for the same receptor under the same conditions. In some embodiments, the effector comprises a fusion protein comprising a first region (e.g., a cytokine polypeptide of Table 50 or a functional variant or fragment thereof) and a second, heterologous region. In some embodiments, the first region is a first cytokine polypeptide of Table 50. In some embodiments, the second region is a second cytokine polypeptide of Table 50, wherein the first and second cytokine polypeptides form a cytokine heterodimer with each other in a wild-type cell. In some embodiments, the polypeptide of Table 50 or functional variant thereof comprises a signal sequence, e.g., a signal sequence that is endogenous to the effector, or a heterologous signal sequence. In some embodiments, an anellovector encoding a cytokine of Table 50, or a functional variant thereof, is used for the treatment of a disease or disorder described herein.

In some embodiments, an effector described herein comprises an antibody molecule (e.g., an scFv) that binds a cytokine of Table 50. In some embodiments, an effector described herein comprises an antibody molecule (e.g., an scFv) that binds a cytokine receptor of Table 50. In some embodiments, the antibody molecule comprises a signal sequence.

Exemplary cytokines and cytokine receptors are described, e.g., in Akdis et al., “Interleukins (from IL-1 to IL-38), interferons, transforming growth factor β, and TNF-α: Receptors, functions, and roles in diseases” October 2016 Volume 138, Issue 4, Pages 984-1010, which is herein incorporated by reference in its entirety, including Table I therein.

TABLE 51
Exemplary polypeptide hormones and receptors

| Hormone | Receptor | Entrez Gene ID | UniProt ID |
|---|---|---|---|
| Natriuretic Peptide, e.g., Atrial | NPRA, NPRB, NPRC | 4878 | P01160 |
| Natriuretic Peptide (ANP) | | | |
| Brain Natriuretic Peptide (BNP) | NPRA, NPRB | 4879 | P16860 |
| C-type natriuretic peptide | NPRB | 4880 | P23582 |
| (CNP) | | | |
| Growth hormone (GH) | GHR | 2690 | P10912 |
| Human growth hormone (hGH) | hGH receptor (human | 2690 | P10912 |
| | GHR) | | |
| Prolactin (PRL) | PRLR | 5617 | P01236 |
| Thyroid-stimulating hormone | TSH receptor | 7253 | P16473 |
| (TSH) | | | |
| Adrenocorticotropic hormone | ACTH receptor | 5443 | P01189 |
| (ACTH) | | | |
| Follicle-stimulating hormone | FSHR | 2492 | P23945 |
| (FSH) | | | |
| Luteinizing hormone (LH) | LHR | 3973 | P22888 |
| Antidiuretic hormone (ADH) | Vasopressin receptors, e.g., | 554 | P30518 |
| | V2; AVPR1A; AVPR1B; | | |
| | AVPR3; AVPR2 | | |
| Oxytocin | OXTR | 5020 | P01178 |
| Calcitonin | Calcitonin receptor (CT) | 796 | P01258 |
| Parathyroid hormone (PTH) | PTH1R and PTH2R | 5741 | P01270 |
| Insulin | Insulin receptor (IR) | 3630 | P01308 |
| Glucagon | Glucagon receptor | 2641 | P01275 |

In some embodiments, an effector described herein comprises a hormone of Table 51, or a functional variant thereof, e.g., a homolog (e.g., ortholog or paralog) or fragment thereof. In some embodiments, an effector described herein comprises a protein having at least 80%, 85%, 90%, 95%, 967%, 98%, 99% sequence identity to an amino acid sequence listed in Table 51 by reference to its UniProt ID. In some embodiments, the functional variant binds to the corresponding receptor with a Kd of no more than 10%, 20%, 30%, 40%, or 50% higher than the Kd of the corresponding wild-type hormone for the same receptor under the same conditions. In some embodiments, the polypeptide of Table 51 or functional variant thereof comprises a signal sequence, e.g., a signal sequence that is endogenous to the effector, or a heterologous signal sequence. In some embodiments, an anellovector encoding a hormone of Table 51, or a functional variant thereof, is used for the treatment of a disease or disorder described herein.

In some embodiments, an effector described herein comprises an antibody molecule (e.g., an scFv) that binds a hormone of Table 51. In some embodiments, an effector described herein comprises an antibody molecule (e.g., an scFv) that binds a hormone receptor of Table 51. In some embodiments, the antibody molecule comprises a signal sequence.

TABLE 52
Exemplary growth factors

| | | |
|---|---|---|
| Growth Factor | Entrez Gene ID | UniProt ID |

PDGF family

| | | | |
|---|---|---|---|
| PDGF (e.g., PDGF-1, | PDGF receptor, e.g., | 5156 | P16234 |
| PDGF-2, or a | PDGFRα, PDGFRβ | | |
| heterodimer thereof) | | | |
| CSF-1 | CSF1R | 1435 | P09603 |
| SCF | CD117 | 3815 | P10721 |

VEGF family

| | | | |
|---|---|---|---|
| VEGF (e.g., isoforms | VEGFR-1, VEGFR- | 2321 | P17948 |
| VEGF 121, VEGF 165, | 2 | | |
| VEGF 189, and VEGF | | | |
| 206) | | | |
| VEGF-B | VEGFR-1 | 2321 | P17949 |
| VEGF-C | VEGFR-2 and | 2324 | P35916 |
| | VEGFR -3 | | |
| PIGF | VEGFR-1 | 5281 | Q07326 |

EGF family

| | | | |
|---|---|---|---|
| EGF | EGFR | 1950 | P01133 |
| TGF-α | EGFR | 7039 | P01135 |
| amphiregulin | EGFR | 374 | P15514 |
| HB-EGF | EGFR | 1839 | Q99075 |
| betacellulin | EGFR, ErbB-4 | 685 | P35070 |
| epiregulin | EGFR, ErbB-4 | 2069 | O14944 |
| Heregulin | EGFR, ErbB-4 | 3084 | Q02297 |

FGF family

| | | | |
|---|---|---|---|
| FGF-1, FGF-2, FGF-3, | FGFR1, FGFR2, | 2246, 2247, 2248, 2249, | P05230, P09038, |
| FGF-4, FGF-5, FGF-6, | FGFR3, and FGFR4 | 2250, 2251, 2252, 2253, | P11487, P08620, |
| FGF-7, FGF-8, FGF-9 | | 2254 | P12034, P10767, |
| | | | P21781, P55075, P31371 |

Insulin family

| | | | |
|---|---|---|---|
| Insulin | IR | 3630 | P01308 |
| IGF-I | IGF-I receptor, IGF- | 3479 | P05019 |
| | II receptor | | |
| IGF-II | IGF-II receptor | 3481 | P01344 |

HGF family

| | | | |
|---|---|---|---|
| HGF | MET receptor | 3082 | P14210 |
| MSP | RON | 4485 | P26927 |

Neurotrophin family

| | | | |
|---|---|---|---|
| NGF | LNGFR, trkA | 4803 | P01138 |
| BDNF | trkB | 627 | P23560 |
| NT-3 | trkA, trkB, trkC | 4908 | P20783 |
| NT-4 | trkA, trkB | 4909 | P34130 |
| NT-5 | trkA, trkB | 4909 | P34130 |

Angiopoietin family

| | | | |
|---|---|---|---|
| ANGPT1 | HPK-6/TEK | 284 | Q15389 |
| ANGPT2 | HPK-6/TEK | 285 | O15123 |
| ANGPT3 | HPK-6/TEK | 9068 | O95841 |
| ANGPT4 | HPK-6/TEK | 51378 | Q9Y264 |

In some embodiments, an effector described herein comprises a growth factor of Table 52, or a functional variant thereof, e.g., a homolog (e.g., ortholog or paralog) or fragment thereof. In some embodiments, an effector described herein comprises a protein having at least 80%, 85%, 90%, 95%, 967%, 98%, 99% sequence identity to an amino acid sequence listed in Table 52 by reference to its UniProt ID. In some embodiments, the functional variant binds to the corresponding receptor with a Kd of no more than 10%, 20%, 30%, 40%, or 50% higher than the Kd of the corresponding wild-type growth factor for the same receptor under the same conditions. In some embodiments, the polypeptide of Table 52 or functional variant thereof comprises a signal sequence, e.g., a signal sequence that is endogenous to the effector, or a heterologous signal sequence. In some embodiments, an anellovector encoding a growth factor of Table 52, or a functional variant thereof, is used for the treatment of a disease or disorder described herein.

In some embodiments, an effector described herein comprises an antibody molecule (e.g., an scFv) that binds a growth factor of Table 52. In some embodiments, an effector described herein comprises an antibody molecule (e.g., an scFv) that binds a growth factor receptor of Table 52. In some embodiments, the antibody molecule comprises a signal sequence.

Exemplary growth factors and growth factor receptors are described, e.g., in Bafico et al., “Classification of Growth Factors and Their Receptors” Holland-Frei Cancer Medicine. 6th edition, which is herein incorporated by reference in its entirety.

TABLE 53
Clotting-associated factors

| Effector | Indication | Entrez Gene ID | UniProt ID |
|---|---|---|---|
| Factor I | Afibrinogenomia | 2243, 2266, 2244 | P02671, P02679, P02675 |
| (fibrinogen) | | | |
| Factor II | Factor II Deficiency | 2147 | P00734 |
| Factor IX | Hemophilia B | 2158 | P00740 |
| Factor V | Owren's disease | 2153 | P12259 |
| Factor VIII | Hemophilia A | 2157 | P00451 |
| Factor X | Stuart-Prower Factor | 2159 | P00742 |
| | Deficiency | | |
| Factor XI | Hemophilia C | 2160 | P03951 |
| Factor XIII | Fibrin Stabilizing factor | 2162, 2165 | P00488, P05160 |
| | deficiency | | |
| vWF | von Willebrand disease | 7450 | P04275 |

In some embodiments, an effector described herein comprises a polypeptide of Table 53, or a functional variant thereof, e.g., a homolog (e.g., ortholog or paralog) or fragment thereof. In some embodiments, an effector described herein comprises a protein having at least 80%, 85%, 90%, 95%, 967%, 98%, 99% sequence identity to an amino acid sequence listed in Table 53 by reference to its UniProt ID. In some embodiments, the functional variant catalyzes the same reaction as the corresponding wild-type protein, e.g., at a rate no less than 10%, 20%, 30%, 40%, or 50% lower than the wild-type protein. In some embodiments, the polypeptide of Table 53 or functional variant thereof comprises a signal sequence, e.g., a signal sequence that is endogenous to the effector, or a heterologous signal sequence. In some embodiments, an anellovector encoding a polypeptide of Table 53, or a functional variant thereof is used for the treatment of a disease or disorder of Table 53.

Exemplary Protein Replacement Therapeutics

Exemplary protein replacement therapeutics are described herein, e.g., in the tables below.

TABLE 54
Exemplary enzymatic effectors and corresponding indications

| Effector | deficiency | Entrez Gene ID | UniProt ID |
|---|---|---|---|
| 3-methylcrotonyl-CoA | 3-methylcrotonyl-CoA | 56922, 64087 | Q96RQ3, Q9HCC0 |
| carboxylase | carboxylase deficiency | | |
| Acetyl-CoA- | Mucopolysaccharidosis MPS | 138050 | Q68CP4 |
| glucosaminide N- | III (Sanfilippo's syndrome) | | |
| acetyltransferase | Type III-C | | |
| ADAMTS13 | Thrombotic | 11093 | Q76LX8 |
| | Thrombocytopenia Purpura | | |
| adenine | Adenine | 353 | P07741 |
| phosphoribosyltransferase | phosphoribosyltransferase | | |
| | deficiency | | |
| Adenosine deaminase | Adenosine deaminase | 100 | P00813 |
| | deficiency | | |
| ADP-ribose protein | Glutamyl ribose-5-phosphate | 26119, 54936 | Q5SW96, Q9NX46 |
| hydrolase | storage disease | | |
| alpha glucosidase | Glycogen storage disease | 2548 | P10253 |
| | type 2 (Pompe's disease) | | |
| Arginase | Familial hyperarginemia | 383, 384 | P05089, P78540 |
| Arylsulfatase A | Metachromatic | 410 | P15289 |
| | leukodystrophy | | |
| Cathepsin K | Pycnodysostosis | 1513 | P43235 |
| Ceramidase | Farber's disease | 125981, 340485, | Q8TDN7, |
| | (lipogranulomatosis) | 55331 | Q5QJU3, QONUN7 |
| Cystathionine B | Homocystinuria | 875 | P35520 |
| synthase | | | |
| Dolichol-P-mannose | Congenital disorders of N- | 8813, 54344 | O60762, Q9P2X0 |
| synthase | glycosylation CDG Ie | | |
| Dolicho-P- | Congenital disorders of N- | 84920 | Q5BKT4 |
| Glc: Man9GlcNAc2-PP- | glycosylation CDG Ic | | |
| dolichol | | | |
| glucosyltransferase | | | |
| Dolicho-P- | Congenital disorders of N- | 10195 | Q92685 |
| Man: Man5GlcNAc2- | glycosylation CDG Id | | |
| PP-dolichol | | | |
| mannosyltransferase | | | |
| Dolichyl-P-glucose: Glc- | Congenital disorders of N- | 79053 | Q9BVK2 |
| 1-Man-9-GlcNAc-2-PP- | glycosylation CDG Ih | | |
| dolichyl-α-3- | | | |
| glucosyltransferase | | | |
| Dolichy1-P- | Congenital disorders of N- | 79087 | Q9BV10 |
| mannose: Man-7- | glycosylation CDG Ig | | |
| GlcNAc-2-PP-dolichyl- | | | |
| α-6-mannosyltransferase | | | |
| Factor II | Factor II Deficiency | 2147 | P00734 |
| Factor IX | Hemophilia B | 2158 | P00740 |
| Factor V | Owren's disease | 2153 | P12259 |
| Factor VIII | Hemophilia A | 2157 | P00451 |
| Factor X | Stuart-Prower Factor | 2159 | P00742 |
| | Deficiency | | |
| Factor XI | Hemophilia C | 2160 | P03951 |
| Factor XIII | Fibrin Stabilizing factor | 2162, 2165 | P00488, P05160 |
| | deficiency | | |
| Galactosamine-6-sulfate | Mucopolysaccharidosis MPS | 2588 | P34059 |
| sulfatase | IV (Morquio's syndrome) | | |
| | Type IV-A | | |
| Galactosylceramide β- | Krabbe's disease | 2581 | P54803 |
| galactosidase | | | |
| Ganglioside β- | GM1 gangliosidosis, | 2720 | P16278 |
| galactosidase | generalized | | |
| Ganglioside β- | GM2 gangliosidosis | 2720 | P16278 |
| galactosidase | | | |
| Ganglioside β- | Sphingolipidosis Type I | 2720 | P16278 |
| galactosidase | | | |
| Ganglioside β- | Sphingolipidosis Type II | 2720 | P16278 |
| galactosidase | (juvenile type) | | |
| Ganglioside β- | Sphingolipidosis Type III | 2720 | P16278 |
| galactosidase | (adult type) | | |
| Glucosidase I | Congenital disorders of N- | 2548 | P10253 |
| | glycosylation CDG IIb | | |
| Glucosylceramide β- | Gaucher's disease | 2629 | P04062 |
| glucosidase | | | |
| Heparan-S-sulfate | Mucopolysaccharidosis MPS | 6448 | P51688 |
| sulfamidase | III (Sanfilippo's syndrome) | | |
| | Type III-A | | |
| homogentisate oxidase | Alkaptonuria | 3081 | Q93099 |
| Hyaluronidase | Mucopolysaccharidosis MPS | 3373, 8692, 8372, | Q12794, Q12891, |
| | IX (hyaluronidase deficiency) | 23553 | O43820, Q2M3T9 |
| Iduronate sulfate | Mucopolysaccharidosis MPS | 3423 | P22304 |
| sulfatase | II (Hunter's syndrome) | | |
| Lecithin-cholesterol | Complete LCAT deficiency, | 3931 | 606967 |
| acyltransferase (LCAT) | Fish-eye disease, | | |
| | atherosclerosis, | | |
| | hypercholesterolemia | | |
| Lysine oxidase | Glutaric acidemia type I | 4015 | P28300 |
| Lysosomal acid lipase | Cholesteryl ester storage | 3988 | P38571 |
| | disease (CESD) | | |
| Lysosomal acid lipase | Lysosomal acid lipase | 3988 | P38571 |
| | deficiency | | |
| lysosomal acid lipase | Wolman's disease | 3988 | P38571 |
| Lysosomal pepstatin- | Ceroid lipofuscinosis Late | 1200 | O14773 |
| insensitive peptidase | infantile form (CLN2, | | |
| | Jansky-Bielschowsky | | |
| | disease) | | |
| Mannose (Man) | Congenital disorders of N- | 4351 | P34949 |
| phosphate (P) isomerase | glycosylation CDG Ib | | |
| Mannosyl-α-1,6- | Congenital disorders of N- | 4247 | Q10469 |
| glycoprotein-β-1,2-N- | glycosylation CDG IIa | | |
| acetylglucosminyltransferase | | | |
| Metalloproteinase-2 | Winchester syndrome | 4313 | P08253 |
| methylmalonyl-CoA | Methylmalonic acidemia | 4594 | P22033 |
| mutase | (vitamin b12 non-responsive) | | |
| N-Acetyl | Mucopolysaccharidosis MPS | 411 | P15848 |
| galactosamine α-4- | VI (Maroteaux-Lamy | | |
| sulfate sulfatase | syndrome) | | |
| (arylsulfatase B) | | | |
| N-acetyl-D- | Mucopolysaccharidosis MPS | 4669 | P54802 |
| glucosaminidase | III (Sanfilippo's syndrome) | | |
| | Type III-B | | |
| N-Acetyl- | Schindler's disease Type I | 4668 | P17050 |
| galactosaminidase | (infantile severe form) | | |
| N-Acetyl- | Schindler's disease Type II | 4668 | P17050 |
| galactosaminidase | (Kanzaki disease, adult-onset | | |
| | form) | | |
| N-Acetyl- | Schindler's disease Type III | 4668 | P17050 |
| galactosaminidase | (intermediate form) | | |
| N-acetyl-glucosaminine- | Mucopolysaccharidosis MPS | 2799 | P15586 |
| 6-sulfate sulfatase | III (Sanfilippo's syndrome) | | |
| | Type III-D | | |
| N-acetylglucosaminyl-1- | Mucolipidosis ML III | 79158 | Q3T906 |
| phosphotransferase | (pseudo-Hurler's | | |
| | polydystrophy) | | |
| N-Acetylglucosaminyl- | Mucolipidosis ML II (I-cell | 79158 | Q3T906 |
| 1-phosphotransferase | disease) | | |
| catalytic subunit | | | |
| N-acetylglucosaminyl-1- | Mucolipidosis ML III | 84572 | Q9UJJ9 |
| phosphotransferase, | (pseudo-Hurler's | | |
| substrate-recognition | polydystrophy) Type III-C | | |
| subunit | | | |
| N- | Aspartylglucosaminuria | 175 | P20933 |
| Aspartylglucosaminidase | | | |
| Neuraminidase 1 | Sialidosis | 4758 | Q99519 |
| (sialidase) | | | |
| Palmitoyl-protein | Ceroid lipofuscinosis Adult | 5538 | P50897 |
| thioesterase-1 | form (CLN4, Kufs' disease) | | |
| Palmitoyl-protein | Ceroid lipofuscinosis | 5538 | P50897 |
| thioesterase-1 | Infantile form (CLN1, | | |
| | Santavuori-Haltia disease) | | |
| Phenylalanine | Phenylketonuria | 5053 | P00439 |
| hydroxylase | | | |
| Phosphomannomutase-2 | Congenital disorders of N- | 5373 | O15305 |
| | glycosylation CDG Ia (solely | | |
| | neurologic and neurologic- | | |
| | multivisceral forms) | | |
| Porphobilinogen | Acute Intermittent Porphyria | 3145 | P08397 |
| deaminase | | | |
| Purine nucleoside | Purine nucleoside | 4860 | P00491 |
| phosphorylase | phosphorylase deficiency | | |
| pyrimidine 5′ | Hemolytic anemia and/or | 51251 | Q9H0P0 |
| nucleotidase | pyrimidine 5′ nucleotidase | | |
| | deficiency | | |
| Sphingomyelinase | Niemann-Pick disease type A | 6609 | P17405 |
| Sphingomyelinase | Niemann-Pick disease type B | 6609 | P17405 |
| Sterol 27-hydroxylase | Cerebrotendinous | 1593 | Q02318 |
| | xanthomatosis (cholestanol | | |
| | lipidosis) | | |
| Thymidine | Mitochondrial | 1890 | P19971 |
| phosphorylase | neurogastrointestinal | | |
| | encephalomyopathy | | |
| | (MNGIE) | | |
| Trihexosylceramide α- | Fabry's disease | 2717 | P06280 |
| galactosidase | | | |
| tyrosinase, e.g., OCA1 | albinism, e.g., ocular albinism | 7299 | P14679 |
| UDP-GlcNAc: dolichyl- | Congenital disorders of N- | 1798 | Q9H3H5 |
| P NAcGlc | glycosylation CDG Ij | | |
| phosphotransferase | | | |
| UDP-N- | Sialuria French type | 10020 | Q9Y223 |
| acetylglucosamine-2- | | | |
| epimerase/N- | | | |
| acetylmannosamine | | | |
| kinase, sialin | | | |
| Uricase | Lesch-Nyhan syndrome, gout | 391051 | No protein |
| uridine diphosphate | Crigler-Najjar syndrome | 54658 | P22309 |
| glucuronyl-transferase | | | |
| (e.g., UGT1A1) | | | |
| α-1,2- | Congenital disorders of N- | 79796 | Q9H6U8 |
| Mannosyltransferase | glycosylation CDG Il | | |
| | (608776) | | |
| α-1,2- | Congenital disorders of N- | 79796 | Q9H6U8 |
| Mannosyltransferase | glycosylation, type I (pre- | | |
| | Golgi glycosylation defects) | | |
| α-1,3- | Congenital disorders of N- | 440138 | Q2TAA5 |
| Mannosyltransferase | glycosylation CDG Ii | | |
| α-D-Mannosidase | α-Mannosidosis, type I | 10195 | Q92685 |
| | (severe) or II (mild) | | |
| α-L-Fucosidase | Fucosidosis | 4123 | Q9NTJ4 |
| α-l-Iduronidase | Mucopolysaccharidosis MPS | 2517 | P04066 |
| | I H/S (Hurler-Scheie | | |
| | syndrome) | | |
| α-l-Iduronidase | Mucopolysaccharidosis MPS | 3425 | P35475 |
| | I-H (Hurler's syndrome) | | |
| α-l-Iduronidase | Mucopolysaccharidosis MPS | 3425 | P35475 |
| | I-S (Scheie's syndrome) | | |
| β-1,4- | Congenital disorders of N- | 3425 | P35475 |
| Galactosyltransferase | glycosylation CDG IId | | |
| β-1,4- | Congenital disorders of N- | 2683 | P15291 |
| Mannosyltransferase | glycosylation CDG Ik | | |
| β-D-Mannosidase | β-Mannosidosis | 56052 | Q9BT22 |
| β-Galactosidase | Mucopolysaccharidosis MPS | 4126 | O00462 |
| | IV (Morquio's syndrome) | | |
| | Type IV-B | | |
| β-Glucuronidase | Mucopolysaccharidosis MPS | 2720 | P16278 |
| | VII (Sly's syndrome) | | |
| β-Hexosaminidase A | Tay-Sachs disease | 2990 | P08236 |
| β-Hexosaminidase B | Sandhoff's disease | 3073 | P06865 |

In some embodiments, an effector described herein comprises an enzyme of Table 54, or a functional variant thereof, e.g., a homolog (e.g., ortholog or paralog) or fragment thereof. In some embodiments, an effector described herein comprises a protein having at least 80%, 85%, 90%, 95%, 967%, 98%, 99% sequence identity to an amino acid sequence listed in Table 54 by reference to its UniProt ID. In some embodiments, the functional variant catalyzes the same reaction as the corresponding wild-type protein, e.g., at a rate no less tan 10%, 20%, 30%, 40%, or 50% lower than the wild-type protein. In some embodiments, an anellovector encoding an enzyme of Table 54, or a functional variant thereof is used for the treatment of a disease or disorder of Table 54. In some embodiments, an anellovector is used to deliver uridine diphosphate glucuronyl-transferase or a functional variant thereof to a target cell, e.g., a liver cell. In some embodiments, an anellovector is used to deliver OCA1 or a functional variant thereof to a target cell, e.g., a retinal cell.

TABLE 55
Exemplary non-enzymatic effectors and corresponding indications

| Effector | Indication | Entrez Gene ID | UniProt ID |
|---|---|---|---|
| Survival motor neuron | spinal muscular atrophy | 6606 | Q16637 |
| protein (SMN) | | | |
| Dystrophin or micro- | muscular dystrophy | 1756 | P11532 |
| dystrophin | (e.g., Duchenne | | |
| | muscular dystrophy or | | |
| | Becker muscular | | |
| | dystrophy) | | |
| Complement protein, | Complement Factor I | 3426 | P05156 |
| e.g., Complement | deficiency | | |
| factor C1 | | | |
| Complement factor H | Atypical hemolytic | 3075 | P08603 |
| | uremic syndrome | | |
| Cystinosin (lysosomal | Cystinosis | 1497 | O60931 |
| cystine transporter) | | | |
| Epididymal secretory | Niemann-Pick disease | 10577 | P61916 |
| protein 1 (HE1; NPC2 | Type C2 | | |
| protein) | | | |
| GDP-fucose | Congenital disorders of | 55343 | Q96A29 |
| transporter-1 | N-glycosylation CDG | | |
| | IIc (Rambam-Hasharon | | |
| | syndrome) | | |
| GM2 activator protein | GM2 activator protein | 2760 | Q17900 |
| | deficiency (Tay-Sachs | | |
| | disease AB variant, | | |
| | GM2A) | | |
| Lysosomal | Ceroid lipofuscinosis | 1207 | Q13286 |
| transmembrane CLN3 | Juvenile form (CLN3, | | |
| protein | Batten disease, Vogt- | | |
| | Spielmeyer disease) | | |
| Lysosomal | Ceroid lipofuscinosis | 1203 | O75503 |
| transmembrane CLN5 | Variant late infantile | | |
| protein | form, Finnish type | | |
| | (CLN5) | | |
| Na phosphate | Infantile sialic acid | 26503 | Q9NRA2 |
| cotransporter, sialin | storage disorder | | |
| Na phosphate | Sialuria Finnish type | 26503 | Q9NRA2 |
| cotransporter, sialin | (Salla disease) | | |
| NPC1 protein | Niemann-Pick disease | 4864 | O15118 |
| | Type C1/Type D | | |
| Oligomeric Golgi | Congenital disorders of | 91949 | P83436 |
| complex-7 | N-glycosylation CDG | | |
| | IIe | | |
| Prosaposin | Prosaposin deficiency | 5660 | P07602 |
| Protective | Galactosialidosis | 5476 | P10619 |
| protein/cathepsin A | (Goldberg's syndrome, | | |
| (PPCA) | combined | | |
| | neuraminidase and β- | | |
| | galactosidase | | |
| | deficiency) | | |
| Protein involved in | Congenital disorders of | 9526 | O75352 |
| mannose-P-dolichol | N-glycosylation CDG If | | |
| utilization | | | |
| Saposin B | Saposin B deficiency | 5660 | P07602 |
| | (sulfatide activator | | |
| | deficiency) | | |
| Saposin C | Saposin C deficiency | 5660 | P07602 |
| | (Gaucher's activator | | |
| | deficiency) | | |
| Sulfatase-modifying | Mucosulfatidosis | 285362 | Q8NBK3 |
| factor-1 | (multiple sulfatase | | |
| | deficiency) | | |
| Transmembrane | Ceroid lipofuscinosis | 54982 | Q9NWW5 |
| CLN6 protein | Variant late infantile | | |
| | form (CLN6) | | |
| Transmembrane | Ceroid lipofuscinosis | 2055 | Q9UBY8 |
| CLN8 protein | Progressive epilepsy | | |
| | with intellectual | | |
| | disability | | |
| vWF | von Willebrand disease | 7450 | P04275 |
| Factor I (fibrinogen) | Afibrinogenomia | 2243, 2244, 2266 | P02671, P02675, |
| | | | P02679 |
| erythropoietin (hEPO) | | | |

In some embodiments, an effector described herein comprises an erythropoietin (EPO), e.g., a human erythropoietin (hEPO), or a functional variant thereof. In some embodiments, an anellovector encoding an erythropoietin, or a functional variant thereof is used for stimulating erythropoiesis. In some embodiments, an anellovector encoding an erythropoietin, or a functional variant thereof is used for the treatment of a disease or disorder, e.g., anemia. In some embodiments, an anellovector is used to deliver EPO or a functional variant thereof to a target cell, e.g., a red blood cell.

In some embodiments, an effector described herein comprises a polypeptide of Table 55, or a functional variant thereof, e.g., a homolog (e.g., ortholog or paralog) or fragment thereof. In some embodiments, an effector described herein comprises a protein having at least 80%, 85%, 90%, 95%, 967%, 98%, 99% sequence identity to an amino acid sequence listed in Table 55 by reference to its UniProt ID. In some embodiments, an anellovector encoding a polypeptide of Table 55, or a functional variant thereof is used for the treatment of a disease or disorder of Table 55. In some embodiments, an anellovector is used to deliver SMN or a functional variant thereof to a target cell, e.g., a cell of the spinal cord and/or a motor neuron. In some embodiments, an anellovector is used to deliver a micro-dystrophin to a target cell, e.g., a myocyte.

Exemplary micro-dystrophins are described in Duan, “Systemic AAV Micro-dystrophin Gene Therapy for Duchenne Muscular Dystrophy.” Mol Ther. 2018 Oct. 3; 26(10):2337-2356. doi: 10.1016/j.ymthe.2018.07.011. Epub 2018 Jul. 17.

In some embodiments, an effector described herein comprises a clotting factor, e.g., a clotting factor listed in Table 54 or Table 55 herein. In some embodiments, an effector described herein comprises a protein that, when mutated, causes a lysosomal storage disorder, e.g., a protein listed in Table 54 or Table 55 herein. In some embodiments, an effector described herein comprises a transporter protein, e.g., a transporter protein listed in Table 55 herein.

In some embodiments, a functional variant of a wild-type protein comprises a protein that has one or more activities of the wild-type protein, e.g., the functional variant catalyzes the same reaction as the corresponding wild-type protein, e.g., at a rate no less than 10%, 20%, 30%, 40%, or 50% lower than the wild-type protein. In some embodiments, the functional variant binds to the same binding partner that is bound by the wild-type protein, e.g., with a Kd of no more than 10%, 20%, 30%, 40%, or 50% higher than the Kd of the corresponding wild-type protein for the same binding partner under the same conditions. In some embodiments, the functional variant has at a polypeptide sequence at least 70%, 75%, 80%, 85%, 90%, 95%, 96%, 97%, 98%, or 99% identical to that of the wild-type polypeptide. In some embodiments, the functional variant comprises a homolog (e.g., ortholog or paralog) of the corresponding wild-type protein. In some embodiments, the functional variant is a fusion protein. In some embodiments, the fusion comprises a first region with at least 70%, 75%, 80%, 85%, 90%, 95%, 96%, 97%, 98%, or 99% identity to the corresponding wild-type protein, and a second, heterologous region. In some embodiments, the functional variant comprises or consists of a fragment of the corresponding wild-type protein.

Regeneration, Repair, and Fibrosis Factors

Therapeutic polypeptides described herein also include growth factors, e.g., as disclosed in Table 56, or functional variants thereof, e.g., a protein having at least 80%, 85%, 90%, 95%, 967%, 98%, 99% identity to a protein sequence disclosed in Table 56 by reference to its UniProt ID. Also included are antibodies or fragments thereof against such growth factors, or miRNAs that promote regeneration and repair.

TABLE 56
Exemplary regeneration, repair, and fibrosis factors

| Target | Gene accession # | Protein accession # |
|---|---|---|
| VEGF-A | NG_008732 | NP_001165094 |
| NRG-1 | NG_012005 | NP_001153471 |
| FGF2 | NG_029067 | NP_001348594 |
| FGF1 | Gene ID: 2246 | NP_001341882 |
| miR-199-3p | MIMAT0000232 | |
| miR-590-3p | MIMAT0004801 | |
| mi-17-92 | MI0000071 | https://www.ncbi.nlm.nih.gov/pmc/ |
| | | articles/PMC2732113/figure/F1/ |
| miR-222 | MI0000299 | |
| miR-302-367 | MIR302A And | https://www.ncbi.nlm.nih.gov/pmc/ |
| | MIR367 | articles/PMC4400607/ |

Transformation Factors

Therapeutic polypeptides described herein also include transformation factors, e.g., protein factors that transform fibroblasts into differentiated cell e.g., factors disclosed in Table 57 or functional variants thereof, e.g., a protein having at least 80%, 85%, 90%, 95%, 967%, 98%, 99% identity to a protein sequence disclosed in Table 57 by reference to its UniProt ID.

TABLE 57
Exemplary transformation factors

| Target | Indication | Gene accession # | Protein accession # |
|---|---|---|---|
| MESP1 | Organ Repair by | Gene ID: 55897 | EAX02066 |
| | transforming fibroblasts | | |
| ETS2 | Organ Repair by | GeneID: 2114 | NP_005230 |
| | transforming fibroblasts | | |
| HAND2 | Organ Repair by | GeneID: 9464 | NP_068808 |
| | transforming fibroblasts | | |
| MYOCARDIN | Organ Repair by | GeneID: 93649 | NP_001139784 |
| | transforming fibroblasts | | |
| ESRRA | Organ Repair by | Gene ID: 2101 | AAH92470 |
| | transforming fibroblasts | | |
| miR-1 | Organ Repair by | MI0000651 | n/a |
| | transforming fibroblasts | | |
| miR-133 | Organ Repair by | MI0000450 | n/a |
| | transforming fibroblasts | | |
| TGFb | Organ Repair by | GeneID: 7040 | NP_000651.3 |
| | transforming fibroblasts | | |
| WNT | Organ Repair by | Gene ID: 7471 | NP_005421 |
| | transforming fibroblasts | | |
| JAK | Organ Repair by | Gene ID: 3716 | NP_001308784 |
| | transforming fibroblasts | | |
| NOTCH | Organ Repair by | GeneID: 4851 | XP_011517019 |
| | transforming fibroblasts | | |

Proteins that Stimulate Cellular Regeneration

Therapeutic polypeptides described herein also include proteins that stimulate cellular regeneration e.g., proteins disclosed in Table 58 or functional variants thereof, e.g., a protein having at least 80%, 85%, 90%, 95%, 967%, 98%, 99% identity to a protein sequence disclosed in Table 58 by reference to its UniProt ID.

TABLE 58
Exemplary proteins that stimulate cellular regeneration

| Target | Gene accession # | Protein accession # |
|---|---|---|
| MST1 | NG_016454 | NP_066278 |
| STK30 | Gene ID: 26448 | NP_036103 |
| MST2 | Gene ID: 6788 | NP_006272 |
| SAV1 | Gene ID: 60485 | NP_068590 |
| LATS1 | Gene ID: 9113 | NP_004681 |
| LATS2 | Gene ID: 26524 | NP_055387 |
| YAP1 | NG_029530 | NP_001123617 |
| CDKN2b | NG_023297 | NP_004927 |
| CDKN2a | NG_007485 | NP_478102 |

STING Modulator Effectors

In some embodiments, a secreted effector described herein modulates STING/cGAS signaling. In some embodiments, the STING modulator is a polypeptide, e.g., a viral polypeptide or a functional variant thereof. For instance, the effector may comprise a STING modulator (e.g., inhibitor) described in Maringer et al. “Message in a bottle: lessons learned from antagonism of STING signalling during RNA virus infection” Cytokine & Growth Factor Reviews Volume 25, Issue 6, December 2014, Pages 669-679, which is incorporated herein by reference in its entirety. Additional STING modulators (e.g., activators) are described, e.g., in Wang et al. “STING activator c-di-GMP enhances the anti-tumor effects of peptide vaccines in melanoma-bearing mice.” Cancer Immunol Immunother. 2015 August; 64(8):1057-66. doi: 10.1007/s00262-015-1713-5. Epub 2015 May 19; Bose “cGAS/STING Pathway in Cancer: Jekyll and Hyde Story of Cancer Immune Response” Int J Mol Sci. 2017 November; 18(11): 2456; and Fu et al. “STING agonist formulated cancer vaccines can cure established tumors resistant to PD-1 blockade” Sci Transl Med. 2015 Apr. 15; 7(283): 283ra52, each of which is incorporated herein by reference in its entirety.

Some examples of peptides include, but are not limited to, fluorescent tag or marker, antigen, peptide therapeutic, synthetic or analog peptide from naturally-bioactive peptide, agonist or antagonist peptide, anti-microbial peptide, a targeting or cytotoxic peptide, a degradation or self-destruction peptide, and degradation or self-destruction peptides. Peptides useful in the invention described herein also include antigen-binding peptides, e.g., antigen binding antibody or antibody-like fragments, such as single chain antibodies, nanobodies (see, e.g., Steeland et al. 2016. Nanobodies as therapeutics: big opportunities for small antibodies. Drug Discov Today: 21(7):1076-113). Such antigen binding peptides may bind a cytosolic antigen, a nuclear antigen, or an intra-organellar antigen.

In some embodiments, the genetic element comprises a sequence that encodes small peptides, peptidomimetics (e.g., peptoids), amino acids, and amino acid analogs. Such therapeutics generally have a molecular weight less than about 5,000 grams per mole, a molecular weight less than about 2,000 grams per mole, a molecular weight less than about 1,000 grams per mole, a molecular weight less than about 500 grams per mole, and salts, esters, and other pharmaceutically acceptable forms of such compounds. Such therapeutics may include, but are not limited to, a neurotransmitter, a hormone, a drug, a toxin, a viral or microbial particle, a synthetic molecule, and agonists or antagonists thereof.

In some embodiments, the composition or anellovector described herein includes a polypeptide linked to a ligand that is capable of targeting a specific location, tissue, or cell.

Gene Editing Components

The genetic element of the anellovector may include one or more genes that encode a component of a gene editing system. Exemplary gene editing systems include the clustered regulatory interspaced short palindromic repeat (CRISPR) system, zinc finger nucleases (ZFNs), and Transcription Activator-Like Effector-based Nucleases (TALEN). ZFNs, TALENs, and CRISPR-based methods are described, e.g., in Gaj et al. Trends Biotechnol. 31.7(2013):397-405; CRISPR methods of gene editing are described, e.g., in Guan et al., Application of CRISPR-Cas system in gene therapy: Pre-clinical progress in animal model. DNA Repair 2016 October;46:1-8. doi: 10.1016/j.dnarep.2016.07.004; Zheng et al., Precise gene deletion and replacement using the CRISPR/Cas9 system in human cells. BioTechniques, Vol. 57, No. 3, September 2014, pp. 115-124.

CRISPR systems are adaptive defense systems originally discovered in bacteria and archaea. CRISPR systems use RNA-guided nucleases termed CRISPR-associated or “Cas” endonucleases (e. g., Cas9 or Cpf1) to cleave foreign DNA. In a typical CRISPR/Cas system, an endonuclease is directed to a target nucleotide sequence (e. g., a site in the genome that is to be sequence-edited) by sequence-specific, non-coding “guide RNAs” that target single- or double-stranded DNA sequences. Three classes (1-111) of CRISPR systems have been identified. The class II CRISPR systems use a single Cas endonuclease (rather than multiple Cas proteins). One class II CRISPR system includes a type II Cas endonuclease such as Cas9, a CRISPR RNA (“crRNA”), and a trans-activating crRNA (“tracrRNA”). The crRNA contains a “guide RNA”, typically about 20-nucleotide RNA sequence that corresponds to a target DNA sequence. The crRNA also contains a region that binds to the tracrRNA to form a partially double-stranded structure which is cleaved by RNase III, resulting in a crRNA/tracrRNA hybrid. The crRNA/tracrRNA hybrid then directs the Cas9 endonuclease to recognize and cleave the target DNA sequence. The target DNA sequence must generally be adjacent to a “protospacer adjacent motif” (“PAM”) that is specific for a given Cas endonuclease; however, PAM sequences appear throughout a given genome.

In some embodiments, the anellovector includes a gene for a CRISPR endonuclease. For example, some CRISPR endonucleases identified from various prokaryotic species have unique PAM sequence requirements; examples of PAM sequences include 5′-NGG (Streptococcus pyogenes), 5′-NNAGAA (Streptococcus thermophilus CRISPR1), 5′-NGGNG (Streptococcus thermophilus CRISPR3), and 5′-NNNGATT (Neisseria meningitidis). Some endonucleases, e. g., Cas9 endonucleases, are associated with G-rich PAM sites, e. g., 5′-NGG, and perform blunt-end cleaving of the target DNA at a location 3 nucleotides upstream from (5′ from) the PAM site. Another class II CRISPR system includes the type V endonuclease Cpf1, which is smaller than Cas9; examples include AsCpf1 (from Acidaminococcus sp.) and LbCpf1 (from Lachnospiraceae sp.). Cpf1 endonucleases, are associated with T-rich PAM sites, e. g., 5′-TTN. Cpf1 can also recognize a 5′-CTA PAM motif. Cpf1 cleaves the target DNA by introducing an offset or staggered double-strand break with a 4- or 5-nucleotide 5′ overhang, for example, cleaving a target DNA with a 5-nucleotide offset or staggered cut located 18 nucleotides downstream from (3′ from) from the PAM site on the coding strand and 23 nucleotides downstream from the PAM site on the complimentary strand; the 5-nucleotide overhang that results from such offset cleavage allows more precise genome editing by DNA insertion by homologous recombination than by insertion at blunt-end cleaved DNA. See, e. g., Zetsche et al. (2015) Cell, 163:759-771.

A variety of CRISPR associated (Cas) genes may be included in the anellovector. Specific examples of genes are those that encode Cas proteins from class II systems including Cas1, Cas2, Cas3, Cas4, Cas5, Cas6, Cas7, Cas8, Cas9, Cas10, Cpf1, C2C1, or C2C3. In some embodiments, the anellovector includes a gene encoding a Cas protein, e.g., a Cas9 protein, may be from any of a variety of prokaryotic species. In some embodiments, the anellovector includes a gene encoding a particular Cas protein, e.g., a particular Cas9 protein, is selected to recognize a particular protospacer-adjacent motif (PAM) sequence. In some embodiments, the anellovector includes nucleic acids encoding two or more different Cas proteins, or two or more Cas proteins, may be introduced into a cell, zygote, embryo, or animal, e.g., to allow for recognition and modification of sites comprising the same, similar or different PAM motifs. In some embodiments, the anellovector includes a gene encoding a modified Cas protein with a deactivated nuclease, e.g., nuclease-deficient Cas9.

Whereas wild-type Cas9 protein generates double-strand breaks (DSBs) at specific DNA sequences targeted by a gRNA, a number of CRISPR endonucleases having modified functionalities are known, for example: a “nickase” version of Cas endonuclease (e.g., Cas9) generates only a single-strand break; a catalytically inactive Cas endonuclease, e.g., Cas9 (“dCas9”) does not cut the target DNA. A gene encoding a dCas9 can be fused with a gene encoding an effector domain to repress (CRISPRi) or activate (CRISPRa) expression of a target gene. For example, the gene may encode a Cas9 fusion with a transcriptional silencer (e.g., a KRAB domain) or a transcriptional activator (e.g., a dCas9-VP64 fusion). A gene encoding a catalytically inactive Cas9 (dCas9) fused to FokI nuclease (“dCas9-FokI”) can be included to generate DSBs at target sequences homologous to two gRNAs. See, e. g., the numerous CRISPR/Cas9 plasmids disclosed in and publicly available from the Addgene repository (Addgene, 75 Sidney St., Suite 550A, Cambridge, MA 02139; addgene.org/crispr/). A “double nickase” Cas9 that introduces two separate double-strand breaks, each directed by a separate guide RNA, is described as achieving more accurate genome editing by Ran et al. (2013) Cell, 154:1380-1389.

CRISPR technology for editing the genes of eukaryotes is disclosed in US Patent Application Publications 2016/0138008A1 and US2015/0344912A1, and in U.S. Pat. Nos. 8,697,359, 8,771,945, 8,945,839, 8,999,641, 8,993,233, 8,895,308, 8,865,406, 8,889,418, 8,871,445, 8,889,356, 8,932,814, 8,795,965, and 8,906,616. Cpf1 endonuclease and corresponding guide RNAs and PAM sites are disclosed in US Patent Application Publication 2016/0208243 A1.

In some embodiments, the anellovector comprises a gene encoding a polypeptide described herein, e.g., a targeted nuclease, e.g., a Cas9, e.g., a wild type Cas9, a nickase Cas9 (e.g., Cas9 D10A), a dead Cas9 (dCas9), eSpCas9, Cpf1, C2C1, or C2C3, and a gRNA. The choice of genes encoding the nuclease and gRNA(s) is determined by whether the targeted mutation is a deletion, substitution, or addition of nucleotides, e.g., a deletion, substitution, or addition of nucleotides to a targeted sequence. Genes that encode a catalytically inactive endonuclease e.g., a dead Cas9 (dCas9, e.g., D10A; H840A) tethered with all or a portion of (e.g., biologically active portion of) an (one or more) effector domain (e.g., VP64) create chimeric proteins that can modulate activity and/or expression of one or more target nucleic acids sequences.

In some embodiments, the anellovector includes a gene encoding a fusion of a dCas9 with all or a portion of one or more effector domains (e.g., a full-length wild-type effector domain, or a fragment or variant thereof, e.g., a biologically active portion thereof) to create a chimeric protein useful in the methods described herein. Accordingly, in some embodiments, the anellovector includes a gene encoding a dCas9-methylase fusion. In other some embodiments, the anellovector includes a gene encoding a dCas9-enzyme fusion with a site-specific gRNA to target an endogenous gene.

In other aspects, the anellovector includes a gene encoding 1, 2, 3, 4, 5, 6, 7, 8, 9, 10, 11, 12, 13, 14, 15, 16, 17, 18, 19, 20, or more effector domains (all or a biologically active portion) fused with dCas9.

### Regulatory Sequences

In some embodiments, the genetic element comprises a regulatory sequence, e.g., a promoter or an enhancer, operably linked to the sequence encoding the effector.

In some embodiments, a promoter includes a DNA sequence that is located adjacent to a DNA sequence that encodes an expression product. A promoter may be linked operatively to the adjacent DNA sequence. A promoter typically increases an amount of product expressed from the DNA sequence as compared to an amount of the expressed product when no promoter exists. A promoter from one organism can be utilized to enhance product expression from the DNA sequence that originates from another organism. For example, a vertebrate promoter may be used for the expression of jellyfish GFP in vertebrates. Hence, one promoter element can enhance the expression of one or more products. Multiple promoter elements are well-known to persons of ordinary skill in the art.

In one embodiment, high-level constitutive expression is desired. Examples of such promoters include, without limitation, the retroviral Rous sarcoma virus (RSV) long terminal repeat (LTR) promoter/enhancer, the cytomegalovirus (CMV) immediate early promoter/enhancer (see, e.g., Boshart et al, Cell, 41:521-530 (1985)), the SV40 promoter, the dihydrofolate reductase promoter, the cytoplasmic .beta.-actin promoter and the phosphoglycerol kinase (PGK) promoter.

In another embodiment, inducible promoters may be desired. Inducible promoters are those which are regulated by exogenously supplied compounds, e.g., provided either in cis or in trans, including without limitation, the zinc-inducible sheep metallothionine (MT) promoter; the dexamethasone (Dex)-inducible mouse mammary tumor virus (MMTV) promoter; the T7 polymerase promoter system (WO 98/10088); the tetracycline-repressible system (Gossen et al, Proc. Natl. Acad. Sci. USA, 89:5547-5551 (1992)); the tetracycline-inducible system (Gossen et al., Science, 268:1766-1769 (1995); see also Harvey et al., Curr. Opin. Chem. Biol., 2:512-518 (1998)); the RU486-inducible system (Wang et al., Nat. Biotech., 15:239-243 (1997) and Wang et al., Gene Ther., 4:432-441 (1997)]; and the rapamycin-inducible system (Magari et al., J. Clin. Invest., 100:2865-2872 (1997); Rivera et al., Nat. Medicine. 2:1028-1032 (1996)). Other types of inducible promoters which may be useful in this context are those which are regulated by a specific physiological state, e.g., temperature, acute phase, or in replicating cells only.

In some embodiments, a native promoter for a gene or nucleic acid sequence of interest is used. The native promoter may be used when it is desired that expression of the gene or the nucleic acid sequence should mimic the native expression. The native promoter may be used when expression of the gene or other nucleic acid sequence must be regulated temporally or developmentally, or in a tissue-specific manner, or in response to specific transcriptional stimuli. In a further embodiment, other native expression control elements, such as enhancer elements, polyadenylation sites or Kozak consensus sequences may also be used to mimic the native expression.

In some embodiments, the genetic element comprises a gene operably linked to a tissue-specific promoter. For instance, if expression in skeletal muscle is desired, a promoter active in muscle may be used. These include the promoters from genes encoding skeletal α-actin, myosin light chain 2A, dystrophin, muscle creatine kinase, as well as synthetic muscle promoters with activities higher than naturally-occurring promoters. See Li et al., Nat. Biotech., 17:241-245 (1999). Examples of promoters that are tissue-specific are known for liver albumin, Miyatake et al. J. Virol., 71:5124-32 (1997); hepatitis B virus core promoter, Sandig et al., Gene Ther. 3:1002-9 (1996); alpha-fetoprotein (AFP), Arbuthnot et al., Hum. Gene Ther., 7:1503-14 (1996)], bone (osteocalcin, Stein et al., Mol. Biol. Rep., 24:185-96 (1997); bone sialoprotein, Chen et al., J. Bone Miner. Res. 11:654-64 (1996)), lymphocytes (CD2, Hansal et al., J. Immunol., 161:1063-8 (1998); immunoglobulin heavy chain; T cell receptor a chain), neuronal (neuron-specific enolase (NSE) promoter, Andersen et al. Cell. Mol. Neurobiol., 13:503-15 (1993); neurofilament light-chain gene, Piccioli et al., Proc. Natl. Acad. Sci. USA, 88:5611-5 (1991); the neuron-specific vgf gene, Piccioli et al., Neuron, 15:373-84 (1995)]; among others.

The genetic element may include an enhancer, e.g., a DNA sequence that is located adjacent to the DNA sequence that encodes a gene. Enhancer elements are typically located upstream of a promoter element or can be located downstream of or within a coding DNA sequence (e.g., a DNA sequence transcribed or translated into a product or products). Hence, an enhancer element can be located 100 base pairs, 200 base pairs, or 300 or more base pairs upstream or downstream of a DNA sequence that encodes the product. Enhancer elements can increase an amount of recombinant product expressed from a DNA sequence above increased expression afforded by a promoter element. Multiple enhancer elements are readily available to persons of ordinary skill in the art.

In some embodiments, the genetic element comprises one or more inverted terminal repeats (ITR) flanking the sequences encoding the expression products described herein. In some embodiments, the genetic element comprises one or more long terminal repeats (LTR) flanking the sequence encoding the expression products described herein. Examples of promoter sequences that may be used, include, but are not limited to, the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, and a Rous sarcoma virus promoter.

### Replication Proteins

In some embodiments, the genetic element of the anellovector, e.g., synthetic anellovector, may include sequences that encode one or more replication proteins. In some embodiments, the anellovector may replicate by a rolling-circle replication method, e.g., synthesis of the leading strand and the lagging strand is uncoupled. In such embodiments, the anellovector comprises three elements additional elements: i) a gene encoding an initiator protein, ii) a double strand origin, and iii) a single strand origin. A rolling circle replication (RCR) protein complex comprising replication proteins binds to the leading strand and destabilizes the replication origin. The RCR complex cleaves the genome to generate a free 3′OH extremity. Cellular DNA polymerase initiates viral DNA replication from the free 3′OH extremity. After the genome has been replicated, the RCR complex closes the loop covalently. This leads to the release of a positive circular single-stranded parental DNA molecule and a circular double-stranded DNA molecule composed of the negative parental strand and the newly synthesized positive strand. The single-stranded DNA molecule can be either encapsidated or involved in a second round of replication. See for example, Virology Journal 2009, 6:60 doi:10.1186/1743-422X-6-60.

The genetic element may comprise a sequence encoding a polymerase, e.g., RNA polymerase or a DNA polymerase.

### Other Sequences

In some embodiments, the genetic element further includes a nucleic acid encoding a product (e.g., a ribozyme, a therapeutic mRNA encoding a protein, an exogenous gene).

In some embodiments, the genetic element includes one or more sequences that affect species and/or tissue and/or cell tropism (e.g. capsid protein sequences), infectivity (e.g. capsid protein sequences), immunosuppression/activation (e.g. regulatory nucleic acids), viral genome binding and/or packaging, immune evasion (non-immunogenicity and/or tolerance), pharmacokinetics, endocytosis and/or cell attachment, nuclear entry, intracellular modulation and localization, exocytosis modulation, propagation, and nucleic acid protection of the anellovector in a host or host cell.

In some embodiments, the genetic element may comprise other sequences that include DNA, RNA, or artificial nucleic acids. The other sequences may include, but are not limited to, genomic DNA, cDNA, or sequences that encode tRNA, mRNA, rRNA, miRNA, gRNA, siRNA, or other RNAi molecules. In one embodiment, the genetic element includes a sequence encoding an siRNA to target a different loci of the same gene expression product as the regulatory nucleic acid. In one embodiment, the genetic element includes a sequence encoding an siRNA to target a different gene expression product as the regulatory nucleic acid.

In some embodiments, the genetic element further comprises one or more of the following sequences: a sequence that encodes one or more miRNAs, a sequence that encodes one or more replication proteins, a sequence that encodes an exogenous gene, a sequence that encodes a therapeutic, a regulatory sequence (e.g., a promoter, enhancer), a sequence that encodes one or more regulatory sequences that targets endogenous genes (siRNA, lncRNAs, shRNA), and a sequence that encodes a therapeutic mRNA or protein.

The other sequences may have a length from about 2 to about 5000 nts, about 10 to about 100 nts, about 50 to about 150 nts, about 100 to about 200 nts, about 150 to about 250 nts, about 200 to about 300 nts, about 250 to about 350 nts, about 300 to about 500 nts, about 10 to about 1000 nts, about 50 to about 1000 nts, about 100 to about 1000 nts, about 1000 to about 2000 nts, about 2000 to about 3000 nts, about 3000 to about 4000 nts, about 4000 to about 5000 nts, or any range therebetween.

Encoded Genes

For example, the genetic element may include a gene associated with a signaling biochemical pathway, e.g., a signaling biochemical pathway-associated gene or polynucleotide. Examples include a disease associated gene or polynucleotide. A “disease-associated” gene or polynucleotide refers to any gene or polynucleotide which is yielding transcription or translation products at an abnormal level or in an abnormal form in cells derived from a disease-affected tissues compared with tissues or cells of a non disease control. It may be a gene that becomes expressed at an abnormally high level; it may be a gene that becomes expressed at an abnormally low level, where the altered expression correlates with the occurrence and/or progression of the disease. A disease-associated gene also refers to a gene possessing mutation(s) or genetic variation that is directly responsible or is in linkage disequilibrium with a gene(s) that is responsible for the etiology of a disease.

Examples of disease-associated genes and polynucleotides are available from McKusick-Nathans Institute of Genetic Medicine, Johns Hopkins University (Baltimore, Md.) and National Center for Biotechnology Information, National Library of Medicine (Bethesda, Md.). Examples of disease-associated genes and polynucleotides are listed in Tables A and B of U.S. Pat. No. 8,697,359, which are herein incorporated by reference in their entirety. Disease specific information is available from McKusick-Nathans Institute of Genetic Medicine, Johns Hopkins University (Baltimore, Md.) and National Center for Biotechnology Information, National Library of Medicine (Bethesda, Md.). Examples of signaling biochemical pathway-associated genes and polynucleotides are listed in Tables A-C of U.S. Pat. No. 8,697,359, which are herein incorporated by reference in their entirety.

Moreover, the genetic elements can encode targeting moieties, as described elsewhere herein. This can be achieved, e.g., by inserting a polynucleotide encoding a sugar, a glycolipid, or a protein, such as an antibody. Those skilled in the art know additional methods for generating targeting moieties.

Viral Sequence

In some embodiments, the genetic element comprises at least one viral sequence. In some embodiments, the sequence has homology or identity to one or more sequence from a single stranded DNA virus, e.g., Anellovirus, Bidnavirus, Circovirus, Geminivirus, Genomovirus, Inovirus, Microvirus, Nanovirus, Parvovirus, and Spiravirus. In some embodiments, the sequence has homology or identity to one or more sequence from a double stranded DNA virus, e.g., Adenovirus, Ampullavirus, Ascovirus, Asfarvirus, Baculovirus, Fusellovirus, Globulovirus, Guttavirus, Hytrosavirus, Herpesvirus, Iridovirus, Lipothrixvirus, Nimavirus, and Poxvirus. In some embodiments, the sequence has homology or identity to one or more sequence from an RNA virus, e.g., Alphavirus, Furovirus, Hepatitis virus, Hordeivirus, Tobamovirus, Tobravirus, Tricornavirus, Rubivirus, Birnavirus, Cystovirus, Partitivirus, and Reovirus.

In some embodiments, the genetic element may comprise one or more sequences from a non-pathogenic virus, e.g., a symbiotic virus, e.g., a commensal virus, e.g., a native virus, e.g., an Anellovirus. Recent changes in nomenclature have classified the three Anelloviruses able to infect human cells into Alphatorquevirus (TT), Betatorquevirus (TTM), and Gammatorquevirus (TTMD) Genera of the Anelloviridae family of viruses. To date Anelloviruses have not been linked to any human disease. In some embodiments, the genetic element may comprise a sequence with homology or identity to a Torque Teno Virus (TT), a non-enveloped, single-stranded DNA virus with a circular, negative-sense genome. In some embodiments, the genetic element may comprise a sequence with homology or identity to a SEN virus, a Sentinel virus, a TTV-like mini virus, and a TT virus. Different types of TT viruses have been described including TT virus genotype 6, TT virus group, TTV-like virus DXL1, and TTV-like virus DXL2. In some embodiments, the genetic element may comprise a sequence with homology or identity to a smaller virus, Torque Teno-like Mini Virus (TTM), or a third virus with a genomic size in between that of TTV and TTMV, named Torque Teno-like Midi Virus (TTMD). In some embodiments, the genetic element may comprise one or more sequences or a fragment of a sequence from a non-pathogenic virus having at least about 60%, 70% 80%, 85%, 90% 95%, 96%, 97%, 98% and 99% nucleotide sequence identity to any one of the nucleotide sequences described herein.

In some embodiments, the genetic element may comprise one or more sequences or a fragment of a sequence from a substantially non-pathogenic virus having at least about 60%, 70% 80%, 85%, 90% 95%, 96%, 97%, 98% and 99% nucleotide sequence identity to any one of the nucleotide sequences described herein, e.g., Table 41.

TABLE 41
Examples of Anelloviruses and their sequences. Accessions numbers
and related sequence information may be obtained at www.ncbi.nlm.nih.gov/genbank/,
as referenced on Dec. 11, 2018.

| Accession # | Description |
|---|---|
| AB017613.1 | Torque teno virus 16 DNA, complete genome, isolate: TUS01 |
| AB026345.1 | TT virus genes for ORF1 and ORF2, complete cds, isolate: TRM1 |
| AB026346.1 | TT virus genes for ORF1 and ORF2, complete cds, isolate: TK16 |
| AB026347.1 | TT virus genes for ORF1 and ORF2, complete cds, isolate: TP1-3 |
| AB028669.1 | TT virus gene for ORF1 and ORF2, complete genome, isolate: TJN02 |
| AB030487.1 | TT virus gene for pORF2a, pORF2b, PORF1, complete cds, clone: JaCHCTC19 |
| AB030488.1 | TT virus gene for pORF2a, pORF2b, pORF1, complete cds, clone: JaBD89 |
| AB030489.1 | TT virus gene for pORF2a, pORF2b, PORF1, complete cds, clone: JaBD98 |
| AB038340.1 | TT virus genes for ORF2s, ORF1, ORF3, complete cds |
| AB038622.1 | TT virus genes for ORF2, ORF1, ORF3, complete cds, isolate: TTVyon-LC011 |
| AB038623.1 | TT virus genes for ORF2, ORF1, ORF3, complete cds, isolate: TTVyon-KC186 |
| AB038624.1 | TT virus genes for ORF2, ORF1, ORF3, complete cds, isolate: TTVyon-KC197 |
| AB041821.1 | TT virus mRNA for VP1, complete cds |
| AB050448.1 | Torque teno virus genes for ORF1, ORF2, ORF3, ORF4, complete cds, isolate: |
| | TYM9 |
| AB060592.1 | Torque teno virus gene for ORF1, ORF2, ORF3, ORF4, clone: SAa-39 |
| AB060593.1 | Torque teno virus gene for ORF1, ORF2, ORF3, ORF4, complete cds, clone: |
| | SAa-38 |
| AB060595.1 | TT virus gene for ORF1, ORF2, ORF3, ORF4, complete cds, clone: SAj-30 |
| AB060596.1 | TT virus gene for ORF1, ORF2, ORF3, ORF4, complete cds, clone: SAf-09 |
| AB064596.1 | Torque teno virus DNA, complete genome, isolate: CT25F |
| AB064597.1 | Torque teno virus DNA, complete genome, isolate: CT30F |
| AB064599.1 | Torque teno virus DNA, complete genome, isolate: JT03F |
| AB064600.1 | Torque teno virus DNA, complete genome, isolate: JT05F |
| AB064601.1 | Torque teno virus DNA, complete genome, isolate: JT14F |
| AB064602.1 | Torque teno virus DNA, complete genome, isolate: JT19F |
| AB064603.1 | Torque teno virus DNA, complete genome, isolate: JT41F |
| AB064604.1 | Torque teno virus DNA, complete genome, isolate: CT39F |
| AB064606.1 | Torque teno virus DNA, complete genome, isolate: JT33F |
| AB290918.1 | Torque teno midi virus 1 DNA, complete genome, isolate: MD1-073 |
| AF079173.1 | TT virus strain TTVCHN1, complete genome |
| AF116842.1 | TT virus strain BDH1, complete genome |
| AF122914.3 | TT virus isolate JA20, complete genome |
| AF122917.1 | TT virus isolate JA4, complete genome |
| AF122919.1 | TT virus isolate JA10 unknown genes |
| AF129887.1 | TT virus TTVCHN2, complete genome |
| AF247137.1 | TT virus isolate TUPB, complete genome |
| AF254410.1 | TT virus ORF2 protein and ORF1 protein genes, complete cds |
| AF298585.1 | TT virus Polish isolate P/1C1, complete genome |
| AF315076.1 | TTV-like virus DXL1 unknown genes |
| AF315077.1 | TTV-like virus DXL2 unknown genes |
| AF345521.1 | TT virus isolate TCHN-G1 Orf2 and Orf1 genes, complete cds |
| AF345522.1 | TT virus isolate TCHN-E Orf2 and Orf1 genes, complete cds |
| AF345525.1 | TT virus isolate TCHN-D2 Orf2 and Orf1 genes, complete cds |
| AF345527.1 | TT virus isolate TCHN-C2 Orf2 and Orf1 genes, complete cds |
| AF345528.1 | TT virus isolate TCHN-F Orf2 and Orf1 genes, complete cds |
| AF345529.1 | TT virus isolate TCHN-G2 Orf2 and Orf1 genes, complete cds |
| AF371370.1 | TT virus ORF1, ORF3, and ORF2 genes, complete cds |
| AJ620212.1 | Torque teno virus, isolate tth6, complete genome |
| AJ620213.1 | Torque teno virus, isolate tth10, complete genome |
| AJ620214.1 | Torque teno virus, isolate tth11g2, complete genome |
| AJ620215.1 | Torque teno virus, isolate tth18, complete genome |
| AJ620216.1 | Torque teno virus, isolate tth20, complete genome |
| AJ620217.1 | Torque teno virus, isolate tth21, complete genome |
| AJ620218.1 | Torque teno virus, isolate tth3, complete genome |
| AJ620219.1 | Torque teno virus, isolate tth9, complete genome |
| AJ620220.1 | Torque teno virus, isolate tth16, complete genome |
| AJ620221.1 | Torque teno virus, isolate tth17, complete genome |
| AJ620222.1 | Torque teno virus, isolate tth25, complete genome |
| AJ620223.1 | Torque teno virus, isolate tth26, complete genome |
| AJ620224.1 | Torque teno virus, isolate tth27, complete genome |
| AJ620225.1 | Torque teno virus, isolate tth31, complete genome |
| AJ620226.1 | Torque teno virus, isolate tth4, complete genome |
| AJ620227.1 | Torque teno virus, isolate tth5, complete genome |
| AJ620228.1 | Torque teno virus, isolate tth14, complete genome |
| AJ620229.1 | Torque teno virus, isolate tth29, complete genome |
| AJ620230.1 | Torque teno virus, isolate tth7, complete genome |
| AJ620231.1 | Torque teno virus, isolate tth8, complete genome |
| AJ620232.1 | Torque teno virus, isolate tth13, complete genome |
| AJ620233.1 | Torque teno virus, isolate tth19, complete genome |
| AJ620234.1 | Torque teno virus, isolate tth22g4, complete genome |
| AJ620235.1 | Torque teno virus, isolate tth23, complete genome |
| AM711976.1 | TT virus sle1957 complete genome |
| AM712003.1 | TT virus sle1931 complete genome |
| AM712004.1 | TT virus sle1932 complete genome |
| AM712030.1 | TT virus sle2057 complete genome |
| AM712031.1 | TT virus sle2058 complete genome |
| AM712032.1 | TT virus sle2072 complete genome |
| AM712033.1 | TT virus sle2061 complete genome |
| AM712034.1 | TT virus sle2065 complete genome |
| AY026465.1 | TT virus isolate L01 ORF2 and ORF1 genes, complete cds |
| AY026466.1 | TT virus isolate L02 ORF2 and ORF1 genes, complete cds |
| DQ003341.1 | Torque teno virus clone P2-9-02 ORF2 (ORF2), ORFIA (ORF1A), and ORF1B |
| | (ORF1B) genes, complete cds |
| DQ003342.1 | Torque teno virus clone P2-9-07 ORF2 (ORF2), ORF1A (ORF1A), and ORF1B |
| | (ORF1B) genes, complete cds |
| DQ003343.1 | Torque teno virus clone P2-9-08 ORF2 (ORF2), ORF1A (ORF1A), and ORF1B |
| | (ORF1B) genes, complete cds |
| DQ003344.1 | Torque teno virus clone P2-9-16 ORF2 (ORF2), ORF1A (ORF1A), and ORF1B |
| | (ORF1B) genes, complete cds |
| DQ186994.1 | Torque teno virus clone P601 ORF2 (ORF2) and ORF1 (ORF1) genes, complete |
| | cds |
| DQ186995.1 | Torque teno virus clone P605 ORF2 (ORF2) and ORF1 (ORF1) genes, complete |
| | cds |
| DQ186996.1 | Torque teno virus clone BM1A-02 ORF2 (ORF2) and ORF1 (ORF1) genes, |
| | complete cds |
| DQ186997.1 | Torque teno virus clone BM1A-09 ORF2 (ORF2) and ORF1 (ORF1) genes, |
| | complete cds |
| DQ186998.1 | Torque teno virus clone BM1A-13 ORF2 (ORF2) and ORF1 (ORF1) genes, |
| | complete cds |
| DQ186999.1 | Torque teno virus clone BM1B-05 ORF2 (ORF2) and ORF1 (ORF1) genes, |
| | complete cds |
| DQ187000.1 | Torque teno virus clone BM1B-07 ORF2 (ORF2) and ORF1 (ORF1) genes, |
| | complete cds |
| DQ187001.1 | Torque teno virus clone BM1B-11 ORF2 (ORF2) and ORF1 (ORF1) genes, |
| | complete cds |
| DQ187002.1 | Torque teno virus clone BM1B-14 ORF2 (ORF2) and ORF1 (ORF1) genes, |
| | complete cds |
| DQ187003.1 | Torque teno virus clone BM1B-08 ORF2 (ORF2) gene, complete cds; and |
| | nonfunctional ORF1 (ORF1) gene, complete sequence |
| DQ187004.1 | Torque teno virus clone BM1C-16 ORF2 (ORF2) and ORF1 (ORF1) genes, |
| | complete cds |
| DQ187005.1 | Torque teno virus clone BM1C-10 ORF2 (ORF2) and ORF1 (ORF1) genes, |
| | complete cds |
| DQ187007.1 | Torque teno virus clone BM2C-25 ORF2 (ORF2) gene, complete cds; and |
| | nonfunctional ORF1 (ORF1) gene, complete sequence |
| DQ361268.1 | Torque teno virus isolate ViPi04 ORF1 gene, complete cds |
| EF538879.1 | Torque teno virus isolate CSC5 ORF2 and ORF1 genes, complete cds |
| EU305675.1 | Torque teno virus isolate LTT7 ORF1 gene, complete cds |
| EU305676.1 | Torque teno virus isolate LTT10 ORF1 gene, complete cds |
| EU889253.1 | Torque teno virus isolate ViPi08 nonfunctional ORF1 gene, complete sequence |
| FJ392105.1 | Torque teno virus isolate TW53A25 ORF2 gene, partial cds; and ORF1 gene, |
| | complete cds |
| FJ392107.1 | Torque teno virus isolate TW53A27 ORF2 gene, partial cds; and ORF1 gene, |
| | complete cds |
| FJ392108.1 | Torque teno virus isolate TW53A29 ORF2 gene, partial cds; and ORF1 gene, |
| | complete cds |
| FJ392111.1 | Torque teno virus isolate TW53A35 ORF2 gene, partial cds; and ORF1 gene, |
| | complete cds |
| FJ392112.1 | Torque teno virus isolate TW53A39 ORF2 gene, partial cds; and ORF1 gene, |
| | complete cds |
| FJ392113.1 | Torque teno virus isolate TW53A26 ORF2 gene, complete cds; and nonfunctional |
| | ORF1 gene, complete sequence |
| FJ392114.1 | Torque teno virus isolate TW53A30 ORF2 and ORF1 genes, complete cds |
| FJ392115.1 | Torque teno virus isolate TW53A31 ORF2 and ORF1 genes, complete cds |
| FJ392117.1 | Torque teno virus isolate TW53A37 ORF1 gene, complete cds |
| FJ426280.1 | Torque teno virus strain SIA109, complete genome |
| FR751500.1 | Torque teno virus complete genome, isolate TTV-HD23a (rheu215) |
| GU797360.1 | Torque teno virus clone 8-17, complete genome |
| HC742700.1 | Sequence 7 from Patent WO2010044889 |
| HC742710.1 | Sequence 17 from Patent WO2010044889 |
| JX134044.1 | TTV-like mini virus isolate TTMV_LY1, complete genome |
| JX134045.1 | TTV-like mini virus isolate TTMV_LY2, complete genome |
| KU243129.1 | TTV-like mini virus isolate TTMV-204, complete genome |
| KY856742.1 | TTV-like mini virus isolate zhenjiang, complete genome |
| LC381845.1 | Torque teno virus Human/Japan/KS025/2016 DNA, complete genome |
| MH648892.1 | Anelloviridae sp. isolate ctdc048, complete genome |
| MH648893.1 | Anelloviridae sp. isolate ctdh007, complete genome |
| MH648897.1 | Anelloviridae sp. isolate ctcb038, complete genome |
| MH648900.1 | Anelloviridae sp. isolate ctfc019, complete genome |
| MH648901.1 | Anelloviridae sp. isolate ctbb022, complete genome |
| MH648907.1 | Anelloviridae sp. isolate ctcf040, complete genome |
| MH648911.1 | Anelloviridae sp. isolate cthi018, complete genome |
| MH648912.1 | Anelloviridae sp. isolate ctea38, complete genome |
| MH648913.1 | Anelloviridae sp. isolate ctbg006, complete genome |
| MH648916.1 | Anelloviridae sp. isolate ctbg020, complete genome |
| MH648925.1 | Anelloviridae sp. isolate ctci019, complete genome |
| MH648932.1 | Anelloviridae sp. isolate ctid031, complete genome |
| MH648946.1 | Anelloviridae sp. isolate ctdb017, complete genome |
| MH648957.1 | Anelloviridae sp. isolate ctch017, complete genome |
| MH648958.1 | Anelloviridae sp. isolate ctbh011, complete genome |
| MH648959.1 | Anelloviridae sp. isolate ctbc020, complete genome |
| MH648962.1 | Anelloviridae sp. isolate ctif015, complete genome |
| MH648966.1 | Anelloviridae sp. isolate ctei055, complete genome |
| MH648969.1 | Anelloviridae sp. isolate ctjg000, complete genome |
| MH648976.1 | Anelloviridae sp. isolate ctcj064, complete genome |
| MH648977.1 | Anelloviridae sp. isolate ctbj022, complete genome |
| MH648982.1 | Anelloviridae sp. isolate ctbf014, complete genome |
| MH648983.1 | Anelloviridae sp. isolate ctbd027, complete genome |
| MH648985.1 | Anelloviridae sp. isolate ctch016, complete genome |
| MH648986.1 | Anelloviridae sp. isolate ctbd020, complete genome |
| MH648989.1 | Anelloviridae sp. isolate ctga035, complete genome |
| MH648990.1 | Anelloviridae sp. isolate cthf001, complete genome |
| MH648995.1 | Anelloviridae sp. isolate ctbd067, complete genome |
| MH648997.1 | Anelloviridae sp. isolate ctce026, complete genome |
| MH648999.1 | Anelloviridae sp. isolate ctfb058, complete genome |
| MH649002.1 | Anelloviridae sp. isolate ctjj046, complete genome |
| MH649006.1 | Anelloviridae sp. isolate ctcf030, complete genome |
| MH649008.1 | Anelloviridae sp. isolate ctbg025, complete genome |
| MH649011.1 | Anelloviridae sp. isolate ctbh052, complete genome |
| MH649014.1 | Anelloviridae sp. isolate ctba003, complete genome |
| MH649017.1 | Anelloviridae sp. isolate ctbb016, complete genome |
| MH649022.1 | Anelloviridae sp. isolate ctch023, complete genome |
| MH649023.1 | Anelloviridae sp. isolate ctbd051, complete genome |
| MH649028.1 | Anelloviridae sp. isolate ctbf9, complete genome |
| MH649038.1 | Anelloviridae sp. isolate ctbi030, complete genome |
| MH649039.1 | Anelloviridae sp. isolate ctca057, complete genome |
| MH649040.1 | Anelloviridae sp. isolate ctch033, complete genome |
| MH649042.1 | Anelloviridae sp. isolate ctjd005, complete genome |
| MH649045.1 | Anelloviridae sp. isolate ctdc021, complete genome |
| MH649051.1 | Anelloviridae sp. isolate ctdg044, complete genome |
| MH649056.1 | Anelloviridae sp. isolate ctcc062, complete genome |
| MH649061.1 | Anelloviridae sp. isolate ctid009, complete genome |
| MH649062.1 | Anelloviridae sp. isolate ctdc018, complete genome |
| MH649063.1 | Anelloviridae sp. isolate ctbf012, complete genome |
| MH649068.1 | Anelloviridae sp. isolate ctcc066, complete genome |
| MH649070.1 | Anelloviridae sp. isolate ctda011, complete genome |
| MH649077.1 | Anelloviridae sp. isolate ctbh034, complete genome |
| MH649083.1 | Anelloviridae sp. isolate ctdg028, complete genome |
| MH649084.1 | Anelloviridae sp. isolate ctii061, complete genome |
| MH649085.1 | Anelloviridae sp. isolate cteh021, complete genome |
| MH649092.1 | Anelloviridae sp. isolate ctbg012, complete genome |
| MH649101.1 | Anelloviridae sp. isolate ctif053, complete genome |
| MH649104.1 | Anelloviridae sp. isolate ctei657, complete genome |
| MH649106.1 | Anelloviridae sp. isolate ctca015, complete genome |
| MH649114.1 | Anelloviridae sp. isolate ctbf050, complete genome |
| MH649122.1 | Anelloviridae sp. isolate ctdc002, complete genome |
| MH649125.1 | Anelloviridae sp. isolate ctbb15, complete genome |
| MH649127.1 | Anelloviridae sp. isolate ctba013, complete genome |
| MH649137.1 | Anelloviridae sp. isolate ctbb000, complete genome |
| MH649141.1 | Anelloviridae sp. isolate ctbc019, complete genome |
| MH649142.1 | Anelloviridae sp. isolate ctid026, complete genome |
| MH649144.1 | Anelloviridae sp. isolate ctfj004, complete genome |
| MH649152.1 | Anelloviridae sp. isolate ctcj13, complete genome |
| MH649156.1 | Anelloviridae sp. isolate ctci006, complete genome |
| MH649157.1 | Anelloviridae sp. isolate ctbd025, complete genome |
| MH649158.1 | Anelloviridae sp. isolate ctbf005, complete genome |
| MH649161.1 | Anelloviridae sp. isolate ctcf045, complete genome |
| MH649165.1 | Anelloviridae sp. isolate ctcc29, complete genome |
| MH649169.1 | Anelloviridae sp. isolate ctib021, complete genome |
| MH649172.1 | Anelloviridae sp. isolate ctbh857, complete genome |
| MH649174.1 | Anelloviridae sp. isolate ctbj049, complete genome |
| MH649178.1 | Anelloviridae sp. isolate ctfc006, complete genome |
| MH649179.1 | Anelloviridae sp. isolate ctbe000, complete genome |
| MH649183.1 | Anelloviridae sp. isolate ctbb031, complete genome |
| MH649186.1 | Anelloviridae sp. isolate ctcb33, complete genome |
| MH649189.1 | Anelloviridae sp. isolate ctcc12, complete genome |
| MH649196.1 | Anelloviridae sp. isolate ctci060, complete genome |
| MH649199.1 | Anelloviridae sp. isolate ctbb017, complete genome |
| MH649203.1 | Anelloviridae sp. isolate cthc018, complete genome |
| MH649204.1 | Anelloviridae sp. isolate ctbj003, complete genome |
| MH649206.1 | Anelloviridae sp. isolate ctbg010, complete genome |
| MH649208.1 | Anelloviridae sp. isolate ctid008, complete genome |
| MH649209.1 | Anelloviridae sp. isolate ctbg056, complete genome |
| MH649210.1 | Anelloviridae sp. isolate ctda001, complete genome |
| MH649212.1 | Anelloviridae sp. isolate ctcf004, complete genome |
| MH649217.1 | Anelloviridae sp. isolate ctbe029, complete genome |
| MH649223.1 | Anelloviridae sp. isolate ctci016, complete genome |
| MH649224.1 | Anelloviridae sp. isolate ctce11, complete genome |
| MH649228.1 | Anelloviridae sp. isolate ctcf013, complete genome |
| MH649229.1 | Anelloviridae sp. isolate ctcb036, complete genome |
| MH649241.1 | Anelloviridae sp. isolate ctda027, complete genome |
| MH649242.1 | Anelloviridae sp. isolate ctbf003, complete genome |
| MH649254.1 | Anelloviridae sp. isolate ctjb007, complete genome |
| MH649255.1 | Anelloviridae sp. isolate ctbb023, complete genome |
| MH649256.1 | Anelloviridae sp. isolate ctca002, complete genome |
| MH649258.1 | Anelloviridae sp. isolate ctcg010, complete genome |
| MH649263.1 | Anelloviridae sp. isolate ctgh3, complete genome |
| MK012439.1 | Anelloviridae sp. isolate cthe000, complete genome |
| MK012440.1 | Anelloviridae sp. isolate ctjd008, complete genome |
| MK012448.1 | Anelloviridae sp. isolate ctch012, complete genome |
| MK012457.1 | Anelloviridae sp. isolate ctda009, complete genome |
| MK012458.1 | Anelloviridae sp. isolate ctcd015, complete genome |
| MK012485.1 | Anelloviridae sp. isolate ctfd011, complete genome |
| MK012489.1 | Anelloviridae sp. isolate ctba003, complete genome |
| MK012492.1 | Anelloviridae sp. isolate ctbb005, complete genome |
| MK012493.1 | Anelloviridae sp. isolate ctcj014, complete genome |
| MK012500.1 | Anelloviridae sp. isolate ctcb001, complete genome |
| MK012504.1 | Anelloviridae sp. isolate ctcj010, complete genome |
| MK012516.1 | Anelloviridae sp. isolate ctcf003, complete genome |
| NC_038336.1 | Torque teno virus 5 isolate TCHN-C1 Orf2 and Orf1 genes, complete cds |
| NC_038338.1 | Torque teno virus 11 isolate TCHN-D1 Orf2 and Orf1 genes, complete cds |
| NC_038339.1 | Torque teno virus 13 isolate TCHN-A Orf2 and Orf1 genes, complete cds |
| NC_038340.1 | Torque teno virus 20 ORF4, ORF3, ORF2, ORF1 genes, complete cds, clone: |
| | SAa-10 |
| NC_038341.1 | Torque teno virus 21 isolate TCHN-B ORF2 and ORF1 genes, complete cds |
| NC_038342.1 | Torque teno virus 23 ORF2, ORF1 genes, complete cds, isolate: s-TTV CH65-2 |
| NC_038343.1 | Torque teno virus 24 ORF4, ORF3, ORF2, ORF1 genes, complete cds, clone: |
| | SAa-01 |
| NC_038344.1 | Torque teno virus 29 ORF2, ORF1, ORF3 genes, complete cds, isolate: TTVyon- |
| | KC009 |
| NC_038345.1 | Torque teno mini virus 10 isolate LIL-y1 ORF2, ORF1, ORF3, and ORF4 genes, |
| | complete cds |
| NC_038346.1 | Torque teno mini virus 11 isolate LIL-y2 ORF2, ORF1, and ORF3 genes, |
| | complete cds |
| NC_038347.1 | Torque teno mini virus 12 isolate LIL-y3 ORF2, ORF1, ORF3, and ORF4 genes, |
| | complete cds |
| NC_038350.1 | Torque teno midi virus 3 isolate 2PoSMA ORF2 and ORF1 genes, complete cds |
| NC_038351.1 | Torque teno midi virus 4 isolate 6PoSMA ORF2, ORF1, and ORF3 genes, |
| | complete cds |
| NC_038352.1 | Torque teno midi virus 5 DNA, complete genome, isolate: MDJHem2 |
| NC_038353.1 | Torque teno midi virus 6 DNA, complete genome, isolate: MDJHem3-1 |
| NC_038354.1 | Torque teno midi virus 7 DNA, complete genome, isolate: MDJHem3-2 |
| NC_038355.1 | Torque teno midi virus 8 DNA, complete genome, isolate: MDJN1 |
| NC_038356.1 | Torque teno midi virus 9 DNA, complete genome, isolate: MDJN2 |
| NC_038357.1 | Torque teno midi virus 10 DNA, complete genome, isolate: MDJN14 |
| NC_038358.1 | Torque teno midi virus 11 DNA, complete genome, isolate: MDJN47 |
| NC_038359.1 | Torque teno midi virus 12 DNA, complete genome, isolate: MDJN51 |
| NC_038360.1 | Torque teno midi virus 13 DNA, complete genome, isolate: MDJN69 |
| NC_038361.1 | Torque teno midi virus 14 DNA, complete genome, isolate: MDJN97 |
| NC_038362.1 | Torque teno midi virus 15 DNA, complete genome, isolate: Pt-TTMDV210 |

In some embodiments, the genetic element comprises one or more sequences with homology or identity to one or more sequences from one or more non-Anelloviruses, e.g., adenovirus, herpes virus, pox virus, vaccinia virus, SV40, papilloma virus, an RNA virus such as a retrovirus, e.g., lentivirus, a single-stranded RNA virus, e.g., hepatitis virus, or a double-stranded RNA virus e.g., rotavirus. Since, in some embodiments, recombinant retroviruses are defective, assistance may be provided order to produce infectious particles. Such assistance can be provided, e.g., by using helper cell lines that contain plasmids encoding all of the structural genes of the retrovirus under the control of regulatory sequences within the LTR. Suitable cell lines for replicating the anellovectors described herein include cell lines known in the art, e.g., A549 cells, which can be modified as described herein. Said genetic element can additionally contain a gene encoding a selectable marker so that the desired genetic elements can be identified.

In some embodiments, the genetic element includes non-silent mutations, e.g., base substitutions, deletions, or additions resulting in amino acid differences in the encoded polypeptide, so long as the sequence remains at least about 70%, 75%, 80%, 85%, 90%, 95%, 96%, 97%, 98%, or 99% identical to the polypeptide encoded by the first nucleotide sequence or otherwise is useful for practicing the present invention. In this regard, certain conservative amino acid substitutions may be made which are generally recognized not to inactivate overall protein function: such as in regard of positively charged amino acids (and vice versa), lysine, arginine and histidine; in regard of negatively charged amino acids (and vice versa), aspartic acid and glutamic acid; and in regard of certain groups of neutrally charged amino acids (and in all cases, also vice versa), (1) alanine and serine, (2) asparagine, glutamine, and histidine, (3) cysteine and serine, (4) glycine and proline, (5) isoleucine, leucine and valine, (6) methionine, leucine and isoleucine, (7) phenylalanine, methionine, leucine, and tyrosine, (8) serine and threonine, (9) tryptophan and tyrosine, (10) and for example tyrosine, tryptophan and phenylalanine. Amino acids can be classified according to physical properties and contribution to secondary and tertiary protein structure. A conservative substitution is recognized in the art as a substitution of one amino acid for another amino acid that has similar properties.

Identity of two or more nucleic acid or polypeptide sequences having the same or a specified percentage of nucleotides or amino acid residues that are the same (e.g., about 60%, 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) may be measured using a BLAST or BLAST 2.0 sequence comparison algorithms with default parameters described below, or by manual alignment and visual inspection (see, e.g., NCBI web site www.ncbi.nlm.nih.gov/BLAST/ or the like). Identity may also refer to, or may be applied to, the compliment of a test sequence. Identity also includes sequences that have deletions and/or additions, as well as those that have substitutions. As described herein, the algorithms account for gaps and the like. Identity may exist over a region that is at least about 10 amino acids or nucleotides in length, about 15 amino acids or nucleotides in length, about 20 amino acids or nucleotides in length, about 25 amino acids or nucleotides in length, about 30 amino acids or nucleotides in length, about 35 amino acids or nucleotides in length, about 40 amino acids or nucleotides in length, about 45 amino acids or nucleotides in length, about 50 amino acids or nucleotides in length, or more. Since the genetic code is degenerate, a homologous nucleotide sequence can include any number of silent base changes, i.e., nucleotide substitutions that nonetheless encode the same amino acid.

Proteinaceous Exterior In some embodiments, the anellovector, e.g., synthetic anellovector, comprises a proteinaceous exterior that encloses the genetic element. The proteinaceous exterior can comprise a substantially non-pathogenic exterior protein that fails to elicit an unwanted immune response in a mammal. The proteinaceous exterior of the anellovectors typically comprises a substantially non-pathogenic protein that may self-assemble into an icosahedral formation that makes up the proteinaceous exterior.

In some embodiments, the proteinaceous exterior protein is encoded by a sequence of the genetic element of the anellovector (e.g., is in cis with the genetic element). In other embodiments, the proteinaceous exterior protein is encoded by a nucleic acid separate from the genetic element of the anellovector (e.g., is in trans with the genetic element) (e.g., a bacmid as described herein).

In some embodiments, the protein, e.g., substantially non-pathogenic protein and/or proteinaceous exterior protein, comprises one or more glycosylated amino acids, e.g., 2, 3, 4, 5, 6, 7, 8, 9, 10, or more.

In some embodiments, the protein, e.g., substantially non-pathogenic protein and/or proteinaceous exterior protein comprises at least one hydrophilic DNA-binding region, an arginine-rich region, a threonine-rich region, a glutamine-rich region, a N-terminal polyarginine sequence, a variable region, a C-terminal polyglutamine/glutamate sequence, and one or more disulfide bridges.

In some embodiments, the protein is a capsid protein, e.g., has a sequence having at least about 60%, 65%, 70%, 75%, 80%, 85%, 90% 95%, 96%, 97%, 98%, 99%, or 100% sequence identity to a protein encoded by any one of the nucleotide sequences encoding a capsid protein described herein, e.g., an Anellovirus ORF1 molecule and/or capsid protein sequence, e.g., as described herein. In some embodiments, the protein or a functional fragment of a capsid protein is encoded by a nucleotide sequence having at least about 60%, 70% 80%, 85%, 90% 95%, 96%, 97%, 98%, 99%, or 100% sequence identity to an Anellovirus ORF1 nucleic acid, e.g., as described herein.

In some embodiments, the anellovector comprises a nucleotide sequence encoding a capsid protein or a functional fragment of a capsid protein or a sequence having at least about 60%, 70% 80%, 85%, 90% 95%, 96%, 97%, 98%, 99%, or 100% sequence identity to an Anellovirus ORF1 molecule as described herein.

In some embodiments, the ranges of amino acids with less sequence identity may provide one or more of the properties described herein and differences in cell/tissue/species specificity (e.g. tropism).

In some embodiments, the anellovector lacks lipids in the proteinaceous exterior. In some embodiments, the anellovector lacks a lipid bilayer, e.g., a viral envelope. In some embodiments, the interior of the anellovector is entirely covered (e.g., 100% coverage) by a proteinaceous exterior. In some embodiments, the interior of the anellovector is less than 100% covered by the proteinaceous exterior, e.g., 95%, 90%, 85%, 80%, 70%, 60%, 50% or less coverage. In some embodiments, the proteinaceous exterior comprises gaps or discontinuities, e.g., permitting permeability to water, ions, peptides, or small molecules, so long as the genetic element is retained in the anellovector.

In some embodiments, the proteinaceous exterior comprises one or more proteins or polypeptides that specifically recognize and/or bind a host cell, e.g., a complementary protein or polypeptide, to mediate entry of the genetic element into the host cell.

In some embodiments, the proteinaceous exterior comprises one or more of the following: an arginine-rich region, jelly-roll region, N22 domain, hypervariable region, and/or C-terminal domain, e.g., of an ORF1 molecule, e.g., as described herein. In some embodiments, the proteinaceous exterior comprises one or more of the following: one or more glycosylated proteins, a hydrophilic DNA-binding region, an arginine-rich region, a threonine-rich region, a glutamine-rich region, a N-terminal polyarginine sequence, a variable region, a C-terminal polyglutamine/glutamate sequence, and one or more disulfide bridges. For example, the proteinaceous exterior comprises a protein encoded by an Anellovirus ORF1 nucleic acid, e.g., as described herein.

In some embodiments, the proteinaceous exterior comprises one or more of the following characteristics: an icosahedral symmetry, recognizes and/or binds a molecule that interacts with one or more host cell molecules to mediate entry into the host cell, lacks lipid molecules, lacks carbohydrates, is pH and temperature stable, is detergent resistant, and is substantially non-immunogenic or non-pathogenic in a host.

### III. Nucleic Acid Constructs

The genetic element described herein may be included in a nucleic acid construct (e.g., a bacmid or donor vector, e.g., as described herein).

In one aspect, the invention includes a nucleic acid genetic element construct (e.g., a bacmid or donor vector) comprising a genetic element comprising (i) a sequence encoding a non-pathogenic exterior protein (e.g., an Anellovirus ORF1 molecule or a splice variant or functional fragment thereof), (ii) an exterior protein binding sequence that binds the genetic element to the non-pathogenic exterior protein, and (iii) a sequence encoding an effector. In some embodiments, the nucleic acid genetic element construct comprises one or more baculovirus elements (e.g., a baculovirus genome). In embodiments, the nucleic acid genetic element construct, when introduced in to a suitable host cell (e.g., an insect cell, e.g., an Sf9 cell) comprises elements sufficient to drive production of a baculovirus (e.g., comprising a nucleic acid construct, e.g., a bacmid) in the host cell.

The genetic element or any of the sequences within the genetic element can be obtained using any suitable method. Various recombinant methods are known in the art, such as, for example screening libraries from cells harboring viral sequences, deriving the sequences from a nucleic acid construct known to include the same, or isolating directly from cells and tissues containing the same, using standard techniques. Alternatively or in combination, part or all of the genetic element can be produced synthetically, rather than cloned.

In some embodiments, the nucleic acid construct includes regulatory elements, nucleic acid sequences homologous to target genes, and/or various reporter constructs for causing the expression of reporter molecules within a viable cell and/or when an intracellular molecule is present within a target cell.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al., 2000 FEBS Letters 479: 79-82). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

In some embodiments, the nucleic acid construct is substantially non-pathogenic and/or substantially non-integrating in a host cell or is substantially non-immunogenic in a host.

In some embodiments, the nucleic acid construct is in an amount sufficient to modulate one or more of phenotype, virus levels, gene expression, compete with other viruses, disease state, etc. at least about 5%, 10%, 15%, 20%, 25%, 30%, 35%, 40%, 45%, 50%, or more.

### IV. Compositions

The anellovectors described herein may also be included in pharmaceutical compositions with a pharmaceutical excipient, e.g., as described herein. In some embodiments, the pharmaceutical composition comprises at least 10⁵, 10⁶, 10⁷, 10⁸, 10⁹, 10¹⁰, 10¹¹, 10¹², 10¹³, 10¹⁴, or 10¹⁵ anellovectors. In some embodiments, the pharmaceutical composition comprises about 10⁵-10¹⁵, 10⁵-10¹⁰, or 10¹⁰-10¹⁵ anellovectors. In some embodiments, the pharmaceutical composition comprises about 10⁸ (e.g., about 10⁵, 10⁶, 10⁷, 10⁸, 10⁹, or 10¹⁰) genomic equivalents/mL of the anellovector. In some embodiments, the pharmaceutical composition comprises 10⁵-10¹⁰, 10⁶-10¹⁰, 10⁷-10¹⁰, 10⁸-10¹⁰, 10⁹-10¹⁰, 10⁵-10⁶, 10⁵-10⁷, 10⁵-10⁸, 10⁵-10⁹, 10⁵-10¹¹, 10⁵-10¹², 10⁵-10¹³, 10⁵-10¹⁴, 10⁵-10¹⁵, or 10¹⁰-10¹⁵ genomic equivalents/mL of the anellovector, e.g., as determined according to the method of Example 18 of PCT/US19/65995. In some embodiments, the pharmaceutical composition comprises sufficient anellovectors to deliver at least 1, 2, 5, or 10, 100, 500, 1000, 2000, 5000, 8,000, 1×10⁴, 1×10⁵, 1×10⁶, 1×10⁷ or greater copies of a genetic element comprised in the anellovectors per cell to a population of the eukaryotic cells. In some embodiments, the pharmaceutical composition comprises sufficient anellovectors to deliver at least about 1×10⁴, 1×10⁵, 1×10⁶, 1× or 10⁷, or about 1×10⁴-1×10⁵, 1×10⁴-1×10⁶, 1×10⁴-1×10⁷, 1×10⁵-1×10⁶, 1×10⁵-1×10⁷, or 1×10⁶-1×10⁷ copies of a genetic element comprised in the anellovectors per cell to a population of the eukaryotic cells.

In some embodiments, the pharmaceutical composition has one or more of the following characteristics: the pharmaceutical composition meets a pharmaceutical or good manufacturing practices (GMP) standard; the pharmaceutical composition was made according to good manufacturing practices (GMP); the pharmaceutical composition has a pathogen level below a predetermined reference value, e.g., is substantially free of pathogens; the pharmaceutical composition has a contaminant level below a predetermined reference value, e.g., is substantially free of contaminants; or the pharmaceutical composition has low immunogenicity or is substantially non-immunogenic, e.g., as described herein.

In some embodiments, the pharmaceutical composition comprises below a threshold amount of one or more contaminants. Exemplary contaminants that are desirably excluded or minimized in the pharmaceutical composition include, without limitation, host cell nucleic acids (e.g., host cell DNA and/or host cell RNA), animal-derived components (e.g., serum albumin or trypsin), replication-competent viruses, non-infectious particles, free viral capsid protein, adventitious agents, and aggregates. In embodiments, the contaminant is host cell DNA. In embodiments, the composition comprises less than about 10 ng of host cell DNA per dose. In embodiments, the level of host cell DNA in the composition is reduced by filtration and/or enzymatic degradation of host cell DNA. In embodiments, the pharmaceutical composition consists of less than 10% (e.g., less than about 10%, 5%, 4%, 3%, 2%, 1%, 0.5%, or 0.1%) contaminant by weight.

In one aspect, the invention described herein includes a pharmaceutical composition comprising:
a) an anellovector comprising a genetic element comprising (i) a sequence encoding a non-pathogenic exterior protein, (ii) an exterior protein binding sequence that binds the genetic element to the non-pathogenic exterior protein, and (iii) a sequence encoding a regulatory nucleic acid; and a proteinaceous exterior that is associated with, e.g., envelops or encloses, the genetic element; and
b) a pharmaceutical excipient.

### Vesicles

In some embodiments, the composition further comprises a carrier component, e.g., a microparticle, liposome, vesicle, or exosome. In some embodiments, liposomes comprise spherical vesicle structures composed of a uni- or multilamellar lipid bilayer surrounding internal aqueous compartments and a relatively impermeable outer lipophilic phospholipid bilayer. Liposomes may be anionic, neutral or cationic. Liposomes are generally biocompatible, nontoxic, can deliver both hydrophilic and lipophilic drug molecules, protect their cargo from degradation by plasma enzymes, and transport their load across biological membranes (see, e.g., Spuch and Navarro, Journal of Drug Delivery, vol. 2011, Article ID 469679, 12 pages, 2011. doi:10.1155/2011/469679 for review).

Vesicles can be made from several different types of lipids; however, phospholipids are most commonly used to generate liposomes as drug carriers. Vesicles may comprise without limitation DOTMA, DOTAP, DOTIM, DDAB, alone or together with cholesterol to yield DOTMA and cholesterol, DOTAP and cholesterol, DOTIM and cholesterol, and DDAB and cholesterol. Methods for preparation of multilamellar vesicle lipids are known in the art (see for example U.S. Pat. No. 6,693,086, the teachings of which relating to multilamellar vesicle lipid preparation are incorporated herein by reference). Although vesicle formation can be spontaneous when a lipid film is mixed with an aqueous solution, it can also be expedited by applying force in the form of shaking by using a homogenizer, sonicator, or an extrusion apparatus (see, e.g., Spuch and Navarro, Journal of Drug Delivery, vol. 2011, Article ID 469679, 12 pages, 2011. doi:10.1155/2011/469679 for review). Extruded lipids can be prepared by extruding through filters of decreasing size, as described in Templeton et al., Nature Biotech, 15:647-652, 1997, the teachings of which relating to extruded lipid preparation are incorporated herein by reference.

As described herein, additives may be added to vesicles to modify their structure and/or properties. For example, either cholesterol or sphingomyelin may be added to the mixture to help stabilize the structure and to prevent the leakage of the inner cargo. Further, vesicles can be prepared from hydrogenated egg phosphatidylcholine or egg phosphatidylcholine, cholesterol, and dicetyl phosphate. (see, e.g., Spuch and Navarro, Journal of Drug Delivery, vol. 2011, Article ID 469679, 12 pages, 2011. doi:10.1155/2011/469679 for review). Also, vesicles may be surface modified during or after synthesis to include reactive groups complementary to the reactive groups on the recipient cells. Such reactive groups include without limitation maleimide groups. As an example, vesicles may be synthesized to include maleimide conjugated phospholipids such as without limitation DSPE-MaL-PEG2000.

A vesicle formulation may be mainly comprised of natural phospholipids and lipids such as 1,2-distearoryl-sn-glycero-3-phosphatidyl choline (DSPC), sphingomyelin, egg phosphatidylcholines and monosialoganglioside. Formulations made up of phospholipids only are less stable in plasma. However, manipulation of the lipid membrane with cholesterol reduces rapid release of the encapsulated cargo or 1,2-dioleoyl-sn-glycero-3-phosphoethanolamine (DOPE) increases stability (see, e.g., Spuch and Navarro, Journal of Drug Delivery, vol. 2011, Article ID 469679, 12 pages, 2011. doi:10.1155/2011/469679 for review).

In embodiments, lipids may be used to form lipid microparticles. Lipids include, but are not limited to, DLin-KC2-DMA4, C12-200 and colipids disteroylphosphatidyl choline, cholesterol, and PEG-DMG may be formulated (see, e.g., Novobrantseva, Molecular Therapy-Nucleic Acids (2012) 1, e4; doi:10.1038/mtna.2011.3) using a spontaneous vesicle formation procedure. The component molar ratio may be about 50/10/38.5/1.5 (DLin-KC2-DMA or C12-200/disteroylphosphatidyl choline/cholesterol/PEG-DMG). Tekmira has a portfolio of approximately 95 patent families, in the U.S. and abroad, that are directed to various aspects of lipid microparticles and lipid microparticles formulations (see, e.g., U.S. Pat. Nos. 7,982,027; 7,799,565; 8,058,069; 8,283,333; 7,901,708; 7,745,651; 7,803,397; 8,101,741; 8,188,263; 7,915,399; 8,236,943 and 7,838,658 and European Pat. Nos. 1766035; 1519714; 1781593 and 1664316), all of which may be used and/or adapted to the present invention.

In some embodiments, microparticles comprise one or more solidified polymer(s) that is arranged in a random manner. The microparticles may be biodegradable. Biodegradable microparticles may be synthesized, e.g., using methods known in the art including without limitation solvent evaporation, hot melt microencapsulation, solvent removal, and spray drying. Exemplary methods for synthesizing microparticles are described by Bershteyn et al., Soft Matter 4:1787-1787, 2008 and in US 2008/0014144 A1, the specific teachings of which relating to microparticle synthesis are incorporated herein by reference.

Exemplary synthetic polymers which can be used to form biodegradable microparticles include without limitation aliphatic polyesters, poly (lactic acid) (PLA), poly (glycolic acid) (PGA), co-polymers of lactic acid and glycolic acid (PLGA), polycarprolactone (PCL), polyanhydrides, poly(ortho)esters, polyurethanes, poly(butyric acid), poly(valeric acid), and poly(lactide-co-caprolactone), and natural polymers such as albumin, alginate and other polysaccharides including dextran and cellulose, collagen, chemical derivatives thereof, including substitutions, additions of chemical groups such as for example alkyl, alkylene, hydroxylations, oxidations, and other modifications routinely made by those skilled in the art), albumin and other hydrophilic proteins, zein and other prolamines and hydrophobic proteins, copolymers and mixtures thereof. In general, these materials degrade either by enzymatic hydrolysis or exposure to water, by surface or bulk erosion.

The microparticles' diameter ranges from 0.1-1000 micrometers (μm). In some embodiments, their diameter ranges in size from 1-750 μm, or from 50-500 μm, or from 100-250 μm. In some embodiments, their diameter ranges in size from 50-1000 μm, from 50-750 μm, from 50-500 μm, or from 50-250 μm. In some embodiments, their diameter ranges in size from 0.05-1000 μm, from 10-1000 μm, from 100-1000 μm, or from 500-1000 μm. In some embodiments, their diameter is about 0.5 μm, about 10 μm, about 50 μm, about 100 μm, about 200 μm, about 300 μm, about 350 μm, about 400 μm, about 450 μm, about 500 μm, about 550 μm, about 600 μm, about 650 μm, about 700 μm, about 750 μm, about 800 μm, about 850 μm, about 900 μm, about 950 μm, or about 1000 μm. As used in the context of microparticle diameters, the term “about” means+/−5% of the absolute value stated.

In some embodiments, a ligand is conjugated to the surface of the microparticle via a functional chemical group (carboxylic acids, aldehydes, amines, sulfhydryls and hydroxyls) present on the surface of the particle and present on the ligand to be attached. Functionality may be introduced into the microparticles by, for example, during the emulsion preparation of microparticles, incorporation of stabilizers with functional chemical groups.

Another example of introducing functional groups to the microparticle is during post-particle preparation, by direct crosslinking particles and ligands with homo- or heterobifunctional crosslinkers. This procedure may use a suitable chemistry and a class of crosslinkers (CDI, EDAC, glutaraldehydes, etc. as discussed in more detail below) or any other crosslinker that couples ligands to the particle surface via chemical modification of the particle surface after preparation. This also includes a process whereby amphiphilic molecules such as fatty acids, lipids or functional stabilizers may be passively adsorbed and adhered to the particle surface, thereby introducing functional end groups for tethering to ligands.

In some embodiments, the microparticles may be synthesized to comprise one or more targeting groups on their exterior surface to target a specific cell or tissue type (e.g., cardiomyocytes). These targeting groups include without limitation receptors, ligands, antibodies, and the like. These targeting groups bind their partner on the cells' surface. In some embodiments, the microparticles will integrate into a lipid bilayer that comprises the cell surface and the mitochondria are delivered to the cell.

The microparticles may also comprise a lipid bilayer on their outermost surface. This bilayer may be comprised of one or more lipids of the same or different type. Examples include without limitation phospholipids such as phosphocholines and phosphoinositols. Specific examples include without limitation DMPC, DOPC, DSPC, and various other lipids such as those described herein for liposomes.

In some embodiments, the carrier comprises nanoparticles, e.g., as described herein.

In some embodiments, the vesicles or microparticles described herein are functionalized with a diagnostic agent. Examples of diagnostic agents include, but are not limited to, commercially available imaging agents used in positron emissions tomography (PET), computer assisted tomography (CAT), single photon emission computerized tomography, x-ray, fluoroscopy, and magnetic resonance imaging (MRI); and contrast agents. Examples of suitable materials for use as contrast agents in MRI include gadolinium chelates, as well as iron, magnesium, manganese, copper, and chromium.

### Carriers

A composition (e.g., pharmaceutical composition) described herein may comprise, be formulated with, and/or be delivered in, a carrier. In one aspect, the invention includes a composition, e.g., a pharmaceutical composition, comprising a carrier (e.g., a vesicle, a liposome, a lipid nanoparticle, an exosome, a red blood cell, an exosome (e.g., a mammalian or plant exosome), a fusosome) comprising (e.g., encapsulating) a composition described herein (e.g., an anellovector, Anellovirus, or genetic element described herein).

In some embodiments, the compositions and systems described herein can be formulated in liposomes or other similar vesicles. Generally, liposomes are spherical vesicle structures composed of a uni- or multilamellar lipid bilayer surrounding internal aqueous compartments and a relatively impermeable outer lipophilic phospholipid bilayer. Liposomes may be anionic, neutral or cationic. Liposomes generally have one or more (e.g., all) of the following characteristics: biocompatibility, nontoxicity, can deliver both hydrophilic and lipophilic drug molecules, can protect their cargo from degradation by plasma enzymes, and can transport their load across biological membranes and the blood brain barrier (BBB) (see, e.g., Spuch and Navarro, Journal of Drug Delivery, vol. 2011, Article ID 469679, 12 pages, 2011. doi:10.1155/2011/469679; and Zylberberg & Matosevic. 2016. Drug Delivery, 23:9, 3319-3329, doi: 10.1080/10717544.2016.1177136).

Vesicles can be made from several different types of lipids; however, phospholipids are most commonly used to generate liposomes as drug carriers. Methods for preparation of multilamellar vesicle lipids are known (see, for example, U.S. Pat. No. 6,693,086, the teachings of which relating to multilamellar vesicle lipid preparation are incorporated herein by reference). Although vesicle formation can be spontaneous when a lipid film is mixed with an aqueous solution, it can also be expedited by applying force in the form of shaking by using a homogenizer, sonicator, or an extrusion apparatus (see, e.g., Spuch and Navarro, Journal of Drug Delivery, vol. 2011, Article ID 469679, 12 pages, 2011. doi:10.1155/2011/469679 for review). Extruded lipids can be prepared by, e.g., extruding through filters of decreasing size, as described in Templeton et al., Nature Biotech, 15:647-652, 1997.

Lipid nanoparticles (LNPs) are another example of a carrier that provides a biocompatible and biodegradable delivery system for the pharmaceutical compositions described herein. See, e.g., Gordillo-Galeano et al. European Journal of Pharmaceutics and Biopharmaceutics. Volume 133, December 2018, Pages 285-308. Nanostructured lipid carriers (NLCs) are modified solid lipid nanoparticles (SLNs) that retain the characteristics of the SLN, improve drug stability and loading capacity, and prevent drug leakage. Polymer nanoparticles (PNPs) are an important component of drug delivery. These nanoparticles can effectively direct drug delivery to specific targets and improve drug stability and controlled drug release. Lipid-polymer nanoparticles (PLNs), a new type of carrier that combines liposomes and polymers, may also be employed. These nanoparticles possess the complementary advantages of PNPs and liposomes. A PLN is composed of a core-shell structure; the polymer core provides a stable structure, and the phospholipid shell offers good biocompatibility. As such, the two components increase the drug encapsulation efficiency rate, facilitate surface modification, and prevent leakage of water-soluble drugs. For a review, see, e.g., Li et al. 2017, Nanomaterials 7, 122; doi:10.3390/nano7060122.

Exosomes can also be used as drug delivery vehicles for the compositions and systems described herein. For a review, see Ha et al. July 2016. Acta Pharmaceutica Sinica B. Volume 6, Issue 4, Pages 287-296; doi.org/10.1016/j.apsb.2016.02.001.

Ex vivo differentiated red blood cells can also be used as a carrier for a composition described herein. See, e.g., WO2015073587; WO2017123646; WO2017123644; WO2018102740; WO2016183482; WO2015153102; WO2018151829; WO2018009838; Shi et al. 2014. Proc Natl Acad Sci USA. 111(28): 10131-10136; U.S. Pat. No. 9,644,180; Huang et al. 2017. Nature Communications 8: 423; Shi et al. 2014. Proc Natl Acad Sci USA. 111(28): 10131-10136.

Fusosome compositions, e.g., as described in WO2018208728, can also be used as carriers to deliver a composition described herein.

### Membrane Penetrating Polypeptides

In some embodiments, the composition further comprises a membrane penetrating polypeptide (MPP) to carry the components into cells or across a membrane, e.g., cell or nuclear membrane. Membrane penetrating polypeptides that are capable of facilitating transport of substances across a membrane include, but are not limited to, cell-penetrating peptides (CPPs)(see, e.g., U.S. Pat. No. 8,603,966), fusion peptides for plant intracellular delivery (see, e.g., Ng et al., PLoS One, 2016, 11:e0154081), protein transduction domains, Trojan peptides, and membrane translocation signals (MTS) (see, e.g., Tung et al., Advanced Drug Delivery Reviews 55:281-294 (2003)). Some MPP are rich in amino acids, such as arginine, with positively charged side chains.

Membrane penetrating polypeptides have the ability of inducing membrane penetration of a component and allow macromolecular translocation within cells of multiple tissues in vivo upon systemic administration. A membrane penetrating polypeptide may also refer to a peptide which, when brought into contact with a cell under appropriate conditions, passes from the external environment in the intracellular environment, including the cytoplasm, organelles such as mitochondria, or the nucleus of the cell, in amounts significantly greater than would be reached with passive diffusion.

Components transported across a membrane may be reversibly or irreversibly linked to the membrane penetrating polypeptide. A linker may be a chemical bond, e.g., one or more covalent bonds or non-covalent bonds. In some embodiments, the linker is a peptide linker. Such a linker may be between 2-30 amino acids, or longer. The linker includes flexible, rigid or cleavable linkers.

### Combinations

In one aspect, the anellovector or composition comprising an anellovector described herein may also include one or more heterologous moiety. In one aspect, the anellovector or composition comprising a anellovector described herein may also include one or more heterologous moiety in a fusion. In some embodiments, a heterologous moiety may be linked with the genetic element. In some embodiments, a heterologous moiety may be enclosed in the proteinaceous exterior as part of the anellovector. In some embodiments, a heterologous moiety may be administered with the anellovector.

In one aspect, the invention includes a cell or tissue comprising any one of the anellovectors and heterologous moieties described herein.

In another aspect, the invention includes a pharmaceutical composition comprising a anellovector and the heterologous moiety described herein.

In some embodiments, the heterologous moiety may be a virus (e.g., an effector (e.g., a drug, small molecule), a targeting agent (e.g., a DNA targeting agent, antibody, receptor ligand), a tag (e.g., fluorophore, light sensitive agent such as KillerRed), or an editing or targeting moiety described herein. In some embodiments, a membrane translocating polypeptide described herein is linked to one or more heterologous moieties. In one embodiment, the heterologous moiety is a small molecule (e.g., a peptidomimetic or a small organic molecule with a molecular weight of less than 2000 daltons), a peptide or polypeptide (e.g., an antibody or antigen-binding fragment thereof), a nanoparticle, an aptamer, or pharmacoagent.

Viruses

In some embodiments, an anellovector or composition (e.g., as described herein) may further comprise one or more components or elements (e.g., nucleic acids or polypeptides) from a virus other than an Anellovirus, e.g., as a heterologous moiety, e.g., a single stranded DNA virus, e.g., Bidnavirus, Circovirus, Geminivirus, Genomovirus, Inovirus, Microvirus, Nanovirus, Parvovirus, and Spiravirus. In some embodiments, the composition may further comprise a double stranded DNA virus, e.g., Adenovirus, Ampullavirus, Ascovirus, Asfarvirus, Baculovirus, Fusellovirus, Globulovirus, Guttavirus, Hytrosavirus, Herpesvirus, Iridovirus, Lipothrixvirus, Nimavirus, and Poxvirus. In some embodiments, the composition may further comprise an RNA virus, e.g., Alphavirus, Furovirus, Hepatitis virus, Hordeivirus, Tobamovirus, Tobravirus, Tricornavirus, Rubivirus, Birnavirus, Cystovirus, Partitivirus, and Reovirus. In some embodiments, the anellovector is administered with a virus as a heterologous moiety.

In some embodiments, the heterologous moiety may comprise a non-pathogenic, e.g., symbiotic, commensal, native, virus. In some embodiments, the non-pathogenic virus is one or more anelloviruses, e.g., Alphatorquevirus (TT), Betatorquevirus (TTM), and Gammatorquevirus (TTMD). In some embodiments, the anellovirus may include a Torque Teno Virus (TT), a SEN virus, a Sentinel virus, a TTV-like mini virus, a TT virus, a TT virus genotype 6, a TT virus group, a TTV-like virus DXL1, a TTV-like virus DXL2, a Torque Teno-like Mini Virus (TTM), or a Torque Teno-like Midi Virus (TTMD). In some embodiments, the non-pathogenic virus comprises one or more sequences having at least at least about 60%, 70% 80%, 85%, 90% 95%, 96%, 97%, 98% and 99% nucleotide sequence identity to any one of the nucleotide sequences described herein.

In some embodiments, the heterologous moiety may comprise one or more viruses that are identified as lacking in the subject. For example, a subject identified as having dyvirosis may be administered a composition comprising an anellovector and one or more viral components or viruses that are imbalanced in the subject or having a ratio that differs from a reference value, e.g., a healthy subject.

In some embodiments, the heterologous moiety may comprise one or more non-anelloviruses, e.g., adenovirus, herpes virus, pox virus, vaccinia virus, SV40, papilloma virus, an RNA virus such as a retrovirus, e.g., lenti virus, a single-stranded RNA virus, e.g., hepatitis virus, or a double-stranded RNA virus e.g., rotavirus. In some embodiments, the anellovector or the virus is defective, or requires assistance in order to produce infectious particles. Such assistance can be provided, e.g., by using helper cell lines that contain a nucleic acid, e.g., plasmids or DNA integrated into the genome, encoding one or more of (e.g., all of) the structural genes of the replication defective anellovector or virus under the control of regulatory sequences within the LTR. Suitable cell lines for replicating the anellovectors described herein include cell lines known in the art, e.g., A549 cells, which can be modified as described herein.

Targeting Moiety

In some embodiments, the composition or anellovector described herein may further comprise a targeting moiety, e.g., a targeting moiety that specifically binds to a molecule of interest present on a target cell. The targeting moiety may modulate a specific function of the molecule of interest or cell, modulate a specific molecule (e.g., enzyme, protein or nucleic acid), e.g., a specific molecule downstream of the molecule of interest in a pathway, or specifically bind to a target to localize the anellovector or genetic element. For example, a targeting moiety may include a therapeutic that interacts with a specific molecule of interest to increase, decrease or otherwise modulate its function.

Tagging or Monitoring Moiety

In some embodiments, the composition or anellovector described herein may further comprise a tag to label or monitor the anellovector or genetic element described herein. The tagging or monitoring moiety may be removable by chemical agents or enzymatic cleavage, such as proteolysis or intein splicing. An affinity tag may be useful to purify the tagged polypeptide using an affinity technique. Some examples include, chitin binding protein (CBP), maltose binding protein (MBP), glutathione-S-transferase (GST), and poly(His) tag. A solubilization tag may be useful to aid recombinant proteins expressed in chaperone-deficient species such as E. coli to assist in the proper folding in proteins and keep them from precipitating. Some examples include thioredoxin (TRX) and poly(NANP). The tagging or monitoring moiety may include a light sensitive tag, e.g., fluorescence. Fluorescent tags are useful for visualization. GFP and its variants are some examples commonly used as fluorescent tags. Protein tags may allow specific enzymatic modifications (such as biotinylation by biotin ligase) or chemical modifications (such as reaction with FlAsH-EDT2 for fluorescence imaging) to occur. Often tagging or monitoring moiety are combined, in order to connect proteins to multiple other components. The tagging or monitoring moiety may also be removed by specific proteolysis or enzymatic cleavage (e.g. by TEV protease, Thrombin, Factor Xa or Enteropeptidase).

Nanoparticles

In some embodiments, the composition or anellovector described herein may further comprise a nanoparticle. Nanoparticles include inorganic materials with a size between about 1 and about 1000 nanometers, between about 1 and about 500 nanometers in size, between about 1 and about 100 nm, between about 50 nm and about 300 nm, between about 75 nm and about 200 nm, between about 100 nm and about 200 nm, and any range therebetween. Nanoparticles generally have a composite structure of nanoscale dimensions. In some embodiments, nanoparticles are typically spherical although different morphologies are possible depending on the nanoparticle composition. The portion of the nanoparticle contacting an environment external to the nanoparticle is generally identified as the surface of the nanoparticle. In nanoparticles described herein, the size limitation can be restricted to two dimensions and so that nanoparticles include composite structure having a diameter from about 1 to about 1000 nm, where the specific diameter depends on the nanoparticle composition and on the intended use of the nanoparticle according to the experimental design. For example, nanoparticles used in therapeutic applications typically have a size of about 200 nm or below.

Additional desirable properties of the nanoparticle, such as surface charges and steric stabilization, can also vary in view of the specific application of interest. Exemplary properties that can be desirable in clinical applications such as cancer treatment are described in Davis et al, Nature 2008 vol. 7, pages 771-782; Duncan, Nature 2006 vol. 6, pages 688-701; and Allen, Nature 2002 vol. 2 pages 750-763, each incorporated herein by reference in its entirety. Additional properties are identifiable by a skilled person upon reading of the present disclosure. Nanoparticle dimensions and properties can be detected by techniques known in the art. Exemplary techniques to detect particles dimensions include but are not limited to dynamic light scattering (DLS) and a variety of microscopies such at transmission electron microscopy (TEM) and atomic force microscopy (AFM). Exemplary techniques to detect particle morphology include but are not limited to TEM and AFM. Exemplary techniques to detect surface charges of the nanoparticle include but are not limited to zeta potential method. Additional techniques suitable to detect other chemical properties comprise by ¹H, ¹¹B, and ¹³C and ¹⁹F NMR, UV/Vis and infrared/Raman spectroscopies and fluorescence spectroscopy (when nanoparticle is used in combination with fluorescent labels) and additional techniques identifiable by a skilled person.

Small Molecules

In some embodiments, the composition or anellovector described herein may further comprise a small molecule. Small molecule moieties include, but are not limited to, small peptides, peptidomimetics (e.g., peptoids), amino acids, amino acid analogs, synthetic polynucleotides, polynucleotide analogs, nucleotides, nucleotide analogs, organic and inorganic compounds (including heterorganic and organometallic compounds) generally having a molecular weight less than about 5,000 grams per mole, e.g., organic or inorganic compounds having a molecular weight less than about 2,000 grams per mole, e.g., organic or inorganic compounds having a molecular weight less than about 1,000 grams per mole, e.g., organic or inorganic compounds having a molecular weight less than about 500 grams per mole, and salts, esters, and other pharmaceutically acceptable forms of such compounds. Small molecules may include, but are not limited to, a neurotransmitter, a hormone, a drug, a toxin, a viral or microbial particle, a synthetic molecule, and agonists or antagonists.

Examples of suitable small molecules include those described in, “The Pharmacological Basis of Therapeutics,” Goodman and Gilman, McGraw-Hill, New York, N.Y., (1996), Ninth edition, under the sections: Drugs Acting at Synaptic and Neuroeffector Junctional Sites; Drugs Acting on the Central Nervous System; Autacoids: Drug Therapy of Inflammation; Water, Salts and Ions; Drugs Affecting Renal Function and Electrolyte Metabolism; Cardiovascular Drugs; Drugs Affecting Gastrointestinal Function; Drugs Affecting Uterine Motility; Chemotherapy of Parasitic Infections; Chemotherapy of Microbial Diseases; Chemotherapy of Neoplastic Diseases; Drugs Used for Immunosuppression; Drugs Acting on Blood-Forming organs; Hormones and Hormone Antagonists; Vitamins, Dermatology; and Toxicology, all incorporated herein by reference. Some examples of small molecules include, but are not limited to, prion drugs such as tacrolimus, ubiquitin ligase or HECT ligase inhibitors such as heclin, histone modifying drugs such as sodium butyrate, enzymatic inhibitors such as 5-aza-cytidine, anthracyclines such as doxorubicin, beta-lactams such as penicillin, anti-bacterials, chemotherapy agents, anti-virals, modulators from other organisms such as VP64, and drugs with insufficient bioavailability such as chemotherapeutics with deficient pharmacokinetics.

In some embodiments, the small molecule is an epigenetic modifying agent, for example such as those described in de Groote et al. Nuc. Acids Res. (2012):1-18. Exemplary small molecule epigenetic modifying agents are described, e.g., in Lu et al. J. Biomolecular Screening 17.5(2012):555-71, e.g., at Table 1 or 2, incorporated herein by reference. In some embodiments, an epigenetic modifying agent comprises vorinostat or romidepsin. In some embodiments, an epigenetic modifying agent comprises an inhibitor of class I, II, III, and/or IV histone deacetylase (HDAC). In some embodiments, an epigenetic modifying agent comprises an activator of SirTI. In some embodiments, an epigenetic modifying agent comprises Garcinol, Lys-CoA, C646, (+)-JQI, I-BET, BICI, MS120, DZNep, UNC0321, EPZ004777, AZ505, AMI-I, pyrazole amide 7b, benzo[d]imidazole 17b, acylated dapsone derivative (e.g, PRMTI), methylstat, 4,4′-dicarboxy-2,2′-bipyridine, SID 85736331, hydroxamate analog 8, tanylcypromie, bisguanidine and biguanide polyamine analogs, UNC669, Vidaza, decitabine, sodium phenyl butyrate (SDB), lipoic acid (LA), quercetin, valproic acid, hydralazine, bactrim, green tea extract (e.g., epigallocatechin gallate (EGCG)), curcumin, sulforphane and/or allicin/diallyl disulfide. In some embodiments, an epigenetic modifying agent inhibits DNA methylation, e.g., is an inhibitor of DNA methyltransferase (e.g., is 5-azacitidine and/or decitabine). In some embodiments, an epigenetic modifying agent modifies histone modification, e.g., histone acetylation, histone methylation, histone sumoylation, and/or histone phosphorylation. In some embodiments, the epigenetic modifying agent is an inhibitor of a histone deacetylase (e.g., is vorinostat and/or trichostatin A).

In some embodiments, the small molecule is a pharmaceutically active agent. In one embodiment, the small molecule is an inhibitor of a metabolic activity or component. Useful classes of pharmaceutically active agents include, but are not limited to, antibiotics, anti-inflammatory drugs, angiogenic or vasoactive agents, growth factors and chemotherapeutic (anti-neoplastic) agents (e.g., tumour suppressers). One or a combination of molecules from the categories and examples described herein or from (Orme-Johnson 2007, Methods Cell Biol. 2007; 80:813-26) can be used. In one embodiment, the invention includes a composition comprising an antibiotic, anti-inflammatory drug, angiogenic or vasoactive agent, growth factor or chemotherapeutic agent.

Peptides or Proteins

In some embodiments, the composition or anellovector described herein may further comprise a peptide or protein. The peptide moieties may include, but are not limited to, a peptide ligand or antibody fragment (e.g., antibody fragment that binds a receptor such as an extracellular receptor), neuropeptide, hormone peptide, peptide drug, toxic peptide, viral or microbial peptide, synthetic peptide, and agonist or antagonist peptide.

Peptides moieties may be linear or branched. The peptide has a length from about 5 to about 200 amino acids, about 15 to about 150 amino acids, about 20 to about 125 amino acids, about 25 to about 100 amino acids, or any range therebetween.

Some examples of peptides include, but are not limited to, fluorescent tags or markers, antigens, antibodies, antibody fragments such as single domain antibodies, ligands and receptors such as glucagon-like peptide-1 (GLP-1), GLP-2 receptor 2, cholecystokinin B (CCKB) and somatostatin receptor, peptide therapeutics such as those that bind to specific cell surface receptors such as G protein-coupled receptors (GPCRs) or ion channels, synthetic or analog peptides from naturally-bioactive peptides, anti-microbial peptides, pore-forming peptides, tumor targeting or cytotoxic peptides, and degradation or self-destruction peptides such as an apoptosis-inducing peptide signal or photosensitizer peptide.

Peptides useful in the invention described herein also include small antigen-binding peptides, e.g., antigen binding antibody or antibody-like fragments, such as single chain antibodies, nanobodies (see, e.g., Steeland et al. 2016. Nanobodies as therapeutics: big opportunities for small antibodies. Drug Discov Today: 21(7):1076-113). Such small antigen binding peptides may bind a cytosolic antigen, a nuclear antigen, an intra-organellar antigen.

In some embodiments, the composition or anellovector described herein includes a polypeptide linked to a ligand that is capable of targeting a specific location, tissue, or cell.

Oligonucleotide Aptamers

In some embodiments, the composition or anellovector described herein may further comprise an oligonucleotide aptamer. Aptamer moieties are oligonucleotide or peptide aptamers. Oligonucleotide aptamers are single-stranded DNA or RNA (ssDNA or ssRNA) molecules that can bind to pre-selected targets including proteins and peptides with high affinity and specificity.

Oligonucleotide aptamers are nucleic acid species that may be engineered through repeated rounds of in vitro selection or equivalently, SELEX (systematic evolution of ligands by exponential enrichment) to bind to various molecular targets such as small molecules, proteins, nucleic acids, and even cells, tissues and organisms. Aptamers provide discriminate molecular recognition, and can be produced by chemical synthesis. In addition, aptamers may possess desirable storage properties, and elicit little or no immunogenicity in therapeutic applications.

Both DNA and RNA aptamers can show robust binding affinities for various targets. For example, DNA and RNA aptamers have been selected for t lysozyme, thrombin, human immunodeficiency virus trans-acting responsive element (HIV TAR), (see en.wikipedia.org/wiki/Aptamer-cite_note-10), hemin, interferon γ, vascular endothelial growth factor (VEGF), prostate specific antigen (PSA), dopamine, and the non-classical oncogene, heat shock factor 1 (HSF1).

Peptide Aptamers

In some embodiments, the composition or anellovector described herein may further comprise a peptide aptamer. Peptide aptamers have one (or more) short variable peptide domains, including peptides having low molecular weight, 12-14 kDa. Peptide aptamers may be designed to specifically bind to and interfere with protein-protein interactions inside cells.

Peptide aptamers are artificial proteins selected or engineered to bind specific target molecules. These proteins include of one or more peptide loops of variable sequence. They are typically isolated from combinatorial libraries and often subsequently improved by directed mutation or rounds of variable region mutagenesis and selection. In vivo, peptide aptamers can bind cellular protein targets and exert biological effects, including interference with the normal protein interactions of their targeted molecules with other proteins. In particular, a variable peptide aptamer loop attached to a transcription factor binding domain is screened against the target protein attached to a transcription factor activating domain. In vivo binding of the peptide aptamer to its target via this selection strategy is detected as expression of a downstream yeast marker gene. Such experiments identify particular proteins bound by the aptamers, and protein interactions that the aptamers disrupt, to cause the phenotype. In addition, peptide aptamers derivatized with appropriate functional moieties can cause specific post-translational modification of their target proteins, or change the subcellular localization of the targets

Peptide aptamers can also recognize targets in vitro. They have found use in lieu of antibodies in biosensors and used to detect active isoforms of proteins from populations containing both inactive and active protein forms. Derivatives known as tadpoles, in which peptide aptamer “heads” are covalently linked to unique sequence double-stranded DNA “tails”, allow quantification of scarce target molecules in mixtures by PCR (using, for example, the quantitative real-time polymerase chain reaction) of their DNA tails.

Peptide aptamer selection can be made using different systems, but the most used is currently the yeast two-hybrid system. Peptide aptamers can also be selected from combinatorial peptide libraries constructed by phage display and other surface display technologies such as mRNA display, ribosome display, bacterial display and yeast display. These experimental procedures are also known as biopannings. Among peptides obtained from biopannings, mimotopes can be considered as a kind of peptide aptamers. All the peptides panned from combinatorial peptide libraries have been stored in a special database with the name MimoDB.

### V. Host Cells

The invention is further directed to a host or host cell comprising an anellovector, bacmid, or donor vector, e.g., as described herein. In some embodiments, the host or host cell is an insect cell (e.g., an Sf9 cell, Sf21 cell, or Hi5 cell). In some embodiments, the insect cell is derived from Bombyx mori, Mamestra brassicae, Spodoptera frugiperda, Trichoplusia ni, or Drosophila melanogaster. In some embodiments, the host or host cell is a bacterial cell (e.g., an E. coli cell, e.g., a DH 10Bac cell). In certain embodiments, as confirmed herein, provided anellovectors infect a range of different target host cells (e.g., a plant cell, an insect cell, a bacterial cell, a fungus cell, or an animal cell, e.g., a mammalian cell, e.g., a human cell). Target host cells include cells of mesodermal, endodermal, or ectodermal origin. Target host cells include, e.g., epithelial cells, muscle cells, white blood cells (e.g., lymphocytes), kidney tissue cells, lung tissue cells.

In some embodiments, the anellovector is substantially non-immunogenic in the host. The anellovector or genetic element fails to produce an undesired substantial response by the host's immune system. Some immune responses include, but are not limited to, humoral immune responses (e.g., production of antigen-specific antibodies) and cell-mediated immune responses (e.g., lymphocyte proliferation).

In some embodiments, a host or a host cell is contacted with (e.g., infected with) an anellovector. In some embodiments, the host is a mammal, such as a human. In some embodiments, the host cell is a mammalian cell, e.g., a human cell. The amount of the anellovector in the host can be measured at any time after administration. In certain embodiments, a time course of anellovector growth in a culture is determined.

In some embodiments, the anellovector, e.g., an anellovector as described herein, is heritable. In some embodiments, the anellovector is transmitted linearly in fluids and/or cells from mother to child. In some embodiments, daughter cells from an original host cell comprise the anellovector. In some embodiments, a mother transmits the anellovector to child with an efficiency of at least 25%, 50%, 60%, 70%, 80%, 85%, 90%, 95%, or 99%, or a transmission efficiency from host cell to daughter cell at least 25%, 50%, 60%, 70%, 80%, 85%, 90%, 95%, or 99%. In some embodiments, the anellovector in a host cell has a transmission efficiency during meiosis of at 25%, 50%, 60%, 70%, 80%, 85%, 90%, 95%, or 99%. In some embodiments, the anellovector in a host cell has a transmission efficiency during mitosis of at least 25%, 50%, 60%, 70%, 80%, 85%, 90%, 95%, or 99%. In some embodiments, the anellovector in a cell has a transmission efficiency between about 10%-20%, 20%-30%, 30%-40%, 40%-50%, 50%-60%, 60%-70%, 70%-75%, 75%-80%, 80%-85%, 85%-90%, 90%-95%, 95%-99%, or any percentage therebetween.

In some embodiments, the anellovector, e.g., anellovector replicates within the host cell. In embodiments, the host cell is an insect cell (e.g., an Sf9 cell, an Sf21 cell, or a Hi5 cell). In one embodiment, the anellovector is capable of replicating in a mammalian cell, e.g., human cell. In other embodiments, the anellovector is replication deficient or replication incompetent.

While in some embodiments the anellovector replicates in the host cell, the anellovector does not integrate into the genome of the host, e.g., with the host's chromosomes. In some embodiments, the anellovector has a negligible recombination frequency, e.g., with the host's chromosomes. In some embodiments, the anellovector has a recombination frequency, e.g., less than about 1.0 cM/Mb, 0.9 cM/Mb, 0.8 cM/Mb, 0.7 cM/Mb, 0.6 cM/Mb, 0.5 cM/Mb, 0.4 cM/Mb, 0.3 cM/Mb, 0.2 cM/Mb, 0.1 cM/Mb, or less, e.g., with the host's chromosomes.

### VI. Methods of Use

The anellovectors and compositions comprising anellovectors described herein may be used in methods of treating a disease, disorder, or condition, e.g., in a subject (e.g., a mammalian subject, e.g., a human subject) in need thereof. Administration of a pharmaceutical composition described herein may be, for example, by way of parenteral (including intravenous, intratumoral, intraperitoneal, intramuscular, intracavity, and subcutaneous) administration. The anellovectors may be administered alone or formulated as a pharmaceutical composition.

The anellovectors may be administered in the form of a unit-dose composition, such as a unit dose parenteral composition. Such compositions are generally prepared by admixture and can be suitably adapted for parenteral administration. Such compositions may be, for example, in the form of injectable and infusable solutions or suspensions or suppositories or aerosols.

In some embodiments, administration of a anellovector or composition comprising same, e.g., as described herein, may result in delivery of a genetic element comprised by the anellovector to a target cell, e.g., in a subject.

An anellovector or composition thereof described herein, e.g., comprising an effector (e.g., an endogenous or exogenous effector), may be used to deliver the effector to a cell, tissue, or subject. In some embodiments, the anellovector or composition thereof is used to deliver the effector to bone marrow, blood, heart, GI or skin. Delivery of an effector by administration of a anellovector composition described herein may modulate (e.g., increase or decrease) expression levels of a noncoding RNA or polypeptide in the cell, tissue, or subject. Modulation of expression level in this fashion may result in alteration of a functional activity in the cell to which the effector is delivered. In some embodiments, the modulated functional activity may be enzymatic, structural, or regulatory in nature.

In some embodiments, the anellovector, or copies thereof, are detectable in a cell 24 hours (e.g., 1 day, 2 days, 3 days, 4 days, 5 days, 6 days, 1 week, 2 weeks, 3 weeks, 4 weeks, 30 days, or 1 month) after delivery into a cell. In embodiments, a anellovector or composition thereof mediates an effect on a target cell, and the effect lasts for at least 1, 2, 3, 4, 5, 6, or 7 days, 2, 3, or 4 weeks, or 1, 2, 3, 6, or 12 months. In some embodiments (e.g., wherein the anellovector or composition thereof comprises a genetic element encoding an exogenous protein), the effect lasts for less than 1, 2, 3, 4, 5, 6, or 7 days, 2, 3, or 4 weeks, or 1, 2, 3, 6, or 12 months.

Examples of diseases, disorders, and conditions that can be treated with the anellovector described herein, or a composition comprising the anellovector, include, without limitation: immune disorders, interferonopathies (e.g., Type I interferonopathies), infectious diseases, inflammatory disorders, autoimmune conditions, cancer (e.g., a solid tumor, e.g., lung cancer, non-small cell lung cancer, e.g., a tumor that expresses a gene responsive to mIR-625, e.g., caspase-3), and gastrointestinal disorders. In some embodiments, the anellovector modulates (e.g., increases or decreases) an activity or function in a cell with which the anellovector is contacted. In some embodiments, the anellovector modulates (e.g., increases or decreases) the level or activity of a molecule (e.g., a nucleic acid or a protein) in a cell with which the anellovector is contacted. In some embodiments, the anellovector decreases viability of a cell, e.g., a cancer cell, with which the anellovector is contacted, e.g., by at least about 10%, 20%, 30%, 40%, 50%, 60%, 70%, 80%, 90%, 95%, 99%, or more. In some embodiments, the anellovector comprises an effector, e.g., an miRNA, e.g., miR-625, that decreases viability of a cell, e.g., a cancer cell, with which the anellovector is contacted, e.g., by at least about 10%, 20%, 30%, 40%, 50%, 60%, 70%, 80%, 90%, 95%, 99%, or more. In some embodiments, the anellovector increases apoptosis of a cell, e.g., a cancer cell, e.g., by increasing caspase-3 activity, with which the anellovector is contacted, e.g., by at least about 10%, 20%, 30%, 40%, 50%, 60%, 70%, 80%, 90%, 95%, 99%, or more. In some embodiments, the anellovector comprises an effector, e.g., an miRNA, e.g., miR-625, that increases apoptosis of a cell, e.g., a cancer cell, e.g., by increasing caspase-3 activity, with which the anellovector is contacted, e.g., by at least about 10%, 20%, 30%, 40%, 50%, 60%, 70%, 80%, 90%, 95%, 99%, or more.

### VII. Administration/Delivery

The composition (e.g., a pharmaceutical composition comprising an anellovector as described herein) may be formulated to include a pharmaceutically acceptable excipient. Pharmaceutical compositions may optionally comprise one or more additional active substances, e.g. therapeutically and/or prophylactically active substances. Pharmaceutical compositions of the present invention may be sterile and/or pyrogen-free. General considerations in the formulation and/or manufacture of pharmaceutical agents may be found, for example, in Remington: The Science and Practice of Pharmacy 21st ed., Lippincott Williams & Wilkins, 2005 (incorporated herein by reference).

Although the descriptions of pharmaceutical compositions provided herein are principally directed to pharmaceutical compositions which are suitable for administration to humans, it will be understood by the skilled artisan that such compositions are generally suitable for administration to any other animal, e.g., to non-human animals, e.g. non-human mammals. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, humans and/or other primates; mammals, including commercially relevant mammals such as cattle, pigs, horses, sheep, cats, dogs, mice, and/or rats; and/or birds, including commercially relevant birds such as poultry, chickens, ducks, geese, and/or turkeys.

Formulations of the pharmaceutical compositions described herein may be prepared by any method known or hereafter developed in the art of pharmacology. In general, such preparatory methods include the step of bringing the active ingredient into association with an excipient and/or one or more other accessory ingredients, and then, if necessary and/or desirable, dividing, shaping and/or packaging the product.

In one aspect, the invention features a method of delivering an anellovector to a subject. The method includes administering a pharmaceutical composition comprising an anellovector as described herein to the subject. In some embodiments, the administered anellovector replicates in the subject (e.g., becomes a part of the virome of the subject).

The pharmaceutical composition may include wild-type or native viral elements and/or modified viral elements. The anellovector may include one or more Anellovirus sequences (e.g., nucleic acid sequences or nucleic acid sequences encoding amino acid sequences thereof) or a sequence with at least about 60%, 65%, 70%, 75%, 80%, 85%, 90% 95%, 96%, 97%, 98% and 99% nucleotide sequence identity thereto. The anellovector may comprise a nucleic acid molecule comprising a nucleic acid sequence with at least about 60%, 65%, 70%, 75%, 80%, 85%, 90% 95%, 96%, 97%, 98% and 99% sequence identity to one or more Anellovirus sequences (e.g., an Anellovirus ORF1 nucleic acid sequence). The anellovector may comprise a nucleic acid molecule encoding an amino acid sequence with at least about 60%, 65%, 70%, 75%, 80%, 85%, 90% 95%, 96%, 97%, 98% and 99% sequence identity to an Anellovirus amino acid sequence (e.g., the amino acid sequence of an Anellovirus ORF1 molecule). The anellovector may comprise a polypeptide comprising an amino acid sequence with at least about 60%, 65%, 70%, 75%, 80%, 85%, 90% 95%, 96%, 97%, 98% and 99% sequence identity to an Anellovirus amino acid sequence (e.g., the amino acid sequence of an Anellovirus ORF1 molecule).

In some embodiments, the anellovector is sufficient to increase (stimulate) endogenous gene and protein expression, e.g., at least about 5%, 10%, 15%, 20%, 25%, 30%, 35%, 40%, 45%, 50%, or more as compared to a reference, e.g., a healthy control. In certain embodiments, the anellovector is sufficient to decrease (inhibit) endogenous gene and protein expression, e.g., at least about 5%, 10%, 15%, 20%, 25%, 30%, 35%, 40%, 45%, 50%, or more as compared to a reference, e.g., a healthy control.

In some embodiments, the anellovector inhibits/enhances one or more viral properties, e.g., tropism, infectivity, immunosuppression/activation, in a host or host cell, e.g., at least about 5%, 10%, 15%, 20%, 25%, 30%, 35%, 40%, 45%, 50%, or more as compared to a reference, e.g., a healthy control.

In some embodiments, the subject is administered the pharmaceutical composition further comprising one or more viral strains that are not represented in the viral genetic information.

In some embodiments, the pharmaceutical composition comprising an anellovector described herein is administered in a dose and time sufficient to modulate a viral infection. Some non-limiting examples of viral infections include adeno-associated virus, Aichi virus, Australian bat lyssavirus, BK polyomavirus, Banna virus, Barmah forest virus, Bunyamwera virus, Bunyavirus La Crosse, Bunyavirus snowshoe hare, Cercopithecine herpesvirus, Chandipura virus, Chikungunya virus, Cosavirus A, Cowpox virus, Coxsackievirus, Crimean-Congo hemorrhagic fever virus, Dengue virus, Dhori virus, Dugbe virus, Duvenhage virus, Eastern equine encephalitis virus, Ebolavirus, Echovirus, Encephalomyocarditis virus, Epstein-Barr virus, European bat lyssavirus, GB virus C/Hepatitis G virus, Hantaan virus, Hendra virus, Hepatitis A virus, Hepatitis B virus, Hepatitis C virus, Hepatitis E virus, Hepatitis delta virus, Horsepox virus, Human adenovirus, Human astrovirus, Human coronavirus, Human cytomegalovirus, Human enterovirus 68, Human enterovirus 70, Human herpesvirus 1, Human herpesvirus 2, Human herpesvirus 6, Human herpesvirus 7, Human herpesvirus 8, Human immunodeficiency virus, Human papillomavirus 1, Human papillomavirus 2, Human papillomavirus 16, Human papillomavirus 18, Human parainfluenza, Human parvovirus B19, Human respiratory syncytial virus, Human rhinovirus, Human SARS coronavirus, Human spumaretrovirus, Human T-lymphotropic virus, Human torovirus, Influenza A virus, Influenza B virus, Influenza C virus, Isfahan virus, JC polyomavirus, Japanese encephalitis virus, Junin arenavirus, KI Polyomavirus, Kunjin virus, Lagos bat virus, Lake Victoria marburgvirus, Langat virus, Lassa virus, Lordsdale virus, Louping ill virus, Lymphocytic choriomeningitis virus, Machupo virus, Mayaro virus, MERS coronavirus, Measles virus, Mengo encephalomyocarditis virus, Merkel cell polyomavirus, Mokola virus, Molluscum contagiosum virus, Monkeypox virus, Mumps virus, Murray valley encephalitis virus, New York virus, Nipah virus, Norwalk virus, O'nyong-nyong virus, Orf virus, Oropouche virus, Pichinde virus, Poliovirus, Punta toro phlebovirus, Puumala virus, Rabies virus, Rift valley fever virus, Rosavirus A, Ross river virus, Rotavirus A, Rotavirus B, Rotavirus C, Rubella virus, Sagiyama virus, Salivirus A, Sandfly fever sicilian virus, Sapporo virus, Semliki forest virus, Seoul virus, Simian foamy virus, Simian virus 5, Sindbis virus, Southampton virus, St. louis encephalitis virus, Tick-borne powassan virus, Torque teno virus, Toscana virus, Uukuniemi virus, Vaccinia virus, Varicella-zoster virus, Variola virus, Venezuelan equine encephalitis virus, Vesicular stomatitis virus, Western equine encephalitis virus, WU polyomavirus, West Nile virus, Yaba monkey tumor virus, Yaba-like disease virus, Yellow fever virus, and Zika Virus. In certain embodiments, the anellovector is sufficient to outcompete and/or displace a virus already present in the subject, e.g., at least about 5%, 10%, 15%, 20%, 25%, 30%, 35%, 40%, 45%, 50%, or more as compared to a reference. In certain embodiments, the anellovector is sufficient to compete with chronic or acute viral infection. In certain embodiments, the anellovector may be administered prophylactically to protect from viral infections (e.g. a provirotic). In some embodiments, the anellovector is in an amount sufficient to modulate (e.g., phenotype, virus levels, gene expression, compete with other viruses, disease state, etc. at least about 5%, 10%, 15%, 20%, 25%, 30%, 35%, 40%, 45%, 50%, or more). In some embodiments, treatment, treating, and cognates thereof comprise medical management of a subject (e.g., by administering an anellovector, e.g., an anellovector made as described herein), e.g., with the intent to improve, ameliorate, stabilize, prevent or cure a disease, pathological condition, or disorder. In some embodiments, treatment comprises active treatment (treatment directed to improve the disease, pathological condition, or disorder), causal treatment (treatment directed to the cause of the associated disease, pathological condition, or disorder), palliative treatment (treatment designed for the relief of symptoms), preventative treatment (treatment directed to preventing, minimizing or partially or completely inhibiting the development of the associated disease, pathological condition, or disorder), and/or supportive treatment (treatment employed to supplement another therapy).

All references and publications cited herein are hereby incorporated by reference.

The following examples are provided to further illustrate some embodiments of the present invention, but are not intended to limit the scope of the invention; it will be understood by their exemplary nature that other procedures, methodologies, or techniques known to those skilled in the art may alternatively be used.

### EXAMPLES

### Table of Contents

Example 1: Production of Anellovirus proteins in a baculovirus expression system
Example 2: Expression of Ring1 ORFs in Sf9 cells
Example 3: Expression of Ring2 ORFs in Sf9 cells
Example 4: Expression of all Ring2 ORFs simultaneously in Sf9 cells
Example 5: Co-delivery and independent expression of anellovirus genomes and recombinant Anellovirus ORFs in Sf9 cells
Example 6: Anellovirus ORF1 associates with DNA in Sf9 cells to form complexes isolated by isopycnic centrifugation
Example 7: Expression of ORF1 protein from a diverse array of Anelloviruses using baculovirus
Example 8: In vitro assembly of baculovirus constructs
Example 9: Preparation of synthetic anellovector constructs
Example 10: Assembly and infection of anellovectors
Example 11: Selectivity of anellovectors
Example 12: Replication-deficient anellovectors and helper viruses
Example 13: Manufacturing process for replication-competent anellovectors
Example 14: Manufacturing process of replication-deficient anellovectors
Example 15: Production of anellovectors using suspension cells
Example 16: Utilizing anellovectors to express an exogenous protein in mice
Example 17: Functional effects of an anellovector expressing an exogenous microRNA sequence
Example 18: Preparation and production of anellovectors to express exogenous non-coding RNAs
Example 19: Expression of an endogenous miRNA from an anellovector and deletion of the endogenous miRNA
Example 20: Anellovector delivery of exogenous proteins in vivo
Example 21: In vitro circularized Anellovirus genomes
Example 22: Production of anellovectors containing chimeric ORF1 with hypervariable domains from different Torque Teno Virus strains
Example 23: Production of chimeric ORF1 containing non-TTV protein/peptides in place of hypervariable domains
Example 24: Anellovectors based on tth8 and LY2 each successfully transduced the EPO gene into lung cancer cells
Example 25: Anellovectors with therapeutic transgenes can be detected in vivo after intravenous (i.v.) administration
Example 26: In vitro circularized genome as input material for producing anellovectors in vitro

### Example 1: Production of Anellovirus Proteins in a Baculovirus Expression System

In this example, a baculovirus expression system from Thermofisher Scientific (Cat. no. A38841) was adapted for expression of Anellovirus proteins. Briefly, a gene of interest (e.g., a gene encoding an Anellovirus ORF as described herein) was cloned into the pFastBac plasmid, which was then transformed into DH10Bac E. coli cells harboring a baculovirus genome. The transformants were grown on indicator plates according to the manufacturer's instructions and white colonies were selected for liquid culture and extraction of bacmid DNA. Recombination of the Anellovirus ORFs into the bacmids was validated by PCR.

Validated bacmid constructs showing successful recombination of the anellovirus ORF gene were then transfected into ExpiSf9 insect cells. The cells were incubated in a 27° C. non-humidified, non-CO₂ atmosphere incubator on an orbital shaker set at 125 rpm. After 72 hours post-transfection, Passage 0 stock (P0) recombinant baculovirus was harvested from the supernatant.

ExpiSf9 cells were infected using 25-100 μL of P0 baculovirus stock to make Passage 1 (P1) baculovirus for protein production. After 96 hours (approx. 4 days) post-infection, the supernatant was collected to obtain P1 baculovirus.

P1 recombinant baculovirus was titered by preparing five 10-fold serial dilutions of the test virus in fresh ExpiSf CD Medium in 1200 μL total volume. 800 μL of Expisf9 cells at 1.25×10⁶ viable cells/mL were seeded in a deep well plate and 1000 μL of the different dilutions of the test virus were added to each well. One well was setup as a negative control. Plate was then incubated overnight at 27° C. in a non-humidified incubator on a shaking platform at 225±5 rpm. After approx. 14-16 hours of incubation, the plate was removed from the incubator and everything was transferred to microcentrifuge tubes and spun at 300×g for 5 minutes. Supernatant was aspirated and each cell pellet was resuspended in 100 μL of dilution buffer (PBS+2% Fetal Bovine Serum) containing Anti-Baculovirus Envelope gp64 APC antibody at a final concentration of 0.15 μg/mL. Tubes were incubated for 30 mins at room temperature. Samples were then washed with 1 mL PBS followed by a 10 min centrifuge spin at 300×g. Supernatant was aspirated and cell pellet was resuspended in 1 mL Dilution Buffer. Samples were analyzed on a flow cytometer using the following parameters: red laser excitation: 633-647 nm; emission: 660 nm. Samples with different viral dilutions expressing percent positive gp64 were noted and used to calculate the viral titer.

For this and following examples, a series of recombinant bacmids and baculovirus vectors was produced for expression using the method described above. As shown in Table X below, various ORFs from LY2, tth8, and other anellovirus strains were cloned into bacmids. The ORFs were either tagged with an N-terminal His-tag with or without a human rhinovirus 3C (HRV 3C) proteolytic cleavage site, a C-terminal His-tag, or were left untagged, as indicated.

TABLE X
Recombinant bacmid constructs produced. “FullORF” = Full ORF-containing region,
with noncoding regions removed; ORF2/3 tagged.

| Construct | | | | | Tag | | | | |
|---|---|---|---|---|---|---|---|---|---|
| #/name | Strain | Ring # | ORF | Tag type | position | pFastBac | Bacmid | Baculovirus | Made |
| tth8 ORF1 | tth8 | Ring1 | ORF1 | no-tag | NA | Yes | Yes | No | in-house |
| tth8 ORF1 N-His | tth8 | Ring1 | ORF1 | 6xHis | N-ter | Yes | Yes | Yes | in-house |
| tth8 ORF1 C-His | tth8 | Ring1 | ORF1 | 6xHis | C-ter | Yes | Yes | Yes | in-house |
| th8 ORF2 | tth8 | Ring1 | ORF2 | no-tag | NA | Yes | Yes | Yes | in-house |
| tth8 ORF2 C-His | tth8 | Ring1 | ORF2 | 6xHis | C-ter | Yes | Yes | Yes | in-house |
| tth8 ORF1/1 | tth8 | Ring1 | ORF1/1 | no-tag | NA | Yes | Yes | Yes | in-house |
| tth8 ORF1/1 C-His | tth8 | Ring1 | ORF1/1 | 6xHis | C-ter | Yes | Yes | Yes | in-house |
| tth8 ORF1/2 | tth8 | Ring1 | ORF1/2 | no-tag | NA | Yes | Yes | Yes | in-house |
| tth8 ORF1/2 C-His | tth8 | Ring1 | ORF1/2 | 6xHis | C-ter | Yes | Yes | Yes | in-house |
| tth8 ORF2/2 | tth8 | Ring1 | ORF2/2 | no-tag | NA | Yes | Yes | Yes | in-house |
| tth8 ORF2/2 C-His | tth8 | Ring1 | ORF2/2 | 6xHis | C-ter | Yes | Yes | Yes | in-house |
| tth8 ORF2/3 | tth8 | Ring1 | ORF2/3 | no-tag | NA | Yes | Yes | Yes | in-house |
| tth8 ORF2/3 C-His | tth8 | Ring1 | ORF2/3 | 6xHis | C-ter | Yes | Yes | Yes | in-house |
| tth8 FullORF | tth8 | Ring1 | FullORF | no-tag | NA | Yes | Yes | Yes | in-house |
| tth8 FullORF C-His | tth8 | Ring1 | FullORF | 6xHis | C-ter | Yes | Yes | Yes | in-house |
| tth8 ORF2 C-His | tth8 | Ring1 | ORF2/ORF1 | 6xHis | C-ter | Yes | Yes | Yes | in-house |
| Ring 3.1 ORF1 | 6B.CD8. | Ring3.1 | ORF1 | no-tag | NA | No | No | No | in-house |
| | contig3 | | | | | | | | |
| Ring 3.1 ORF1 C-His | 6B.CD8. | Ring3.1 | ORF1 | 6xHis | C-ter | Yes | Yes | Yes | in-house |
| | contig3 | | | | | | | | |
| Ring 3.1 ORF2 | 6B.CD8. | Ring3.1 | ORF2 | no-tag | NA | No | No | No | in-house |
| | contig3 | | | | | | | | |
| Ring 3.1 ORF2 C-His | 6B.CD8. | Ring3.1 | ORF2 | 6xHis | C-ter | Yes | Yes | Yes | in-house |
| | contig3 | | | | | | | | |
| Ring 3.1 ORF2/ORF1 | 6B.CD8. | Ring3.1 | ORF2/ORF1 | 6xHis | C-ter | Yes | Yes | Yes | in-house |
| C-His | contig3 | | | | | | | | |
| LY2 FullORF | LY2 | Ring2 | FullORF | no-tag | NA | Yes | Yes | No | in-house |
| LY2 FullORF N-His | LY2 | Ring2 | FullORF | 6xHis | N-ter | Yes | Yes | Yes | in-house |
| LY2 FullORF C-His | LY2 | Ring2 | FullORF | 6xHis | C-ter | Yes | Yes | Yes | in-house |
| LY2 ORF1 | LY2 | Ring2 | ORF1 | no-tag | NA | Yes | Yes | No | in-house |
| LY2 ORF1 N-His | LY2 | Ring2 | ORF1 | 6xHis | N-ter | Yes | Yes | Yes | in-house |
| LY2 ORF1 C-His | LY2 | Ring2 | ORF1 | 6xHis | C-ter | Yes | Yes | Yes | in-house |
| LY2 ORF1(dR) | LY2 | Ring2 | ORF1 (delta- | no-tag | NA | Yes | No | No | in-house |
| | | | arginine rich | | | | | | |
| | | | region) | | | | | | |
| LY2 ORF1(dR) N-His | LY2 | Ring2 | ORF1 (delta- | 6xHis | N-ter | Yes | Yes | Yes | in-house |
| | | | arginine rich | | | | | | |
| | | | region) | | | | | | |
| LY2 ORF1(dR) C-His | LY2 | Ring2 | ORF1 (delta- | 6xHis | C-ter | Yes | Yes | Yes | in-house |
| | | | arginine rich | | | | | | |
| | | | region) | | | | | | |
| LY2 ORF1/1 | LY2 | Ring2 | ORF1/1 | no-tag | NA | Yes | Yes | No | in-house |
| LY2 ORF1/1 N-His | LY2 | Ring2 | ORF1/1 | 6xHis | N-ter | Yes | Yes | Yes | in-house |
| LY2 ORF1/1 C-His | LY2 | Ring2 | ORF1/1 | 6xHis | C-ter | Yes | Yes | Yes | in-house |
| LY2 ORF1/2 | LY2 | Ring2 | ORF1/2 | no-tag | NA | Yes | Yes | No | in-house |
| LY2 ORF1/2 N-His | LY2 | Ring2 | ORF1/2 | 6xHis | N-ter | Yes | Yes | Yes | in-house |
| LY2 ORF1/2 C-His | LY2 | Ring2 | ORF1/2 | 6xHis | C-ter | Yes | Yes | Yes | in-house |
| LY2 ORF2 | LY2 | Ring2 | ORF2 | no-tag | NA | Yes | Yes | No | in-house |
| LY2 ORF2 N-His | LY2 | Ring2 | ORF2 | 6xHis | N-ter | Yes | Yes | Yes | in-house |
| LY2 ORF2 C-His | LY2 | Ring2 | ORF2 | 6xHis | C-ter | Yes | Yes | Yes | in-house |
| LY2 ORF2/2 | LY2 | Ring2 | ORF2/2 | no-tag | NA | Yes | Yes | No | in-house |
| LY2 ORF2/2 N-His | LY2 | Ring2 | ORF2/2 | 6xHis | N-ter | Yes | Yes | Yes | in-house |
| LY2 ORF2/2 C-His | LY2 | Ring2 | ORF2/2 | 6xHis | C-ter | Yes | Yes | Yes | in-house |
| LY2 ORF2/3 | LY2 | Ring2 | ORF2/3 | no-tag | NA | Yes | Yes | No | in-house |
| LY2 ORF2/3 N-His | LY2 | Ring2 | ORF2/3 | 6xHis | N-ter | Yes | Yes | Yes | in-house |
| LY2 ORF2/3 C-His | LY2 | Ring2 | ORF2/3 | 6xHis | C-ter | Yes | Yes | Yes | in-house |
| LY2 ORF2/ORF1 C- | LY2 | Ring2 | ORF2/ORF1 | 6xHis | C-ter | Yes | Yes | Yes | in-house |
| His | | | | | | | | | |
| LY2 ORF1 HisE354 | LY2 | Ring2 | ORF1 | 6xHis | After E354 | Yes | Yes | No | in-house |
| LY2 ORF1 HisN299 | LY2 | Ring2 | ORF1 | 6xHis | After N299 | Yes | Yes | No | in-house |
| LY2 ORF1 HisL267 | LY2 | Ring2 | ORF1 | 6xHis | After L267 | Yes | Yes | No | in-house |
| tth8 ORF1 (JA20 | tth8 | Ring1 | ORF1 (with | 6xHis | C-ter | Yes | No | No | in-house |
| HVR) | | | JA20's | | | | | | |
| | | | hypervariable | | | | | | |
| | | | region) | | | | | | |
| tth8 ORF1 (TJN02 | tth8 | Ring1 | ORF1 (with | 6xHis | C-ter | Yes | No | No | in-house |
| HVR) | | | TJN02's | | | | | | |
| | | | hypervariable | | | | | | |
| | | | region) | | | | | | |
| tth8 ORF1 (TTV16 | tth8 | Ring1 | ORF1 (with | 6xHis | C-ter | Yes | No | No | in-house |
| HVR) | | | TTV16's | | | | | | |
| | | | hypervariable | | | | | | |
| | | | region) | | | | | | |
| Ring2 ORF1 (CodOpt) | LY2 | Ring2 | ORF1 (codon | no-tag | NA | Yes | Yes | Yes | Medigen |
| | | | optimized) | | | | | | |
| Ring2 ORF1 (CodOpt) | LY2 | Ring2 | ORF1 (codon | 6xHis | C-ter | Yes | Yes | Yes | Medigen |
| HRV3C-6His | | | optimized) | | | | | | |
| Ring4 ORF1 (CodOpt) | 6B.CD8. | Ring4 | ORF1 (codon | no-tag | NA | Yes | Yes | Yes | Medigen |
| | contig2 | | optimized) | | | | | | |
| RIng4 ORF1 (CodOpt) | 6B.CD8. | Ring4 | ORF1 (codon | 6xHis | C-ter | Yes | Yes | Yes | Medigen |
| HRV3C-6His | contig2 | | optimized) | | | | | | |
| RIng5.2 ORF1 | CT30F | Ring5.2 | ORF1 (codon | no-tag | NA | Yes | Yes | Yes | Medigen |
| (CodOpt) | | | optimized) | | | | | | |
| Ring5.2 ORF1 | CT30F | RIng5.2 | ORF1 (codon | 6xHis | C-ter | Yes | Yes | Yes | Medigen |
| (CodOpt) HRV3C- | | | optimized) | | | | | | |
| 6His | | | | | | | | | |
| Ring6 ORF1 (CodOpt) | 190783.3 | Ring6 | ORF1 (codon | no-tag | NA | Yes | Yes | Yes | Medigen |
| | | | optimized) | | | | | | |
| Ring6 ORF1 (CodOpt) | 190783.3 | Ring6 | ORF1 (codon | 6xHis | C-ter | Yes | Yes | Yes | Medigen |
| HRV3C-6His | | | optimized) | | | | | | |
| Ring1 ORF1 (CosOpt) | tth8 | Ring1 | ORF1 (codon | 6xHis | C-ter | Yes | Yes | Yes | Medigen |
| His | | | optimized) | | | | | | |
| Rig3.1 ORF1 | 6B.CD8. | Ring3.1 | ORF1 (codon | 6xHis | C-ter | Yes | Yes | Yes | Medigen |
| (CodOpt) His | contig3 | | optimized) | | | | | | |
| Ring7 ORF1 (CodOpt) | 190783.4 | Ring7 | ORF1 (codon | 6xHis | C-ter | Yes | Yes | Yes | Medigen |
| His | | | optimized) | | | | | | |
| Ring2 (CodOpt) N-His | LY2 | Ring2 | ORF1 (codon | 6xHis | N-ter | Yes | Yes | Yes | Medigen |
| | | | optimized) | | | | | | |
| Ring2 (CodOpt) N-His | LY2 | Ring2 | ORF1 (codon | 6xHis- | N-ter | Yes | Yes | Yes | Medigen |
| (PS) | | | optimized) | PreScision | | | | | |
| | | | | Protease | | | | | |
| | | | | recognition | | | | | |
| | | | | sequence) | | | | | |
| Ring2 tandem | LY2 | Ring2 | 2x whole | no-tag | NA | Yes | Yes | Yes | Medigen |
| | | | genome | | | | | | |
| | | | (without | | | | | | |
| | | | Polyhedrin | | | | | | |
| | | | promoter) | | | | | | |
| WTLY2 | LY2 | Ring2 | whole genome | no-tag | NA | Yes | Yes | Yes | in-house |
| WTtth8 | tth8 | Ring1 | whole genome | no-tag | NA | Yes | Yes | Yes | in-house |
| WTtth8 (Reverse) | tth8 | Ring1 | whole genome | no-tag | NA | Yes | Yes | Yes | in-house |
| | | | (with | | | | | | |
| | | | Reversed 5' | | | | | | |
| | | | Polyhedrin | | | | | | |
| | | | promoter) | | | | | | |
| LoxPWTLY2 | LY2 | Ring2 | LoxP-whole | no-tag | NA | Yes | Yes | Yes | in-house |
| | | | genome-LoxP | | | | | | |
| Cre-R | NA | NA | Cre | no-tag | NA | Yes | Yes | Yes | in-house |
| | | | recombinase | | | | | | |

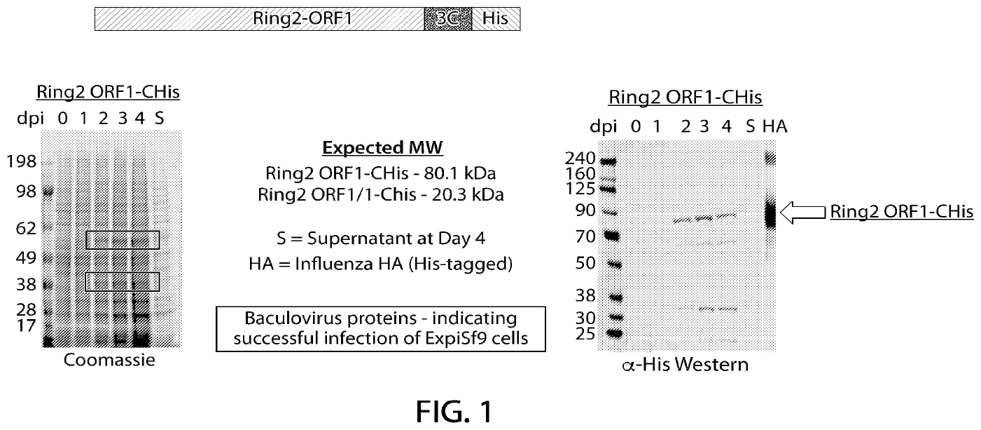

On the day before infection, ExpiSf9 cells were seeded at 5×10⁶ cells/ml in 25 ml room temperature ExpiSf9 CD Medium in 125 ml Nalgene Single-Use PETG Erlenmeyer Plain Bottom Flask [Thermofisher Scientific Catalog no: 4115-0125]. The cell viability was monitored to ensure that it was maintained at or above 95%. 100 μL of ExpiSf Enhancer solution was added to the cells in a dropwise manner. Cells were incubated with shaking overnight in a 27° C. non-humidified, air-regulated, non-CO₂ atmosphere incubator using an orbital shaker at 125±5 rpm. On Day 1, approximately 18-24 hours after adding ExpiSf Enhancer, cells were infected with the indicated baculovirus at a multiplicity of infection (MOI) of 5 and incubated under the same conditions. Cells were harvested 72 hours post infection and viability was found to range between 60 and 80%. To analyze samples, cells were lysed by adding 1× Bolt LDS sample buffer [Invitrogen Catalog No.: B0007] and 1× Bolt reducing agent [Invitrogen Catalog No.: B0009] and sonicating for 2.5 minutes. As shown in FIG. 1, C-His-tagged LY2 ORF1 was successfully expressed in infected ExpiSf9 cells by day 2 post-infection as determined by western blotting using an anti-poly-histidine antibody. In addition, baculovirus proteins were detected by Coomassie staining, indicating a successful infection.

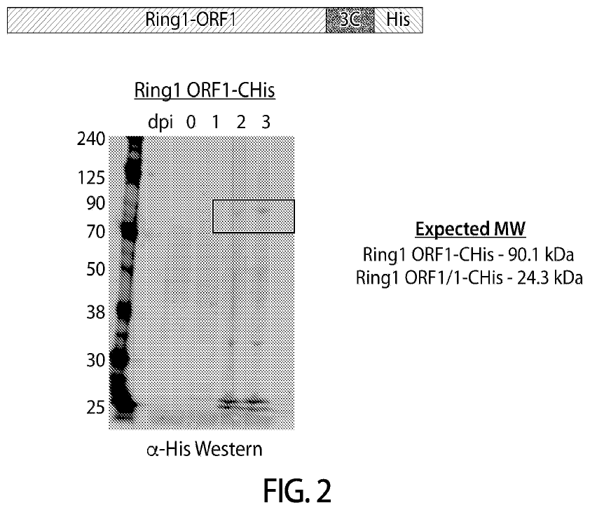

As shown in FIG. 2, C-His-tagged tth8 ORF1 and ORF1/1 were also successfully expressed in infected ExpiSf9 cells by day 2 post-infection.

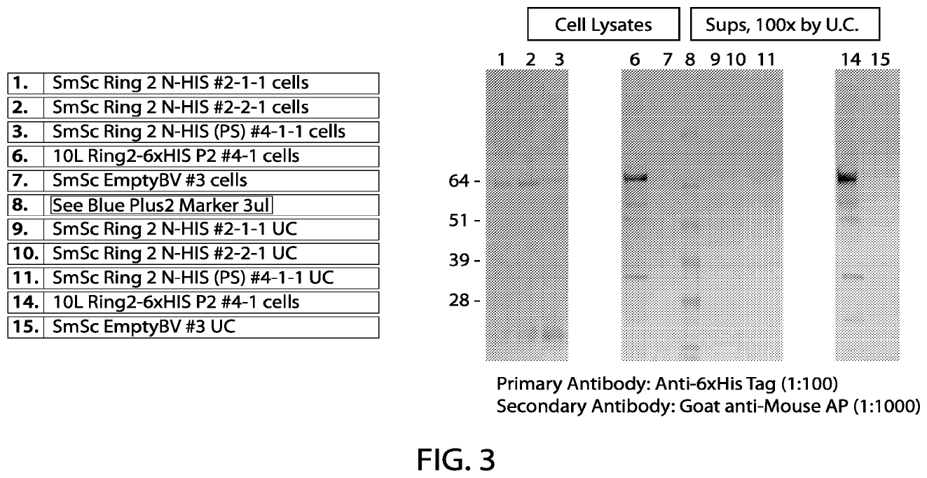

N-terminally His-tagged LY2 ORF1 expression was also detected in infected ExpiSf9 cells (FIG. 3). Here, constructs either comprised an N-terminal His-tag which was immediately followed by the wildtype ORF1 sequence (lanes 1, 2, 9, 10, or 14), or an N-terminal His-tag which was followed by a rhinovirus 3C cleavage sequence (lanes 3, 11). Samples in lanes 1 to 7 are lysates loaded directly onto the gel, whereas samples in lanes 9-15 were prepared by first pelleting protein from conditioned medium via ultracentrifugation and resuspending the pellet in a 100-fold smaller volume. Samples shown in lanes 1-3 and 9-11 were grown at a small (5 mL) scale. Samples in lanes 6 and 14 were obtained from a 10 L culture. Thus, this example shows that production of ORF1 from a plurality of strains with N or C terminal poly-histidine tags can be successfully carried out at a scale ranging from 5 mL to 10 L, and that ORF1 can be found in Sf9 lysate or culture supernatant (conditioned medium).

### Example 2: Expression of Ring1 ORFs in Sf9 Cells

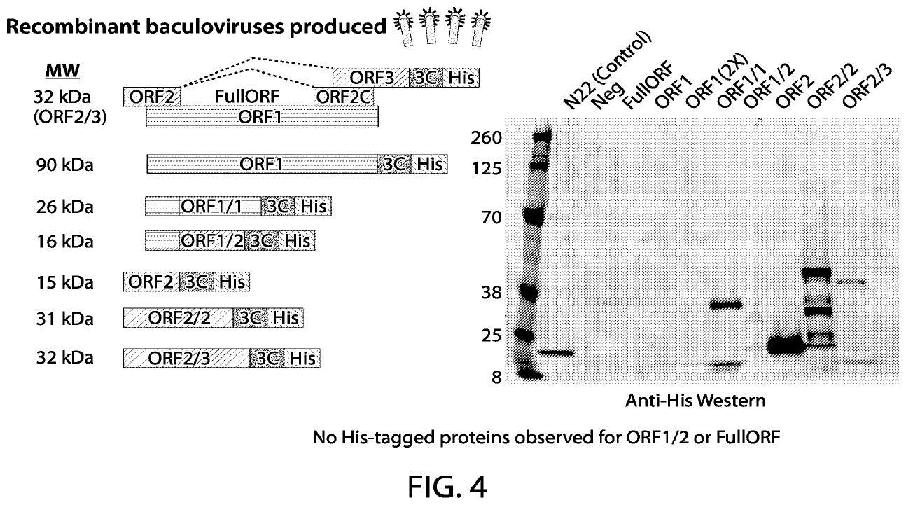

In this example, a series of recombinant baculoviruses were produced with alternate arrangements of tth8 ORFs, each tagged with a C-terminal poly-histidine (FIG. 4). The recombinant baculovirus designs included one baculovirus construct for each of the tth8 ORF splice variants (i.e., ORF1, ORF1/1, ORF1/2, ORF2, ORF2/2, and ORF2/3), as well as a “FullORF” construct containing the full ORF region from tth8, driven by the baculovirus polyhedrin promoter. These baculoviruses were produced as described in Example 1.

Protein expression was then detected by western blot using anti-poly-histidine antibody. As shown in FIG. 4, His-tagged tth8 ORFs ORF1/1, ORF1/2, ORF2, ORF2/2 and ORF2/3 were detected.

### Example 3: Expression of Ring2 ORFs in Sf9 Cells

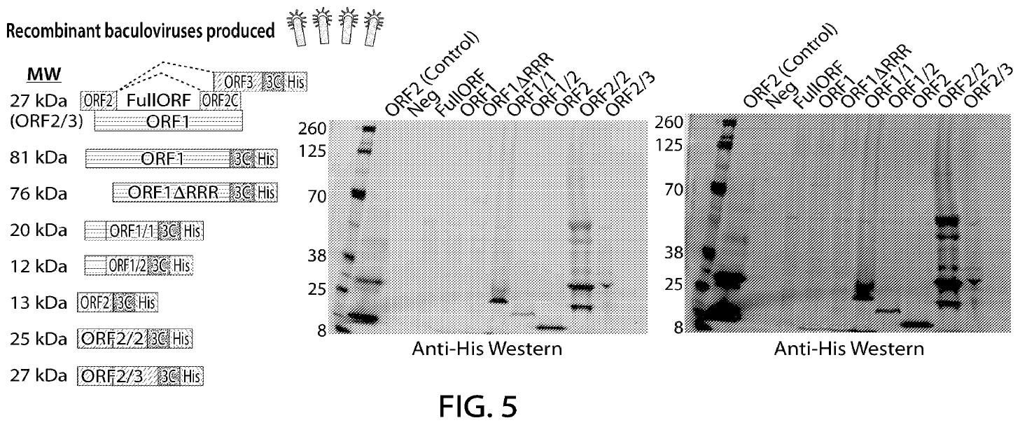

In one example, a series of recombinant baculoviruses were produced with alternate arrangements of Ring2 ORFs, each tagged with a poly-histidine tag at the C terminus (FIG. 5). The recombinant baculovirus designs included one baculovirus construct for each of the Ring2 ORF splice variants (i.e., ORF1, ORF1/1, ORF1/2, ORF2, ORF2/2, and ORF2/3), a variant in which the N-terminal arginine-rich region (RRR) is deleted (ORF1ΔRRR), as well as a “FullORF” construct containing the full ORF region from Ring2 driven by the baculovirus polyhedrin promoter. For each experimental condition, ExpiSf9 cells were infected with recombinant baculoviruses expressing individual Ring2 variants at an MOI of 5. The experimental conditions for this were as described in Examples 1 and 2.

Protein expression was then detected by western blot using anti-His. As shown in FIG. 5, His-tagged Ring2 ORFs ORF1, ORF1ΔRRR, ORF1/1, ORF1/2, ORF2, ORF2/2, and ORF2/3 were all detected.

In a further experiment as part of this example, recombinant baculoviruses comprising a Ring2 ORF1-encoding sequence and/or a Ring2 ORF2 splice variant-encoding sequence were used to infect Sf9 cells. The expression conditions tested included ORF1 alone, or co-infection of ORF1+“FullORF”, ORF1+ORF2, ORF1+ORF2/2, and ORF1+ORF2/3, as well as a negative control labeled ‘Neg’. ExpiSf9 cells were co-infected with baculoviruses at a MOI of 5 for each condition. Experimental conditions were as described in Examples 1 and 2. Protein expression of ORF1, ORF2, ORF2/2, and ORF2/3 was then assessed for each condition by western blot using either anti-His or anti-Ring2 N22. The latter is a monoclonal antibody that was obtained by immunizing mice with the N22 fragment of Ring2 ORF1 produced in E. coli, and then generating hybridomas.

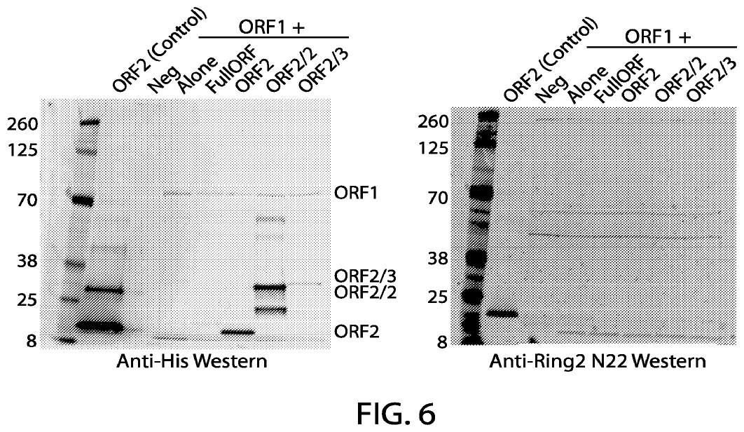

As shown in FIG. 6, both Westerns detected ORF1 as a band at ˜81 kD in each of the ORF1-infected conditions. The ORF1 band is highlighted by a dashed box in the anti-N22 Western, and is not visible in the negative control (Neg) sample. The lower molecular weight (˜10 kD) band detected by both antibodies is thought to be a C-terminal fragment of ORF1. ORF2, ORF2/2, and ORF2/3 were also detected in the corresponding samples (anti-His blot). Thus, this example illustrates that both ORF1 and individual splice variants of ORF2 can be co-expressed in insect cells.

### Example 4: Expression of all Ring2 ORFs Simultaneously in Sf9 Cells

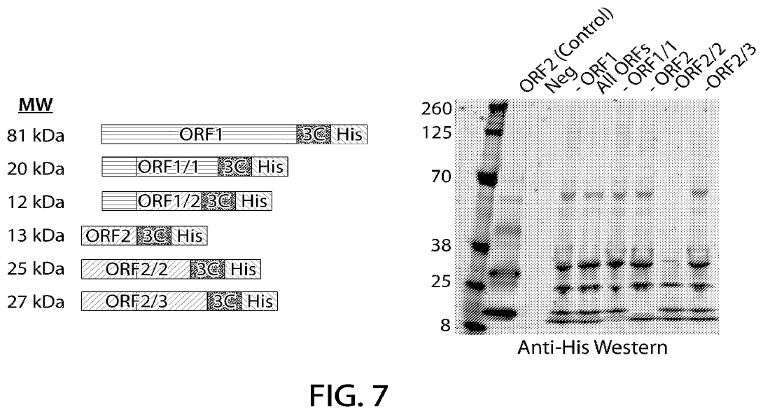

In one example, a series of six recombinant baculoviruses were produced, each designed to express a particular Ring2 ORF (i.e., ORF1, ORF1/1, ORF1/2, ORF2, ORF2/2, and ORF2/3), each tagged with a His tag (FIG. 7), as described in Example 3. Sf9 cells were infected with various combinations of the Ring2 ORF baculoviruses—specifically, each condition involved infecting cells with all but one ORF construct, as indicated in FIG. 7. Protein expression was then detected by western blot of whole cell suspension using anti-His. As shown in FIG. 7, His-tagged Ring2 ORFs were detected in the expected pattern. Either all ORFs were detected, or all except for the omitted one.

### Example 5: Co-Delivery and Independent Expression of Anellovirus Genomes and Recombinant Anellovirus ORFs in Sf9 Cells

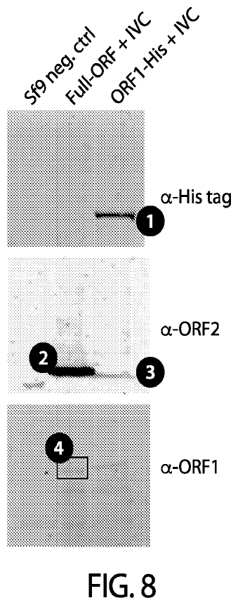

In this example, anellovirus ORFs and genomes were co-delivered in Sf9 cells by transfecting an in vitro circularized (IVC) anellovirus genome and infecting the cells with baculovirus encoding ORF1 tagged at its C-terminus with hexa-histidine (FIG. 8). Protein expression was then detected by western blot using anti-His, anti-ORF2, and anti-ORF1 monoclonal antibody targeting the N22 fragment. As shown (FIG. 8, bullet 1), His-tagged ORF1 was detected in this preparation showing successful recombinant ORF1 expression from the baculovirus vector. Consistent with this result, the same ORF1 protein was detected using the anti-ORF1 antibody (FIG. 8, bottom panel, right-most lane).

In the same sample of treated cells, the native anellovirus promoter was shown to be transcriptionally active in Sf9 cells because ORF2 expression was detected (FIG. 8, bullet 3) and could only have been produced by the IVC genome which was transfected into the cells.

In addition, Anellovirus ORFs were co-delivered and expressed in Sf9 cells using an in vitro circularized (IVC) construct and a FullORF baculovirus. Protein expression was then detected by western blot using anti-His, anti-Ring2 ORF2, and anti-Ring2 ORF1 N22. ORF1 protein was detected in the cells (FIG. 8, bullet 4) and could be the product of either the IVC or the FullORF baculovirus construct. Surprisingly, ORF2 protein was readily detected and its intensity suggests the expression is derived from the FullORF baculovirus construct (FIG. 8, bullet 2).

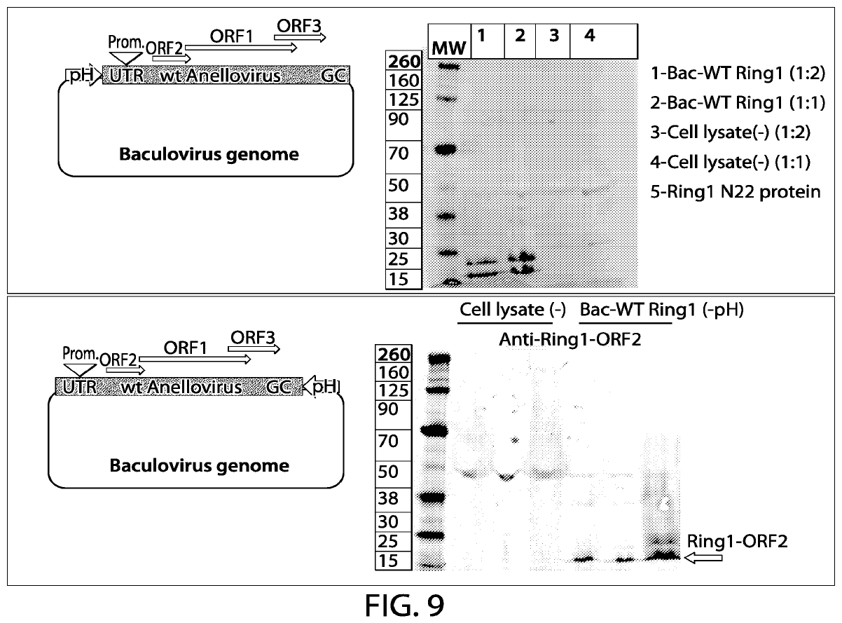

As a further test of the ability of the anellovirus genome to express its genes in insect cells, the tth8 anellovirus coding region was cloned into the pFastBac vector in both orientations. This yielded ‘FullORF’ tth8 baculovirus constructs in which the polyhedrin promoter was positioned upstream of either the sense or the anti-sense direction of the coding region. The latter configuration is highly unlikely to initiate transcription of the anellovirus genes. Consistent with our surprising observations in Ring2, expression of tth8 ORF2 was independent of the orientation of the coding region relative to the baculovirus polyhedrin promoter, suggesting that expression is driven by the anellovirus promoter (FIG. 9, bands at ˜15 and 20 kDa).

This example shows that IVC transfections and baculovirus infections can co-deliver functional anellovirus genes to Sf9 insect cells and that the native anellovirus promoter is active in these cells.

### Example 6: Anellovirus ORF1 Associates with DNA in Sf9 Cells to Form Complexes Isolated by Isopycnic Centrifugation

In this example, Sf9 cells were transfected with IVC anellovirus genome LY2, infected with a baculovirus encoding LY2 ORF1 with a C-terminal poly-histidine tag, and then fractionated to determine whether ORF1 expressed using the baculoviral expression system forms protein-DNA complexes that can be isolated in vitro.

CsCl gradients were prepared by adding 8 ml of 1.2 g/ml CsCl solution (in TN buffer; 20 mM Tris pH 8.0, 140 mM NaCl) to ultracentrifuge tubes (Ultra-Clear 17 ml-Beckman #344061) for SW32.1 Ti rotor. Tubes were underlayed with 8 ml of 40% CsCl (in TN buffer), then capped with topper and run on Gradient Master program 5-50% for 13 minutes to prepare linear gradient. The caps were removed and the gradients overlayed with 0.5 ml-2 ml of Sf9 lysate to each tube and topped off to near the top with TN buffer containing 0.001% Poloxamer-188. Ultracentrifugation was for 18.5 hours at 22,500×RPM. Fractions were collected from the gradient by piercing the bottom of the tube and allowing ˜600 ul fractions to flow into wells of a deep well block. The refractive index of each sample was measured to determine their density.

Anelloviral DNA content in the fractions was then determined by first extracting DNA from the fractions, and then by carrying out qPCR. Pure Link Viral DNA extraction Kit [Thermofisher Scientific Catalog no. 12280050] was used to purify viral DNA from 50 uL of the fractions. The samples were treated with Proteinase K and lysed using Lysis buffer by incubating at 56-C for 15 min., washed with 99% ethanol, and transferred to a Viral Spin Column. Samples were centrifuged at 6800×g, washed twice with 500 uL Wash buffer provided with the kit and centrifuged again. 100 uL of RNase-free water was added to the column to elute the DNA.

For qPCR, 2× TaqMan Gene Expression Master Mix, 100 uM LY2 primers Forward (AGCAACAGGTAATGGAGGAC), 100 uM LY2 Reverse (TGAAGCTGGGGTCTTTAAC) along with 100 uM LY2 Probe (TCTACCTAGGTGCAAAGGGCC) were diluted in 5.83 uL Nuclease Free water for each reaction. The following conditions were used for each qPCR cycle: 50° C. hold for 2 minutes, 95° C. hold for 10 minutes followed by 40 cycles of 95° C. for 15 seconds and 60° C. for 1 minute on an Applied Biosystems Quant Studio 3 Real-Time PCR Machine. Each sample was run in triplicate and the entire assay was repeated thrice and used to plot the graph.

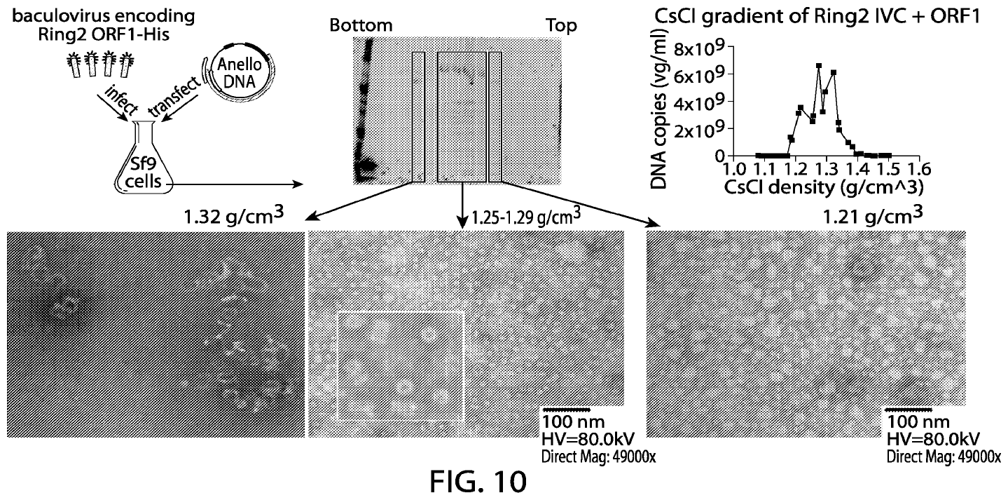

As shown in FIG. 10, isopycnic fractions were characterized by western blotting, quantitative PCR, and transmission electron microscopy. Anti-his western blotting of gradient fractions showed clear bands of the expected molecular weight for LY2 ORF1 in fractions having densities of 1.32 g/mL and 1.21 g/mL. In addition, fractions ranging from 1.25 to 1.29 g/mL had clear bands of higher and lower molecular weights than expected. Also, qPCR indicates the presence of LY2 genomic DNA in certain fractions, with peaks at approximately 1.21 g/mL, 1.29 g/mL, and 1.32 g/mL.

Negative stain transmission electron microscopy was carried out on the 1.32 g/mL and 1.21 g/mL fractions, as well as a pool of fractions ranging from 1.25 to 1.29 g/mL. The pool shows an abundance of particles, including several having the appearance of proteasomes. The presence of proteasomes may explain the western blot bands at low and high molecular weights. The former may be due to proteolytic degradation and the latter due to ubiquitylated ORF1, or ORF1 fragments covalently associated with proteasome proteins in the course of degradation. The 1.21 g/mL fraction shows particles of various sizes, including several which appear to be consistent with lipid-based particles. The 1.32 g/mL fraction shows remarkable DNA-like structures that stain differently than naked DNA, suggesting association with macromolecules such as protein.

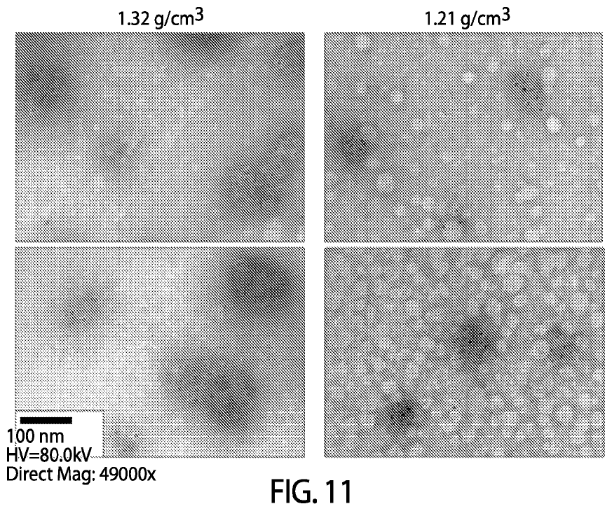

To determine if LY2 ORF1 is associated with the structures observed in the electron micrographs, immunogold detection using an anti-poly-histidine antibody was carried out. FIG. 11 shows gold label accumulating on the structures observed in the 1.32 and 1.21 g/mL fractions, consistent with the presence of ORF1-His in association with the DNA seen in the 1.32 g/mL fraction, and in the particles seen in the 1.21 g/mL fraction.

Taken together, these results show that ORF1 expressed in Sf9 cells can associate with DNA to form complexes having a density consistent with anellovirus particles.

### Example 7: Expression of ORF1 Protein from a Diverse Array of Anelloviruses Using Baculovirus

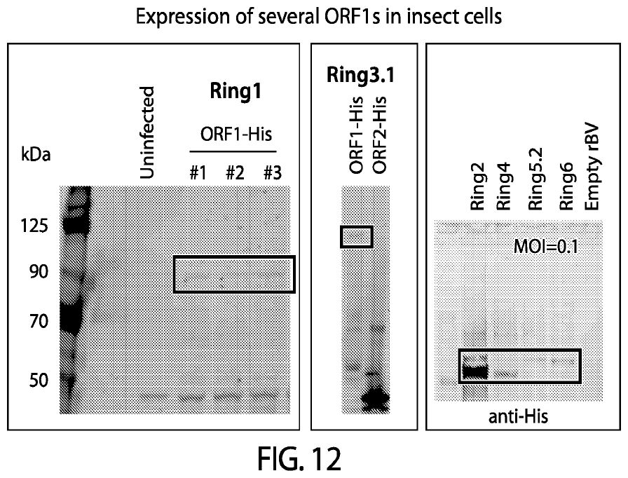

In this example, Sf9 cells were infected with baculoviruses engineered to express C-terminal His-tagged ORF1 proteins from anellovirus strains Ring3.1, Ring4, Ring5.2, Ring6, as well as Ring1 and Ring2. As shown in FIG. 12, ORF1 protein originating from each of the Anellovirus strains were successfully expressed in Sf9 cells. As shown in Table Y, Anellovirus ORF1 from the strains representing all three genera (Alphatorquevirus, Betatorquevirus, and Gammatorquevirus), were tested and their expression level is seen in FIGS. 1, 2, 3, and 12. In general, we find that the level of expression in this system is highest for ORF1 from Betatorqueviruses, intermediate from Gammatorqueviruses, and lowest from Alphatorqueviruses.

TABLE Y
Strains for which recombinant ORF1 expression was successful

| Name | Genus |
|---|---|
| Ring1 | Alpha |
| Ring2 | Beta |
| Ring3.1 | Gamma |
| Ring4 | Gamma |
| Ring5.2 | Alpha |
| Ring6 | Alpha |
| HLH | Beta |
| ctgh3 | Beta |
| LY1 | Beta |

### Example 8: In Vitro Assembly of Baculovirus Constructs

In this example, baculovirus constructs suitable for expression of Anellovirus proteins (e.g., ORF1) are generated by in vitro assembly.

DNA encoding Anellovirus ORF1 (wildtype protein, chimeric protein or fragments thereof) which may be untagged or contain tags fused N-terminally, C-terminally, or harbor mutations within the ORF1 protein itself to introduce a tag to aid in purification and/or identity determination through immunostaining assays (such as, but not limited to, ELISA or Western Blot) is expressed in insect cell lines (Sf9 and/or HighFive). Anellovirus ORF1 may be expressed alone or in combination with any number of helper proteins including, but not limited to, Anellovirus ORF2 and/or ORF3 proteins.

Protein is purified using developed purification techniques potentially including but not limited to chelating purification, heparin purification, gradient sedimentation purification and/or size exclusion purification. ORF1 is evaluated for its ability to form capsomers or VLPs and used in subsequent steps for nucleic acid encapsidation.

In one example, DNA encoding Ring2 ORF1 fused to an N-terminal HIS₆-tag (HIS-ORF1) was codon optimized for insect expression and cloned into the baculovirus expression vector pFASTbac system to generate a baculovirus expressing Ring2 ORF-HIS recombinant protein using the Bac-to-BAC expression system according to manufacturer's method (ThermoFisher Scientific). 10 liters of insect cells (Sf9) were infected with Ring2 HIS-ORF1 baculovirus and the cells were harvested 3-days post-infection by centrifugation. The cells were lysed, and the lysate was purified using a chelating resin column using standard art in the field. The elution fraction containing HIS-ORF1 was dialyzed and treated with DNAse to digest host cell DNA. The resulting material was purified again using a chelating resin column and fractions containing ORF1 were retained for nucleic acid encapsidation and viral vector purification.

Nucleic acid encapsidation and viral vector purification: Ring ORF1 (wildtype protein, chimeric protein or fragments thereof) is treated with conditions sufficient to dissociate VLPs or viral capsids to enable reassembly with nucleic acid cargo. Nucleic acid cargo can be defined as double stranded DNA, single stranded DNA, or RNA which encodes a gene of interest that one wants to deliver as a therapeutic agent. Potential conditions sufficient to dissociate VLPs or viral capsids can, but are not limited to, buffers of different pH, conditions of defined conductivity (salt content), conditions containing detergents (such as SDS, Tween, Triton), conditions containing chaotropic agents (such as Urea) or conditions involving defined temperature and time (reannealing temperatures). Nucleic acid cargo of defined concentration is combined with Ring ORF1 of defined concentration and treated with conditions sufficient to permit nucleic acid encapsidation. The resulting particle, defined as viral vector, is subsequently purified, e.g., using developed standard viral purification procedures.

In one example, single stranded circular DNA of a GFP-expression plasmid is added to a solution of Ring 2 HIS-ORF1 and the resulting sample is treated with 0.1% SDS in 50 mM Tris pH 8 buffer at 37C for 30 minutes. The resulting solution is further purified using a heparin column and the viral vector eluted from the column using a gradient of increasing NaCl concentration. The integrity of the viral vector is tested by transducing the cell lines EKVX and HEK293, and observing GFP production in at least one of the cell lines by fluorescence microscopy, demonstrating encapsidation of the nucleic acid cargo by the ORF1 protein to form the viral vector.

### Example 9: Preparation of Synthetic Anellovector Constructs

This example demonstrates in vitro production of a synthetic anellovector construct.

DNA sequences from LY1 and LY2 strains of TTMiniV (Eur Respir J. 2013 August; 42(2):470-9), between the EcoRV restriction enzyme sites, were cloned into a kanamycin vector (Integrated DNA Technologies). The resultant genetic element constructs based on DNA sequences from the LY1 and LY2 strains of TTMiniV are referred to as Anellovector 1 (Anello 1) and Anellovector 2 (Anello 2) respectively, in Examples 6 and 7. Cloned constructs were transformed into 10-Beta competent E. coli. (New England Biolabs Inc.), followed by plasmid purification (Qiagen) according to the manufacturer's protocol.

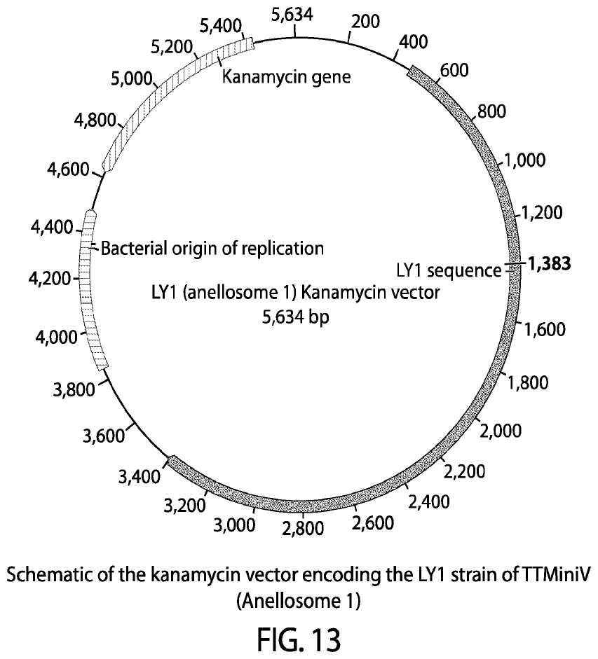

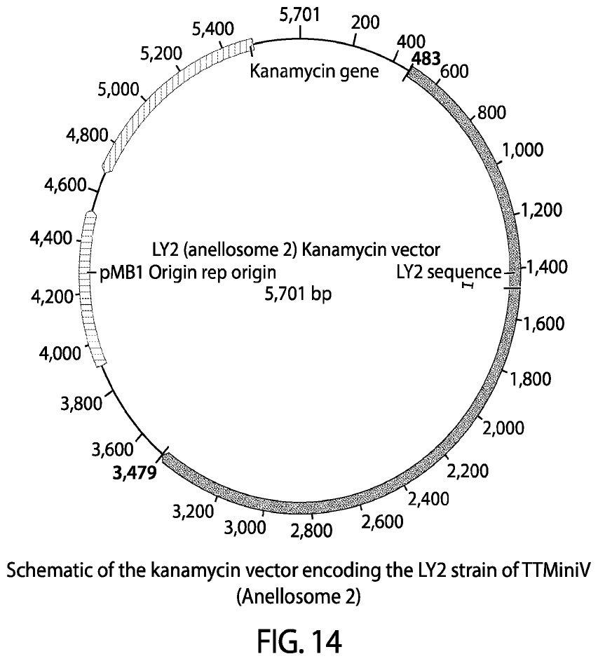

DNA constructs (FIG. 13 and FIG. 14) were linearized with EcoRV restriction digest (New England Biolabs, Inc.) at 37 degree Celsius for 6 hours, yielding double-stranded linear DNA fragments containing the TTMiniV genome, and excluding bacterial backbone elements (such as the origin of replication and selectable markers). This was followed by agarose gel electrophoresis, excision of a correctly size DNA band for the TTMiniV genome fragment (2.9 kilobase pairs), and gel purification of DNA from excised agarose bands using a gel extraction kit (Qiagen) according to the manufacturer's protocol.

In some embodiments, a method according to this Example can be performed wherein the DNA constructs can comprise one or more baculovirus elements. In some embodiments, the DNA constructs may be bacmids, e.g., suitable for transfection into an insect cell (e.g., an Sf9 cell), e.g., for production of anellovectors as described herein.

### Example 10: Assembly and Infection of Anellovectors

This example demonstrates successful in vitro production of infectious anellovectors using synthetic DNA sequences as described in Example 5.

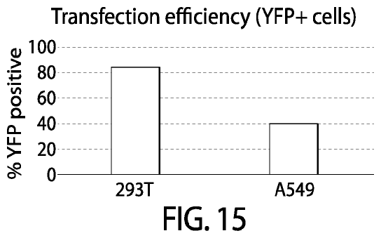

The double-stranded linearized gel-purified Anellovirus genome DNA (obtained in Example 5) was transfected into either HEK293T cells (human embryonic kidney cell line) or A549 cells (human lung carcinoma cell line), either in an intact plasmid or in linearized form, with lipid transfection reagent (Thermo Fisher Scientific). 6 ug of plasmid or 1.5 ug of linearized Anellovirus genome DNA was used for transfection of 70% confluent cells in T25 flasks. Empty vector backbone lacking the viral sequences included in the anellovector was used as a negative control. Six hours post-transfection, cells were washed with PBS twice and were allowed to grow in fresh growth medium at 37 degrees Celsius and 5% carbon dioxide. DNA sequences encoding the human Ef1alpha promoter followed by YFP gene were synthesized from IDT. This DNA sequence was blunt end ligated into a cloning vector (Thermo Fisher Scientific). The resulting vector was used as a control to assess transfection efficiency. YFP was detected using a cell imaging system (Thermo Fisher Scientific) 72 hours post transfection. The transfection efficiencies of HEK293T and A549 cells were calculated as 85% and 40% respectively (FIG. 15). In some embodiments, anellovectors could alternatively be made by inserting the Anellovirus genome DNA into a bacmid backbone and introducing the resultant bacmid into an insect cell (e.g., an Sf9 cell). In further embodiments, the Anellovirus genome DNA could be introduced into an insect cell (e.g., an Sf9 cell), e.g., comprising a bacmid comprising one or more sequences encoding an Anellovirus ORF molecule (e.g., an ORF1 molecule), or a functional fragment thereof.

Supernatants of 293T and A549 cells transfected with anellovectors were harvested 96 hours post transfection. The harvested supernatants were spun down at 2000 rpm for 10 minutes at 4 degrees Celsius to remove any cell debris. Each of the harvested supernatants was used to infect new 293T and A549 cells, respectively, that were 70% confluent in wells of 24 well plates. Supernatants were washed away after 24 hours of incubation at 37 degrees Celsius and 5% carbon dioxide, followed by two washes of PBS, and replacement with fresh growth medium. Following incubation of these cells at 37 degrees and 5% carbon dioxide for another 48 hours, cells were individually harvested for genomic DNA extraction. Genomic DNA from each of the samples was harvested using a genomic DNA extraction kit (Thermo Fisher Scientific), according to manufacturer's protocol.

To confirm the successful infection of 293T and A549 cells by anellovectors produced in vitro, 100 ng of genomic DNA harvested as described herein was used to perform quantitative polymerase chain reaction (qPCR) using primers specific for beta-torqueviruses or LY2 specific sequences. SYBR green reagent (Thermo Fisher Scientific) was used to perform qPCR, as per manufacturer's protocol. qPCR for primers specific to genomic DNA sequence of GAPDH was used for normalization. The sequences for all the primers used are listed in Table 42.

TABLE 42

| | Primer sequence (5′ > 3′) | |
|---|---|---|
| Target | Forward | Reverse |
| Betatorqueviruses | ATTCGAATGGCTGAGTTTATGC | CCTTGACTACGGTGGTTTCAC |
| | (SEQ ID NO: 690) | (SEQ ID NO: 693) |
| LY2 TTMiniV | CACGAATTAGCCAAGACTGGGCAC | TGCAGGCATTCGAGGGCTTGTT |
| strain | (SEQ ID NO: 691) | (SEQ ID NO: 694) |
| GAPDH | GCTCCCACTCCTGATTTCTG (SEQ | TTTAACCCCCTAGTCCCAGG |
| | ID NO: 692) | (SEQ ID NO: 695) |

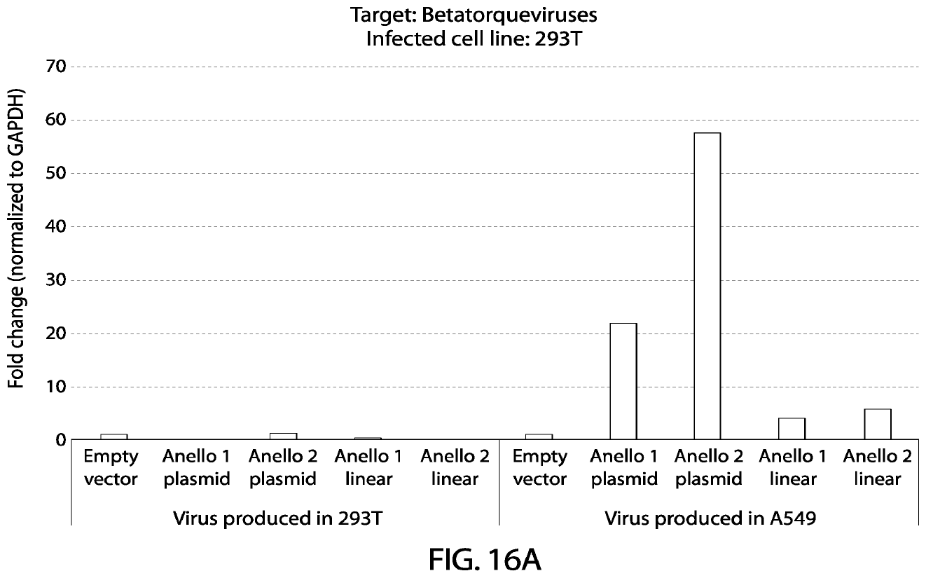

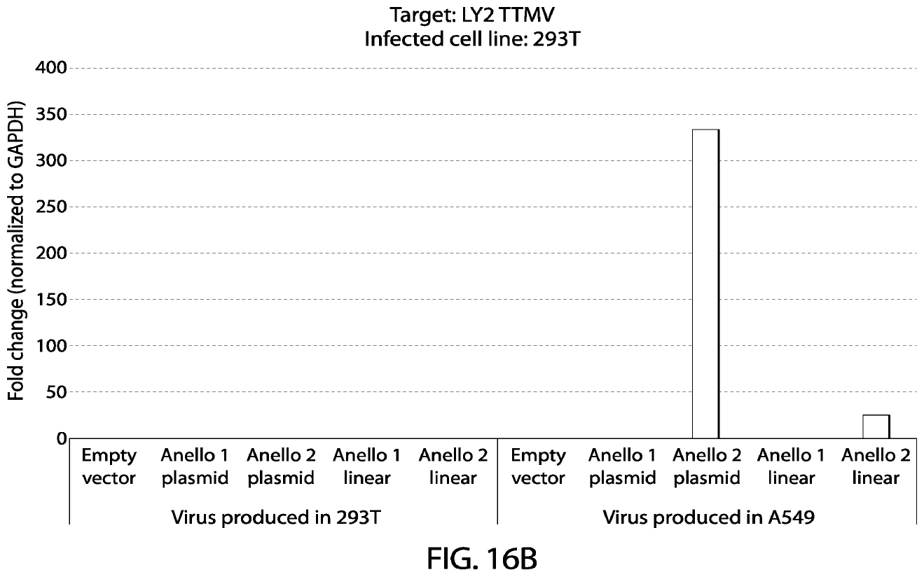

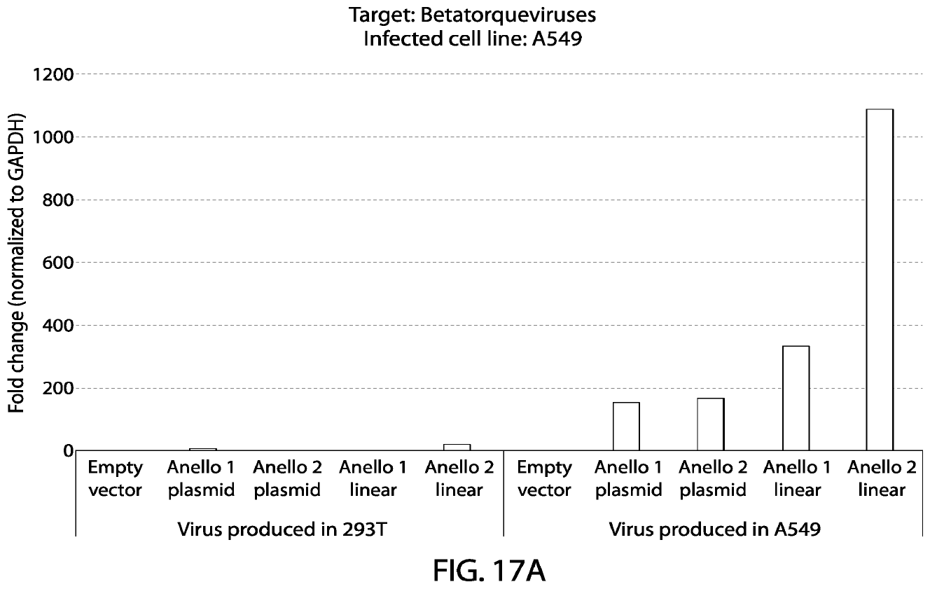

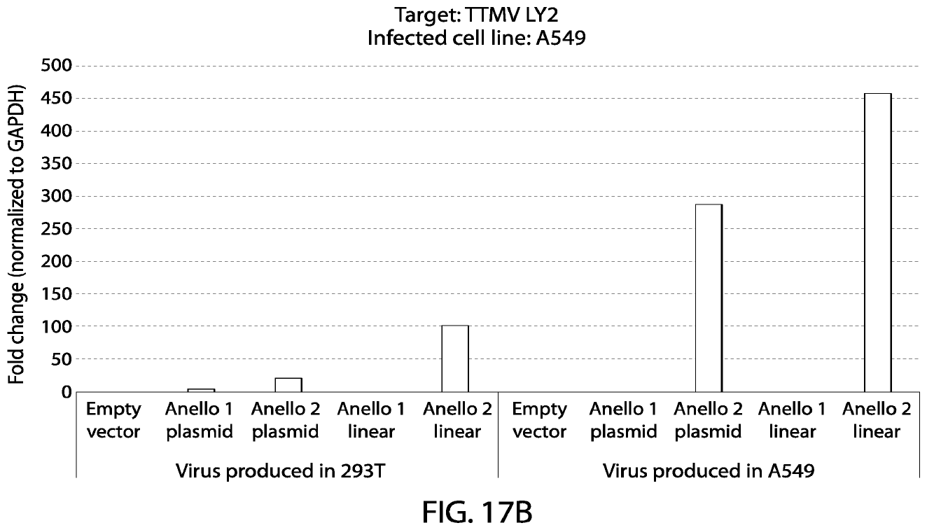

As shown in the qPCR results depicted in FIGS. 16A, 16B, 17A, and 17B, the anellovectors produced in vitro and as described in this example were infectious.

### Example 11: Selectivity of Anellovectors

This example demonstrates the ability of synthetic anellovectors produced in vitro to infect cell lines of a variety of tissue origins.

Supernatants with the infectious TTMiniV anellovectors (described in Example 5) were incubated with 70% confluent 293T, A549, Jurkat (an acute T cell leukemia cell line), Raji (a Burkitt's lymphoma B cell line), and Chang cell lines at 37 degrees and 5% carbon dioxide in wells of 24 well plates. Cells were washed with PBS twice, 24 hours post infection, followed by replacement with fresh growth medium. Cells were then incubated again at 37 degrees and 5% carbon dioxide for another 48 hours, followed by harvest for genomic DNA extraction. Genomic DNA from each of the samples was harvested using a genomic DNA extraction kit (Thermo Fisher Scientific), according to manufacturer's protocol.

To confirm successful infection of these cell lines by anellovectors produced in the previous Example, 100 ng of genomic DNA harvested as described herein was used to perform quantitative polymerase chain reaction (qPCR) using primers specific for beta-torqueviruses or LY2 specific sequences. SYBR green reagent (Thermo Fisher Scientific) was used to perform qPCR, as per manufacturer's protocol. qPCR for primers specific to genomic DNA sequence of GAPDH was used for normalization. The sequences for all the primers used are listed in Table 42.

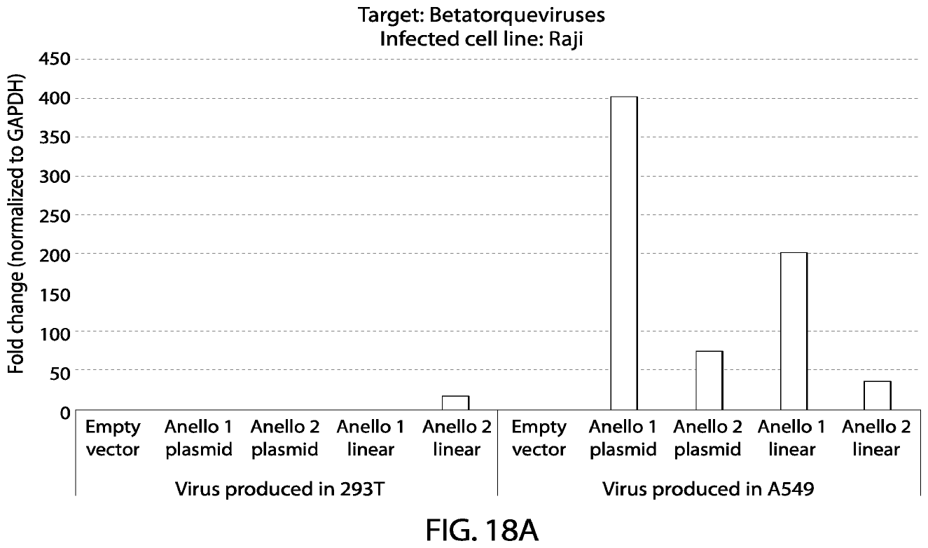

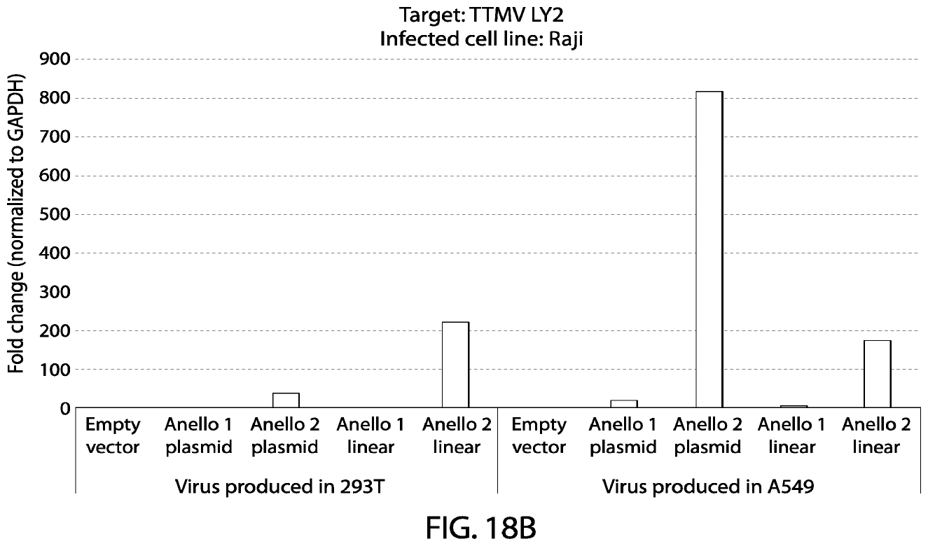

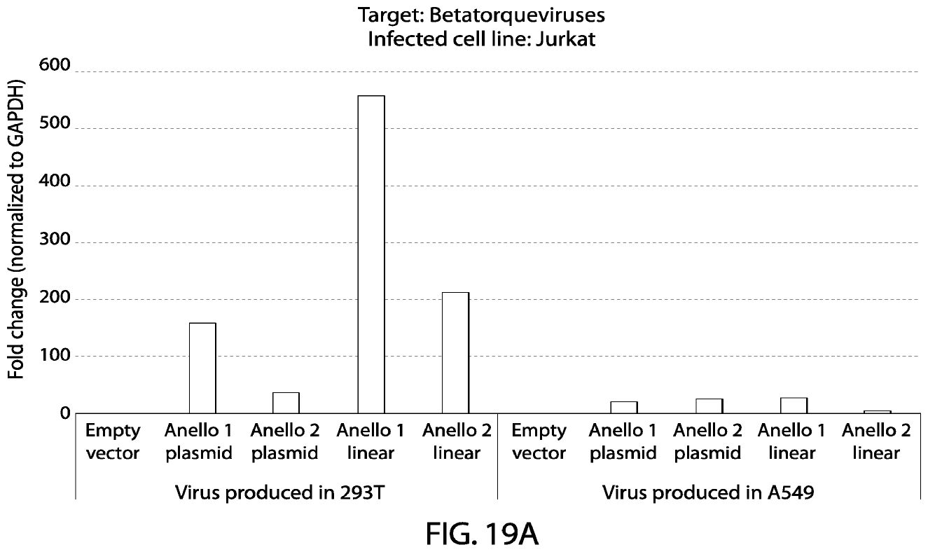

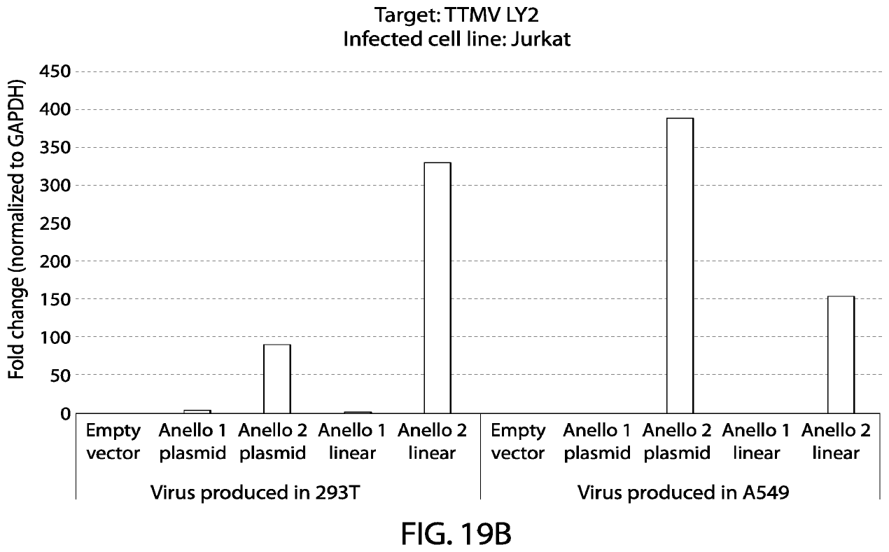

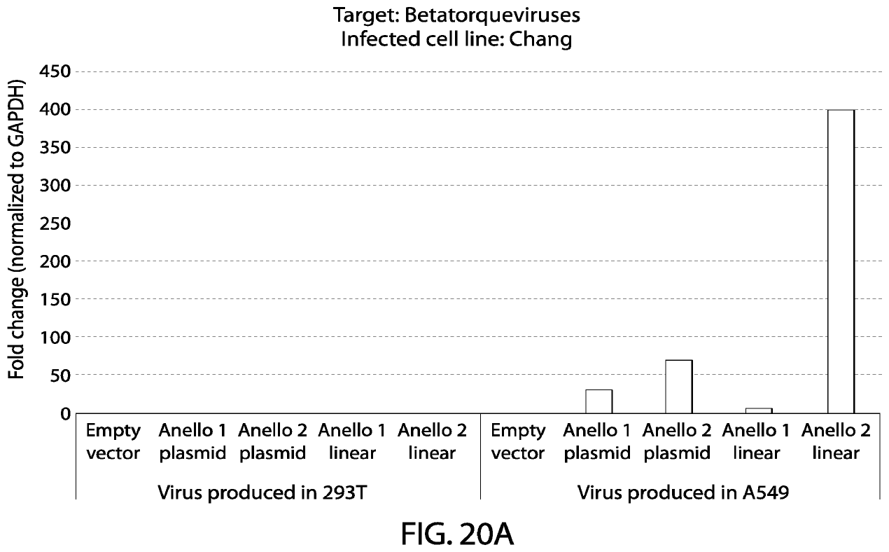

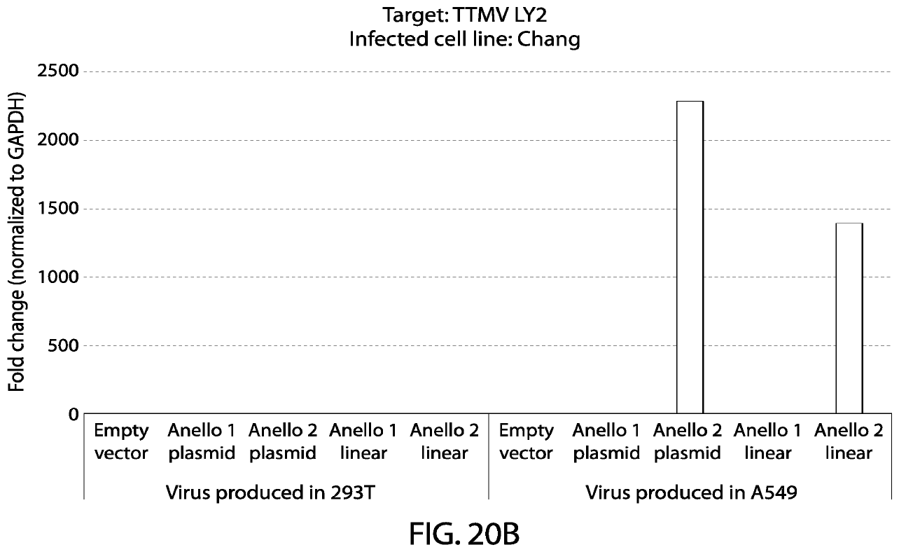

As shown in the qPCR results depicted in FIGS. 16A-20B, not only were anellovectors produced in vitro infectious, they were able to infect a variety of cell lines, including examples of epithelial cells, lung tissue cells, liver cells, carcinoma cells, lymphocytes, lymphoblasts, T cells, B cells, and kidney cells. It was also observed that a synthetic anellovector was able to infect HepG2 cells (a liver cell line), resulting in a greater than 100-fold increase relative to a control.

Alternatively, the method of this Example can be performed with anellovectors produced using a bacmid/insect cell system, e.g., as described herein.

### Example 12: Replication-Deficient Anellovectors and Helper Viruses

For replication and packaging of an anellovector, some elements can be provided in trans. These include proteins or non-coding RNAs that direct or support DNA replication or packaging. Trans elements can, in some instances, be provided from a source alternative to the anellovector, such as a bacmid, helper virus, plasmid, or from the cellular genome. In some embodiments, the elements are provided in trans in a separate construct (e.g., in a separate bacmid). In some embodiments, the trans elements and the genetic element are comprised in an insect cell (e.g., an Sf9 cell).

Other elements are typically provided in cis. These elements can be, for example, sequences or structures in the anellovector DNA that act as origins of replication (e.g., to allow amplification of anellovector DNA) or packaging signals (e.g., to bind to proteins to load the genome into the capsid). Generally, a replication deficient virus or anellovector will be missing one or more of these elements, such that the DNA is unable to be packaged into an infectious virion or anellovector even if other elements are provided in trans. In some embodiments, the cis elements are comprised in a genetic element construct (e.g., a bacmid or a non-bacmid construct), e.g., as described herein.

Replication deficient viruses can be useful as helper viruses, e.g., for controlling replication of an anellovector (e.g., a replication-deficient or packaging-deficient anellovector) in the same cell. In some instances, the helper virus will lack cis replication or packaging elements, but express trans elements such as proteins and non-coding RNAs. Generally, the therapeutic anellovector would lack some or all of these trans elements and would therefore be unable to replicate on its own, but would retain the cis elements. When co-transfected/infected into cells, the replication-deficient helper virus would drive the amplification and packaging of the anellovector. The packaged particles collected would thus be comprised solely of therapeutic anellovector, without helper virus contamination.

To develop a replication deficient anellovector, conserved elements in the non-coding regions of Anellovirus will be removed. In particular, deletions of the conserved 5′ UTR domain and the GC-rich domain will be tested, both separately and together. Both elements are contemplated to be important for viral replication or packaging. Additionally, deletion series will be performed across the entire non-coding region to identify previously unknown regions of interest.

Successful deletion of a replication element will result in reduction of anellovector DNA amplification within the cell, e.g., as measured by qPCR, but will support some infectious anellovector production, e.g., as monitored by assays on infected cells that can include any or all of qPCR, western blots, fluorescence assays, or luminescence assays. Successful deletion of a packaging element will not disrupt anellovector DNA amplification, so an increase in anellovector DNA will be observed in transfected cells by qPCR. However, the anellovector genomes will not be encapsulated, so no infectious anellovector production will be observed.

### Example 13: Manufacturing Process for Replication-Competent Anellovectors

This example describes a method for recovery and scaling up of production of replication-competent anellovectors. Anellovectors are replication competent when they encode in their genome all the required genetic elements and ORFs necessary to replicate in cells. Since these anellovectors are not defective in their replication they do not need a complementing activity provided in trans. They might, however need helper activity, such as enhancers of transcriptions (e.g. sodium butyrate) or viral transcription factors (e.g. adenoviral E1, E2 E4, VA; HSV Vp16 and immediate early proteins). In some embodiments, the replication-competent anellovector is produced an insect cell (e.g., an Sf9 cell), e.g., using a bacmid as described herein.

In this example, double-stranded DNA encoding the full sequence of a synthetic anellovector either in its linear or circular form is introduced into 5E+05 adherent mammalian cells in a T75 flask by chemical transfection or into 5E+05 cells in suspension by electroporation. After an optimal period of time (e.g., 3-7 days post transfection), cells and supernatant are collected by scraping cells into the supernatant medium. A mild detergent, such as a biliary salt, is added to a final concentration of 0.5% and incubated at 37° C. for 30 minutes. Calcium and Magnesium Chloride is added to a final concentration of 0.5 mM and 2.5 mM, respectively. Endonuclease (e.g., DNAse I, Benzonase), is added and incubated at 25-37° C. for 0.5-4 hours. Anellovector suspension is centrifuged at 1000×g for 10 minutes at 4° C. The clarified supernatant is transferred to a new tube and diluted 1:1 with a cryoprotectant buffer (also known as stabilization buffer) and stored at −80° C. if desired. This produces passage 0 of the anellovector (P0). To bring the concentration of detergent below the safe limit to be used on cultured cells, this inoculum is diluted at least 100-fold or more in serum-free media (SFM) depending on the anellovector titer.

A fresh monolayer of mammalian cells in a T225 flask is overlaid with the minimum volume sufficient to cover the culture surface and incubated for 90 minutes at 37° C. and 5% carbon dioxide with gentle rocking. The mammalian cells used for this step may or may not be the same type of cells as used for the P0 recovery. After this incubation, the inoculum is replaced with 40 ml of serum-free, animal origin-free culture medium. Cells are incubated at 37° C. and 5% carbon dioxide for 3-7 days. 4 ml of a 10× solution of the same mild detergent previously utilized is added to achieve a final detergent concentration of 0.5%, and the mixture is then incubated at 37° C. for 30 minutes with gentle agitation. Endonuclease is added and incubated at 25-37° C. for 0.5-4 hours. The medium is then collected and centrifuged at 1000×g at 4° C. for 10 minutes. The clarified supernatant is mixed with 40 ml of stabilization buffer and stored at −80° C. This generates a seed stock, or passage 1 of anellovector (P1).

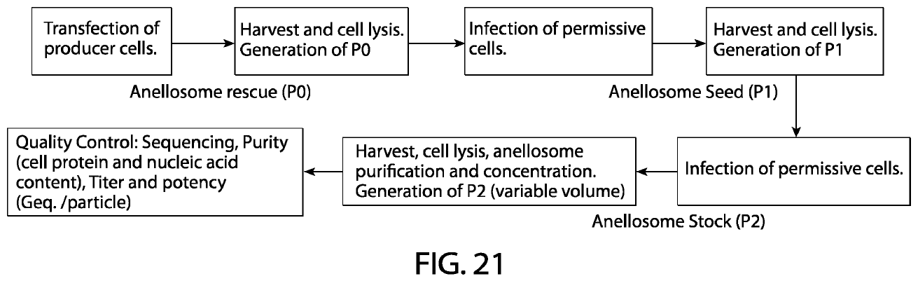

Depending on the titer of the stock, it is diluted no less than 100-fold in SFM and added to cells grown on multilayer flasks of the required size. Multiplicity of infection (MOI) and time of incubation is optimized at smaller scale to ensure maximal anellovector production. After harvest, anellovectors may then be purified and concentrated as needed. A schematic showing a workflow, e.g., as described in this example, is provided in FIG. 21.

### Example 14: Manufacturing Process of Replication-Deficient Anellovectors

This example describes a method for recovery and scaling up of production of replication-deficient anellovectors.

Anellovectors can be rendered replication-deficient by deletion of one or more ORFs (e.g., ORF1, ORF1/1, ORF1/2, ORF2, ORF2/2, ORF2/3, and/or ORF2t/3) involved in replication. Replication-deficient anellovectors can be grown in a complementing cell line. Such cell line constitutively expresses components that promote anellovector growth but that are missing or nonfunctional in the genome of the anellovector. In some embodiments, replication-deficient anellovectors are grown in an insect cell line (e.g., Sf9 cells), e.g., comprising bacmids as described herein.

In one example, the sequence(s) of any ORF(s) involved in anellovector propagation are cloned into a lentiviral expression system suitable for the generation of stable cell lines that encode a selection marker, and lentiviral vector is generated as described herein. A mammalian cell line capable of supporting anellovector propagation is infected with this lentiviral vector and subjected to selective pressure by the selection marker (e.g., puromycin or any other antibiotic) to select for cell populations that have stably integrated the cloned ORFs. Once this cell line is characterized and certified to complement the defect in the engineered anellovector, and hence to support growth and propagation of such anellovectors, it is expanded and banked in cryogenic storage. During expansion and maintenance of these cells, the selection antibiotic is added to the culture medium to maintain the selective pressure. Once anellovectors are introduced into these cells, the selection antibiotic may be withheld.

Once this cell line is established, growth and production of replication-deficient anellovectors is carried out, e.g., as described in Example 15.

### Example 15: Production of Anellovectors Using Suspension Cells

This example describes the production of anellovectors in cells in suspension.

In this example, an A549 or 293T producer cell line that is adapted to grow in suspension conditions is grown in animal component-free and antibiotic-free suspension medium (Thermo Fisher Scientific) in WAVE bioreactor bags at 37 degrees and 5% carbon dioxide. These cells, seeded at 1×10⁶ viable cells/mL, are transfected using lipofectamine 2000 (Thermo Fisher Scientific) under current good manufacturing practices (cGMP), with a plasmid comprising anellovector sequences, along with any complementing plasmids suitable or required to package the anellovector (e.g., in the case of a replication-deficient anellovector, e.g., as described in Example 16). The complementing plasmids can, in some instances, encode for viral proteins that have been deleted from the anellovector genome (e.g., an anellovector genome based on a viral genoe, e.g., an Anellovirus genome, e.g., as described herein) but are useful or required for replication and packaging of the anellovectors. Transfected cells are grown in the WAVE bioreactor bags and the supernatant is harvested at the following time points: 48, 72, and 96 hours post transfection. The supernatant is separated from the cell pellets for each sample using centrifugation. The packaged anellovector particles are then purified from the harvested supernatant and the lysed cell pellets using ion exchange chromatography.

The genome equivalents in the purified prep of the anellovectors can be determined, for example, by using a small aliquot of the purified prep to harvest the anellovector genome using a viral genome extraction kit (Qiagen), followed by qPCR using primers and probes targeted towards the anellovector DNA sequence, e.g., as described in Example 18.

The infectivity of the anellovectors in the purified prep can be quantified by making serial dilutions of the purified prep to infect new A549 cells. These cells are harvested 72 hours post transfection, followed by a qPCR assay on the genomic DNA using primers and probes that are specific to the anellovector DNA sequence.

In some embodiments, the anellovectors are produced in insect cells (e.g., Sf9 cells), e.g., comprising bacmids as described herein, in suspension.

### Example 16: Utilizing Anellovectors to Express an Exogenous Protein in Mice

This example describes the usage of an anellovector in which the Torque Teno Mini Virus (TTMV) genome is engineered to express the firefly luciferase protein in mice.

The plasmid encoding the DNA sequence of the engineered TTMV encoding the firefly-luciferase gene is introduced into A549 cells (human lung carcinoma cell line) by chemical transfection. 18 ug of plasmid DNA is used for transfection of 70% confluent cells in a 10 cm tissue culture plate. Empty vector backbone lacking the TTMV sequences is used as a negative control. Five hours post-transfection, cells are washed with PBS twice and are allowed to grow in fresh growth medium at 37° C. and 5% carbon dioxide.

Transfected A549 cells, along with their supernatant, are harvested 96 hours post transfection. Harvested material is treated with 0.5% deoxycholate (weight in volume) at 37° C. for 1 hour followed by endonuclease treatment. Anellovector particles are purified from this lysate using ion exchange chromatography. To determine anellovector concentration, a sample of the anellovector stock is run through a viral DNA purification kit and genome equivalents per ml are measured by qPCR using primers and probes targeted towards the anellovector DNA sequence.

A dose-range of genome equivalents of anellovectors in 1× phosphate-buffered saline is performed via a variety of routes of injection (e.g. intravenous, intraperitoneal, subcutaneous, intramuscular) in mice at 8-10 weeks of age. Ventral and dorsal bioluminescence imaging is performed on each animal at 3, 7, 10 and 15 days post injection. Imaging is performed by adding the luciferase substrate (Perkin-Elmer) to each animal intraperitoneally at indicated time points, according to the manufacturer's protocol, followed by intravital imaging.

In some embodiments, the anellovectors are produced using bacmids and insect cells, e.g., as described herein.

### Example 17: Functional Effects of an Anellovector Expressing an Exogenous microRNA Sequence

This example demonstrates the successful expression of an exogenous miRNA (miR-625) from anellovector genome using a native promoter.

500 ng of following plasmid DNAs were transfected into 60% confluent wells of HEK293T cells in a 24 well plate:
i) Empty plasmid backbone
ii) Plasmid containing TTV-tth8 genome in which endogenous miRNA is knocked out (KO)
iii) TTV-tth8 in which endogenous miRNA is replaced with a non-targeting scramble miRNA
iv) TTV-tth8 in which endogenous miRNA sequence is replaced with miRNA encoding miR-625

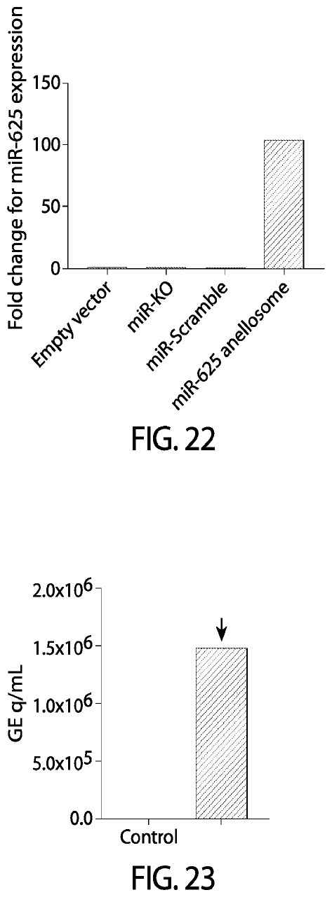

72 hours post transfection, total miRNA was harvested from the transfected cells using the Qiagen miRNeasy kit, followed by reverse transcription using miRNA Script RT II kit. Quantitative PCR was performed on the reverse transcribed DNA using primer that should specifically detect miRNA-625 or RNU6 small RNA. RNU6 small RNA was used as a housekeeping gene and data is plotted in FIG. 22 as a fold change relative to empty vector. As shown in FIG. 22, miR-625 anellovector resulted in approximately 100-fold increase in miR-625 expression, whereas no signal was detected for empty vector, miR-knockout (KO), and scrambled miR.

In some embodiments, the cells instead can be infected with anellovectors produced using bacmids and insect cells, e.g., as described herein.

### Example 18: Preparation and Production of Anellovectors to Express Exogenous Non-Coding RNAs

This example describes the synthesis and production of anellovectors to express exogenous small non-coding RNAs.

The DNA sequence from the tth8 strain of TTV (Jelcic et al, Journal of Virology, 2004) is synthesized and cloned into a vector containing the bacterial origin of replication and bacterial antibiotic resistance gene. In this vector, the DNA sequence encoding the TTV miRNA hairpin is replaced by a DNA sequence encoding an exogenous small non-coding RNA such as miRNA or shRNA. The engineered construct is then transformed into electro-competent bacteria, followed by plasmid isolation using a plasmid purification kit according to the manufacturer's protocols. In some embodiments, the vector is a bacmid or a donor vector, e.g., as described herein.

The anellovector DNA encoding the exogenous small non-coding RNAs is transfected into an eukaryotic producer cell line (e.g., an insect cell) to produce anellovector particles. The supernatant of the transfected cells containing the anellovector particles is harvested at different time points post transfection. Anellovector particles, either from the filtered supernatant or after purification, are used for downstream applications, e.g., as described herein.

### Example 19: Expression of an Endogenous miRNA from an Anellovector and Deletion of the Endogenous miRNA

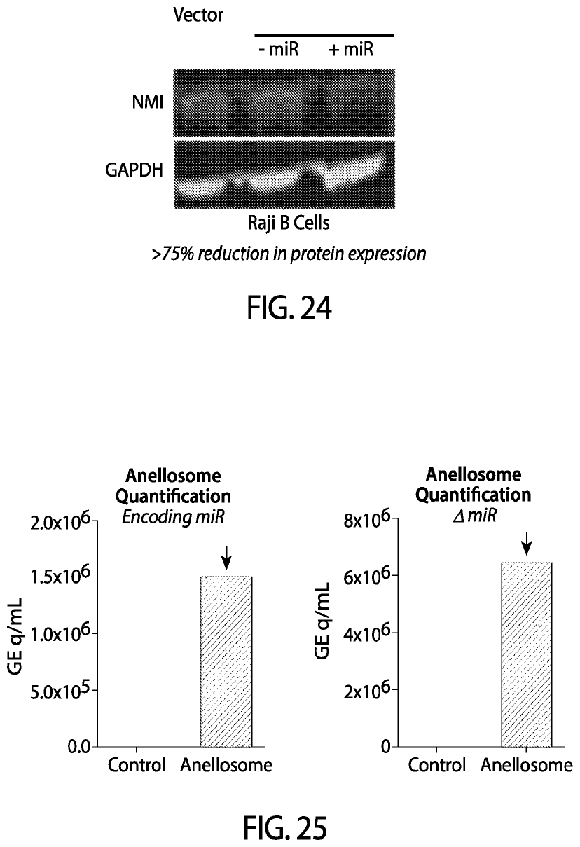

In one example, anellovectors comprising a modified TTV-tth8 genome, in which the TTV-tth8 genome was modified with a deletion in the GC-rich region as described in Example 27, were used to infect Raji B cells in culture. These anellovectors comprised a sequence encoding the endogenous payload of the TTV-tth8 Anellovirus, which is a miRNA targeting the mRNA encoding n-myc interacting protein (NMI), and were produced by introducing a plasmid comprising the Anellovirus genome into a host cell. NMI operates downstream of the JAK/STAT pathway to regulate the transcription of various intracellular signals, including interferon-stimulated genes, proliferation and growth genes, and mediators of the inflammatory response. As shown in FIG. 23, viral genomes were detected in target Raji B cells. Successful knockdown of NMI was also observed in target Raji B cells compared to control cells (FIG. 24). Anellovector comprising the miRNA against NMI induced a greater than 75% reduction in NMI protein levels compared to control cells. This example demonstrates that an anellovector with a native Anellovirus miRNA can knock down a target molecule in host cells.

In another example, the endogenous miRNA of an Anellovirus-based anellovector was deleted. The resultant anellovector (A miR) was then incubated with host cells. Genome equivalents of A miR anellovector genetic elements was then compared to that of corresponding anellovectors in which the endogenous miRNA was retained. As shown in FIG. 25, anellovector genomes in which the endogenous miRNA were deleted were detected in cells at levels comparable to those observed for anellovector genomes in which the endogenous miRNA was still present. This example demonstrates that the endogenous miRNA of an Anellovirus-based anellovector can be mutated, or deleted entirely and the anellovector genome can still be detected in target cells.

In some embodiments, the cells instead can be infected with anellovectors produced using bacmids and insect cells, e.g., as described herein.

### Example 20: Anellovector Delivery of Exogenous Proteins In Vivo

This example demonstrates in vivo effector function (e.g. expression of proteins) of anellovectors after administration.

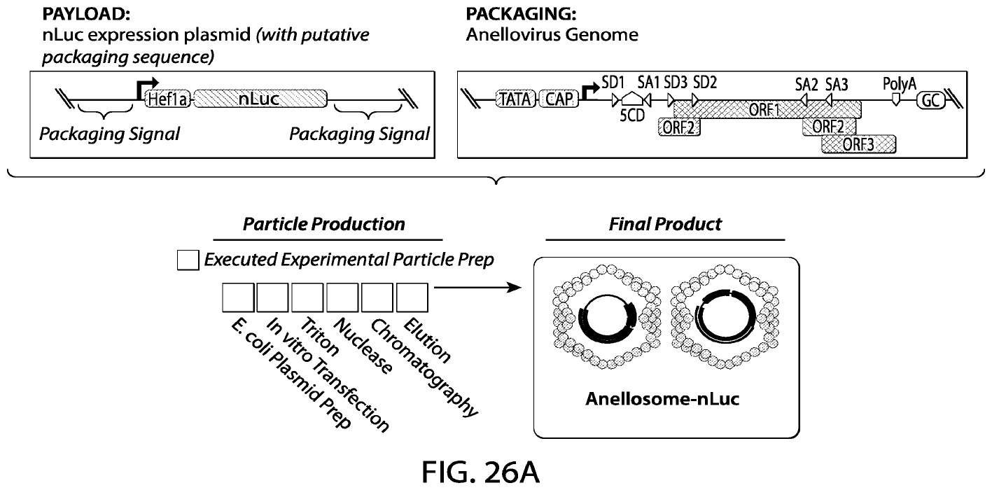

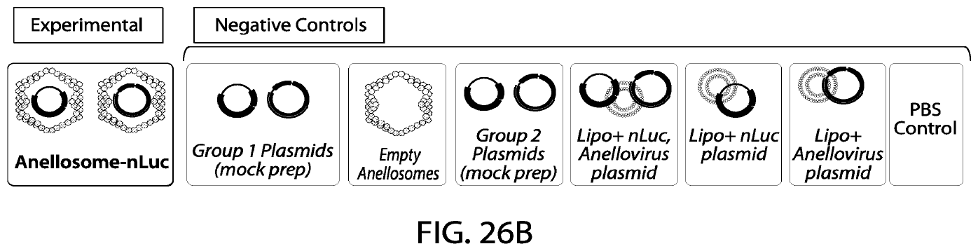

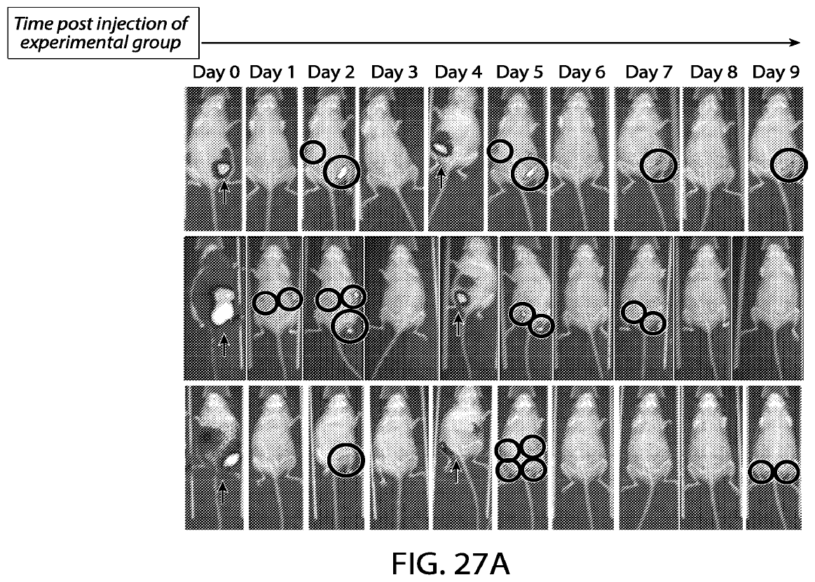

Anellovectors comprising a transgene encoding nano-luciferase (nLuc) (FIGS. 26A-26B) were prepared. Briefly, double-stranded DNA plasmids harboring the TTMV-LY2 non-coding regions and an nLuc expression cassette were transfected into HEK293T cells along with double-stranded DNA plasmids encoding the full TTMV-LY2 genome to act as trans replication and packaging factors. After transfection, cells were incubated to permit anellovector production and anellovector material was harvested and enriched via nuclease treatment, ultrafiltration/diafiltration, and sterile filtration. Additional HEK293T cells were transfected with non-replicating DNA plasmids harboring nLuc expression cassettes and TTMV-LY2 ORF transfection cassettes, but lacking non-coding domains essential for replication and packaging, to act as a “non-viral” negative control. The non-viral samples were prepared following the same protocol as the anellovector material.

Anellovector preparation was administered to a cohort of three healthy mice intramuscularly, and monitored by IVIS Lumina imaging (Bruker) over the course of nine days (FIG. 27A). As a non-viral control, the non-replicating preparation was administered to three additional mice (FIG. 27B). Injections of 25 μL of anellovector or non-viral preparations were administered to the left hind leg on Day 0, and re-administered to the right hind leg on Day 4 (See arrows in FIGS. 27A and 27B). After 9 days of IVIS imaging, more occurrences of nLuc luminescent signal were observed in mice injected with the anellovector preparation (FIG. 27A) than the non-viral preparation (FIG. 27B), which is consistent with trans gene expression after in vivo anellovector transduction.

In some embodiments, the mice instead can be administered anellovectors produced using bacmids and insect cells, e.g., as described herein.

### Example 21: In Vitro Circularized Anellovirus Genomes

This example describes constructs comprising circular, double stranded Anelloviral genome DNA with minimal non-viral DNA. These circular viral genomes more closely match the double-stranded DNA intermediates found during wild-type Anellovirus replication. When introduced into a cell, such circular, double stranded Anelloviral genome DNA with minimal non-viral DNA can undergo rolling circle replication to produce, for example, a genetic element as described herein.

In one example, plasmids harboring TTV-tth8 variants and TTMV-LY2 were digested with restriction endonucleases recognizing sites flanking the genomic DNA. The resulting linearized genomes were then ligated to form circular DNA. These ligation reactions were done with varying DNA concentrations to optimize the intramolecular ligations. The ligated circles were either directly transfected into mammalian cells, or further processed to remove non-circular genome DNA by digesting with restriction endonucleases to cleave the plasmid backbone and exonucleases to degrade linear DNA. For TTV-tth8, XmaI endonuclease was used to linearize the DNA; the ligated circle contained 53 bp of non-viral DNA between the GC-rich region and the 5′ non-coding region. For TTMV-LY2, the type IIS restriction enzyme Esp3I was used, yielding a viral genomic DNA circle with no non-viral DNA. This protocol was adapted from previously published circularizations of TTV-tth8 (Kincaid et al., 2013, PLoS Pathogens 9(12): e1003818). To demonstrate the improvements in Anellovirus production, circularized TTV-tth8 and TTMV-LY2 were transfected into HEK293T cells. After 7 days of incubation, cells were lysed, and qPCR was performed to compare the levels of anellovirus genome between circularized and plasmid-based anelloviral genomes. Increased levels of Anelloviral genomes show that circularization of the viral DNA is a useful strategy for increasing Anellovirus production.

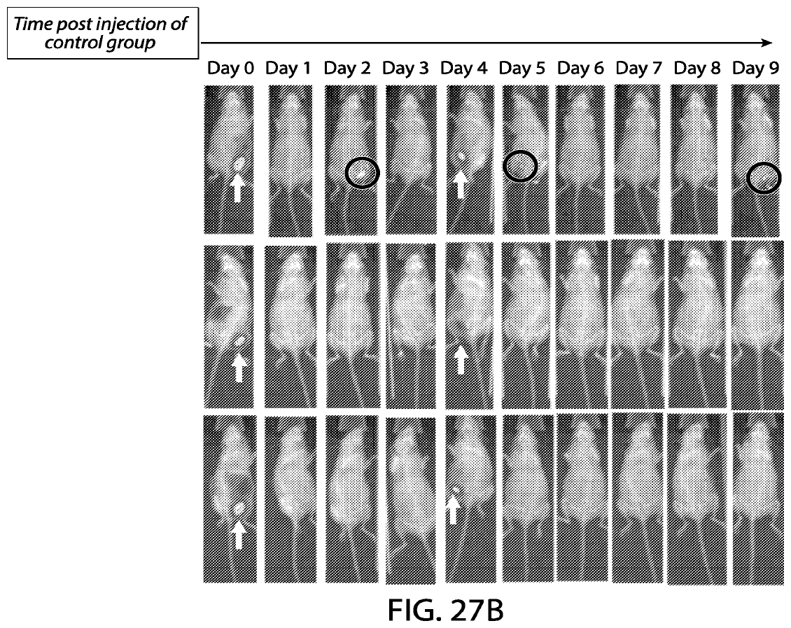

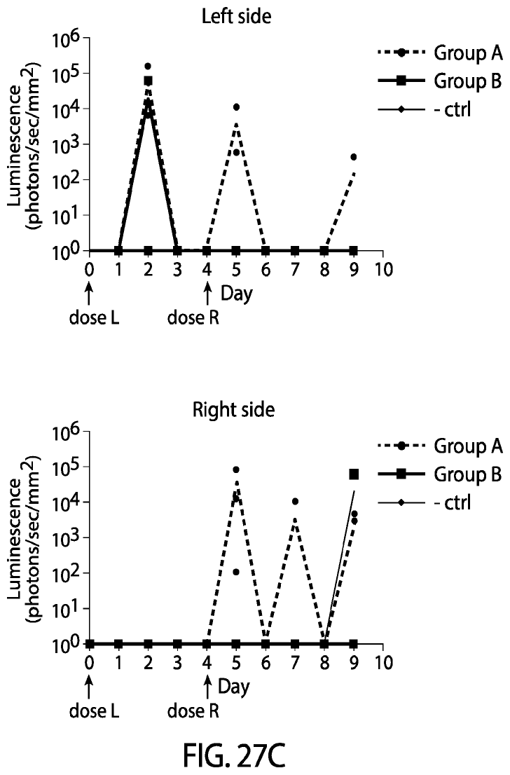

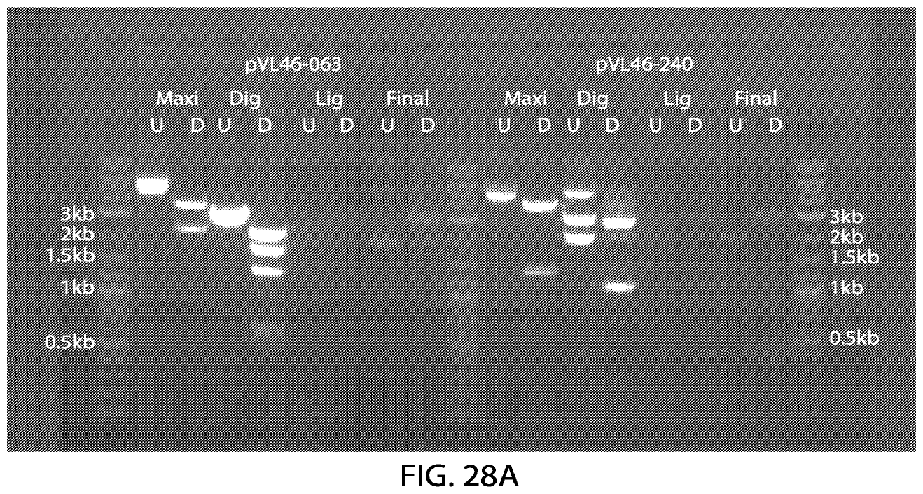

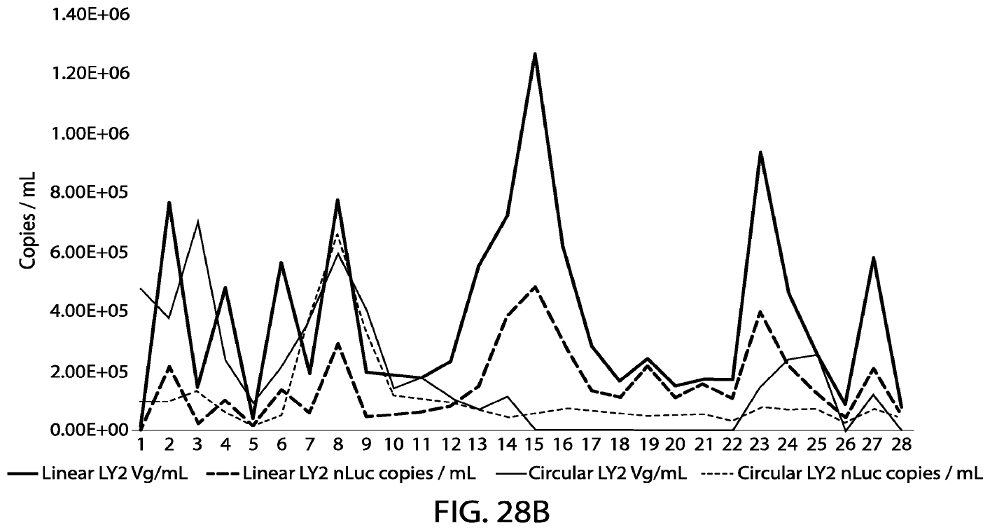

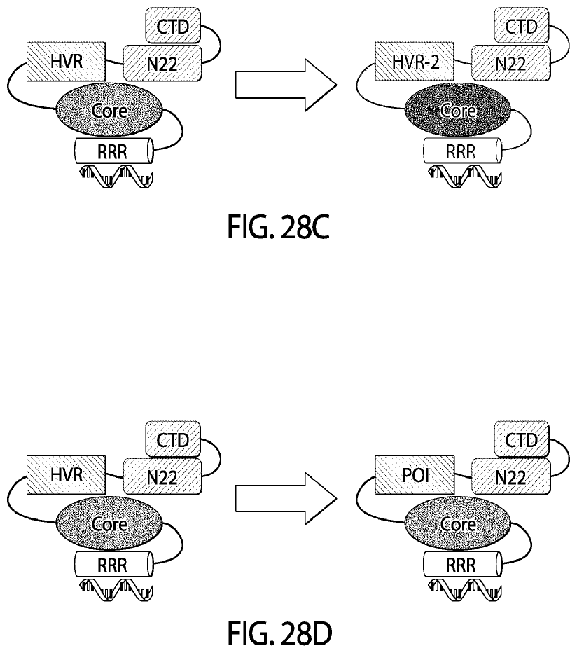

In another example, TTMV-LY2 plasmid (pVL46-240) and TTMV-LY2-nLuc were linearized with Esp3I or EcoRV-HF, respectively. Digested plasmid was purified on 1% agarose gels prior to electroelution or Qiagen column purification and ligation with T4 DNA Ligase. Circularized DNA was concentrated on a 100 kDa UF/DF membrane before transfection. Circularization was confirmed by gel electrophoresis, as shown in FIG. 28A. T-225 flasks were seeded with HEK293T at 3×10⁴ cells/cm² one day prior to lipofection with Lipofectamine 2000. Nine micrograms of circularized TTMV-LY2 DNA and 50 μg of circularized TTMV-LY2-nLuc were co-transfected one day post flask seeding. As a comparison, an additional T-225 flask was co-transfected with 50 μg of linearized TTMV-LY2 and 50 μg of linearized TTMV-LY2-nLuc.

Anellovector production proceeded for eight days prior to cell harvest in Triton X-100 harvest buffer. Generally, anellovectors can be enriched, e.g., by lysis of host cells, clarification of the lysate, filtration, and chromatography. In this example, harvested cells were nuclease treated prior to sodium chloride adjustment and 1.2 m/0.45 m normal flow filtration. Clarified harvest was concentrated and buffer exchanged into PBS on a 750 kDa MWCO mPES hollow fiber membrane. The TFF retentate was filtered with a 0.45 m filter before loading on a Sephacryl S-500 HR SEC column pre-equilibrated in PBS. Anellovectors were processed across the SEC column at 30 cm/hr. Individual fractions were collected and assayed by qPCR for viral genome copy number and transgene copy number, as shown in FIG. 28B. Viral genomes and transgene copies were observed beginning at the void volume, Fraction 7, of the SEC chromatogram. A residual plasmid peak was observed at Fraction 15. Copy number for TTMV-LY2 genomes and TTMV-LY2-nLuc transgene were in good agreement for Anellovectors produced using circularized input DNA at Fraction 7-Fraction 10, indicating packaged Anellovectors containing nLuc transgene. SEC fractions were pooled and concentrated using a 100 kDa MWCO PVDF membrane and then 0.2 m filtered prior to in vivo administration.

Circularization of input Anellovector DNA resulted a threefold increase in a percent recovery of nuclease protected genomes throughout the purification process when compared to linearized Anellovector DNA, indicating improved manufacturing efficiency using the circularized input Anellovector DNA as shown in Table 46.

In some embodiments, IVC anellovector genomes can be introduced into insect cells (e.g., Sf9 cells) and enclosed within proteinaceous exterior proteins expressed from a bacmid, e.g., as described herein.

TABLE 46
Purification Process Yields

| | Linearized TTMV-LY2 | | Circularized TTMV-LY2 | |
|---|---|---|---|---|
| | | Total nLuc | | Total nLuc |
| | Total viral | transgene | Total viral | transgene |
| | genome | genome | genome | genome |
| Step | copies | copies | copies | copies |
| Harvest pre- | 2.78E+12 | 2.17E+12 | 1.04E+11 | 4.39E+11 |
| nuclease | | | | |
| Clarified | 9.96E+09 | 5.48E+09 | 6.55E+08 | 9.81E+08 |
| Harvest | | | | |
| TFF | 1.01E+10 | 7.66E+09 | 2.58E+08 | 3.56E+08 |
| SEC | 3.18E+07 | 8.73E+06 | 9.16E+06 | 7.75E+06 |
| UF/DF | 8.82E+06 | 3.25E+06 | 1.78E+06 | 2.73E+06 |
| Sterile | 5.60E+06 | 2.64E+06 | 8.66E+05 | 1.63E+06 |
| Filtration | | | | |
| Purification | 0.0002% | 0.0001% | 0.0006% | 0.0004% |
| Process | | | | |
| Yield (%) | | | | |

### Example 22: Production of Anellovectors Containing Chimeric ORF1 with Hypervariable Domains from Different Torque Teno Virus Strains

This example describes domain swapping of hypervariable regions of ORF1 to produce chimeric anellovectors containing the ORF1 arginine-rich region, jelly-roll domain, N22, and C-terminal domain of one TTV strain, and the hypervariable domain from an ORF1 protein of a different TTV strain.

The full-length genome LY2 strain of Betatorquevirus has been cloned into expression vectors for expression in mammalian cells. This genome is mutated to remove the hypervariable domain of LY2 and replace it with the hypervariable domain of a distantly related Betatorqueviruses (FIG. 28C). The plasmid containing the LY2 genome with the swapped hypervariable domain (pTTMV-LY2-HVRa-z) is then linearized and circularized using previously published methods (Kincaid et al., PLoS Pathogens 2013). HEK293T cells are transfected with the circularized genome and incubated for 5-7 days to allow anellovector production. After the incubation period anellovectors are purified from the supernatant and cell pellet of transfected cells by gradient ultracentrifugation.

To determine if the chimeric anellovectors are still infectious, the isolated viral particles are added to uninfected cells. The cells are incubated for 5-7 days to allow viral replication. After incubation the ability of the chimeric anellovectors to establish infection will be monitored by immunofluorescence, western blot, and qPCR. The structural integrity of the chimeric viruses is assessed by negative stain and cryo-electron microscopy. Chimeric anellovectors can further be tested for ability to infect cells in vivo. Establishment of the ability to produce functional chimeric anellovectors through hypervariable domain swapping could allow for engineering of viruses to alter tropism and potentially evade immune detection.

In some embodiments, chimeric anellovectors and/or chimeric ORF1 can be introduced into insect cells (e.g., Sf9 cells) and enclosed within proteinaceous exterior proteins expressed from a bacmid, e.g., as described herein.

### Example 23: Production of Chimeric ORF1 Containing Non-TTV Protein/Peptides in Place of Hypervariable Domains

This example describes the replacement of the hypervariable regions of ORF1 with other proteins or peptides of interest to produce chimeric ORF1 protein containing the arginine-rich region, jelly-roll domain, N22, and C-terminal domain of one TTV strain, and a non-TTV protein/peptide in place of the hypervariable domain.

As shown in example B, the hypervariable domain of LY2 is deleted from the genome and a protein or peptide of interest may be inserted into this region (FIG. 28D). Examples of types of sequences that could be introduced into this region include but are not limited to, affinity tags, single chain variable regions (scFv) of antibodies, and antigenic peptides. Mutated genomes in the plasmid (pTTMV-LY2-ΔHVR-POI) are linearized and circularized as described in example B. Circularized genomes are transfected into HEK293T cells and incubated for 5-7 days. Following incubation, the chimeric anellovectors containing the POI are purified from the supernatant and cell pellet via ultracentrifugation and/or affinity chromatography where appropriate.

The ability to produce functional chimeric anellovectors containing POIs is assessed using a variety of techniques. First, purified virus is added to uninfected cells to determine if chimeric anellovectors can replicate and/or deliver payload to naïve cells. Additionally, structural integrity of chimeric anellovectors is assessed using electron microscopy. For chimeric anellovectors that are functional in vitro, the ability of replicate/delivery payload in vivo is also assessed.

In some embodiments, chimeric anellovectors and/or chimeric ORF1 can be introduced into insect cells (e.g., Sf9 cells) and enclosed within proteinaceous exterior proteins expressed from a bacmid, e.g., as described herein.

### Example 24: Anellovectors Based on Tth8 and LY2 Each Successfully Transduced the EPO Gene into Lung Cancer Cells

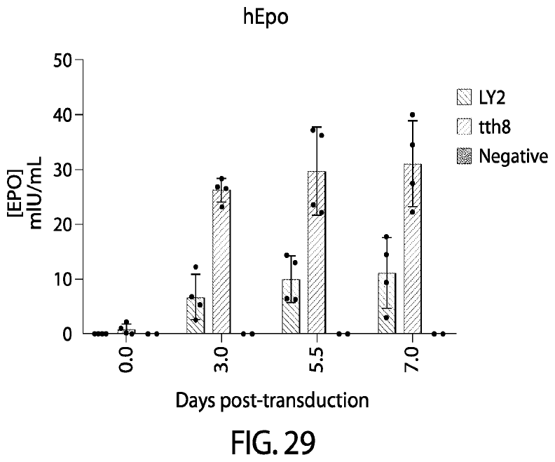

In this example, a non-small cell lung cancer line (EKVX) was transduced using two different anellovectors carrying the erythropoietin gene (EPO). The anellovectors were generated by in vitro circularization, as described herein, and included two types of anellovectors based on either an LY2 or tth8 backbone. Each of the LY2-EPO and tth8-EPO anellovectors included a genetic element that included the EPO-encoding cassette and non-coding regions of the LY2 or tth8 genome (5′ UTR, GC-rich region), respectively, but did not include Anellovirus ORFs, e.g., as described in Example 39. Cells were inoculated with purified anellovectors or a positive control (AAV2-EPO at high dose or at the same dose as the anellovectors) and incubated for 7 days. Anellovirus ORFs were provided in trans in a separate in vitro circularized DNA. Culture supernatant was sampled 3, 5.5, and 7 days post-inoculation and assayed using a commercial ELISA kit to detect EPO. Both LY2-EPO and tth8-EPO anellovectors successfully transduced cells, showing significantly higher EPO titers compared to untreated (negative) control cells (P<0.013 at all time points) (FIG. 29).

In some embodiments, the cells instead can be infected with anellovectors produced using bacmids and insect cells, e.g., as described herein.

### Example 25: Anellovectors with Therapeutic Transgenes can be Detected In Vivo after Intravenous (i.v.) Administration

In this example, anellovectors encoding human growth hormone (hGH) were detected in vivo after intravenous (i.v.) administration. Replication-deficient anellovectors, based on a LY2 backbone and encoding an exogenous hGH (LY2-hGH), were generated by in vitro circularization as described herein. The genetic element of the LY2-hGH anellovectors included LY2 non-coding regions (5′ UTR, GC-rich region) and the hGH-encoding cassette, but did not include Anellovirus ORFs, e.g., as described in Example 39. LY2-hGH anellovectors were administered to mice intravenously. The Anellovirus ORFs were provided in trans in a separate in vitro circularized DNA. Briefly, anellovectors (LY2-hGH) or PBS was injected intravenously at day 0 (n=4 mice/group). Anellovectors were administered to independent animal groups at 4.66E+07 anellovector genomes per mouse.

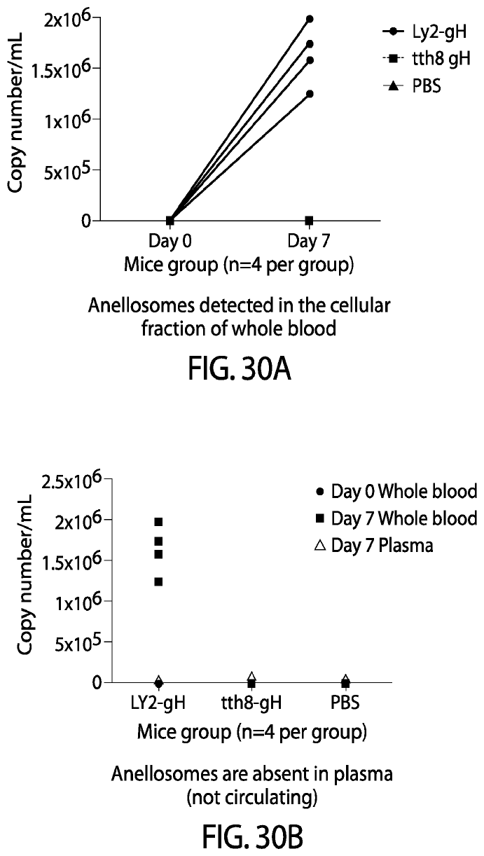

In a first example, anellovector viral genome DNA copies were detected. At day 7, blood and plasma were collected and analyzed for the hGH DNA amplicon by qPCR. LY2-hGH anellovectors were present in the cellular fraction of whole blood after 7 days post infection in vivo (FIG. 30A). Furthermore, the absence of anellovectors in plasma demonstrated the inability of these anellovectors to replicate in vivo (FIG. 30B).

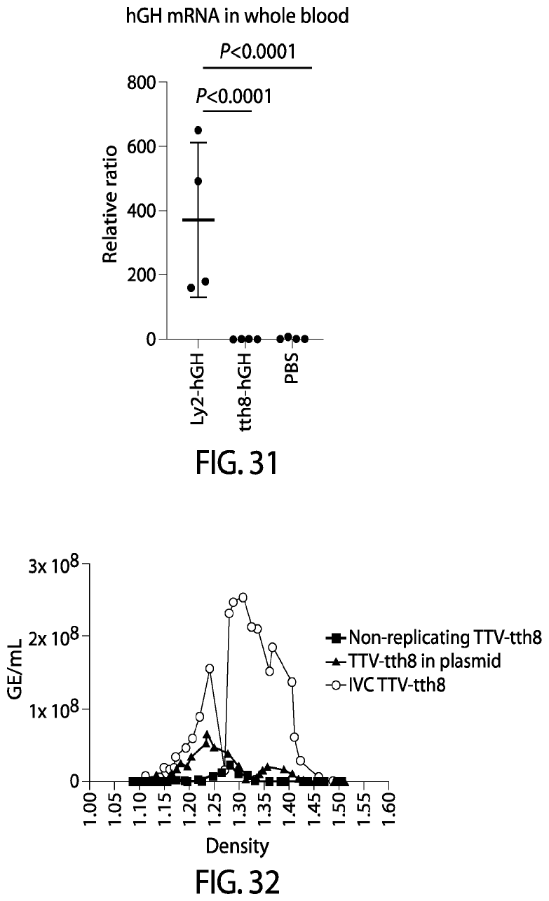

In a second example, hGH mRNA transcripts were detected after in vivo transduction. At day 7, blood was collected and analyzed for the hGH mRNA transcript amplicon by qRT-PCR. GAPDH was used as a control housekeeping gene. hGH mRNA transcripts in were measured in the cellular fraction of whole blood. mRNA from the anellovector-encoded transgene was detected in vivo (FIG. 31).

In some embodiments, the mice instead can be administered anellovectors produced using bacmids and insect cells, e.g., as described herein.

### Example 26: In Vitro Circularized Genome as Input Material for Producing Anellovectors In Vitro

This example demonstrates that in vitro circularized (IVC) double stranded anellovirus DNA, as source material for an anellovector genetic element as described herein, is more robust than an anellovirus genome DNA in a plasmid to yield packaged anellovector genomes of the expected density.

1.2E+07 HEK293T cells (human embryonic kidney cell line) in T75 flasks were transfected with 11.25 ug of either, (i) in vitro circularized double stranded TTV-tth8 genome (IVC TTV-tth8), (ii) TTV-tth8 genome in a plasmid backbone, or (iii) plasmid containing just the ORF1 sequence of TTV-tth8 (non-replicating TTV-tth8). Cells were harvested 7 days post transfection, lysed with 0.1% Triton, and treated with 100 units per ml of Benzonase. The lysates were used for cesium chloride density analysis; density was measured and TTV-tth8 copy quantification was performed for each fraction of the cesium chloride linear gradient. As shown in FIG. 32, IVC TTV-tth8 yielded dramatically more viral genome copies at the expected density of 1.33 as compared to TTV-tth8 plasmid.

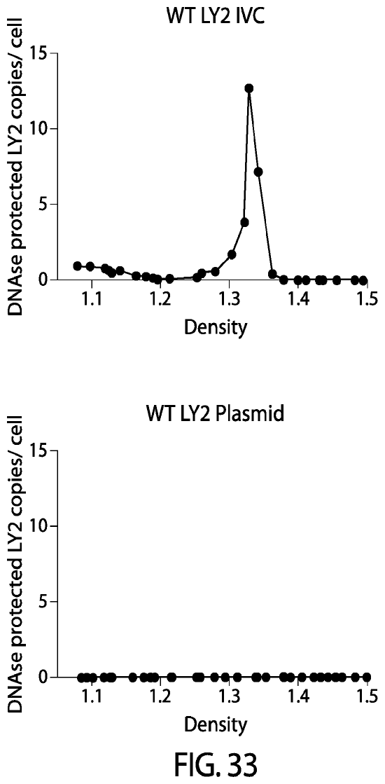

1E+07 Jurkat cells (human T lymphocyte cell line) were nucleofected with either in-vitro circularized LY2 genome (LY2 IVC) or LY2 genome in plasmid. Cells were harvested 4 days post transfection and lysed using a buffer containing 0.5% triton and 300 mM sodium chloride, followed by two rounds of instant freeze-thaw. The lysates were treated with 100 units/ml benzonase, followed by cesium chloride density analysis. Density measurement and LY2 genome quantification was performed on each fraction of the cesium chloride linear gradient. As shown in FIG. 33, transfection of in vitro circularized LY2 genome in Jurkat cells led to a sharp peak at the expected density, as compared to the transfection of plasmid containing the LY2 genome, which showed no detectable peak in FIG. 33.

In some embodiments, IVC anellovector genetic element constructs can be introduced into insect cells (e.g., Sf9 cells) and enclosed within proteinaceous exterior proteins expressed from a bacmid, e.g., as described herein.

What is claimed is:

1. A composition comprising
(i) a nucleic acid (e.g., DNA) construct comprising:
a) a promoter operably linked to a sequence encoding an Anellovirus ORF molecule (e.g., an ORF1, ORF2, ORF2/2, ORF2/3, ORF1/1, and/or ORF1/2 molecule); and
b) a backbone region suitable for replication of the nucleic acid construct in insect cells (e.g., a Baculovirus backbone region), optionally wherein the backbone region is also suitable for replication of the nucleic acid construct in bacterial cells; and
(ii) an Anellovirus genetic element comprising a promoter operably linked to a sequence encoding an exogenous effector.

2. A nucleic acid (e.g., DNA) construct comprising:
a) an Anellovirus genetic element region comprising a promoter operably linked to a sequence encoding an exogenous effector; and
b) a backbone region suitable for replication of the nucleic acid construct in insect cells (e.g., a Baculovirus backbone region), optionally wherein the backbone region is also suitable for replication of the nucleic acid construct in bacterial cells.

3. A bacterial cell comprising the nucleic acid construct or nucleic acid preparation of claim 1 or 2.

4. An insect cell comprising the nucleic acid construct or nucleic acid preparation of any of claims 1-3.

5. An insect cell comprising an Anellovirus ORF1 molecule comprising an Anellovirus ORF1 Arginine-rich region and an Anellovirus ORF1 C-terminal domain.

6. An insect cell comprising the nucleic acid construct of claim 2, further comprising a nucleic acid construct comprising:
a) a sequence encoding an Anellovirus ORF molecule (e.g., an ORF1, ORF2, ORF2/2, ORF2/3, ORF1/1, and/or ORF1/2 molecule); and
b) a backbone region suitable for replication of the nucleic acid construct in insect cells (e.g., a Baculovirus backbone region), optionally wherein the backbone region is also suitable for replication of the nucleic acid construct in bacterial cells.

7. A composition comprising an isolated Anellovirus ORF1 molecule comprising an Anellovirus ORF1 Arginine-rich region and an Anellovirus C-terminal domain, and not comprising detectable levels of an Anellovirus genetic element.

8. A composition comprising an isolated Anellovirus ORF1 molecule having a molecular weight of at least 101 kDa, and not comprising detectable levels of an Anellovirus genetic element.

9. A baculovirus particle comprising the nucleic acid construct of claim 2.

10. A population of baculovirus particles comprising the nucleic acid construct of claim 2.

11. A reaction mixture comprising the population of baculovirus particles of claim 10 and a plurality of insect cells.

12. A nucleic acid construct comprising, in order:
a) a first transposase recognition sequence (e.g., a Tn7R sequence);
b) an Anellovirus genetic element region comprising a promoter operably linked to a sequence encoding an exogenous effector;
c) a second transposase recognition sequence (e.g., a Tn7L sequence); and
d) optionally, a backbone region, e.g., wherein the backbone region is suitable for replication of the DNA construct.

13. A nucleic acid construct comprising, in order:
a) a first transposase recognition sequence (e.g., a Tn7R sequence);
b) a promoter operably linked to a sequence encoding an Anellovirus ORF molecule (e.g., an ORF1, ORF2, ORF2/2, ORF2/3, ORF1/1, and/or ORF1/2 molecule);
c) a second transposase recognition sequence (e.g., a Tn7L sequence); and
d) optionally, a backbone region, e.g., wherein the backbone region is suitable for replication of the DNA construct.

14. A method of making a baculovirus particle, the method comprising:
(i) providing an insect cell (e.g., an Sf9 cell) that comprises the nucleic acid construct of claim 2; and
(ii) incubating the insect cell under conditions suitable for production of a baculovirus particle comprising the nucleic acid construct or a copy thereof;
thereby making a baculovirus particle.

15. A method of making an insect cell comprising a bacmid construct comprising an Anellovirus genetic element, the method comprising:
(i) providing an insect cell (e.g., an Sf9 cell) that comprises a nucleic acid construct of claim 2 and an empty bacmid construct; and
(ii) incubating the insect cell under conditions suitable for recombination between the nucleic acid construct and the empty bacmid construct;
thereby making an insect cell comprising a bacmid construct comprising an Anellovirus genetic element.

16. A method of making an Anellovector, the method comprising:
(i) providing an insect cell comprising:
a) an Anellovirus genetic element comprising a promoter operably linked to a sequence encoding an exogenous effector, and
b) an Anellovirus ORF1 molecule;
(ii) incubating the insect cell under conditions suitable for enclosure of the Anellovirus genetic element in a proteinaceous exterior comprising the Anellovirus ORF1 molecule.
